(19) United States
(12) Patent Application Publication
(10) Pub. No.: US 2024/0252612 A1
(43) Pub. Date: Aug. 1, 2024

(54) IMMUNOGENIC COMPOSITIONS AND USES THEREOF

(71) Applicant: PFIZER INC., NEW YORK, NY (US)

(72) Inventors: Fanyu Meng, Whippany, NJ (US); Lynn Marie Phelan, Lake Hiawatha, NJ (US); Pirada Suphaphiphat Allen, Pearl River, NY (US); Nicholas Luksha, North Andover, MA (US); Bakul Subodh Bhatnagar, North Reading, MA (US); Ramin Darvari, Lexington, MA (US); Jayesh Vijay Sonje, Arlington, MA (US); Serguei Tchessalov, Billerica, MA (US); Advait Vijay Badkar, Westford, MA (US); Michael David Bruchsaler, St. Charles, MO (US); Wouter Joris Knapen, Puurs (BE); Parag Kolhe, Westford, MA (US); Marjoh Anne Nauta, Puurs (BE); Naomi Sasha Ramesar, Wilmington, MA (US); Vincent Jurgen L. Van Deyck, Puurs (BE); Bert Van Meervenne, Puurs (BE); Thijs Vanslembrouck, Puurs (BE); Sarah Elizabeth Weiser, Andover, MA (US)

(73) Assignee: PFIZER INC., NEW YORK, NY (US)

(21) Appl. No.: 18/533,167

(22) Filed: Dec. 7, 2023

Related U.S. Application Data
(60) Provisional application No. 63/431,743, filed on Dec. 11, 2022.
(60) Provisional application No. 63/480,099, filed on Jan. 16, 2023.
(60) Provisional application No. 63/484,746, filed on Feb. 13, 2023.
(60) Provisional application No. 63/496,395, filed on Apr. 15, 2023.
(60) Provisional application No. 63/507,755, filed on Jun. 13, 2023.
(60) Provisional application No. 63/587,036, filed on Sep. 29, 2023.
(60) Provisional application No. 63/602,567, filed on Nov. 24, 2023.

(51) Int. Cl. A61K 39/145; A61K 9/00; A61K 9/14; A61K 9/19; A61K 39/00; A61K 47/26

(52) U.S. Cl. CPC A61K 39/145; A61K 9/0019; A61K 9/146; A61K 9/19; A61K 47/26; A61K 2039/55555; A61K 2039/575

(57) ABSTRACT

The present disclosure relates to compositions comprising RNA molecules encoding an antigen derived from influenza, wherein the RNA may be formulated in a lipid nanoparticle (LNP), and wherein the composition further includes a polypeptide antigen derived from respiratory syncytial virus (RSV) and/or RNA molecules encoding an antigen derived from RSV, wherein the RSV RNA may be may be formulated in an LNP. The present disclosure further relates to the use of the RNA molecules, RNA-LNPs and compositions for the prevention of influenza and RSV infection-induced illness.

### CROSS-REFERENCE TO RELATED APPLICATIONS

This application claims priority to each of the following applications, the disclosure of each of which is hereby incorporated by reference in its entirety: U.S. Provisional Patent Application 63/431,743, filed on Dec. 11, 2022; U.S. Provisional Patent Application 63/480,099, filed on Jan. 16, 2023; U.S. Provisional Patent Application 63/484,746, filed on Feb. 13, 2023; U.S. Provisional Patent Application 63/496,395, filed on Apr. 15, 2023; U.S. Provisional Patent Application 63/507,755, filed Jun. 13, 2023; U.S. Provisional Patent Application 63/587,036, filed on Sep. 29, 2023; and U.S. Provisional Patent Application 63/602,567, filed on Nov. 24, 2023.

### INCORPORATION BY REFERENCE OF SEQUENCE LISTING

This application includes an electronically submitted sequence listing in .xml format. The .xml file contains a replacement sequence listing entitled “PC072915.xml” created on Mar. 21, 2024 and having a size of 82 KB. The sequence listing contained in this .xml file is part of the specification and is incorporated herein by reference in its entirety.

### FIELD

The present disclosure relates to compositions and methods for the preparation, manufacture and therapeutic use of ribonucleic acid vaccines comprising polynucleotide molecules encoding one or more influenza antigens, such as hemagglutinin (HA) and/or any one of the following antigens: neuraminidase (NA), nucleoprotein (NP), matrix protein 1 (M1), matrix protein 2 (M2), non-structural protein 1 (NS1) and non-structural protein 2 (NS2); wherein the compositions, methods, and uses further include antigens derived from respiratory syncytial virus (RSV) polypeptides and/or polynucleotide molecules encoding the RSV antigens.

### BACKGROUND

Influenza viruses are members of the orthomyxoviridae family, and are classified into three types (A, B, and C), based on antigenic differences between their nucleoprotein (NP) and matrix (M) protein. A challenge for therapy and prophylaxis against influenza and other infections using traditional vaccines is the limitation of vaccines in breadth, providing protection only against closely related subtypes. In addition, the length of time required to complete current standard influenza virus vaccine production processes inhibits the rapid development and production of an adapted vaccine in a pandemic situation. There is a need for improved immunogenic compositions against influenza.

Respiratory syncytial virus (RSV) is a respiratory virus that infects the lungs and breathing passages. RSV is the leading cause of serious viral lower respiratory tract illness in infants worldwide and an important cause of respiratory illness in the elderly. However, no vaccine has been approved for preventing RSV infection.

RSV is a member of the Paramyxoviridae family. Its genome consists of a single-stranded, negative-sense RNA molecule that encodes 11 proteins, including nine structural proteins (three glycoproteins and six internal proteins) and two non-structural proteins. The structural proteins include three transmembrane surface glycoproteins: the attachment protein G, fusion protein F, and the small hydrophobic small hydrophobic (SH) protein. There are two subtypes of RSV, A and B. They differ primarily in the G glycoprotein, while the sequence of the F glycoprotein is more conserved between the two subtypes. The mature F glycoprotein has three general domains: ectodomain (ED), transmembrane domain (TM), and a cytoplasmic tail (CT). CT contains a single palmitoylated cysteine residue. The F glycoprotein of human RSV is initially translated from the mRNA as a single 574-amino acid polypeptide precursor (referred to “F0” or “F0 precursor”), which contains a signal peptide sequence (amino acids 1-25) at the N-terminus. Upon translation the signal peptide is removed by a signal peptidase in the endoplasmic reticulum. The remaining portion of the F0 precursor (i.e., residues 26-574) may be further cleaved at two polybasic sites (a.a. 109/110 and 136/137) by cellular proteases (in particular furin), removing a 27-amino acid intervening sequence designated pep27 (amino acids 110-136) and generating two linked fragments designated F1 (C-terminal portion; amino acids 137-574) and F2 (N-terminal portion; amino acids 26-109). F1 contains a hydrophobic fusion peptide at its N-terminus and two heptad-repeat regions (HRA and HRB). HRA is near the fusion peptide, and HRB is near the TM domain. The F1 and F2 fragments are linked together through two disulfide bonds. Either the uncleaved F0 protein without the signal peptide sequence or a F1-F2 heterodimer can form a RSV F protomer. Three such protomers assemble to form the final RSV F protein complex, which is a homotrimer of the three protomers. The F proteins of subtypes A and B are about 90 percent identical in amino acid sequence. An example sequence of the F0 precursor polypeptide for the A subtype is provided in SEQ ID NO: 1 (A2 strain; GenBank GI: 138251; Swiss Prot P03420), and for the B subtype is provided in SEQ ID NO: 2 (18537 strain; GenBank GI: 138250; Swiss Prot P13843). SEQ ID NO: 1 and SEQ ID NO: 2 are both 574 amino acid sequences. The signal peptide sequence for SEQ ID NO: 1 and SEQ ID NO: 2 has also been reported as amino acids 1-25 (GenBank and UniProt). In both sequences the TM domain is from approximately amino acids 530 to 550 but has alternatively been reported as 525-548. The cytoplasmic tail begins at either amino acid 548 or 550 and ends at amino acid 574, with the palmitoylated cysteine residue located at amino acid 550. RSV F protein is a primary antigen explored for RSV vaccines. The RSV F protein trimer mediates fusion between the virion membrane and the host cellular membrane and also promotes the formation of syncytia. In the virion prior to fusion with the membrane of the host cell, the largest population of F molecules forms a lollipop-shaped structure, with the TM domain anchored in the viral envelope. This conformation is referred to as the pre-fusion conformation. Pre-fusion RSV F is recognized by monoclonal antibodies (mAbs) D25, AM22, and MPE8, without discrimination between oligomeric states. Pre-fusion F trimers are specifically recognized by mAb AM14. During RSV entry into cells, the F protein rearranges from the pre-fusion state (which may be referred to herein as “pre-F”), through an intermediate extended structure, to a post-fusion state (“post-F”). During this rearrangement, the C-terminal coiled-coil of the pre-fusion molecule dissociates into its three constituent strands, which then wrap around the globular head and join three additional helices to form the post-fusion six helix bundle. If a pre-fusion RSV F trimer is subjected to increasingly harsh chemical or physical conditions, such as elevated temperature, it undergoes structural changes. Initially, there is loss of trimeric structure (at least locally within the molecule), and then rearrangement to the post-fusion form, and then denaturation of the domains. To prevent viral entry, F-specific neutralizing antibodies presumably must bind the pre-fusion conformation of F on the virion, or potentially the extended intermediate, before the viral envelope fuses with a cellular membrane. Thus, the pre-fusion form of the F protein is considered the preferred conformation as the desired vaccine antigen. Mutants of the RSV F protein have been provided to increase pre-fusion stability (see for example PCT application No WO2017/109629) and are promising vaccine candidates. RSV vaccines that incorporate F protein antigen have been under development. Clinical studies have shown that some F protein subunit-based vaccine candidates are safe and immunogenic, though improvements in protective efficacy and durability of protection are desirable.

Accordingly, improved immunogenic compositions to protect against both RSV infection and influenza are needed.

### SUMMARY

The present disclosure describes compositions and methods thereof that satisfy these unmet needs, among other things. In one aspect, the disclosure provides a composition including one or more RNAs, each comprising a nucleotide sequence that encodes one or more antigenic polypeptides associated with influenza, wherein the composition disclosed herein further comprises one or more antigenic polypeptides associated with respiratory syncytial virus (RSV).

In some embodiments, a composition comprises one or more RNAs, each encoding an RSV polypeptide. In some embodiments, the composition comprises one or more RSV polypeptides.

In some embodiments, a composition comprises one or more RNAs, each encoding an RSV F protein, a variant thereof, or an immunogenic fragment of an RSV F protein or a variant thereof.

In some embodiments, a composition comprises one or more RSV F proteins, an immunogenic variant thereof, or an immunogenic fragment of an RSV F protein or a variant thereof. In some embodiments, a composition described herein comprises: (i) one or more RNAs, each encoding a polypeptide of an RSV subtype A virus (e.g., an F protein, a variant thereof, or an immunogenic fragment of an F protein or a variant thereof), and one or more RNAs, each encoding a polypeptide of an RSV subtype B virus (e.g., an F protein, a variant thereof, or an immunogenic fragment of an F protein or a variant thereof); or (ii) one or more polypeptides of an RSV subtype A virus (e.g., an F protein, a variant thereof, or an immunogenic fragment of an F protein or a variant thereof) and one or more polypeptides of an RSV subtype B virus (e.g., an F protein, a variant thereof, or an immunogenic fragment of an F protein or a variant thereof). In some embodiments, an RSV F protein, variant, or immunogenic fragment is stabilized in a prefusion confirmation. In some embodiments, a composition comprises or describes RSVpreF (also known as Abrysvo™) and Arexvy™.

In some embodiments, a composition comprises: one or more RNAs, each encoding a polypeptide of a first infectious agent; and one or more polypeptides of a second infectious agent. In some embodiments, the composition comprises one or more polypeptides of an influenza virus. In some embodiments, the composition comprises one or more polypeptides of one or more influenza viruses (e.g., one or more polypeptides of two or more influenza virus strains (e.g., one or more polypeptides of four or more influenza virus strains that are prevalent or which have been predicted to be prevalent in a relevant area)). In some embodiments, the composition comprises a commercially available influenza virus vaccine (e.g., a recombinant commercially available influenza virus vaccine or an inactivated influenza virus vaccine). In some embodiments, the commercially available influenza virus vaccine is Flublok or Fluzone. In some embodiments, the composition comprises one or more polypeptides derived from RSV. In some embodiments, the composition comprises one or more polypeptides associated with a first RSV subtype and one or more polypeptides associated with a second RSV subtype. In some embodiments, the composition comprises one or more RSV F proteins, variants thereof, or immunogenic fragments of RSV F proteins or variants thereof. In some embodiments, the composition comprises an RSV F protein comprising one or more mutations that stabilize a prefusion confirmation of the F protein. In some embodiments, a composition comprises Arexvy™ or ABRYSVO^{T}M.

In some embodiments, a composition comprises: one or more RNAs, each encoding one or more polypeptides derived from influenza virus, a variant thereof, or an immunogenic fragment thereof; and one or more RSV F prefusion-stabilized proteins. In some embodiments, the composition comprises an RSV vaccine comprising a prefusion-stabilized F protein (e.g., an RSV vaccine described herein (e.g., Arexvy™ or ABRYSVO™)).

In some embodiments, a combination comprises an influenza vaccine and an RSV vaccine, each of which is provided in a separate container (e.g., separate vials and/or syringes). In some embodiments, the combination comprises: (a) an influenza vaccine provided in a single container and an RSV vaccine provided in a separate container.

In some embodiments, the combination comprises an RSV vaccine that comprises a prefusion-stabilized F protein (e.g., an RSV vaccine described herein (e.g., RSVpreF or ABRYSVO™)).

In one aspect, the disclosure provides a composition including: (i) a first ribonucleic acid (RNA) polynucleotide including an open reading frame encoding a first antigen, said antigen including at least one influenza virus antigenic polypeptide or an immunogenic fragment thereof, wherein the first RNA polynucleotide is formulated in a lipid nanoparticle (LNP); and (ii) a first RSV F protein trimer in the prefusion conformation. In some embodiments, the RNA-LNP element and the RSV polypeptide element are present in the composition as a combination, e.g., as an admixture, such that more than one element is mixed together to form a combination. For purposes of the present disclosure, “admixture” refers to the mixture of two or more compounds at any time prior or subsequent to, or concomitant with, administration. In some embodiments, the composition further includes (iii) a second RNA polynucleotide including an open reading frame encoding a second antigen, said second antigen including at least one influenza virus antigenic polypeptide or an immunogenic fragment thereof. In some embodiments, the antigens include hemagglutinin (HA), or an immunogenic fragment or variant thereof. In some embodiments, the antigens each include an HA, or an immunogenic fragment thereof, that are from different subtypes of influenza virus. In some embodiments, the composition further includes a third RNA polynucleotide including an open reading frame encoding an antigen including at least one influenza virus antigenic polypeptide or an immunogenic fragment thereof. In some embodiments, the third antigen is from an influenza virus different from the strain of influenza virus of both the first and second antigens. In some embodiments, the first, second and third RNA polynucleotides are formulated in a lipid nanoparticle. In some embodiments, the composition further includes a fourth RNA polynucleotide including an open reading frame encoding a fourth antigen, said antigen including at least one influenza virus antigenic polypeptide or an immunogenic fragment thereof. In some embodiments, the fourth antigen is from influenza virus but is from a different strain of influenza virus to the first, second and third antigens. In some embodiments, the first, second, third, and fourth RNA polynucleotides are formulated in a lipid nanoparticle. In some embodiments, the RNA polynucleotides are present in about equal ratios. In some embodiments, any one of the RNA polynucleotides includes a modified nucleotide. In some embodiments, the modified nucleotide is selected from the group consisting of pseudouridine, 1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine. In some embodiments, each RNA polynucleotide includes a 5′ terminal cap, a 5′ UTR, a 3′UTR, and a 3′ polyadenylation tail. In some embodiments, the 5′ terminal cap includes:

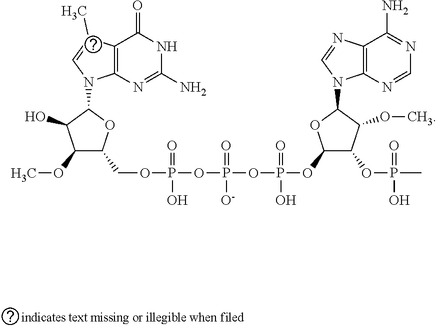

In some embodiments, the 5′ UTR includes SEQ ID NO: 1. In some embodiments, the 3′ UTR includes SEQ ID NO: 2. In some embodiments, the 3′ polyadenylation tail includes SEQ ID NO: 3. In some embodiments, the RNA polynucleotide has an integrity greater than 85%. In some embodiments, the RNA polynucleotide has a purity of greater than 85%. In some embodiments, the lipid nanoparticle includes 20-60 mol % ionizable cationic lipid, 5-25 mol % neutral lipid, 25-55 mol % cholesterol, and 0.5-5 mol % polymer-modified lipid. In some embodiments, the cationic lipid includes:

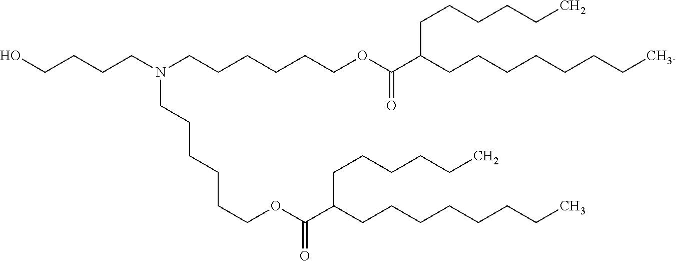 [SMILES: CCCCCCCCC(CCCCCC)C(=O)OCCCCCCN(CCCCO)CCCCCCOC(=O)C(CCCCCC)CCCCCCCC]

In some embodiments, the PEG-modified lipid includes:

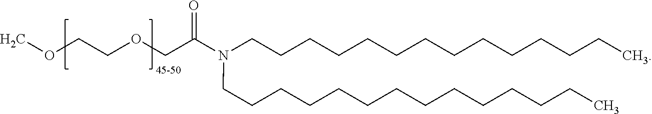

In some embodiments, the first antigen is HA from influenza A subtype H1 or an immunogenic fragment or variant thereof and the second antigen is HA from a different H1 strain to the first antigen or an immunogenic fragment or variant thereof. In some embodiments, the first and second antigens are HA from influenza A subtype H3 or an immunogenic fragment or variant thereof and wherein both antigens are derived from different strains of H3 influenza virus. In some embodiments, the first and second antigens are HA from influenza A subtype H1 or an immunogenic fragment or variant thereof and the third and fourth antigens are from influenza A subtype H3 or an immunogenic fragment or variant thereof and wherein the first and second antigens are derived from different strains of H1 virus and the third and fourth antigens are from different strains of H3 influenza virus. In some embodiments, at least the first and second RNA polynucleotides are formulated in a single lipid nanoparticle. In some embodiments, the first, second, and third RNA polynucleotides are formulated in a single lipid nanoparticle. In some embodiments, the first, second, third, and fourth RNA polynucleotides are formulated in a single LNP. In some embodiments, each of the RNA polynucleotides is formulated in a single LNP, wherein each single LNP encapsulates the RNA polynucleotide encoding one antigen. In some embodiments, the first RNA polynucleotide is formulated in a first LNP; and the second RNA polynucleotide is formulated in a second LNP. In some embodiments, the first RNA polynucleotide is formulated in a first LNP; the second RNA polynucleotide is formulated in a second LNP; and the third RNA polynucleotide is formulated in a third LNP. In some embodiments, the first RNA polynucleotide is formulated in a first LNP; the second RNA polynucleotide is formulated in a second LNP; the third RNA polynucleotide is formulated in a third LNP; and the fourth RNA polynucleotide is formulated in a fourth LNP. In some embodiments, any one of the compositions described herein are for use in the eliciting an immune response against influenza in a subject.

In some embodiments, the first RSV F protein is an F protein of subtype A. In some embodiments, the first RSV F protein includes a combination of mutations relative to the corresponding wild-type RSV F protein, wherein the combination of mutations is selected from the group consisting of: (1) combination of 103C, 148C, 1901, and 486S, preferably A103C, 1148C, S1901, and D486S; (2) combination of 54H, 55C, 188C, and 486S, preferably T54H, S55C, L188C, and D486S; (3) combination of 54H, 103C, 148C, 1901, 2961, and 486S, preferably T54H, A103C, 1148C, S1901, V2961, and D486S; (4) combination of 54H, 55C, 142C, 188C, 2961, and 371C, preferably T54H, S55C, L142C, L188C, V2961, and N371C; (5) combination of 55C, 188C, and 486S, preferably S55C, L188C, and D486S; (6) combination of 54H, 55C, 188C, and 1901, preferably T54H, S55C, L188C, and S1901; (7) combination of 55C, 188C, 1901, and 486S, preferably S55C, L188C, S1901, and D486S; (8) combination of 54H, 55C, 188C, 1901, and 486S, preferably T54H, S55C, L188C, S1901, and D486S; (9) combination of 155C, 1901, 290C, and 486S, preferably S155C, S1901, S290C, and D486S; (10) combination of 54H, 55C, 142C, 188C, 2961, 371C, 486S, 487Q, and 489S, preferably T54H, S55C, L142C, L188C, V2961, N371C, D486S, E487Q, and D489S; (11) combination of 54H, 155C, 1901, 290C, and 2961, preferably T54H, S155C, S1901, S290C, and V2961, and (12) combination of 155C, 190F, 290C, and 207L, preferably S155C, S190F, S290C, and V207L. In some embodiments, the first RSV F protein includes a combination of mutations relative to the corresponding wild-type RSV F protein, wherein the combination of mutations is selected from the group consisting of: (1) combination of 215P and 486N, preferably S215P and D486N, (2) combination of 66E, 215P, and 486N, preferably K66E, S215P, and D486N, (3) combination of 66E, 76V, 215P, and 486N, preferably K66E, 176V, S215P, and D486N, and, (4) combination of 66E, 671, 76V, 215P, and 486N, preferably K66E, N671, 176V, S215P, and D486N.

In some embodiments, the first RSV F protein includes a trimerization domain. In some embodiments, the composition further includes a second RSV F protein trimer in the prefusion conformation. In some embodiments, the second RSV F protein is an F protein of subtype B. In some embodiments, the second RSV F protein includes a combination of mutations relative to the corresponding wild-type RSV F protein, wherein the combination of mutations is selected from the group consisting of: (1) combination of 103C, 148C, 1901, and 486S, preferably A103C, 1148C, S1901, and D486S; (2) combination of 54H, 55C, 188C, 486S, preferably T54H, S55C, L188C, and D486S; (3) combination of 54H, 103C, 148C, 1901, 2961, and 486S, preferably T54H, A103C, 1148C, S1901, V2961, and D486S; (4) combination of 54H, 55C, 142C, 188C, 2961, and 371C, preferably T54H, S55C, L142C, L188C, V2961, and N371C; (5) combination of 55C, 188C, and 486S, preferably S55C, L188C, and D486S; (6) combination of 54H, 55C, 188C, and 1901, preferably T54H, S55C, L188C, and S1901; (7) combination of 55C, 188C, 1901, and 486S, preferably S55C, L188C, S1901, and D486S; (8) combination of 54H, 55C, 188C, 1901, and 486S, preferably T54H, S55C, L188C, S1901, and D486S; (9) combination of 155C, 1901, 290C, and 486S, preferably S155C, S1901, S290C, and D486S; (10) combination of 54H, 55C, 142C, 188C, 2961, 371C, 486S, 487Q, and 489S, preferably T54H, S55C, L142C, L188C, V2961, N371C, D486S, E487Q, and D489S; (11) combination of 54H, 155C, 1901, 290C, and 2961, preferably T54H, S155C, S1901, S290C, and V2961, and (12) combination of 155C, 190F, 290C, and 207L, preferably S155C, S190F, S290C, and V207L.

In some embodiments, the second RSV F protein includes a combination of mutations relative to the corresponding wild-type RSV F protein, wherein the combination of mutations is selected from the group consisting of: (1) combination of 215P and 486N, preferably S215P and D486N, (2) combination of 66E, 215P, and 486N, preferably K66E, S215P, and D486N, (3) combination of 66E, 76V, 215P, and 486N, preferably K66E, 176V, S215P, and D486N, and, (4) combination of 66E, 671, 76V, 215P, and 486N, preferably K66E, N671, 176V, S215P, and D486N. In some embodiments, the second RSV F protein includes a trimerization domain. In some embodiments, the first RSV F protein trimer is subtype A; wherein the composition further includes a second RSV F protein trimer in the prefusion conformation, said second RSV F protein trimer is subtype B. In some embodiments, the composition further includes sodium chloride at a concentration of between about 20 mM and about 250 mM; (iii) at least one of sucrose, mannitol and glycine at a concentration of between about 5 mg/mL and about 100 mg/mL; and (iv) a buffer; wherein the pH of said composition is between about 7 and about 8.

In some embodiments, the LNP is in a liquid state and the first RSV F protein trimer is lyophilized. In some embodiments, the LNP is in a liquid state and the first RSV F protein trimer is an aqueous solution. In some embodiments, the osmolality of the composition is at most 500 mOsm/kg. In some embodiments, the compositions preferably has an osmolality of between 200 mOsm/kg and 400 mOsm/kg, preferably between 240-360 mOsm/kg, and more preferably fall within the range of 290-310 mOsm/kg.

In some embodiments, each RNA has at least 50% integrity, as measured by fragment analyzer. In some embodiments, each LNP has at least 80% encapsulation efficiency for at least 4 hours. In some embodiments, the composition further includes sodium chloride at a concentration of between about 20 mM and about 250 mM; (iii) at least one of sucrose, mannitol and glycine at a concentration of between about 5 mg/mL and about 100 mg/mL; and (iv) a buffer; wherein the pH of said composition is between about 7 and about 8. In some embodiments, the RNA polynucleotide is purified and is substantially free of contaminants including short abortive RNA species, long abortive RNA species, double-stranded RNA (dsRNA), residual plasmid DNA, residual in vitro transcription enzymes, residual solvent and/or residual salt. In some embodiments, the RSV polypeptide is purified and is substantially free of contaminants. In some embodiments, the composition is preferably sterile. The composition is preferably non-pyrogenic, e.g., containing <1 EU (endotoxin unit, a standard measure) per dose, and preferably <0.1 EU per dose. The composition is preferably gluten free. Human vaccines are typically administered in a dosage volume of about 0.5 ml, although a half dose (i.e., about 0.25 ml) may be administered to children. Preferably, the compositions disclosed herein does not further include any one of the following oil-in-water emulsion, a cytokine-inducing agent, or benzonaphthyridine compounds, QS-21, CpG sequences, and 3dMPL (also known as 3 de-O-acylated monophosphoryl lipid A or 3-O-desacyl-4′-monophosphoryl lipid A). In some preferred embodiments, the composition does not further include QS-21 or saponin-containing adjuvants. In other preferred embodiments, the composition does not further include aluminum-containing compounds, e.g., aluminum hydroxide and AlPO₄.

In one aspect, disclosed herein is a composition including (i) a first ribonucleic acid (RNA) polynucleotide including an open reading frame encoding a first antigen, said antigen including at least one influenza virus antigenic polypeptide or an immunogenic fragment thereof, wherein the first RNA polynucleotide is formulated in a lipid nanoparticle (LNP); (ii) a second RNA polynucleotide including an open reading frame encoding a second antigen, said second antigen including at least one influenza virus antigenic polypeptide or an immunogenic fragment thereof; (iii) a third RNA polynucleotide including an open reading frame encoding an antigen including at least one influenza virus antigenic polypeptide or an immunogenic fragment thereof; (iv) a fourth RNA polynucleotide including an open reading frame encoding a fourth antigen, said antigen including at least one influenza virus antigenic polypeptide or an immunogenic fragment thereof; and (v) an RNA polynucleotide including at least one open reading frame encoding at least one respiratory syncytial virus (RSV) antigenic polypeptide or an immunogenic fragment thereof. In some embodiments, the RSV antigenic polypeptide has at least 90%, 95%, 96%, 97%, 98% or 99% identity to the amino acid sequence selected from SEQ ID NO: 1 to 6. In some embodiments, the RNA polynucleotide includes at least one open reading frame encoding at least one respiratory syncytial virus (RSV) antigenic polypeptide or an immunogenic fragment thereof includes the sequence set forth in any one of SEQ ID NO: 13, SEQ ID NO: 14, SEQ ID NO: 15, and SEQ ID NO: 16. In some embodiments, the RSV antigenic polypeptide is derived from RSV subtype A and/or RSV subtype B. In some embodiments, each of said RNA polynucleotide includes a 5′ cap, 5′ UTR, 3′ UTR, and poly-A tail. In some embodiments, any one of said RNA polynucleotide includes at least one modified nucleotide selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine OR 2′-O-methyl uridine.

In some embodiments, each of said RNA polynucleotide is encapsulated in a lipid nanoparticle (LNP). In some embodiments, the LNP includes a cationic lipid, a polymer-lipid, a neutral lipid, and a steroid or steroid analog.

In some aspects, the disclosed herein is a method of eliciting an immune response against influenza in a subject, including administering an effective amount of any one composition described herein. In some aspects, the disclosed herein is a method of eliciting an immune response against influenza and RSV in a subject, including administering an effective amount of any one composition described herein. In some aspects, the disclosed herein is a method of preventing, treating or ameliorating an infection, disease or condition associated with influenza and/or RSV in a subject, including administering to a subject an effective amount of any one composition described herein. In some embodiments, the subject is less than about 1 year of age, about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older. In some embodiments, any one composition described herein is administered by intradermal or intramuscular injection.

### BRIEF DESCRIPTION OF THE DRAWINGS

FIG. 1A to 1E show immunogenicity of modRNA-LNP formulations of RSV 847 in mice. Female BALB/c mice (10/group) were immunized intramuscularly at day 0 and 21 with RSV 847 constructs either as bivalent protein subunit (RSV 847A+B) or modRNA-LNP formulation either as monovalent (RSV 847A) or bivalent (RSV 847A+B) at indicated dose. On day 35 (2 weeks post dose 2, PD2), serum was collected for RSV neutralizing assay and spleen for T-cell assays (ELISpot and Intracellular Cytokine Staining, ICS assays). FIG. 1A and FIG. 1B show neutralization assay results for RSV A and B expressed as 50% neutralizing titers (each symbol represents a titer from an individual animal. Bars represent geometric mean titer (GMT)). FIG. 1C shows ELISpot assay results that measure the number of RSV A+B F-specific cells secreting IFN-γ and expressed as spot forming cells (SFC) per million cells. FIG. 1D and FIG. 1E show ICS assay results that measured RSV A+B F-specific IFN-γ-expressing cells within CD4+ and CD8+ T cells expressed as percentage of IFN-γ+ cells. Bars and errors bars depict median with interquartile range. NA: not analyzed.

FIG. 2—depicts Integrity of the flu mRNA component of the RSV subunit-Flu mRNA combination by Fragment Analyzer (FA); Target: ≥50% Intact RNA

FIG. 3—illustrates that the percentage of encapsulation efficiency is maintained for the flu mRNA component of the RSV subunit-Flu mRNA combination. Target: RNA Concentration: T0±20%; EE %: ≥80% FIG. 4—illustrates that the RSV Relative Prefusion F Content by ELISA is maintained. Target: 50-150%.

FIG. 5—influenza HA A/Wisconsin strain in vitro expression data from a combination composition comprising of RSV-subunit and Flu quadrivalent HA modRNA-encapsulated LNP; IVE Assay using 293F Suspension Cells; DAI Study T0 T2hT4h;

FIG. 6—influenza HA A/Darwin strain in vitro expression data from a combination composition comprising RSV-subunit and Flu quadrivalent HA modRNA-encapsulated LNP; IVE Assay using 293F Suspension Cells; DAI Study T0 T2hT4h.

FIG. 7—Virus Neutralization Titers Against RSV Subtypes A and B at 3 Weeks Post-Dose 1 (Day 21)

FIG. 8—Virus Neutralization Titers Against Four Influenza Strains at 3 Weeks Post-Dose 1 (Day 21)

FIG. 9—Virus Neutralization Titers Against RSV Subtypes A and B at 2 Weeks Post-Dose 2 (Day 42)

FIG. 10—Neutralization Titers Against Four Influenza Strains at 2 Weeks Post-Dose 2 (Day 42)

FIG. 11A-B—After post-dose 1 (PD1), 3 weeks post-dose 1 data in mice: No or Minimal Interference in Flu Immunogenicity and Trend Towards Enhanced RSV Immunogenicity for modRNA Flu/RSV Subunit Combo Vaccine Candidate Compared to Standalone Vaccines in Mice; (FIG. 11A) Bivalent RSV vaccine responses; (FIG. 11B) Quadrivalent Flu vaccine responses.

FIG. 12A-B—After post-dose 2 (PD2), 2 weeks post-dose 2 data in mice: No Interference in Flu Immunogenicity and Similar or Higher RSV Immunogenicity for modRNA Flu/RSV Subunit Combo Vaccine Candidate Compared to Standalone Vaccines in Mice; (FIG. 12A) Bivalent RSV vaccine responses; (FIG. 12B) Quadrivalent Flu vaccine responses.

### DETAILED DESCRIPTION

Surprisingly, the inventors discovered that a Flu modRNA-LNP composition may be formulated in combination with (e.g., being in a mix and/or being in association with) an aqueous RSV subunit composition, and that the resulting combination was compatible and maintained stability of the respective antigens for at least 4 hours. The flu antigen was expressed from an mRNA-encapsulated LNP despite being formulated in the presence of the RSV subunit component. As a result of the discovery, there may be an option for medical professionals to administer different ratios of RSV and flu antigens in a single dose, while maintaining stability of the antigens as compared to the stability of the respective antigens when administered as a standalone formulation, in the absence of the second antigen. In some embodiments, the RSV subunit composition is lyophilized and subsequently reconstituted with a buffer solution prior to being mixed and/or in association with a Flu modRNA-LNP composition.

Embodiments of the present disclosure provide RNA (e.g., mRNA) vaccines that include polynucleotide encoding an influenza virus antigen. Influenza virus RNA vaccines, as provided herein may be used to induce a balanced immune response, comprising both cellular and humoral immunity, without many of the risks associated with DNA vaccination.

In some embodiments, the virus is a strain of Influenza A or Influenza B or combinations thereof.

In one aspect, the disclosure relates to an immunogenic composition including: (i) a first ribonucleic acid (RNA) polynucleotide having an open reading frame encoding a first antigen, said antigen including at least one influenza virus antigenic polypeptide or an immunogenic fragment thereof, and (ii) a second RNA polynucleotide having an open reading frame encoding a second antigen, said second antigen including at least one influenza virus antigenic polypeptide or an immunogenic fragment thereof, wherein the first and second RNA polynucleotides are formulated in a lipid nanoparticle (LNP). In some embodiments, the first and second antigens include hemagglutinin (HA), or an immunogenic fragment or variant thereof. In some embodiments, the first antigen includes an HA from a different subtype of influenza virus to the influenza virus antigenic polypeptide or an immunogenic fragment thereof of the second antigen. In some embodiments, the composition further includes (iii) a third antigen including at least one influenza virus antigenic polypeptide or an immunogenic fragment thereof, wherein the third antigen is from influenza virus but is from a different strain of influenza virus to both the first and second antigens. In some embodiments, the first, second and third RNA polynucleotides are formulated in a lipid nanoparticle.

In some embodiments, the composition further includes (iv) a fourth RNA polynucleotide having an open reading frame encoding a fourth antigen, said antigen including at least one influenza virus antigenic polypeptide or an immunogenic fragment thereof, wherein the fourth antigen is from influenza virus but is from a different strain of influenza virus to the first, second and third antigens. In some embodiments, the first, second, third, and fourth RNA polynucleotides are formulated in a lipid nanoparticle.

In some embodiments, the RNA polynucleotides are mixed in desired ratios in a single vessel and are subsequently formulated into lipid nanoparticles. The inventors surprisingly discovered that the initial input of different RNA polynucleotides at a known ratio to be formulated in a single LNP process surprisingly resulted in LNPs encapsulating the different RNA polynucleotides in about the same ratio as the input ratio. The results were surprising in view of the potential for the manufacturing process to favor one RNA polynucleotide to another when encapsulating the RNA polynucleotides into an LNP. Such embodiments may be referred herein as “pre-mix”. Accordingly, in some embodiments, first and second RNA polynucleotides are formulated in a single lipid nanoparticle. In some embodiments, the first, second, third, and fourth RNA polynucleotides are formulated in a single LNP. In some embodiments, the first, second, third, fourth, and fifth RNA polynucleotides are formulated in a single LNP. In some embodiments, the first, second, third, fourth, fifth, and sixth RNA polynucleotides are formulated in a single LNP. In some embodiments, the first, second, third, fourth, fifth, sixth, and seventh RNA polynucleotides are formulated in a single LNP. In some embodiments, the first, second, third, fourth, fifth, sixth, seventh, and eighth RNA polynucleotides are formulated in a single LNP.

In some embodiments, the molar ratio of the first RNA polynucleotide to the second RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the second RNA polynucleotide is greater than 1:1.

In some embodiments, the molar ratio of the first RNA polynucleotide to the third RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the third RNA polynucleotide is greater than 1:1.

In some embodiments, the molar ratio of the first RNA polynucleotide to the fourth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the fourth RNA polynucleotide is greater than 1:1. In some embodiments, the molar ratio of the first RNA polynucleotide to the fifth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the fifth RNA polynucleotide is greater than 1:1. In some embodiments, the molar ratio of the first RNA polynucleotide to the sixth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the sixth RNA polynucleotide is greater than 1:1. In some embodiments, the molar ratio of the first RNA polynucleotide to the seventh RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the seventh RNA polynucleotide is greater than 1:1. In some embodiments, the molar ratio of the first RNA polynucleotide to the eighth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the eighth RNA polynucleotide is greater than 1:1.

In alternative embodiments, each RNA polynucleotide encoding a particular antigen is formulated in an individual LNP, such that each LNP encapsulates an RNA polynucleotide encoding identical antigens. Such embodiments may be referred herein as “post-mix”.

Accordingly, in some embodiments, the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; the fifth RNA polynucleotide is formulated in a fifth LNP; the sixth RNA polynucleotide is formulated in a sixth LNP; the seventh RNA polynucleotide is formulated in a seventh LNP; and the eighth RNA polynucleotide is formulated in an eighth LNP.

In some embodiments, the molar ratio of the first LNP to the second LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the second LNP is greater than 1:1.

In some embodiments, the molar ratio of the first LNP to the third LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the third LNP is greater than 1:1.

In some embodiments, the molar ratio of the first LNP to the fourth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the fourth LNP is greater than 1:1. In some embodiments, the molar ratio of the first LNP to the fifth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the fifth LNP is greater than 1:1. In some embodiments, the molar ratio of the first LNP to the sixth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the sixth LNP is greater than 1:1. In some embodiments, the molar ratio of the first LNP to the seventh LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the seventh LNP is greater than 1:1. In some embodiments, the molar ratio of the first LNP to the eighth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the eighth LNP is greater than 1:1.

In some embodiments, the antigenic polypeptide encodes a hemagglutinin protein or immunogenic fragment thereof. In some embodiments, the hemagglutinin protein is H1, H2, H3, H4, H5, H6, H7, H8, H9, H10, H11, H12, H13, H14, H15, H16, H17, H18, or an immunogenic fragment thereof. In some embodiments, the hemagglutinin protein does not comprise a head domain. In some embodiments, the hemagglutinin protein comprises a portion of the head domain. In some embodiments, the hemagglutinin protein does not comprise a cytoplasmic domain. In some embodiments, the hemagglutinin protein comprises a portion of the cytoplasmic domain. In some embodiments, the truncated hemagglutinin protein comprises a portion of the transmembrane domain.

Some embodiments provide influenza vaccines comprising one or more RNA polynucleotides having an open reading frame encoding a hemagglutinin protein and a pharmaceutically acceptable carrier or excipient, formulated within a cationic lipid nanoparticle. In some embodiments, the hemagglutinin protein is selected from H1, H7 and H10. In some embodiments, the RNA polynucleotide further encodes neuraminidase (NA) protein. In some embodiments, the hemagglutinin protein is derived from a strain of Influenza A virus or Influenza B virus or combinations thereof. In some embodiments, the Influenza virus is selected from H1N1, H3N2, H7N9, and H10N8.

In some embodiments, the virus is a strain of Influenza A or Influenza B or combinations thereof. In some embodiments, the strain of Influenza A or Influenza B is associated with birds, pigs, horses, dogs, humans, or non-human primates. In some embodiments, the antigenic polypeptide encodes a hemagglutinin protein or fragment thereof. In some embodiments, the hemagglutinin protein is H7 or H10 or a fragment thereof. In some embodiments, the hemagglutinin protein comprises a portion of the head domain (HA1). In some embodiments, the hemagglutinin protein comprises a portion of the cytoplasmic domain. In some embodiments, the truncated hemagglutinin protein. In some embodiments, the protein is a truncated hemagglutinin protein comprises a portion of the transmembrane domain. In some embodiments, the virus is selected from the group consisting of H7N9 and H10N8. Protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length.

In some embodiments, an Influenza RNA composition includes an RNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the influenza antigen. Antigenic fusion proteins, in some embodiments, retain the functional property from each original protein.

Some embodiments provide methods of preventing or treating influenza viral infection comprising administering to a subject any of the vaccines described herein. In some embodiments, the antigen specific immune response comprises a T cell response. In some embodiments, the antigen specific immune response comprises a B cell response. In some embodiments, the antigen specific immune response comprises both a T cell response and a B cell response. In some embodiments, the method of producing an antigen specific immune response involves a single administration of the vaccine. In some embodiments, the vaccine is administered to the subject by intradermal, intramuscular injection, subcutaneous injection, intranasal inoculation, or oral administration.

In some embodiments, the RNA (e.g., mRNA) polynucleotides or portions thereof may encode one or more polypeptides or fragments thereof of an influenza strain as an antigen.

The present disclosure further provides for an RNA molecule (e.g., RNA polynucleotide) comprising at least one open reading frame (ORF) encoding a respiratory syncytial virus (RSV) antigen. In some aspects, the RSV antigen is a RSV polypeptide. In some aspects, the RSV polypeptide is a RSV F polypeptide. In some aspects, the RSV polypeptide comprises an amino acid sequence of Table 30. In some aspects, the RNA molecules comprise an ORF transcribed from at least one DNA nucleic acid sequence of Table 31. In some aspects, the RNA molecules comprise an ORF comprising an RNA nucleic acid sequence of Table 32. In some aspects the RNA molecule comprises at least one of a 5′ cap, 5′ UTR, 3′ UTR and poly-A tail. In other aspects the RNA molecule comprises at least one of a 5′ cap, 3′ UTR and poly-A tail. The present disclosure provides for an RNA molecule comprising modified nucleotides. The present disclosure provides for an immunogenic composition comprising any one of the RNA molecules encoding a RSV polypeptide described herein complexed with, encapsulated in, or formulated with one or more lipids, and forming lipid nanoparticles (RNA-LNPs). The present disclosure further provides for an immunogenic composition comprising any one of the RNA molecules comprising at least one RNA nucleic acid described herein complexed with, encapsulated in, or formulated with one or more lipids, and forming RNA-LNPs. The present disclosure further provides for a method of preventing, an infection, disease or condition (e.g., RSVinfection-related Respiratory tract illness, including pneumonia and bronchitis) in a subject via administering to a subject an effective amount of an RNA molecule, RNA-LNP or an immunogenic composition described herein. The present disclosure further provides for the use of the RNA molecule, RNA-LNP and/or an immunogenic composition described herein as a vaccine.

The present disclosure may be understood more readily by reference to the following detailed description of the embodiments of the disclosure and the Examples included herein. It is to be understood that this invention is not limited to specific methods of making that may of course vary. It is to be also understood that the terminology used herein is for the purpose of describing specific embodiments only and is not intended to be limiting.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

### I. Examples of Definitions

Unless otherwise defined herein, scientific and technical terms used in connection with the present disclosure have the meanings that are commonly understood by those of ordinary skill in the art.

Throughout this application, the term “about” is used according to its plain and ordinary meaning in the area of cell and molecular biology to indicate a deviation of ±10% of the value(s) to which it is attached.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the term “comprising” may mean “one,” but it is also consistent with the meaning of “one or more,” “at least one,” and “one or more than one.”

The phrase “and/or” means “and” or “or.” To illustrate, A, B, and/or C includes: A alone, B alone, C alone, a combination of A and B, a combination of A and C, a combination of B and C, or a combination of A, B, and C. In other words, “and/or” operates as an inclusive or.

The phrase “essentially all” is defined as “at least 95%”; if essentially all members of a group have a certain property, then at least 95% of members of the group have that property. In some aspects, essentially all means equal to any one of, at least any one of, or between any two of 95, 96, 97, 98, 99, or 100% of members of the group have that property.

The compositions and methods for their use may “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that aspects described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “inhibiting,” “decreasing,” or “reducing” or any variation of these terms includes any measurable decrease (e.g., a 5%, 10%, 15%, 20%, 25%, 30%, 35%, 40%, 45%, 50%, 55%, 60%, 65%, 70%, 75%, 80%, 85%, 90%, 95%, or 99% decrease) or complete inhibition to achieve a desired result. The terms “improve,” “promote,” or “increase” or any variation of these terms includes any measurable increase (e.g., a 5%, 10%, 15%, 20%, 25%, 30%, 35%, 40%, 45%, 50%, 55%, 60%, 65%, 70%, 75%, 80%, 85%, 90%, 95%, or 99% increase) to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “isolated” may refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that may be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state and/or that is altered or removed from the natural state through human intervention. For example, a DNA naturally present in a living animal is not “isolated,” but a synthetic DNA, or a DNA partially or completely separated from the coexisting materials of its natural state is “isolated.” An isolated nucleic acid may exist in substantially purified form, or may exist in a non-native environment such as, for example, a cell into which the nucleic acid has been delivered.

A “nucleic acid,” as used herein, is a molecule comprising nucleic acid components and refers to DNA or RNA molecules. It may be used interchangeably with the term “polynucleotide.”A nucleic acid molecule is a polymer comprising or consisting of nucleotide monomers, which are covalently linked to each other by phosphodiester-bonds of a sugar/phosphate-backbone. Nucleic acids may also encompass modified nucleic acid molecules, such as base-modified, sugar-modified or backbone-modified etc. DNA or RNA molecules. Nucleic acids may exist in a variety of forms such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, saRNA, modRNA and complementary sequences of the foregoing described herein. Nucleic acids may encode an epitope to which antibodies may bind.

The term “epitope” refers to a moiety that is specifically recognized by an immunoglobulin (e.g., antibody or receptor) binding component. In some aspects, an epitope is comprised of a plurality of chemical atoms or groups on an antigen. In some aspects, such chemical atoms or groups are surface-exposed when the antigen adopts a relevant three-dimensional conformation. In some aspects, such chemical atoms or groups are physically near to each other in space when the antigen adopts such a conformation. In some aspects, at least some such chemical atoms are groups are physically separated from one another when the antigen adopts an alternative conformation (e.g., is linearized).

Nucleic acids may be single-stranded or double-stranded and may comprise RNA and/or DNA nucleotides and artificial variants thereof (e.g., peptide nucleic acids). In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide.

The term “polynucleotide” refers to a nucleic acid molecule that may be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA, or synthetic), analogs thereof, or a combination thereof. Additional coding or non-coding sequences may, but need not, be present within a polynucleotide.

In certain aspects, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some aspects, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids may be any length. They may be, for example, equal to any one of, at least any one of, at most any one of, or between any two of5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or may comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

As used herein, the term “expression” of a nucleic acid sequence refers to the generation of any gene product from the nucleic acid sequence. In some aspects, a gene product may be a transcript. In some aspects, a gene product may be a polypeptide. In some aspects, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

The term “DNA,” as used herein, means a nucleic acid molecule comprising nucleotides such as deoxy-adenosine-monophosphate, deoxy-thymidine-monophosphate, deoxy-guanosine-monophosphate and deoxy-cytidine-monophosphate monomers which are composed of a sugar moiety (deoxyribose), a base moiety and a phosphate moiety, and polymerize by a characteristic backbone structure. The backbone structure is, typically, formed by phosphodiester bonds between the sugar moiety of the nucleotide, e.g., deoxyribose, of a first and a phosphate moiety of a second, adjacent monomer. The specific order of the monomers, e.g., the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing. DNA may contain all, or a majority of, deoxyribonucleotide residues. As used herein, the term “deoxyribonucleotide” means a nucleotide lacking a hydroxyl group at the 2′ position of a β-D-ribofuranosyl group. Without any limitation, DNA may encompass double stranded DNA, antisense DNA, single stranded DNA, isolated DNA, synthetic DNA, DNA that is recombinantly produced, and modified DNA.

The term “RNA,” as used herein, means a nucleic acid molecule comprising nucleotides such as adenosine-monophosphate, uridine-monophosphate, guanosine-monophosphate and cytidine-monophosphate monomers which are connected to each other along a so-called backbone. The backbone is formed by phosphodiester bonds between the sugar, e.g., ribose, of a first and a phosphate moiety of a second, adjacent monomer. RNA may be obtainable by transcription of a DNA-sequence, e.g., inside a cell. In eukaryotic cells, transcription is typically performed inside the nucleus or the mitochondria. In vivo, transcription of DNA may result in premature RNA which is processed into messenger-RNA (mRNA). Processing of the premature RNA, e.g. in eukaryotic organisms, comprises various posttranscriptional modifications such as splicing, 5′ capping, polyadenylation, export from the nucleus or the mitochondria. Mature messenger RNA is processed and provides the nucleotide sequence that may be translated into an amino acid sequence of a peptide or protein. A mature mRNA may comprise a 5′ cap, a 5′ UTR, an open reading frame, a 3′ UTR and a poly-A tail sequence. RNA may contain all, or a majority of, ribonucleotide residues. As used herein, the term “ribonucleotide” means a nucleotide with a hydroxyl group at the 2′ position of a β-D-ribofuranosyl group. In one aspect, RNA may be messenger RNA (mRNA) that relates to a RNA transcript which encodes a peptide or protein. As known to those of skill in the art, mRNA generally contains a 5′ untranslated region (5′ UTR), a polypeptide coding region, and a 3′ untranslated region (3′ UTR). Without any limitation, RNA may encompass double stranded RNA, antisense RNA, single stranded RNA, isolated RNA, synthetic RNA, RNA that is recombinantly produced, and modified RNA (modRNA).

An “isolated RNA” is defined as an RNA molecule that may be recombinant or has been isolated from total genomic nucleic acid. An isolated RNA molecule or protein may exist in substantially purified form, or may exist in a non-native environment such as, for example, a host cell.

A “modified RNA” or “modRNA” refers to an RNA molecule having at least one addition, deletion, substitution, and/or alteration of one or more nucleotides as compared to naturally occurring RNA. Such alterations may refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide may replace one or more uridine and/or cytidine nucleotides. For example, these replacements may occur for every instance of uridine and/or cytidine in the RNA sequence, or may occur for only select uridine and/or cytidine nucleotides. Such alterations to the standard nucleotides in RNA may include non-standard nucleotides, such as chemically synthesized nucleotides or deoxynucleotides. For example, at least one uridine nucleotide may be replaced with N1-methylpseudouridine in an RNA sequence. Other such altered nucleotides are known to those of skill in the art. Such altered RNA molecules are considered analogs of naturally-occurring RNA. In some aspects, the RNA is produced by in vitro transcription using a DNA template, where DNA refers to a nucleic acid that contains deoxyribonucleotides. In some aspects, the RNA may be replicon RNA (replicon), in particular self-replicating RNA, or self-amplifying RNA (saRNA).

As contemplated herein, without any limitations, RNA may be used as a therapeutic modality to treat and/or prevent a number of conditions in mammals, including humans. Methods described herein comprise administration of the RNA described herein to a mammal, such as a human. For example, in one aspect such methods of use for RNA include an antigen-coding RNA vaccine to induce robust neutralizing antibodies and accompanying/concomitant T-cell response to achieve protective immunization. In some aspects, minimal vaccine doses are administered to induce robust neutralizing antibodies and accompanying/concomitant T-cell response to achieve protective immunization. In one aspect, the RNA administered is in vitro transcribed RNA. For example, such RNA may be used to encode at least one antigen intended to generate an immune response in said mammal. Pathogenic antigens are peptide or protein antigens derived from a pathogen associated with infectious disease.

In specific aspects, the pathogenic are peptide or protein antigens derived from RSV.

Conditions and/or diseases that may be treated with RNA disclosed herein include, but are not limited to, those caused and/or impacted by viral infection. Such viruses include, but are not limited to, RSV.

“Prevent” or “prevention,” as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder, or condition has been delayed for a predefined period of time.

As will be understood from context, “risk” of a disease, disorder, and/or condition refers to a likelihood that a particular individual will develop the disease, disorder, and/or condition. In some aspects, risk is expressed as a percentage. In some aspects, risk is, is at least, or is at most from 0, 1, 2, 3, 4, 5, 6, 7, 8, 9, 10, 20, 30, 40, 50, 60, 70, 80, 90 up to 100%. In some aspects risk is expressed as a risk relative to a risk associated with a reference sample or group of reference samples. In some aspects, a reference sample or group of reference samples have a known risk of a disease, disorder, condition and/or event. In some aspects a reference sample or group of reference samples are from individuals comparable to a particular individual. In some aspects, risk may reflect one or more genetic attributes, e.g., which may predispose an individual toward development (or not) of a particular disease, disorder and/or condition. In some aspects, risk may reflect one or more epigenetic events or attributes and/or one or more lifestyle or environmental events or attributes. Susceptible to: An individual who is “susceptible to” a disease, disorder, and/or condition is one who has a higher risk of developing the disease, disorder, and/or condition than does a member of the general public. In some aspects, an individual who is susceptible to a disease, disorder and/or condition may not have been diagnosed with the disease, disorder, and/or condition. In some aspects, an individual who is susceptible to a disease, disorder, and/or condition may exhibit symptoms of the disease, disorder, and/or condition. In some aspects, an individual who is susceptible to a disease, disorder, and/or condition may not exhibit symptoms of the disease, disorder, and/or condition. In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition.

The terms “protein,” “polypeptide,” or “peptide” are used herein as synonyms and refer to a polymer of amino acid monomers, e.g., a molecule comprising at least two amino acid residues. Polypeptides may include gene products, naturally occurring polypeptides, synthetic polypeptides, homologs, orthologs, paralogs, fragments and other equivalents, variants, and analogs of the foregoing. Polypeptides may be a single molecule or may be a multi-molecular complex such as a dimer, trimer or tetramer. A protein comprises one or more peptides or polypeptides, and may be folded into a 3-dimensional form, which may be required for the protein to exert its biological function.

As used herein, the term “wild type” or “WT” or “native” refers to the endogenous version of a molecule that occurs naturally in an organism. In some aspects, wild type versions of a protein or polypeptide are employed, however, in other aspects of the disclosure, a modified protein or polypeptide is employed to generate an immune response. The terms described above may be used interchangeably.

A “modified protein” or “modified polypeptide” or a “variant” refers to a protein or polypeptide whose chemical structure, particularly its amino acid sequence, is altered with respect to the wild type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild type activity or function in other respects, such as immunogenicity. Where a protein is specifically mentioned herein, it is in general a reference to a native (wild type) or recombinant (modified) protein. The protein may be isolated directly from the organism of which it is native, produced by recombinant DNA/exogenous expression methods, produced by solid-phase peptide synthesis (SPPS), or other in vitro methods. In particular aspects, there are isolated nucleic acid segments and recombinant vectors incorporating nucleic acid sequences that encode a polypeptide (e.g., an antigen or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The term “fragment,” with reference to an amino acid sequence (peptide or protein), relates to a part of an amino acid sequence, e.g., a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90%, or at least 99% of the amino acid residues from an amino acid sequence. In the present disclosure, a fragment of a polypeptide, DNA nucleic acid or RNA nucleic acid sequence refers to a sequence having sequence identity of at least, at most, exactly, or between any two of 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99% with a polypeptide, DNA nucleic acid or RNA nucleic acid sequence, from which it is derived.

In one aspect, a fragment of a polypeptide, DNA nucleic acid or RNA nucleic acid sequence refers to a sequence having sequence identity of at least 70% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 80% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 85% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 90% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 95% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 97% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 99% with a polypeptide, DNA nucleic acid or RNA nucleic acid sequence, from which it is derived.

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some aspects, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some aspects, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some aspects, a variant polypeptide or nucleic acid shows an overall sequence identity with a reference polypeptide or nucleic acid that is at least, at most, exactly, or between any two of 85%, 86%, 87%, 88%, 89%, 90%, 91%, 92%, 93%, 94%, 95%, 96%, 97%, or 99%. In some aspects, a variant polypeptide or nucleic acid does not share at least one characteristic sequence element with a reference polypeptide or nucleic acid. In some aspects, a reference polypeptide or nucleic acid has one or more biological activities. In some aspects, a variant polypeptide or nucleic acid shares one or more of the biological activities of the reference polypeptide or nucleic acid. In some aspects, a variant polypeptide or nucleic acid lacks one or more of the biological activities of the reference polypeptide or nucleic acid. In some aspects, a variant polypeptide or nucleic acid shows a reduced level of one or more biological activities as compared to the reference polypeptide or nucleic acid. In some aspects, a polypeptide or nucleic acid of interest is considered to be a “variant” of a reference polypeptide or nucleic acid if it has an amino acid or nucleotide sequence that is identical to that of the reference but for a small number of sequence alterations at particular positions. Preferably, the variant polypeptide or nucleic acid sequence has at least one modification compared to the reference polypeptide or nucleic acid sequence, e.g., from 1 to about 20 modifications. In one aspect, the variant polypeptide or nucleic acid sequence has from 1 to about 10 modifications compared to the reference polypeptide or nucleic acid sequence. In one aspect, the variant polypeptide or nucleic acid sequence has from 1 to about 5 modifications compared to the reference polypeptide or nucleic acid sequence. In one aspect, the variant polypeptide or nucleic acid sequence has from 1 to about 4 modifications compared to the reference polypeptide or nucleic acid sequence. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. Often, a variant polypeptide or nucleic acid comprises a very small number (e.g., fewer than about 5, about 4, about 3, about 2, or about 1) number of substituted, inserted, or deleted, functional residues (e.g., residues that participate in a particular biological activity) relative to the reference. In some aspects, a variant polypeptide or nucleic acid comprises about 10, about 9, about 8, about 7, about 6, about 5, about 4, about 3, about 2, or about 1 substituted residues as compared to a reference. In some aspects, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some aspects, a variant polypeptide or nucleic acid comprises not more than about 5, about 4, about 3, about 2, or about 1 addition or deletion, and, in some aspects, comprises no additions or deletions, as compared to the reference.

In some aspects, a reference polypeptide or nucleic acid is a “wild type” or “WT” or “native” sequence found in nature, including allelic variations. A wild type polypeptide or nucleic acid sequence has a sequence that has not been intentionally modified. For the purposes of the present disclosure, “variants” of an amino acid sequence (peptide, protein, or polypeptide) comprise amino acid insertion variants, amino acid addition variants, amino acid deletion variants and/or amino acid substitution variants. “Variants” of a nucleotide sequence comprise nucleotide insertion variants, nucleotide addition variants, nucleotide deletion variants and/or nucleotide substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid or nucleic acid sequence.

Changes may be introduced by mutation into a nucleic acid, thereby leading to changes in the amino acid sequence of a polypeptide (e.g., an antigen or antibody or antibody derivative) that it encodes. Mutations may be introduced using any technique known in the art. In one aspect, one or more particular amino acid residues are changed using, for example, a site-directed mutagenesis protocol. In another aspect, one or more randomly selected residues are changed using, for example, a random mutagenesis protocol. In some aspects, however it is made, a mutant polypeptide may be expressed and screened for a desired property.

Mutations may be introduced into a nucleic acid without significantly altering the biological activity of a polypeptide that it encodes. For example, one may make nucleotide substitutions leading to amino acid substitutions at non-essential amino acid residues. Alternatively, one or more mutations may be introduced into a nucleic acid that selectively changes the biological activity of a polypeptide that it encodes. For example, the mutation may quantitatively or qualitatively change the biological activity. Examples of quantitative changes include increasing, reducing or eliminating the activity. Examples of qualitative changes include altering the antigen specificity of an antibody.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group). In some aspects, percent identity of two sequences is determined using the BLASTN or BLASTP algorithm, as available on the United States National Center for Biotechnology Information (NCBI) website.

Percentage identity is obtained by determining the number of identical positions at which the sequences to be compared correspond, dividing this number by the number of positions compared (e.g., the number of positions in the reference sequence) and multiplying this result by 100.

In some aspects, the degree of similarity or identity is given for a region that is at least, at most, exactly, or between any two of about 50%, about 60%, about 70%, about 80%, about 90%, or about 100% of the entire length of the reference sequence. For example, if the reference nucleic acid sequence consists of 200 nucleotides, the degree of identity is given for at least, at most, exactly, or between any two of about 100, about 120, about 140, about 160, about 180, or about 200 nucleotides, in some aspects, continuous nucleotides. In some aspects, the degree of similarity or identity is given for the entire length of the reference sequence.

Homologous amino acid sequences may exhibit at least, at most, exactly, or between any two of 40%, 50%, 60%, 70%, 80%, 90%, 95%, 98%, or 99% identity of the amino acid residues. In one aspect, homologous amino acid sequences exhibit at least 95% identity of the amino acid residues. In one aspect, homologous amino acid sequences exhibit at least 98% identity of the amino acid residues. In one aspect, homologous amino acid sequences exhibit at least 99% identity of the amino acid residues.

A fragment or variant of an amino acid sequence (peptide or protein) may be a “functional fragment” or “functional variant.” The term “functional fragment” or “functional variant” of an amino acid sequence relates to any fragment or variant exhibiting one or more functional properties identical or similar to those of the amino acid sequence from which it is derived, e.g., it is functionally equivalent. With respect to antigens or antigenic sequences, one particular function is one or more immunogenic activities displayed by the amino acid sequence from which the fragment or variant is derived. The term “functional fragment” or “functional variant,” as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one aspect, the modifications in the amino acid sequence of the parent molecule or sequence do not significantly affect or alter the characteristics of the molecule or sequence.

The term “mutant” of a wild-type RSV F protein, “mutant” of a RSV F protein, “RSV F protein mutant,” or “modified RSV F protein” refers to a polypeptide that displays introduced mutations relative to a wild-type F protein and is immunogenic against the wild-type F protein.

An amino acid sequence (peptide, protein, or polypeptide) “derived from” a designated amino acid sequence (peptide, protein, or polypeptide) refers to the origin of the first amino acid sequence. Preferably, the amino acid sequence which is derived from a particular amino acid sequence has an amino acid sequence that is identical, essentially identical, or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In the present disclosure, a vector refers to a nucleic acid molecule, such as an artificial nucleic acid molecule. A vector may be used to incorporate a nucleic acid sequence, such as a nucleic acid sequence comprising an open reading frame. Vectors include, but are not limited to, storage vectors, expression vectors, cloning vectors, transfer vectors. A vector may be an RNA vector or a DNA vector. In some aspects the vector is a DNA molecule. In some aspects, the vector is a plasmid vector. In some aspects, the vector is a viral vector. Typically, an expression vector will contain a desired coding sequence and appropriate other sequences necessary for the expression of the operably linked coding sequence in a particular host organism (e.g., bacteria, yeast, plant, insect, or mammal) or in in vitro expression systems. Cloning vectors are generally used to engineer and amplify a certain desired fragment (typically a DNA fragment), and may lack functional sequences needed for expression of the desired fragment(s).

As used herein, the term “pharmaceutical composition” refers to an active agent, formulated together with one or more pharmaceutically acceptable carriers. Pharmaceutical compositions may be immunogenic compositions. In some aspects, active agent is present in unit dose amount appropriate for administration in a therapeutic regimen that shows a statistically significant probability of achieving a predetermined therapeutic effect when administered to a relevant population. In some aspects, pharmaceutical compositions may be specially formulated for parenteral administration, for example, by subcutaneous, intramuscular, intravenous or epidural injection as, for example, a sterile solution or suspension, or sustained-release formulation.

As used herein, the term “vaccination” refers to the administration of an immunogenic composition intended to generate an immune response, for example to a disease-associated (e.g., disease-causing) agent (e.g., a virus). In some aspects, vaccination may be administered before, during, and/or after exposure to a disease-associated agent, and in certain aspects, before, during, and/or shortly after exposure to the agent. In some aspects, vaccination includes multiple administrations, appropriately spaced in time, of a vaccine composition. In some aspects, vaccination generates an immune response to an infectious agent. In some aspects, vaccination generates an immune response to a tumor; in some such aspects, vaccination is “personalized” in that it is partly or wholly directed to epitope(s) (e.g., which may be or include one or more neoepitopes) determined to be present in a particular individual's tumors.

An immune response refers to a humoral response, a cellular response, or both a humoral and cellular response in an organism. An immune response may be measured by assays that include, but are not limited to, assays measuring the presence or amount of antibodies that specifically recognize a protein or cell surface protein, assays measuring T-cell activation or proliferation, and/or assays that measure modulation in terms of activity or expression of one or more cytokines.

As used herein, the term “combination therapy” refers to those situations in which a subject is simultaneously exposed to two or more therapeutic regimens (e.g., two or more therapeutic agents). In some aspects, the two or more regimens may be administered simultaneously; in some aspects, such regimens may be administered sequentially (e.g., all “doses” of a first regimen are administered prior to administration of any doses of a second regimen); in some aspects, such agents are administered in overlapping dosing regimens. In some aspects, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some aspects, two or more agents, or active moieties thereof, may be administered together in a combination composition, or even in a combination compound (e.g., as part of a single chemical complex or covalent entity).

Those skilled in the art will appreciate that the term “dosing regimen” may be used to refer to a set of unit doses (typically more than one) that are administered individually to a subject, typically separated by periods of time. In some aspects, a given therapeutic agent has a recommended dosing regimen, which may involve one or more doses. In some aspects, a dosing regimen comprises a plurality of doses each of which is separated in time from other doses. In some aspects, individual doses are separated from one another by a time period of the same length; in some aspects, a dosing regimen comprises a plurality of doses and at least two different time periods separating individual doses. In some aspects, all doses within a dosing regimen are of the same unit dose amount. In some aspects, different doses within a dosing regimen are of different amounts. In some aspects, a dosing regimen comprises a first dose in a first dose amount, followed by one or more additional doses in a second dose amount different from the first dose amount. In some aspects, a dosing regimen comprises a first dose in a first dose amount, followed by one or more additional doses in a second dose amount same as the first dose amount. In some aspects, a dosing regimen is correlated with a desired or beneficial outcome when administered across a relevant population (e.g., is a therapeutic dosing regimen).

### II. Influenza Compositions

There may be situations in which persons are at risk for infection with more than one strain of influenza virus. RNA (e.g., mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one strain of influenza, a combination vaccine can be administered that includes RNA (e.g., mRNA) encoding at least one antigenic polypeptide protein (or antigenic portion thereof) of a first influenza virus or organism and further includes RNA encoding at least one antigenic polypeptide protein (or antigenic portion thereof) of a second influenza virus or organism. RNA (e.g., mRNA) can be co-formulated, for example, in a single lipid nanoparticle (LNP) or can be formulated in separate LNPs for co-administration.

Some embodiments of the present disclosure provide influenza virus (influenza) vaccines (or compositions or immunogenic compositions) that include at least one RNA polynucleotide having an open reading frame encoding at least one influenza antigenic polypeptide or an immunogenic fragment thereof (e.g., an immunogenic fragment capable of inducing an immune response to influenza).

In some embodiments, the at least one antigenic polypeptide is one of the defined antigenic subdomains of HA, termed HA1, HA2, or a combination of HA1 and HA2, and at least one antigenic polypeptide selected from neuraminidase (NA), nucleoprotein (NP), matrix protein 1 (M1), matrix protein 2 (M2), non-structural protein 1 (NS1) and non-structural protein 2 (NS2).

In some embodiments, the at least one antigenic polypeptide is HA or derivatives thereof comprising antigenic sequences from HA1 and/or HA2, and at least one antigenic polypeptide selected from NA, NP, M1, M2, NS1 and NS2.

In some embodiments, the at least one antigenic polypeptide is HA or derivatives thereof comprising antigenic sequences from HA1 and/or HA2 and at least two antigenic polypeptides selected from NA, NP, M1, M2, NS1 and NS2.

In some embodiments, a vaccine comprises at least one RNA (e.g., mRNA) polynucleotide having an open reading frame encoding an influenza virus protein, or an immunogenic fragment thereof.

In some embodiments, a vaccine comprises at least one RNA (e.g., mRNA) polynucleotide having an open reading frame encoding multiple influenza virus proteins, or immunogenic fragments thereof.

In some embodiments, a vaccine comprises at least one RNA (e.g., mRNA) polynucleotide having an open reading frame encoding a HA protein, or an immunogenic fragment thereof (e.g., at least one HA1, HA2, or a combination of both).

In some embodiments, a vaccine comprises at least one RNA (e.g., mRNA) polynucleotide having an open reading frame encoding a HA protein, or an immunogenic fragment thereof (e.g., at least one HA1, HA2, or a combination of both, of any one of or a combination of any or all of H1, H2, H3, H4, H5, H6, H7, H8, H9, H10, H11, H12, H13, H14, H15, H16, H17, and/or H18) and at least one other RNA (e.g., mRNA) polynucleotide having an open reading frame encoding a protein selected from a NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one any one of or a combination of any or all of H1, H2, H3, H4, H5, H6, H7, H8, H9, H10, H11, H12, H13, H14, H15, H16, H17, and/or H18) and at least two other RNAs (e.g., mRNAs) polynucleotides having two open reading frames encoding two proteins selected from a NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least three other RNAs (e.g., mRNAs) polynucleotides having three open reading frames encoding three proteins selected from a NP protein, a NA protein, a M protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least four other RNAs (e.g., mRNAs) polynucleotides having four open reading frames encoding four proteins selected from a NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least five other RNAs (e.g., mRNAs) polynucleotides having five open reading frames encoding five proteins selected from a NP protein, a NA protein, a M1 protein, a M2 protein, a NS1 protein and a NS2 protein obtained from influenza virus.

In some embodiments, a vaccine comprises at least one RNA (e.g., mRNA) polynucleotide having an open reading frame encoding a HA protein or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18), a NP protein or an immunogenic fragment thereof, a NA protein or an immunogenic fragment thereof, a M1 protein or an immunogenic fragment thereof, a M2 protein or an immunogenic fragment thereof, a NS1 protein or an immunogenic fragment thereof and a NS2 protein or an immunogenic fragment thereof obtained from influenza virus.

Some embodiments of the present disclosure provide the following novel influenza virus polypeptide sequences: H1HA10-Foldon_ANgly1; H1HA10TM-PR8 (H1 A/Puerto Rico/8/34 HA); H1HA10-PR8-DS (H1 A/Puerto Rico/8/34 HA; pH1HA10-CaI04-DS (H1 A/California/04/2009 HA); Pandemic H1HA10 from California 04; pH1HA10-ferritin; HA10; Pandemic H1HA10 from California 04; Pandemic H1HA10 from California 04 strain/without foldon and with K68C/R76C mutation for trimerization; H1HA10 from A/Puerto Rico/8/34 strain, without foldon and with Y94D/N95L mutation for trimerization; H1HA10 from A/Puerto Rico/8/34 strain, without foldon and with K68C/R76C mutation for trimerization; H1N1 A/Viet Nam/850/2009; H3N2 A/Wisconsin/67/2005; H7N9 (A/Anhui/1/2013); H9N2 A/Hong Kong/1073/99; H10N8 A/JX346/2013.

Some embodiments of the present disclosure provide influenza virus (influenza) vaccines that include at least one RNA polynucleotide having an open reading frame encoding at least one influenza antigenic polypeptide or an immunogenic fragment of the novel influenza virus polypeptide sequences described above (e.g., an immunogenic fragment capable of inducing an immune response to influenza). In some embodiments, an influenza vaccine comprises at least one RNA (e.g., mRNA) polynucleotide having an open reading frame encoding at least one influenza antigenic polypeptide comprising a modified sequence that is at least 75% (e.g., any number between 75% and 100%, inclusive, e.g., 70%, 80%, 85%, 90%, 95%, 99%, and 100%) identity to an amino acid sequence of the novel influenza virus sequences described above. The modified sequence can be at least 75% (e.g., any number between 75% and 100%, inclusive, e.g., 70%, 80%, 85%, 90%, 95%, 99%, and 100%) identical to an amino acid sequence of the novel influenza virus sequences described above.

Some embodiments of the present disclosure provide an isolated nucleic acid comprising a sequence encoding the novel influenza virus polypeptide sequences described above; an expression vector comprising the nucleic acid; and a host cell comprising the nucleic acid. The present disclosure also provides a method of producing a polypeptide of any of the novel influenza virus sequences described above. A method may include culturing the host cell in a medium under conditions permitting nucleic acid expression of the novel influenza virus sequences described above, and purifying from the cultured cell or the medium of the cell a novel influenza virus polypeptide. The present disclosure also provides antibody molecules, including full length antibodies and antibody derivatives, directed against the novel influenza virus sequences.

In some embodiments, an open reading frame of a RNA (e.g., mRNA) vaccine is codon-optimized. In some embodiments, the open reading frame which the influenza polypeptide or fragment thereof is encoded is codon-optimized. Some embodiments provide use of an influenza vaccine that includes at least one ribonucleic acid (RNA) polynucleotide having an open reading frame encoding at least one influenza antigenic polypeptide or an immunogenic fragment thereof, wherein at least 80% (e.g., 85%, 90%, 95%, 98%, 99%, 100%) of the uracil in the open reading frame have a chemical modification, optionally wherein the vaccine is formulated in a lipid nanoparticle. In some embodiments, 100% of the uracil in the open reading frame have a chemical modification. In some embodiments, a chemical modification is in the 5-Use Gap Code position of the uracil. In some preferred embodiments, a chemical modification is a N1-methyl pseudouridine.

In some embodiments, a RNA (e.g., mRNA) vaccine further comprising an adjuvant.

In some embodiments, at least one RNA polynucleotide encodes at least one influenza antigenic polypeptide that attaches to cell receptors.

In some embodiments, at least one RNA polynucleotide encodes at least one influenza antigenic polypeptide that causes fusion of viral and cellular membranes.

In some embodiments, at least one RNA polynucleotide encodes at least one influenza antigenic polypeptide that is responsible for binding of the virus to a cell being infected.

Some embodiments of the present disclosure provide a vaccine that includes at least one ribonucleic acid (RNA) (e.g., mRNA) polynucleotide having an open reading frame encoding at least one influenza antigenic polypeptide, at least one 5′ terminal cap and at least one chemical modification, formulated within a lipid nanoparticle.

In some embodiments, a 5′ terminal cap is 7mG(5′)ppp(5′)NlmpNp. In some preferred embodiments, the 5′ cap comprises:

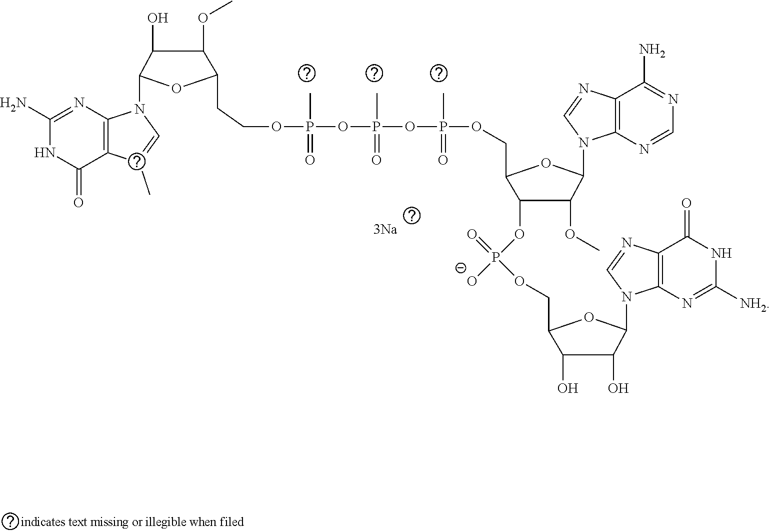 [SMILES: COC1C(CCOP(C)(=O)OP(C)(=O)OP(C)(=O)OCC2OC(n3cnc4c(N)ncnc43)C(OC)C2OP(=O)([O-])OCC2OC(n3cnc4c(=O)[nH]c(N)nc43)C(O)C2O)OC(n2cc(C)c3c(=O)[nH]c(N)nc32)C1O]

In some embodiments, at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In some embodiments, the chemical modification is in the 5-position of the uracil. In some embodiments, the chemical modification is a N1-methylpseudouridine. In some embodiments, the chemical modification is a N1-ethylpseudouridine.

In some embodiments, a lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl]heptadecan-8-amine (L530).

Some embodiments of the present disclosure provide a vaccine that includes at least one RNA (e.g., mRNA) polynucleotide having an open reading frame encoding at least one influenza antigenic polypeptide, wherein at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil.

In some embodiments, an open reading frame of a RNA (e.g., mRNA) polynucleotide encodes at least one influenza antigenic polypeptides. In some embodiments, the open reading frame encodes at least two, at least five, or at least ten antigenic polypeptides. In some embodiments, the open reading frame encodes at least 100 antigenic polypeptides. In some embodiments, the open reading frame encodes 1-100 antigenic polypeptides.

In some embodiments, a vaccine comprises at least two RNA (e.g., mRNA) polynucleotides, each having an open reading frame encoding at least one influenza antigenic polypeptide. In some embodiments, the vaccine comprises at least five or at least ten RNA (e.g., mRNA) polynucleotides, each having an open reading frame encoding at least one antigenic polypeptide or an immunogenic fragment thereof. In some embodiments, the vaccine comprises at least 100 RNA (e.g., mRNA) polynucleotides, each having an open reading frame encoding at least one antigenic polypeptide. In some embodiments, the vaccine comprises 2-100 RNA (e.g., mRNA) polynucleotides, each having an open reading frame encoding at least one antigenic polypeptide.

Also provided herein is an influenza RNA (e.g., mRNA) vaccine of any one of the foregoing paragraphs formulated in a nanoparticle (e.g., a lipid nanoparticle).

In some embodiments, the nanoparticle has a mean diameter of 50-200 nm. In some embodiments, the nanoparticle is a lipid nanoparticle. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol.

In some embodiments, the nanoparticle has a polydispersity value of less than 0.4 (e.g., less than 0.3, 0.2 or 0.1).

In some embodiments, the nanoparticle has a net neutral charge at a neutral pH value.

In some embodiments, the RNA (e.g., mRNA) vaccine is multivalent.

Some embodiments of the present disclosure provide methods of inducing an antigen specific immune response in a subject, comprising administering to the subject any of the RNA (e.g., mRNA) vaccine as provided herein in an amount effective to produce an antigen-specific immune response. In some embodiments, the RNA (e.g., mRNA) vaccine is an influenza vaccine. In some embodiments, the RNA (e.g., mRNA) vaccine is a combination vaccine comprising a combination of influenza vaccines (a broad spectrum influenza vaccine).

In some embodiments, an antigen-specific immune response comprises a T cell response or a B cell response.

In some embodiments, a method of producing an antigen-specific immune response comprises administering to a subject a single dose (no booster dose) of an influenza RNA (e.g., mRNA) vaccine of the present disclosure.

In some embodiments, a method further comprises administering to the subject a second (booster) dose of an influenza RNA (e.g., mRNA) vaccine. Additional doses of an influenza RNA (e.g., mRNA) vaccine may be administered.

In some embodiments, the subjects exhibit a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, an influenza RNA (e.g., mRNA) vaccine is administered to a subject by intradermal injection, intramuscular injection, or by intranasal administration. In some embodiments, an influenza RNA (e.g., mRNA) vaccine is administered to a subject by intramuscular injection.

Some embodiments, of the present disclosure provide methods of inducing an antigen specific immune response in a subject, including administering to a subject an influenza RNA (e.g., mRNA) vaccine in an effective amount to produce an antigen specific immune response in a subject. Antigen-specific immune responses in a subject may be determined, in some embodiments, by assaying for antibody titer (for titer of an antibody that binds to an influenza antigenic polypeptide) following administration to the subject of any of the influenza RNA (e.g., mRNA) vaccines of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered a RNA (e.g., mRNA) vaccine of the present disclosure. In some embodiments, the control is an anti-antigenic polypeptide antibody titer produced in a subject who has been administered a live attenuated or inactivated influenza, or wherein the control is an anti-antigenic polypeptide antibody titer produced in a subject who has been administered a recombinant or purified influenza protein vaccine. In some embodiments, the control is an anti-antigenic polypeptide antibody titer produced in a subject who has been administered an influenza virus-like particle (VLP) vaccine.

A RNA (e.g., mRNA) vaccine of the present disclosure is administered to a subject in an effective amount (an amount effective to induce an immune response). In some embodiments, the effective amount is a dose equivalent to an at least 2-fold, at least 4-fold, at least 10-fold, at least 100-fold, at least 1000-fold reduction in the standard of care dose of a recombinant influenza protein vaccine, wherein the anti-antigenic polypeptide antibody titer produced in the subject is equivalent to an anti-antigenic polypeptide antibody titer produced in a control subject administered the standard of care dose of a recombinant influenza protein vaccine, a purified influenza protein vaccine, a live attenuated influenza vaccine, an inactivated influenza vaccine, or an influenza VLP vaccine. In some embodiments, the effective amount is a dose equivalent to 2-1000-fold reduction in the standard of care dose of a recombinant influenza protein vaccine, wherein the anti-antigenic polypeptide antibody titer produced in the subject is equivalent to an anti-antigenic polypeptide antibody titer produced in a control subject administered the standard of care dose of a recombinant influenza protein vaccine, a purified influenza protein vaccine, a live attenuated influenza vaccine, an inactivated influenza vaccine, or an influenza VLP vaccine.

In some embodiments, the control is an anti-antigenic polypeptide antibody titer produced in a subject who has been administered a virus-like particle (VLP) vaccine comprising structural proteins of influenza.

In some embodiments, the RNA (e.g., mRNA) vaccine is formulated in an effective amount to produce an antigen specific immune response in a subject.

In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times.

In some embodiments, the efficacy (or effectiveness) of a RNA (e.g., mRNA) vaccine is greater than 60%. In some embodiments, the RNA (e.g., mRNA) polynucleotide of the vaccine at least one Influenza antigenic polypeptide.

In some embodiments, the efficacy (or effectiveness) of a RNA (e.g., mRNA) vaccine is at least 65%, at least 70%, at least 75%, at least 80%, at least 85%, or at least 90%.

In some embodiments, the vaccine immunizes the subject against Influenza for up to 2 years. In some embodiments, the vaccine immunizes the subject against Influenza for more than 2 years, more than 3 years, more than 4 years, or for 5-10 years.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a RNA (e.g., mRNA) vaccine may be administered later in life, for example, at the age of about 6 months to about 5 years, or older.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject has been exposed to influenza (e.g., C. trachomatis); the subject is infected with influenza (e.g., C. trachomatis); or subject is at risk of infection by influenza (e.g., C. trachomatis).

In some embodiments, the subject has been exposed to betacoronavirus (e.g., SARS-CoV-2); the subject is infected with betacoronavirus (e.g., SARS-CoV-2); or subject is at risk of infection by betacoronavirus (e.g., SARS-CoV-2).

In some embodiments, the subject has received at least one dose of an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine; the subject has received at least two doses of an immunogenic composition against betacoronavirus (e.g., SARS-CoV-2); the subject is receiving at least one dose of an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine; or the subject is being administered an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine at risk of infection by betacoronavirus (e.g., SARS-CoV-2) concomitantly, simultaneously, or within 12-48 hours of any one of the immunogenic compositions against influenza disclosed herein.

In some embodiments, the subject is immunocompromised (has an impaired immune system, e.g., has an immune disorder or autoimmune disorder).

In some embodiments the nucleic acid vaccines described herein are chemically modified. In other embodiments the nucleic acid vaccines are unmodified.

Yet other aspects provide compositions for and methods of vaccinating a subject comprising administering to the subject a nucleic acid vaccine comprising one or more RNA polynucleotides having an open reading frame encoding a first virus antigenic polypeptide, wherein the RNA polynucleotide does not include a stabilization element, and wherein an adjuvant is not coformulated or co-administered with the vaccine.

In other aspects the disclosed herein is a composition for or method of vaccinating a subject comprising administering to the subject a nucleic acid vaccine comprising one or more RNA polynucleotides having an open reading frame encoding a first antigenic polypeptide wherein a dosage of between 10 μg/kg and 400 μg/kg of the nucleic acid vaccine is administered to the subject. In some embodiments the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty-one.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100-fold higher level in the local lymph node in comparison with the distal lymph node. In other embodiments the nucleic acid vaccine is chemically modified and in other embodiments the nucleic acid vaccine is not chemically modified.

Aspects of the disclosure provide a nucleic acid vaccine comprising one or more RNA polynucleotides having an open reading frame encoding a first antigenic polypeptide, wherein the RNA polynucleotide does not include a stabilization element, and a pharmaceutically acceptable carrier or excipient, wherein an adjuvant is not included in the vaccine. In some embodiments, the stabilization element is a histone stem-loop. In some embodiments, the stabilization element is a nucleic acid sequence having increased GC content relative to wild type sequence.

Aspects of the disclosure provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some embodiments, the antibody titer produced by the mRNA vaccines of the disclosure is a neutralizing antibody titer. In some embodiments the neutralizing antibody titer is greater than a protein vaccine. In other embodiments the neutralizing antibody titer produced by the mRNA vaccines of the disclosure is greater than an adjuvanted protein vaccine. In yet other embodiments the neutralizing antibody titer produced by the mRNA vaccines of the disclosure is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

Also provided are nucleic acid vaccines comprising one or more RNA polynucleotides having an open reading frame encoding a first antigenic polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide.

In some embodiments, the RNA polynucleotide is formulated to produce a neutralizing antibodies within one week of a single administration. In some embodiments, the adjuvant is selected from a cationic peptide and an immunostimulatory nucleic acid. In some embodiments, the cationic peptide is protamine. In some preferred embodiments, the lyophilized composition does not comprise protamine.

Aspects provide nucleic acid vaccines comprising one or more RNA polynucleotides having an open reading frame comprising at least one chemical modification or optionally no modified nucleotides, the open reading frame encoding a first antigenic polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no modified nucleotides, the open reading frame encoding a first antigenic polypeptide, wherein the vaccine has at least 10-fold less RNA polynucleotide than is required for an unmodified mRNA vaccine to produce an equivalent antibody titer. In some embodiments, the RNA polynucleotide is present in a dosage of 25-100 micrograms.

Aspects of the disclosure also provide a unit of use vaccine, comprising between 10 ug and 400 ug of one or more RNA polynucleotides having an open reading frame comprising at least one chemical modification or optionally no modified nucleotides, the open reading frame encoding a first antigenic polypeptide, and a pharmaceutically acceptable carrier or excipient, formulated for delivery to a human subject. In some embodiments, the vaccine further comprises a cationic lipid nanoparticle.

Aspects of the disclosure provide methods of creating, maintaining or restoring antigenic memory to a virus strain in an individual or population of individuals comprising administering to said individual or population an antigenic memory booster nucleic acid vaccine comprising (a) at least one RNA polynucleotide, said polynucleotide comprising at least one chemical modification or optionally no modified nucleotides and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration, and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

In some aspects, methods of inducing an antigen specific immune response in a subject are provided. The method includes administering to the subject an influenza RNA composition in an amount effective to produce an antigen specific immune response. In some embodiments, an antigen specific immune response comprises a T cell response or a B cell response. In some embodiments, an antigen specific immune response comprises a T cell response and a B cell response. In some embodiments, a method of producing an antigen specific immune response involves a single administration of the vaccine. In some embodiments, a method further includes administering to the subject a booster dose of the vaccine. In some embodiments, a vaccine is administered to the subject by intradermal or intramuscular injection.

In exemplary embodiments of the disclosure, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.) In exemplary aspects of the disclosure, antigen-specific antibodies are measured in units of μg/ml or are measured in units of IU/L (International Units per liter) or mlU/ml (milli International Units per ml). In exemplary embodiments of the disclosure, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the disclosure, an efficacious vaccine produces >10 mlU/ml, >20 mlU/ml, >50 mlU/ml, >100 mlU/ml, >200 mlU/ml, >500 mlU/ml or >1000 mlU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

### III. Respiratory Syncytial Virus (Rsv)

In some aspects, the present disclosure provides mutants of wild-type RSV F proteins, wherein the mutants display introduced mutations in the amino acid sequence relative to the amino acid sequence of the corresponding wild-type RSV F protein and are immunogenic against the wild-type RSV F protein or against a virus comprising the wild-type F protein. The amino acid mutations in the mutants include amino acid substitutions, deletions, or additions relative to a wild-type RSV F protein.

In some embodiments, the present disclosure provides mutants of a wild-type RSV F protein, wherein the introduced amino acid mutations are mutation of a pair of amino acid residues in a wild-type RSV F protein to a pair of cysteines (“engineered disulfide mutation”). The introduced pair of cysteine residues allows for formation of a disulfide bond between the cysteine residues that stabilize the protein's conformation or oligomeric state, such as the pre-fusion conformation. Examples of specific pairs of such mutations include: 55C and 188C; 155C and 290C; 103C and 148C; and 142C and 371C, such as S55C and L188C; S155C and S290C; T103C and 1148C; and L142C and N371C.

In some embodiments, the mutant of a wild-type RSV F protein comprises a F1 polypeptide and a F2 polypeptide, wherein the mutant comprises at least one introduced amino acid mutation relative to the amino acid sequence of the wild-type RSV F protein, wherein the introduced amino acid mutation is a pair of cysteine mutations selected from the group consisting of: (1) 55C and 188C; (2) 103C and 148C; and (3) 142C and 371C, and wherein amino acid positions are numbered according to SEQ ID NO: 1. In some embodiments, the mutant further comprises at least one cavity filling mutation, and at least one electrostatic mutation, wherein the cavity filling mutation is selected from the group consisting of: (1) substitution of the amino acid at position 62, 155, 190, or 290 with I, Y, L, H, or M; (2) substitution of the amino acid at position 54, 58, 189, 219, or 397 with I, Y, L, H, or M; (3) substitution of the amino acid at position 151 with A or H; (4) substitution of the amino acid at position 147 or 298 with I, L, H, or M; and (5) substitution of the amino acid at position 164, 187, 192, 207, 220, 296, 300, or 495 with I, Y, or H, and wherein the electrostatic mutation is selected from the group consisting of: (1) substitution of the amino acid at position 82, 92, or 487 by D, F, Q, T, S, L, or H; (2) substitution of the amino acid at position 315, 394, or 399 by F, M, R, S, L, I, Q, or T; (3) substitution of the amino acid at position 392, 486, or 489 by H, S, N, T, or P; and (4) substitution of the amino acid at position 106 or 339 by F, Q, N, or W.

In some embodiments, a mutant of a wild-type respiratory syncytial virus (RSV) F protein comprises a F1 polypeptide and a F2 polypeptide, wherein the mutant comprises at least one introduced amino acid mutation relative to the amino acid sequence of the wild-type RSV F protein, and wherein the introduced amino acid mutation comprises: (i) the pair of cysteine mutations 155C and 290C; and (ii) one or more cavity filling mutations selected from the group consisting of: 1) substitution of the amino acid at position 190 with I; 2) substitution of the amino acid at position 54 with I, Y, L, H, or M; 3) substitution of the amino acid at position 296 with I, Y, H, and wherein amino acid positions are numbered according to SEQ ID NO:1. In some embodiments, the cavity filling mutation is selected from the group consisting of: (1) substitution of the amino acid at position 190 with I; (2) substitution of the amino acid at position 54 with H; and (3) substitution of the amino acid at position 296 with I. In some embodiments, the mutant is in the form of a trimer. In some embodiments, the mutant has increased stability as compared with the corresponding wild-type RSV F protein, wherein the stability is measured by binding of the mutant with antibody AM14. In some embodiments, the wild-type RSV is subtype A or subtype B. In some embodiments, the cavity filing mutation is selected from the group consisting of: 54H, 1901, and 2961. In some embodiments, the mutant further comprises an electrostatic mutation. In some embodiments, the electrostatic mutation is selected from the group consisting of: (1) substitution of the amino acid at position 82, 92, or 487 by D, F, Q, T, S, L, or H; (2) substitution of the amino acid at position 315, 394, or 399 by F, M, R, S, L, I, Q, or T; (3) substitution of the amino acid at position 392, 486, or 489 by H, S, N, T, or P; and (4) Substitution of the amino acid at position 106 or 339 by F, Q, N, or W. In some embodiments, a mutant of a wild-type RSV F protein comprises a F1 polypeptide and a F2 polypeptide, wherein the mutant comprises at least one introduced amino acid mutation relative to the amino acid sequence of the wild-type RSV F protein, and wherein the introduced amino acid mutation comprises: (i) the pair of cysteine mutations 155C and 290C; (ii) an cavity filling mutation; and (iii) an electrostatic mutation, wherein the cavity filling mutation is selected from the group consisting of: (1) substitution of the amino acid at position 62 with I, Y, L, H, or M; (2) substitution of the amino acid at position 190 with I; (3) substitution of the amino acid at position 54, 58, 189, 219, or 397 with I, Y, L, H, or M; (4) substitution of the amino acid at position 151 with A or H; (5) substitution of the amino acid at position 147 or 298 with I, L, H, or M; and (6) substitution of the amino acid at position 164, 187, 192, 207, 220, 296, 300, or 495 with I, Y, H, wherein the electrostatic mutation is 486S, and wherein the amino acid positions are numbered according to SEQ ID NO:1. In some embodiments, the mutant comprises a combination of amino acid mutations selected from the group consisting of: (1) combination of 155C, 290C, and 54H; (2) combination of 155C, 290C, 2961; (3) combination of 155C, 290C, 54H, and 296Y; (4) combination of 155C, 290C, and 1901; (5) combination of 155C, 290C, 54H, and 1901; (6) combination of 155C, 290C, 54H, 496S; (7) combination of 155C, 290C, 1901, and 486S; (7) combination of 155C, 290C, 2961; and 486S; (9) combination of 155C, 290C, 54H, 1901; and 486S; (10) combination of 155C, 290C, 54H, 2961, and 486S (11) combination of 155C, 290C, 1901, 2961, and 485S; (12) combination of 155C, 290C, 54H, 1901, 2961, and 486S; (13) combination of 155C, 290C, 1901, and 2961; and (14) combination of 155C, 290C, 54H, 1901, and 2961. In some embodiments, a mutant of a wild-type RSV F protein comprises a F1 polypeptide and a F2 polypeptide, wherein the mutant comprises at least one introduced amino acid mutation relative to the amino acid sequence of the wild-type RSV F protein, and wherein the introduced amino acid mutation comprises: (i) the pair of cysteine mutations 155C and 290C; (ii) at least one cavity filling mutation; and (iii) at least one pair of cysteine mutations in the HRB region, wherein the cavity filling mutation is selected from the group consisting of: (1) substitution of the amino acid at position 62 with I, Y, L, H, or M; (2) substitution of the amino acid at position 190 with I; (3) substitution of the amino acid at position 54, 58, 189, 219, or 397 with I, Y, L, H, or M; (4) substitution of the amino acid at position 151 with A or H; (5) substitution of the amino acid at position 147 or 298 with I, L, H, or M; and (6) substitution of the amino acid at position 164, 187, 192, 207, 220, 296, 300, or 495 with I, Y, H, wherein the at least one pair of cysteine mutations in the HRB region is selected from the group consisting of: (1) 508C and 509C; (2) 515C and 516C; and (3) 522C and 523C, and wherein the amino acid positions are numbered according to SEQ ID NO:1. In some embodiments, the disclosure provides a pharmaceutical composition comprising (i) an RSV F protein mutant described herein and (ii) a pharmaceutically acceptable carrier. In some embodiments, the F1 polypeptide and F2 polypeptide of the pharmaceutical composition are derived from the F protein of RSV subtype B. In some embodiments, the F1 polypeptide and F2 polypeptide of the pharmaceutical composition are derived from the F protein of RSV subtype A. In some embodiments, the pharmaceutical composition further comprises a second mutant of a wild-type RSV F protein as described herein, wherein the F1 polypeptide and F2 polypeptide of the second mutant are from the F protein of RSV subtype B. In some embodiments, the pharmaceutical composition is a vaccine. In some embodiments, the present disclosure provides a nucleic acid molecule (e.g., as described below) comprising a nucleotide sequence that encodes an amino acid sequence of an RSV F protein mutant according to claim 1.

In some embodiments, the present disclosure provides a method of preventing RSV infection in a subject, comprising administering to the subject an effective amount of the pharmaceutical composition described herein. In some embodiments, the present disclosure provides a pharmaceutical composition comprising (i) an RSV F protein mutant as described herein and (ii) a pharmaceutically acceptable carrier. In some embodiments, the present disclosure provides a method of preventing RSV infection in a subject, comprising administering to the subject an effective amount of the pharmaceutical composition as described herein.

The present disclosure provides for RNA molecules (e.g., RNA polynucleotides) comprising at least one open reading frame encoding a respiratory syncytial virus (RSV) polypeptide. The present disclosure further provides for an immunogenic composition comprising at least one RNA molecule encoding an RSV polypeptide complexed with, encapsulated in, or formulated with one or more lipids, and forming lipid nanoparticles (LNPs). The RSV polypeptide to be included in the immunogenic composition disclosed herein can be any RSV F protein in the prefusion conformation.

The term “prefusion conformation” refers to a structural conformation adopted by an RSV F protein or mutant thereof that can be specifically bound by (i) antibody D25 or AM22 when the RSV F protein or mutant is in the form of a monomer or trimer, or (ii) by antibody AM14 when the RSV F protein mutant is in the form of a trimer. The prefusion trimer conformation is a subset of prefusion conformations.

The term “postfusion conformation” refers to a structural conformation adopted by the RSV F protein that is not specifically bound by D25, AM22, or AM14. Native F protein adopts the postfusion conformation subsequent to the fusion of the virus envelope with the host cellular membrane. RSV F protein may also assume the postfusion conformation outside the context of a fusion event, for example, under stress conditions such as heat and low osmolality, when extracted from a membrane, when expressed as an ectodomain, or upon storage. The term “AM14” refers to an antibody described in WO 2008/147196 A2, which is hereby incorporated by reference in its entirety. The term “AM22” refers to an antibody described in WO 2011/043643 A1, which is hereby incorporated by reference in its entirety. The term “D25” refers to an antibody described in WO 2008/147196 A2, which is hereby incorporated herein by reference in its entirety.

In some embodiments, the RSV F protein is an RSV F protein of subtype A. In some embodiments, the RSV F protein is an RSV F protein of subtype B. In some embodiments, the RSV F protein is a mutant of wild type RSV F protein. In some embodiments, the RSV F protein is a mutant of wild type RSV F protein of subtype A. In some embodiments, the RSV F protein is a mutant of wild type RSV F protein of subtype B. In some embodiments, the mutants display introduced mutations in the amino acid sequence relative to the amino acid sequence of the corresponding wild-type RSV F protein and are immunogenic against the wild-type RSV F protein in the prefusion conformation or against a virus comprising the wild-type F protein. The amino acid mutations in the mutants include amino acid substitutions, deletions, or additions relative to a wild-type RSV F protein.

In some embodiments, the RSV F protein is an RSV protein mutant as described in WO2017/109629, which is hereby incorporated by reference in its entirety.

In some embodiments, the RSV F protein is a mutant of a wild-type RSV F protein, wherein the introduced amino acid mutations are mutation of a pair of amino acid residues in a wild-type RSV F protein to a pair of cysteines (“engineered disulfide mutation”). The introduced pair of cysteine residues allows for formation of a disulfide bond between the cysteine residues that stabilize the protein's conformation or oligomeric state, such as the prefusion conformation. Examples of specific pairs of such mutations include: 55C and 188C; 155C and 290C; 103C and 148C; and 142C and 371C, such as S55C and L188C; S155C and S290C; A103C and 1148C; and L142C and N371C.

In still other embodiments, the RSV F protein mutants comprise amino acid mutations that are one or more cavity filling mutations. Examples of amino acids that may be replaced with the goal of cavity filling include small aliphatic (e.g. Gly, Ala, and Val) or small polar amino acids (e.g. Ser and Thr) and amino acids that are buried in the prefusion conformation, but exposed to solvent in the postfusion conformation. Examples of the replacement amino acids include large aliphatic amino acids (Ile, Leu and Met) or large aromatic amino acids (His, Phe, Tyr and Trp). In some specific embodiments, the RSV F protein mutant comprises a cavity filling mutation selected from the group consisting of: (1) substitution of S at positions 55, 62, 155, 190, or 290 with I, Y, L, H, or M; (2) substitution of T at position 54, 58, 189, 219, or 397 with I, Y, L, H, or M; (3) substitution of G at position 151 with A or H; (4) substitution of A at position 147 or 298 with I, L, H, or M; (5) substitution of V at position 164, 187, 192, 207, 220, 296, 300, or 495 with I, Y, H; and (6) substitution of R at position 106 with W.

In some particular embodiments, the RSV F protein mutant comprises at least one cavity filling mutation selected from the group consisting of: T54H, S1901, and V2961.

In still other embodiments, the RSV F protein mutants comprise electrostatic mutations, which decrease ionic repulsion or increase ionic attraction between resides in a protein that are proximate to each other in the folded structure. In several embodiments, the RSV F protein mutant includes an electrostatic substitution that reduces repulsive ionic interactions or increases attractive ionic interactions with acidic residues of Glu487 and Asp489 from another protomer of RSV F trimer. In some specific embodiments, the RSV F protein mutant comprises an electrostatic mutation selected from the group consisting of: (1) substitution of E at position 82, 92, or 487 by D, F, Q, T, S, L, or H; (2) substitution of K at position 315, 394, or 399 by F, M, R, S, L, I, Q, or T; (3) substitution of D at position 392, 486, or 489 by H, S, N, T, or P; and (4) substitution of R at position 106 or 339 by F, Q, N, or W.

In still other embodiments, the RSV F protein mutants comprise a combination of two or more different types of mutations selected from engineered disulfide mutations, cavity filling mutations, and electrostatic mutations. In some particular embodiments, the RSV F protein mutants comprise a combination of mutations relative to the corresponding wild-type RSV F protein, wherein the combination of mutations is selected from the group consisting of: (1) combination of A103C, 1148C, S1901, and D486S; (2) combination of T54H S55C L188C D486S; (3) combination of T54H, A103C, 1148C, S1901, V2961, and D486S; (4) combination of T54H, S55C, L142C, L188C, V2961, and N371C; (5) combination of S55C, L188C, and D486S; (6) combination of T54H, S55C, L188C, and S1901; (7) combination of S55C, L188C, S1901, and D486S; (8) combination of T54H, S55C, L188C, S1901, and D486S; (9) combination of S155C, S1901, S290C, and D486S; (10) combination of T54H, S55C, L142C, L188C, V2961, N371C, D486S, E487Q, and D489S; (11) combination of T54H, S155C, S1901, S290C, and V2961, and, (12) combination of S155C, S190F, S290C, and V207L.

In some embodiments, the RSV F protein is of subtype A and comprises the mutations S155C, S190F, S290C, and V207L. In some embodiments, the RSV F protein is of subtype B and comprises the mutations S155C, S190F, S290C, and V207L. In some embodiments, the RSV F protein is of subtype A and comprises the mutations A103C, 1148C, S1901, and D486S. In some embodiments, the RSV F protein is of subtype B and comprises the mutations A103C, 1148C, S1901, and D486S.

In view of the substantial conservation of RSV F sequences, a person of ordinary skill in the art can easily compare amino acid positions between different native RSV F sequences to identify corresponding RSV F amino acid positions between different RSV strains and subtypes. For example, across nearly all identified native RSV F0 precursor proteins, the furin cleavage sites fall in the same amino acid positions. Thus, the conservation of native RSV F protein sequences across strains and subtypes allows use of a reference RSV F sequence for comparison of amino acids at particular positions in the RSV F protein. For the purposes of this disclosure (unless context indicates otherwise), the RSV F protein amino acid positions are given with reference to the amino acid sequence of the full length native F precursor polypeptide of the RSV A2 strain; corresponding to GenInfo Identifier GI 138251 and Swiss Prot identifier P03420 (SEQ ID NO: 1).

In some embodiments, the RSV F protein is in the mature form of the RSV F protein, which comprises two separate polypeptide chains, namely the F1 polypeptide and F2 polypeptide. In some other embodiments, the F2 polypeptide is linked to the F1 polypeptide by one or two disulfide bonds to form a F2/F1 heterodimer. In still other embodiments, the RSV F mutants are in the form a single chain protein, wherein the F2 polypeptide is linked to the F1 polypeptide by a peptide bond or peptide linker. Any suitable peptide linkers for joining two polypeptide chains together may be used. Examples of such linkers include G, GG, GGG, GS, and SAIG linker sequences. The linker may also be the full length pep27 sequence or a fragment thereof.

The F1 polypeptide chain of the mutant may be of the same length as the full length F1 polypeptide of the corresponding wild-type RSV F protein; however, it may also have deletions, such as deletions of 1 up to 60 amino acid residues from the C-terminus of the full-length F1 polypeptide. A full-length F1 polypeptide of the RSV F mutants corresponds to amino acid positions 137-574 of the native RSV F0 precursor, and includes (from N- to C-terminus) an extracellular region (residues 137-524), a transmembrane domain (residues 525-550), and a cytoplasmic domain (residues 551-574). It should be noted that amino acid residues 514 onwards in a native F1 polypeptide sequence are optional sequences in a F1 polypeptide of the RSV F protein to be included in the immunogenic composition provided herein, and therefore may be absent from the F1 polypeptide of the mutant.

In some embodiments, the F1 polypeptide of the RSV F mutants lacks the entire cytoplasmic domain. In other embodiments, the F1 polypeptide lacks the cytoplasmic domain and a portion of or all entire transmembrane domain. In some specific embodiments, the mutant comprises a F1 polypeptide wherein the amino acid residues from position 510, 511, 512, 513, 514, 515, 520, 525, or 530 through 574 are absent. Typically, for mutants that are linked to trimerization domain, such as a foldon, amino acids 514 through 754 can be absent. Thus, in some specific embodiment, amino acid residues 514 through 574 are absent from the F1 polypeptide of the mutant. In still other specific embodiments, the F1 polypeptide of the RSV F mutants comprises or consists of amino acid residues 137-513 of a native F0 polypeptide sequence, such as any of alternative F0 precursor sequence such as those disclosed in SEQ ID Nos: 1, 2, 4, 6, and 81-270 of WO2017109629, which is hereby incorporated by reference in its entirety.

The F1 polypeptide and F2 polypeptide of the RSV F protein mutants to which one or more mutations are introduced can be from any wild-type RSV F proteins known in the art or discovered in the future, including, without limitations, the F protein amino acid sequence of RSV subtype A, and subtype B strains, including A2 Ontario and Buenos Aires, or any other subtype. In some embodiments, the RSV F mutant comprises a F1 and/or a F2 polypeptide from a RSV A virus, for example, a F1 and/or F2 polypeptide from a RSV F0 precursor protein set forth in any one of SEQ ID NOs: 1, 2, 4, 6, and 81-270 of WO2017109629 to which one or more mutations are introduced. In some other embodiments, the RSV F mutant comprises a F1 and/or a F2 polypeptide from a RSV B virus, for example, a F1 and/or F2 polypeptide from a RSV F0 precursor protein set forth in any one of SEQ ID NOs:2, and 211-263 of WO2017/109629 to which one or more mutations are introduced. In still other embodiments, the RSV F mutant comprises a F1 and/or a F2 polypeptide from a RSV bovine virus, for example, a F1 and/or F2 polypeptide from a RSV F0 precursor protein set forth in any one of SEQ ID NOs:264-270 of WO2017109629 to which one or more mutations are introduced.

The term “F0 polypeptide” (F0) refers to the precursor polypeptide of the RSV F protein, which is composed of a signal polypeptide sequence, a F1 polypeptide sequence, a pep27 polypeptide sequence, and a F2 polypeptide sequence. With rare exceptions the F0 polypeptides of the known RSV strains consist of 574 amino acids.

The term “F1 polypeptide” (F1) refers to a polypeptide chain of a mature RSV F protein. Native F1 includes approximately residues 137-574 of the RSV F0 precursor and is composed of (from N- to C-terminus) an extracellular region (approximately residues 137-524), a transmembrane domain (approximately residues 525-550), and a cytoplasmic domain (approximately residues 551-574). As used herein, the term encompasses both native F1 polypeptides and F1 polypeptides including modifications (e.g., amino acid substitutions, insertions, or deletions) from the native sequence, for example, modifications designed to stabilize an RSV F protein mutant or to enhance the immunogenicity of an RSV F protein mutant.

The term “F2 polypeptide” (F2) refers to the polypeptide chain of a mature RSV F protein. Native F2 includes approximately residues 26-109 of the RSV F0 precursor. As used herein, the term encompasses both native F2 polypeptides and F2 polypeptides including modifications (e.g., amino acid substitutions, insertions, or deletions) from the native sequence, for example, modifications designed to stabilize an RSV F protein mutant in a prefusion conformation or to enhance the immunogenicity of an RSV F protein mutant. In native RSV F protein, the F2 polypeptide is linked to the F1 polypeptide by two disulfide bonds to form a F2-F1 heterodimer. The term “foldon” or “foldon domain” refers to an amino acid sequence that is capable of forming trimers. One example of such foldon domains is the peptide sequence derived from bacteriophage T4 fibritin, which has the sequence of GYIPEAPRDGQAYVRKDGEWVLLSTFL (SEQ ID NO: 45).

In some aspects, the RNA molecule encodes an RSV F protein mutant as disclosed WO2009/079796, WO2010/149745, WO2011/008974, WO2014/160463, WO2014/174018, WO2014/202570, WO2015/013551, WO2015/177312, WO2017/005848, WO2017/174564, WO2017/005844 and WO2018/109220. The RSV F proteins disclosed in these references are hereby incorporated by reference in their entirety.

Antibodies to RSV F protein are prevalent after natural infection and following vaccination and have been shown to neutralize viral activity in vitro. As used herein, the term “respiratory syncytial virus” or “RSV” is not limited to any particular strain or variant.

In some aspects, the RNA molecule comprises an open reading frame encoding a RSV antigen. In some aspects, the RSV antigen is a RSV polypeptide. In some aspects, the RSV polypeptide is a RSV glycoprotein or a fragment or a variant thereof. In some aspects, the RNA molecule encodes a RSV F protein.

In some aspects, the RSV polypeptide is a full-length RSV polypeptide. In some aspects, the RSV polypeptide is a truncated RSV polypeptide. In some aspects, the RSV polypeptide is a variant of a RSV polypeptide. In some aspects, the RSV polypeptide is a fragment of a RSV polypeptide.

In some aspects, the RSV polypeptide is a full-length RSV F protein. In some aspects, the RSV polypeptide is a truncated RSV F protein. In some aspects, the RSV polypeptide is a variant of a RSV F protein. In some aspects, the RSV polypeptide is a fragment of a RSV F protein.

In some aspects, the RSV F protein comprises at least one mutation. In some aspects, the RSV F protein comprises at least two mutations. In some aspects, the RSV F protein comprises at least three mutations. In some aspects, the RSV F protein comprises at least four mutations. In some aspects, the RSV F protein comprises 4 mutations.

In some aspects, the RNA molecule encodes a RSV F protein of Table 30. In some aspects, the RNA molecule encodes a RSV F protein comprising an amino acid sequence of any of SEQ ID NO: 1 to 6, or fragment or variant thereof. In some aspects, RSV F protein may have at least, at most, exactly, or between any two of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to any of the amino acid sequences of Table 30, for example, any of SEQ ID NO: 1 to 6. In some aspects, RSV F protein consists of any of the amino acid sequences of Table 30, for example, any of SEQ ID NO: 1 to 6.

In some aspects, the RNA molecule sequence is transcribed from a DNA nucleic acid sequence (DNA polynucleotide) of Table 31. In some aspects, the RNA molecule comprises an ORF transcribed from a nucleic acid sequence of any of SEQ ID NO: 7 to 10, or fragment or variant thereof. In some aspects, the RNA molecule comprises an ORF transcribed from a nucleic acid sequence that may have at least, at most, exactly, or between any two of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to any of the nucleic sequences of Table 31, for example, any of SEQ ID NO: 7 to 10. In some aspects, the RNA molecule comprises an ORF transcribed from a nucleic acid sequence that consists of any of the nucleic sequences of Table 31, for example, any of SEQ ID NO:7 to 10.

In some aspects, the RNA molecule comprises an ORF comprising an RNA nucleic acid sequence (RNA polynucleotide) of Table 32. In some aspects, the RNA molecule comprises an ORF comprising a nucleic acid sequence of any of SEQ ID NO: 11 to 16, or fragment or variant thereof. In some aspects, the RNA molecule comprises an ORF comprising a nucleic acid sequence that may have at least, at most, exactly, or between any two of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to any of the RNA nucleic acid sequences of Table 32, for example, any of SEQ ID NO: 11 to 16. In some aspects, the RNA molecule comprises an ORF comprising a nucleic acid sequence that consists of any of the RNA nucleic acid sequences of Table 32, for example, any of SEQ ID NO: 11 to 16.

In some aspects, the RNA molecule comprises stabilized RNA. In some aspects, the RNA molecule comprises a nucleic acid sequence having at least one uridine replaced by N1-methylpseudouridine. In some aspects, the RNA molecule comprises a sequence having all uridines replaced by N1-methylpseudouridine (designated as “W”). In some aspects, the RNA molecule comprises an ORF comprising a nucleic acid sequence of any of SEQ ID NO: 11 to 16, wherein all uridines have been replaced by N1-methylpseudouridine (designated as “4”).

In some aspects, the RNA molecule comprises an open reading frame encoding a RSV F protein amino acid sequence that may be at least, at most, exactly, or between any two of 70%, 80%, 85%, 86%, 87%, 88%, 89%, 90%, 91%, 92%, 93%, 94%, 95%, 96%, 97%, 98%, or 99% identical to any of the RSV F protein sequences of SEQ ID NO: 1 to 6 or other RSV prefusion F proteins described herein. In some aspects, the RNA molecule comprises an open reading frame encoding a RSV F protein amino acid sequence that consists of any of the RSV F protein sequences of SEQ ID NO: 1 to 6 or other RSV prefusion F protein described herein.

In some aspects, the RNA molecule comprises an open reading frame transcribed from a DNA nucleic acid sequence that may be at least, at most, exactly, or between any two of 70%, 80%, 85%, 86%, 87%, 88%, 89%, 90%, 91%, 92%, 93%, 94%, 95%, 96%, 97%, 98%, or 99% identical to any of the nucleic acid sequences of SEQ ID NO: 7 to 10 or other nucleic acid described herein. In some aspects, the RNA molecule comprises an open reading frame transcribed from a DNA nucleic acid sequence that consists of any of the nucleic acid sequences of SEQ ID NO: 7 to 10 or other nucleic acid described herein.

In some aspects, the RNA molecule comprises an open reading frame comprising an RNA nucleic acid sequence that may be at least, at most, exactly, or between any two of 70%, 80%, 85%, 86%, 87%, 88%, 89%, 90%, 91%, 92%, 93%, 94%, 95%, 96%, 97%, 98%, or 99% identical to any of the nucleic acid sequences of SEQ ID NO: 11 to 16 or other nucleic acid described herein. In some aspects, the RNA molecule comprises an open reading frame comprising an RNA nucleic acid sequence that consists of any of the nucleic acid sequences of SEQ ID NO: 11 to 16 (Table 3) or other nucleic acid described herein. In some aspects, the RNA molecule comprises an ORF comprising a nucleic acid sequence of any of SEQ ID NO: 11 to 16 (Table 3), wherein all uridines have been replaced by N1-methylpseudouridine (designated as “4”).

### IV. RNA Molecule

In some aspects, the RNA molecule described herein is a coding RNA molecule. Coding RNA includes a functional RNA molecule that may be translated into a peptide or polypeptide. In some aspects, the coding RNA molecule includes at least one open reading frame (ORF) coding for at least one peptide or polypeptide. An open reading frame comprises a sequence of codons that is translatable into a peptide or protein. The coding RNA molecule may include one (monocistronic), two (bicistronic) or more (multicistronic) OFRs, which may be a sequence of codons that is translatable into a polypeptide or protein of interest.

The coding RNA molecule may be a messenger RNA (mRNA) molecule, viral RNA molecule, or self-amplifying RNA molecule (saRNA, also referred to as a replicon). In some aspects, the RNA molecule is an mRNA. Preferably, the RNA molecule of the present disclosure is an mRNA. In some aspects, the RNA molecule is modRNA. In some aspects, the RNA molecule is a saRNA. In some aspects, the saRNA molecule may be a coding RNA molecule.

The RNA molecule may encode one polypeptide of interest or more, such as an antigen or more than one antigen, e.g., two, three, four, five, six, seven, eight, nine, ten or more polypeptides. Alternatively, or in addition, one RNA molecule may also encode more than one polypeptide of interest, such as an antigen, e.g., a bicistronic, or tricistronic RNA molecule that encodes different or identical antigens.

The sequence of the RNA molecule may be codon optimized or deoptimized for expression in a desired host, such as a human cell. In some aspects, a gene of interest (e.g., an antigen) described herein is encoded by a coding sequence which is codon-optimized and/or the guanosine/cytidine (G/C) content of which is increased compared to wild type coding sequence. In some aspects, one or more sequence regions of the coding sequence are codon-optimized and/or increased in the G/C content compared to the corresponding sequence regions of the wild type coding sequence. In some aspects, codon-optimization and/or increasing the G/C content does not change the sequence of the encoded amino acid sequence.

The term “codon-optimized” is understood by those in the art to refer to alteration of codons in the coding region of a nucleic acid molecule to reflect the typical codon usage of a host organism without altering the amino acid sequence encoded by the nucleic acid molecule. Within the context of the present disclosure, in some aspects, coding regions are codon-optimized for optimal expression in a subject to be treated using an RNA polynucleotide described herein. Codon-optimization is based on the finding that the translation efficiency is also determined by a different frequency in the occurrence of tRNA molecules in cells. Thus, the sequence of RNA may be modified such that codons for which frequently occurring tRNA molecules are available are inserted in place of “rare codons.”

In some aspects, G/C content of a coding region (e.g., of a gene of interest sequence) of an RNA is increased compared to the G/C content of the corresponding coding sequence of a wild type RNA encoding the gene of interest, wherein in some aspects, the amino acid sequence encoded by the RNA is not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability may be determined (so-called alternative codon usage). Depending on the amino acid to be encoded by the RNA, there are various possibilities for modification of the RNA sequence, compared to its wild type sequence. In particular, codons which contain A and/or U nucleosides may be modified by substituting these codons by other codons, which code for the same amino acids but contain no A and/or U or contain a lower content of A and/or U nucleosides. Thus, in some aspects, G/C content of a coding region of an RNA described herein is increased by at least, at most, exactly, or between any two of 10%, 20%, 30%, 40%, 50%, 55%, or even more compared to the G/C content of a coding region of a wild type RNA.

In some aspects, the RNA molecule includes from about 20 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

In some aspects, the RNA molecule has at least, at most, exactly, or between any two of about 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, 9000, 9200, 9400, 9600, 9800, 10000, 10000, 12000, 14000, 16000, 18000, 20000, 22000, 24000, 26000, 28000, 30000, 32000, 34000, 36000, 38000, 40000, 42000, 44000, 46000, 48000, 50000, 52000, 54000, 56000, 58000, 60000, 62000, 64000, 66000, 68000, 70000, 72000, 74000, 76000, 78000, 80000, 82000, 84000, 86000, 88000, 90000, 92000, 94000, 96000, 98000, or 100000 nucleotides.

In some aspects, the RNA molecule includes at least 100 nucleotides. For example, in some aspects, the RNA has a length between 100 and 15,000 nucleotides; between 7,000 and 16,000 nucleotides; between 8,000 and 15,000 nucleotides; between 9,000 and 12,500 nucleotides; between 11,000 and 15,000 nucleotides; between 13,000 and 16,000 nucleotides; between 7,000 and 25,000 nucleotides. In some aspects, the RNA molecule has at least, at most, exactly, or between any two of about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, 6500, 6550, 6600, 6650, 6700, 6750, 6800, 6850, 6900, 6950, 7000, 7050, 7100, 7150, 7200, 7250, 7300, 7350, 7400, 7450, 7500, 7550, 7600, 7650, 7700, 7750, 7800, 7850, 7900, 7950, 8000, 8050, 8100, 8150, 8200, 8250, 8300, 8350, 8400, 8450, 8500, 8550, 8600, 8650, 8700, 8750, 8800, 8850, 8900, 8950, 9000, 9050, 9100, 9150, 9200, 9250, 9300, 9350, 9400, 9450, 9500, 9550, 9600, 9650, 9700, 9750, 9800, 9850, 9900, 9950, 10000, 10050, 10100, 10150, 10200, 10250, 10300, 10350, 10400, 10450, 10500, 10550, 10600, 10650, 10700, 10750, 10800, 10850, 10900, 10950, 11000, 11050, 11100, 11150, 11200, 11250, 11300, 11350, 11400, 11450, 11500, 11550, 11600, 11650, 11700, 11750, 11800, 11850, 11900, 11950, 12000, 12050, 12100, 12150, 12200, 12250, 12300, 12350, 12400, 12450, 12500, 12550, 12600, 12650, 12700, 12750, 12800, 12850, 12900, 12950, 13000, 13050, 13100, 13150, 13200, 13250, 13300, 13350, 13400, 13450, 13500, 13550, 13600, 13650, 13700, 13750, 13800, 13850, 13900, 13950, 14000, 14050, 14100, 14150, 14200, 14250, 14300, 14350, 14400, 14450, 14500, 14550, 14600, 14650, 14700, 14750, 14800, 14850, 14900, 14950, or 15000 nucleotides.

In some aspects of the present disclosure, an RNA is or comprises messenger RNA (mRNA) that relates to an RNA transcript which encodes a polypeptide. In some aspects, an RNA disclosed herein comprises: a 5′ cap comprising a 5′ cap disclosed herein; a 5′ untranslated region comprising a cap proximal sequence (5′ UTR), a sequence encoding a payload (e.g., a RSV prefusion F protein and/or an antigen derived from influenza); a 3′ untranslated region (3′ UTR); and/or a polyadenylate (Poly A) sequence.

In some aspects, an RNA disclosed herein comprises the following components in 5′ to 3′ orientation: a 5′ cap comprising a 5′ cap disclosed herein; a 5′ untranslated region comprising a cap proximal sequence (5′ UTR), a sequence encoding a payload (e.g., a RSV prefusion F protein and/or an antigen derived from influenza); a 3′ untranslated region (3′ UTR); and a Poly-A sequence.

### 1. Modified Nucleobases

In the present disclosure the RNA molecules may comprise modified nucleobases which may be incorporated into modified nucleosides and nucleotides. In some aspects, the RNA molecule may include one or more modified nucleotides. Naturally occurring nucleotide modifications are known in the art.

In some aspects, the RNA molecule may include a modified nucleotide. Non-limiting examples of modified nucleotides that may be included in the RNA molecule include pseudouridine, N1-methylpseudouridine, 5-methyluridine, 3-methyl-uridine, 5-methoxy-uridine, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), uridine 5-oxyacetic acid, uridine 5-oxyacetic acid methyl ester, 5-carboxymethyl-uridine, 1-carboxymethyl-pseudouridine, 5-carboxy hydroxymethyl-uridine, 5-carboxy hydroxy methyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 1-ethyl-pseudouridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-2-thio-uridine, 1-methyl-4-thio-pseudouridine, 4-thio-1-methyl-pseudouridine, 3-methyl-1-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine, 1-methyl-3-(3-amino-3-carboxypropyl)pseudouridine, 5-(isopentenylaminomethyl)uridine, 5-(isopentenylaminomethyl)-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, any other modified uridine known in the art, or combinations thereof.

In some aspects of the present disclosure, modified nucleotides include any one of N1-methylpseudouridine or pseudouridine.

In some aspects, the RNA molecule comprises nucleotides that are N1-methylpseudouridine modified. In some aspects, the RNA molecule comprises nucleotides that are a pseudouridine modified.

In some aspects, an RNA comprises a modified nucleoside in place of at least one uridine. In some aspects, an RNA comprises a modified nucleoside in place of each uridine. In some aspects, the RNA molecule comprises a sequence having at least one uridine replaced by N1-methylpseudouridine. In some aspects, the RNA molecule comprises a sequence having all uridines replaced by N1-methylpseudouridine. N1-methylpseudouridine is designated in sequences as “W”. The term “uracil,” as used herein, describes one of the nucleobases that may occur in the nucleic acid of RNA. The term “uridine,” as used herein, describes one of the nucleosides that may occur in RNA. “Pseudouridine” is one example of a modified nucleoside that is an isomer of uridine, where the uracil is attached to the pentose ring via a carbon-carbon bond instead of a nitrogen-carbon glycosidic bond.

In some aspects, the RNA molecule comprises a nucleic acid sequence having at least one uridine replaced by pseudouridine. In some aspects, the RNA molecule comprises a nucleic acid sequence having at least, at most, exactly, or between any two of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of uridines replaced by pseudouridine. In some aspects, the RNA molecule comprises a nucleic acid sequence having all uridines replaced by pseudouridine.

Modifications that may be present in the RNA molecules further include, for example, m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); mil (1-methylinosine); m′lm (1,2′-O-dimethylinosine); m3C (3-methylcytidine); Cm (2T-O-methylcytidine); s2C (2-thiocytidine); ac4C (N4-acetylcytidine); f5C (5-formylcytosine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); rn62Am (N6,N6,O-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C6)-alkyluracil, 5-methyluracil, 5-(C2-Ce)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U.

In some aspects, the RNA molecule may include phosphoramidate, phosphorothioate, and/or methylphosphonate linkages.

The sequence of the RNA molecule may be modified if desired, for example to increase the efficacy of expression or replication of the RNA, or to provide additional stability or resistance to degradation. For example, the RNA sequence may be modified with respect to its codon usage, for example, to increase translation efficacy and half-life of the RNA.

In some aspects, the RNA molecule of the present disclosure comprises an open reading frame having at least one codon modified sequence. A codon modified sequence relates to coding sequences that differ in at least one codon (triplets of nucleotides coding for one amino acid) compared to the corresponding wild type coding sequence. A codon modified sequence may show improved resistance to degradation, improved stability, and/or improved translatability.

The sequence of the RNA molecule may be codon optimized or deoptimized for expression in a desired host, such as a human cell.

In some aspects, the RNA molecules may include one or more structural and/or chemical modifications or alterations which impart useful properties to the polynucleotide including, in some aspects, the lack of a substantial induction of the innate immune response of a cell into which the polynucleotide is introduced. As used herein, a “structural” feature or modification is one in which two or more linked nucleotides are inserted, deleted, duplicated, inverted or randomized in an RNA molecule without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some aspects, the RNA molecule may include one or more modified nucleotides in addition to any 5′ cap structure. Naturally occurring nucleotide modifications are known in the art.

In some aspects, the RNA molecule does not include modified nucleotides, e.g., does not include modified nucleobases, and all of the nucleotides in the RNA molecule are conventional standard ribonucleotides A, U, G and C, with the exception of an optional 5′ cap that may include, for example, 7-methylguanosine, which is further described below. In some aspects, the RNA may include a 5′ cap comprising a 7′-methylguanosine, and the first 1, 2 or 3 5′ ribonucleotides may be methylated at the 2′ position of the ribose.

In some aspects, the RNA molecule described herein is a non-coding RNA molecule. A non-coding RNA (ncRNA) molecule includes a functional RNA molecule that is not translated into a peptide or polypeptide. Non-coding RNA molecules may include highly abundant and functionally important RNA molecules. In some aspects, the non-coding RNA is a functional mRNA molecule that is not translated into a peptide or polypeptide. The non-coding RNA may include modified nucleotides as described herein. Preferably, the RNA molecule is an mRNA

The RNA molecules of the present disclosure may be prepared by any method know in the art, including chemical synthesis and in vitro methods, such as RNA in vitro transcription. In some of the aspects, the RNA of the present disclosure is prepared using in vitro transcription.

In some aspects, the RNA molecule of the present disclosure is purified, e.g., such as by filtration that may occur via, e.g., ultrafiltration, diafiltration, or, e.g., tangential flow ultrafiltration/diafiltration.

In some aspects, the RNA molecule of the present disclosure is lyophilized to be temperature stable. 2. 5′ CAP In some aspects, the RNA molecule described herein includes a 5′ cap which generally “caps” the 5′ end of the RNA and stabilizes the RNA molecule.

In some aspects, the 5′ cap moiety is a natural 5′ cap. A “natural 5′ cap” is defined as a cap that includes 7-methylguanosine connected to the 5′ end of an mRNA molecule through a 5′ to 5′ triphosphate linkage. In some aspects, a guanosine nucleoside included in a 5′ cap may be modified, for example, by methylation at one or more positions (e.g., at the 7-position) on a base (guanine), and/or by methylation at one or more positions of a ribose. In some aspects, a guanosine nucleoside included in a 5′ cap comprises a 3′O methylation at a ribose (3′OMeG). In some aspects, a guanosine nucleoside included in a 5′ cap comprises methylation at the 7-position of guanine (m7G). In some aspects, a guanosine nucleoside included in a 5′ cap comprises methylation at the 7-position of guanine and a 3′O methylation at a ribose (m7(3′OMeG)). The 5′ cap may be incorporated during RNA synthesis (e.g., co-transcriptional capping) or may be enzymatically engineered after RNA transcription (e.g., post-transcriptional capping). In some aspects, co-transcriptional capping with a cap disclosed herein improves the capping efficiency of an RNA compared to co-transcriptional capping with an appropriate reference comparator. In some aspects, improving capping efficiency may increase a translation efficiency and/or translation rate of an RNA, and/or increase expression of an encoded polypeptide. In some aspects, capping is performed after purification, e.g., tangential flow filtration, of the RNA molecule.

In some aspects, an RNA described herein comprises a 5′ cap or a 5′ cap analog, e.g., a Cap 0, a Cap 1 or a Cap 2. In some aspects, a provided RNA does not have uncapped 5′-triphosphates. In some aspects, the 5′ end of the RNA is capped with a modified ribonucleotide.

In some aspects, the 5′ cap moiety is a 5′ cap analog. In some aspects, an RNA may be capped with a 5′ cap analog. Cap structures include, but are not limited to, 7mG(5′)ppp(5′)N,pN2p (Cap 0) and 7mG(5′)ppp(5′)N1mpNp (Cap 1). In some aspects, an RNA described herein comprises a Cap 0. Cap 0 is a N7-methyl guanosine connected to the 5′ nucleotide through a 5′ to 5′ triphosphate linkage, typically referred to as m7G cap or m7Gppp. In the cell, the Cap 0 structure is essential for efficient translation of the mRNA that carries the cap. An additional methylation on the 2′O position of the initiating nucleotide generates Cap 1, or referred to as m7GpppNm, wherein Nm denotes any nucleotide with a 2′O methylation. In some aspects, an RNA described herein comprises a Cap 1, e.g., as described herein. In some aspects, an RNA described herein comprises a Cap 2.

In some aspects, a Cap 0 structure comprises a guanosine nucleoside methylated at the 7-position of guanine (m7G). In some aspects, a Cap 0 structure is connected to an RNA via a 5′ to 5′-triphosphate linkage and is also referred to herein as m7Gppp or m7G(5′)ppp(5′). ⋅A 5′ cap may be methylated with the structure m7G (5′) ppp (5′) N (cap-0 structure) or a derivative thereof, wherein N is the terminal 5′ nucleotide of the nucleic acid carrying the 5′ cap, typically the 5′-end of an mRNA. An exemplary enzymatic reaction for capping may include use of Vaccinia Virus Capping Enzyme (VCE) that includes mRNA triphosphatase, guanylyl-transferase and guanine-7-methytransferase, which catalyzes the construction of N7-monomethylated Cap 0 structures. Cap 0 structure plays an important role in maintaining the stability and translational efficacy of the RNA molecule.

The 5′ cap of the RNA molecule may be further modified by a 2′-O-Methyltransferase which results in the generation of a Cap 1 structure (m7Gppp [m2′-O] N), which may further increase translation efficacy. In some aspects, a Cap 1 structure comprises a guanosine nucleoside methylated at the 7-position of guanine (m7G) and a 2′O methylated first nucleotide in an RNA (2′OmeN₁). In some aspects, a Cap 1 structure is connected to an RNA via a 5′- to 5′-triphosphate linkage and is also referred to herein as m7Gppp(2′OMeN₁) or m7G(5′)ppp(5′)(2′OMeN₁). In some aspects, N₁ is chosen from A, C, G, or U. In some aspects, N₁ is A. In some aspects, N₁ is C. In some aspects, N₁ is G. In some aspects, N₁ is U. In some aspects, a m7G(5′)ppp(5′)(2′OmeN₁) Cap 1 structure comprises a second nucleotide, N₂, which is a cap proximal nucleotide at position 2 and is chosen from A, G, C, or U (m7G(5′)ppp(5′)(2′OmeN₁)N₂). In some aspects, N₂ is A. In some aspects, N₂ is C. In some aspects, N₂ is G. In some aspects, N₂ is U.

In some aspects, a Cap 1 structure comprises a guanosine nucleoside methylated at the 7-position of guanine (m7G) and one or more additional modifications, e.g., methylation on a ribose, and a 2′O methylated first nucleotide in an RNA. In some aspects, a Cap 1 structure comprises a guanosine nucleoside methylated at the 7-position of guanine, a 3′O methylation at a ribose (m7(3′OMeG)), and a 2′O methylated first nucleotide in an RNA (2′OMeN₁). In some aspects, a Cap 1 structure is connected to an RNA via a 5′- to 5′-triphosphate linkage and is also referred to herein as m7(3′OMeG)ppp(2′OMeN₁) or m7(3′OMeG)(5′)ppp(5′)(2′OMeN₁). In some aspects, N₁ is chosen from A, C, G, or U. In some aspects, N₁ is A. In some aspects, N₁ is C. In some aspects, N₁ is G. In some aspects, N₁ is U. In some aspects, a m7(3′OMeG)(5′)ppp(5′)(2′OMeN₁) Cap 1 structure comprises a second nucleotide, N₂, which is a cap proximal nucleotide at position 2 and is chosen from A, G, C, or U (m7(3′OMeG)(5′)ppp(5′)(2′OmeN₁)N₂). In some aspects, N₂ is A. In some aspects, N₂ is C. In some aspects, N₂ is G. In some aspects, N₂ is U.

In some aspects, a second nucleotide in a Cap 1 structure may comprise one or more modifications, e.g., methylation. In some aspects, a Cap 1 structure comprising a second nucleotide comprising a 2′O methylation is a Cap 2 structure.

In some aspects, the RNA molecule may be enzymatically capped at the 5′ end using Vaccinia guanylyltransferase, guanosine triphosphate, and S-adenosyl-L-methionine to yield Cap 0 structure. An inverted 7-methylguanosine cap is added via a 5′ to 5′ triphosphate bridge. Alternatively, use of a 2′O-methyltransferase with Vaccinia guanylyltransferase yields the Cap 1 structure where in addition to the Cap 0 structure, the 2′OH group is methylated on the penultimate nucleotide. S-adenosyl-L-methionine (SAM) is a cofactor utilized as a methyl transfer reagent. Non-limiting examples of 5′ cap structures are those which, among other things, have enhanced binding of cap binding polypeptides, increased half-life, reduced susceptibility to 5′ endonucleases and/or reduced 5′ decapping, as compared to synthetic 5′ cap structures known in the art (or to a wild type, natural or physiological 5′ cap structure).

For example, recombinant Vaccinia Virus Capping Enzyme and recombinant 2′ O-methyltransferase enzyme may create a canonical 5′-5′-triphosphate linkage between the 5′-terminal nucleotide of an mRNA and a guanine cap nucleotide wherein the cap guanine includes an N7 methylation and the 5′-terminal nucleotide of the mRNA includes a 2′-O-methyl. Such a structure is termed the Cap 1 structure. This cap results in a higher translational-competency and cellular stability and a reduced activation of cellular pro-inflammatory cytokines, as compared, e.g., to other 5′ cap analog structures known in the art.

In some aspects, the 5′ terminal cap includes a cap analog, for example, a 5′ terminal cap may include a guanine analog. Exemplary guanine analogs include, but are not limited to, inosine, N1-methyl-guanosine, 2′-fluoro-guanosine, 7-deaza-guanosine, 8-oxo-guanosine, 2-amino-guanosine, LNA-guanosine, and 2-azido-guanosine.

In some aspects, the capping region may include a single cap or a series of nucleotides forming the cap. In this aspect the capping region may be from 1 to 10, e.g. 2-9, 3-8, 4-7, 1-5, 5-10, or at least 2, or 10 or fewer nucleotides in length. In this aspect the capping region is at least, at most, exactly, or between any two of 1, 2, 3, 4, 5, 6, 7, 8, 9, or 10 nucleotides in length. In some aspects, the cap is absent. In some aspects, the first and second operational regions may range from 3 to 40, e.g., 5-30, 10-20, 15, or at least 4, or 30 or fewer nucleotides in length and may comprise, in addition to a Start and/or Stop codon, one or more signal and/or restriction sequences. In some aspects, the first and second operational regions are at least, at most, exactly, or between any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length and may comprise, in addition to a Start and/or Stop codon, one or more signal and/or restriction sequences.

Further examples of 5′ cap structures include, but are not limited to, glyceryl, inverted deoxy abasic residue (moiety), 4′, 5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

In some aspects, the RNA molecule of the present disclosure comprises at least one 5′ cap structure. In some aspects, the RNA molecule of the present disclosure does not comprise a 5′ cap structure.

In one aspect, the 5′ capping structure comprises a modified 5′ Cap 1 structure (m⁷G⁺m³′-5′-ppp-5′-Am). In one aspect, the 5′ capping structure comprises is (3′OMe)-m₂^{7,3-O}Gppp (m₁^{2′-O})ApG (Trilink). This molecule is identical to the natural RNA cap structure in that it starts with a guanosine methylated at N7, and is linked by a 5′ to 5′ triphosphate linkage to the first coded nucleotide of the transcribed RNA (in this case, an adenosine). This guanosine is also methylated at the 3′ hydroxyl of the ribose to mitigate possible reverse incorporation of the cap molecule. The 2′ hydroxyl of the ribose on the adenosine is methylated, conferring a Cap1 structure.

### 3. Untranslated Regions (UTRS)

The 5′ UTR is a regulatory region situated at the 5′ end of a protein open reading frame that is transcribed into mRNA but not translated into an amino acid sequence or to the corresponding region in an RNA polynucleotide, such as an mRNA molecule. An untranslated region (UTR) may be present 5′ (upstream) of an open reading frame (5′ UTR) and/or 3′ (downstream) of an open reading frame (3′ UTR).

In some aspects, the UTR is derived from an mRNA that is naturally abundant in a specific tissue (e.g., lymphoid tissue), to which the mRNA expression is targeted. In some aspects, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). Accordingly, the UTR sequence may prolong protein synthesis in a tissue-specific manner.

In some aspects, the 5′ UTR and the 3′ UTR sequences are computationally derived. In some aspects, the 5′ UTR and the 3′ UTRs are derived from a naturally abundant mRNA in a tissue. The tissue may be, for example, liver, a stem cell or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte. In some aspects, the 5′ UTR and the 3′ UTR are derived from an alphavirus. In some aspects, the 5′ UTR and the 3′ UTR are from a wild type alphavirus.

### D. 5′UTRS

In some aspects, an RNA disclosed herein comprises a 5′ UTR. A 5′ UTR, if present, is located at the 5′ end and starts with the transcriptional start site upstream of the start codon of a protein encoding region. A 5′ UTR is downstream of the 5′ cap (if present), e.g. directly adjacent to the 5′ cap. The 5′ UTR may contain various regulatory elements, e.g., 5′ cap structure, stem-loop structure, and an internal ribosome entry site (IRES), which may play a role in the control of translation initiation.

In some aspects, a 5′ UTR disclosed herein comprises a cap proximal sequence, e.g., as disclosed herein. In some aspects, a cap proximal sequence comprises a sequence adjacent to a 5′ cap. In some aspects, a cap proximal sequence comprises nucleotides in positions+1, +2, +3, +4, and/or +5 of an RNA polynucleotide.

In some aspects, a Cap structure comprises one or more polynucleotides of a cap proximal sequence. In some aspects, a Cap structure comprises an m7 Guanosine cap and nucleotide+1 (N₁) of an RNA polynucleotide. In some aspects, a Cap structure comprises an m7 Guanosine cap and nucleotide+2 (N₂) of an RNA polynucleotide. In some aspects, a Cap structure comprises an m7 Guanosine cap and nucleotides+1 and +2 (N₁ and N₂) of an RNA polynucleotide.

Those skilled in the art, reading the present disclosure, will appreciate that, in some aspects, one or more residues of a cap proximal sequence (e.g., one or more of residues+1, +2, +3, +4, and/or +5) may be included in an RNA by virtue of having been included in a cap entity that (e.g., a Cap 1 structure, etc); alternatively, in some aspects, at least some of the residues in a cap proximal sequence may be enzymatically added (e.g., by a polymerase such as a T7 polymerase). For example, in certain exemplified aspects where a (m₂^{7,3′-O})Gppp(m^{2′-O})ApG cap is utilized, +1 and +2 residues are the (m₂^{7,3′-O}) A and G residues of the cap, and +3, +4, and +5 residues are added by polymerase (e.g., T7 polymerase).

In some aspects, a cap proximal sequence comprises N₁ and/or N₂ of a Cap structure, wherein N₁ and N₂ are any nucleotide, e.g., A, C, G or U. In some aspects, N₁ is A. In some aspects, N₁ is C. In some aspects, N₁ is G. In some aspects, N₁ is U. In some aspects, N₂ is A.

In some aspects, N₂ is C. In some aspects, N₂ is G. In some aspects, N₂ is U. In some aspects, a cap proximal sequence comprises N₁ and N₂ of a Cap structure and N₃, N₄ and N₅, wherein N₁ to N₅ correspond to positions +1, +2, +3, +4, and/or +5 of an RNA polynucleotide. In some aspects, N₁, N₂, N₃, N₄, or N₅ are any nucleotide, e.g., A, C, G or U. In some aspects, N₁N2 comprises any one of the following: AA, AC, AG, AU, CA, CC, CG, CU, GA, GC, GG, GU, UA, UC, UG, or UU. In some aspects, N₁N₂ comprises AG and N₃N₄N₅ comprises any one of the following: AAA, ACA, AGA, AUA, AAG, AGG, ACG, AUG, AAC, ACC, AGC, AUC, AAU, ACU, AGU, AUU, CAA, CCA, CGA, CUA, CAG, CGG, CCG, CUG, CAC, CCC, CGC, CUC, CAU, CCU, CGU, CUU, GAA, GCA, GGA, GUA, GAG, GGG, GCG, GUG, GAC, GCC, GGC, GUC, GAU, GCU, GGU, GUU, UAA, UCA, UGA, UUA, UAG, UGG, UCG, UUG, UAC, UCC, UGC, UUC, UAU, UCU, UGU, or UUU.

In some aspects, a cap proximal sequence comprises N₁ and N₂ of a Cap structure, and a sequence comprising: A₃A₄X₅ (; wherein X₅ is A, G, C, or U), where N₁ and N₂ are each independently chosen from: A, C, G, or U. In some aspects, N₁ is A and N₂ is G. In some aspects, X₅ is chosen from A, C, G or U. In some aspects, X₅ is A. In some aspects, X₅ is C. In some aspects, X₅ is G. In some aspects, X₅ is U.

In some aspects, a cap proximal sequence comprises N₁ and N₂ of a Cap structure, and a sequence comprising: C₃A₄X₅ (; wherein X₅ is A, G, C, or U), where N₁ and N₂ are each independently chosen from: A, C, G, or U. In some aspects, N₁ is A and N₂ is G. In some aspects, X₅ is chosen from A, C, G or U. In some aspects, X₅ is A. In some aspects, X₅ is C. In some aspects, X₅ is G. In some aspects, X₅ is U.

In some aspects, a cap proximal sequence comprises N₁ and N₂ of a Cap structure, and a sequence comprising X₃Y₄X₅ (; wherein X₃ or X₅ are each independently chosen from A, G, C, or U; and Y₄ is not C). In some aspects, N₁ and N₂ are each independently chosen from: A, C, G, or U. In some aspects, N₁ is A and N₂ is G. In some aspects, X₃ and X₅ is each independently chosen from A, C, G or U. In some aspects, X₃ and/or X₅ is A. In some aspects, X₃ and/or X₅ is C. In some aspects, X₃ and/or X₅ is G. In some aspects, X₃ and/or X₅ is U. In some aspects, Y₄ is C. In other aspects, Y₄ is not C. In some aspects, Y₄ is A. In some aspects, Y₄ is G. In other aspects, Y₄ is not G. In some aspects, Y₄ is U.

In some aspects, a cap proximal sequence comprises N₁ and N₂ of a Cap structure, and a sequence comprising A₃C₄A₅ ( ). In some aspects, N₁ and N₂ are each independently chosen from: A, C, G, or U. In some aspects, N₁ is A and N₂ is G.

In some aspects, a cap proximal sequence comprises N₁ and N₂ of a Cap structure, and a sequence comprising A₃U₄G₅ ( ). In some aspects, N₁ and N₂ are each independently chosen from: A, C, G, or U. In some aspects, N₁ is A and N₂ is G.

In one aspect, an RNA disclosed herein comprises a 5′ UTR comprising a sequence having at least, at most, exactly, or between any two of 99%, 98%, 97%, 96%, 95%, 90%, 85%, or 80% identity to a 5′ UTR provided in any of SEQ ID NO: 17 to 19 in which the transcribed 5′ cap structure is underlined. In one aspect, the 5′ UTR comprises a sequence of any of SEQ ID NO: 17 to 19, in which the transcribed 5′ cap structure is underlined.

In one aspect, an RNA disclosed herein comprises a 5′ UTR comprising a sequence having at least, at most, exactly, or between any two of 99%, 98%, 97%, 96%, 95%, 90%, 85%, or 80% identity to a 5′ UTR provided in any of SEQ ID NO: 46 to 48. In one aspect, the 5′ UTR comprises a sequence of any of SEQ ID NO: 46 to 48, in which the transcribed 5′ cap structure is underlined.

(RNA)
SEQ ID NO: 48
GAΨAGGCGGCGCAΨGAGAGAAGCCCAGACCAAΨΨACCΨACCCAAA

### 1. 3′ UTRS

In some aspects, an RNA disclosed herein comprises a 3′ UTR. A 3′ UTR, if present, is situated downstream of a protein coding sequence open reading frame, e.g., downstream of the termination codon of a protein-encoding region. A 3′ UTR is typically the part of an mRNA which is located between the protein coding sequence and the poly-A tail of the mRNA. Thus, in some aspects, the 3′ UTR is upstream of the poly-A sequence (if present), e.g. directly adjacent to the poly-A sequence. The 3′ UTR may be involved in regulatory processes including transcript cleavage, stability and polyadenylation, translation, and mRNA localization.

A 3′ UTR may also comprise elements, which are not encoded in the template, from which an RNA is transcribed, but which are added after transcription during maturation, e.g. a poly-A tail. A 3′ UTR of the mRNA is not translated into an amino acid sequence. In some aspects, an RNA disclosed herein comprises a 3′ UTR comprising an F element and/or an I element. In some aspects, a 3′ UTR or a proximal sequence thereto comprises a restriction site. In some aspects, a restriction site is a BamHI site. In some aspects, a restriction site is a Xhol site.

In some aspects, an RNA disclosed herein comprises a 3′ UTR comprising a sequence having at least, at most, exactly, or between any two of 99%, 98%, 97%, 96%, 95%, 90%, 85%, or 80% identity to a 3′ UTR provided in any of SEQ ID NO: 20 to 22. In one aspect, the 3′ UTR comprises a sequence of any of SEQ ID NO: 20 to 22. SEQ ID NO: 22 (RNA) is set forth as:

CΨCGAGCΨGGΨACΨGCAΨGCACGCAAΨGCΨAGCΨGCCCCΨΨΨCCCGΨCC
ΨGGGΨACCCCGAGΨCΨCCCCCGACCΨCGGGΨCCCAGGΨAΨGCΨCCCACC
ΨCCACCΨGCCCCACΨCACCACCΨCΨGCΨAGΨΨCCAGACACCΨCCCAAGC
ACGCAGCAAΨGCAGCΨCAAAACGCΨΨAGCCΨAGCCACACCCCCACGGGA
AACAGCAGΨGAΨΨAACCΨΨΨAGCAAΨAAACGAAAGΨΨΨAACΨAAGCΨAΨ
ACΨAACCCCAGGGΨΨGGΨCAAΨΨΨCGΨGCCAGCCACACCCΨGGAGCΨAG
C.

In one aspect, an RNA disclosed herein comprises a 3′ UTR comprising a sequence having at least, at most, exactly, or between any two of 99%, 98%, 97%, 96%, 95%, 90%, 85%, or 80% identity to a 3′ UTR provided in any of SEQ ID NO: 23 to 25. In one aspect, the 3′ UTR comprises a sequence of any of SEQ ID NO: 23 to 25. SEQ ID NO: 25 (RNA) is set forth as:

AΨACAGCAGCAAΨΨGGCAAGCΨGCΨΨACAΨAGAACΨCGCGGCGAΨΨGGC
AΨGCCGCCΨΨAAAAΨΨΨΨΨAΨΨΨΨAΨΨΨΨΨCΨΨΨΨCΨΨCCGAAΨCGGAΨ
ΨΨΨGΨΨΨΨΨAAΨAΨΨΨC.

### 2. Open Reading Frame (ORF)

The 5′ and 3′ UTRs may be operably linked to an open reading frame (ORF), which may be a sequence of codons that is capable of being translated into a polypeptide of interest. An open reading frame may be a sequence of several DNA or RNA nucleotide triplets, which may be translated into a peptide or protein. An ORF may begin with a start codon, e.g., a combination of three subsequent nucleotides coding usually for the amino acid methionine (ATG or AUG), at its 5′ end and a subsequent region, which usually exhibits a length which is a multiple of 3 nucleotides. An open reading frame may terminate with at least one stop codon, including but not limited to TAA, TAG, TGA or UAA, UAG or UGA, or any combination thereof. In some aspects, an open reading frame may terminate with one, two, three, four or more stop codons, including but not limited to TAATAA (SEQ ID NO: 27), TAATAG (SEQ ID NO: 28), TAATGA (SEQ ID NO: 29), TAGTGA (SEQ ID NO: 30), TAGTAA (SEQ ID NO: 31), TAGTAG (SEQ ID NO: 32), TGATGA (SEQ ID NO: 33), TGATAG (SEQ ID NO: 34), TGATAA (SEQ ID NO: 35) or UAAUAA (SEQ ID NO: 36), UAAUAG (SEQ ID NO: 37), UAAUGA (SEQ ID NO: 38), UAGUGA (SEQ ID NO: 39), UAGUAA (SEQ ID NO:40), UAGUAG (SEQ ID NO: 41), UGAUGA (SEQ ID NO: 42), UGAUAG (SEQ ID NO: 43), UGAUAA (SEQ ID NO: 44), or any combination thereof. An open reading frame may be isolated or it may be incorporated in a longer nucleic acid sequence, e.g. in a vector or an mRNA. An open reading frame may also be termed “(protein) coding region” or “coding sequence”.

As stated herein, the RNA molecule may include one (monocistronic), two (bicistronic) or more (multicistronic) open reading frames.

In some aspects, the ORF encodes a non-structural viral gene. In some aspects, the ORF further includes one or more subgenomic promoters. In some aspects, the RNA molecule includes a subgenomic promoter operably linked to the ORF. In some aspects, a first RNA molecule does not include an ORF encoding any polypeptide of interest, whereas a second RNA molecule includes an ORF encoding a polypeptide of interest. In some aspects, the first RNA molecule does not include a subgenomic promoter.

The present disclosure provides for an RNA molecule comprising at least one open reading frame encoding a respiratory syncytial virus (RSV) polypeptide. In some aspects, an RNA molecule comprising at least one open reading frame encoding a RSV F protein.

The present disclosure provides for an RNA molecule comprising at least one open reading frame encoding a polypeptide derived from influenza, such as, for example HA and/or NA. In some aspects, an RNA molecule comprising at least one open reading frame encoding influenza HA and/or NA.

### 3. Genes of Interest

The RNA molecules described herein may include a gene of interest. The gene of interest encodes a polypeptide of interest. Non-limiting examples of polypeptides of interest include, e.g., biologics, antibodies, vaccines, therapeutic polypeptides or peptides, cell penetrating peptides, secreted polypeptides, plasma membrane polypeptides, cytoplasmic or cytoskeletal polypeptides, intracellular membrane bound polypeptides, nuclear polypeptides, polypeptides associated with human disease, targeting moieties, those polypeptides encoded by the human genome for which no therapeutic indication has been identified but which nonetheless have utility in areas of research and discovery, or combinations thereof. The sequence for a particular gene of interest is readily identified by one of skill in the art using public and private databases, e.g., GENBANK®.

In some aspects, the RNA molecules include a coding region for a gene of interest. In some aspects, a gene of interest is or comprises an antigenic polypeptide or an immunogenic variant or an immunogenic fragment thereof. In some aspects, an antigenic polypeptide comprises one epitope from an antigen. In some aspects, an antigenic polypeptide comprises a plurality of distinct epitopes from an antigen. In some aspects, an antigenic polypeptide comprising a plurality of distinct epitopes from an antigen is polyepitopic. In some aspects, an antigenic polypeptide comprises: an antigenic polypeptide from an allergen, a viral antigenic polypeptide, a bacterial antigenic polypeptide, a fungal antigenic polypeptide, a parasitic antigenic polypeptide, an antigenic polypeptide from an infectious agent, an antigenic polypeptide from a pathogen, a tumor antigenic polypeptide, or a self-antigenic polypeptide.

The term “antigen” may refer to a substance, which is capable of being recognized by the immune system, e.g. by the adaptive immune system, and which is capable of eliciting an antigen-specific immune response, e.g. by formation of antibodies and/or antigen-specific T cells as part of an adaptive immune response. An antigen may be or may comprise a peptide or protein, which may be presented by the MHC to T-cells. An antigen may be the product of translation of a provided nucleic acid molecule, e.g. an RNA molecule comprising at least one coding sequence as described herein. In addition, fragments, variants and derivatives of an antigen, such as a peptide or a protein, comprising at least one epitope are understood as antigens.

In some aspects, an RNA encoding a gene of interest, e.g., an antigen, is expressed in cells of a subject treated to provide a gene of interest, e.g., an antigen. In some aspects, the RNA is transiently expressed in cells of the subject. In some aspects, expression of a gene of interest, e.g., an antigen, is at the cell surface. In some aspects, a gene of interest, e.g., an antigen, is expressed and presented in the context of MHC. In some aspects, expression of a gene of interest, e.g., an antigen, is into the extracellular space, e.g., the antigen is secreted.

In some aspects, the RNA molecules include a coding region for a gene of interest, e.g., an antigen. In some aspects, the RNA molecules include a coding region for a gene of interest, e.g., an antigen, that is derived from a pathogen associated with an infectious disease. In some aspects, the RNA molecules include a coding region for a gene of interest, e.g., an antigen, that is derived from respiratory syncytial virus (RSV) and/or an antigen derived from influenza.

In some aspects, an RNA polynucleotide described herein or a composition or medical preparation comprising the same comprises a nucleotide sequence disclosed herein. In some aspects, an RNA polynucleotide comprises a sequence having at least 80% identity to a nucleotide sequence disclosed herein. In some aspects, an RNA polynucleotide comprises a sequence encoding a polypeptide having at least 80% identity to a polypeptide sequence disclosed herein. In some aspects, an RNA polynucleotide described herein or a composition or medical preparation comprising the same is transcribed by a DNA template. In some aspects, a DNA template used to transcribe an RNA polynucleotide described herein comprises a sequence complementary to an RNA polynucleotide. In some aspects, a gene of interest described herein is encoded by an RNA polynucleotide described herein comprising a nucleotide sequence disclosed herein. In some aspects, an RNA polynucleotide encodes a polypeptide having at least 80% identity to a polypeptide sequence disclosed herein. In some aspects, a polypeptide described herein is encoded by an RNA polynucleotide transcribed by a DNA template comprising a sequence complementary to an RNA polynucleotide.

In some aspects, the RNA molecule encodes a RSV F protein comprising the sequence of any one of SEQ ID NOs: 1-6, or a fragment or variant thereof.

In some aspects, the RNA molecule encodes a RSV F protein synthesized from the nucleic acid sequence comprising any one of SEQ ID NOs: 7 to 10, or fragment or variant thereof.

### 4. Poly-a Tail

In some aspects, RNA molecules disclosed herein comprise a poly-adenylate (poly-A) sequence, e.g., as described herein. In some aspects, a poly-A sequence is situated downstream of a 3′ UTR, e.g., adjacent to a 3′ UTR. A “poly-A tail” or “poly-A sequence” refers to a stretch of consecutive adenine residues, which may be attached to the 3′ end of the RNA molecule. Poly-A sequences are known to those of skill in the art and may follow the 3′ UTR in the RNA molecules described herein. The poly-A tail may increase the half-life of the RNA molecule.

RNA molecules disclosed herein may have a poly-A sequence attached to the free 3′-end of the RNA by a template-independent RNA polymerase after transcription or a poly-A sequence encoded by DNA and transcribed by a template-dependent RNA polymerase. In some aspects, a poly-A sequence is attached during RNA transcription, e.g., during preparation of in vitro transcribed RNA, based on a DNA template comprising repeated dT nucleotides (deoxythymidylate) in the strand complementary to the coding strand.

The DNA sequence encoding a poly-A sequence (coding strand) is referred to as poly-A cassette. In some aspects, the poly-A cassette present in the coding strand of DNA essentially consists of dA nucleotides, but is interrupted by a random sequence of the four nucleotides (dA, dC, dG, and dT). Such a random sequence may be at least, at most, exactly, or between any two of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. Such a cassette is disclosed in WO 2016/005324 A₁, which is hereby incorporated by reference in its entirety. Any poly-A cassette disclosed in WO 2016/005324 A₁ may be used in the present invention. A poly-A cassette that essentially consists of dA nucleotides, but is interrupted by a random sequence having an equal distribution of the four nucleotides (dA, dC, dG, dT) and having a length of e.g., 5 to 50 nucleotides, shows, on DNA level, constant propagation of plasmid DNA in E. coli and is still associated, on RNA level, with the beneficial properties with respect to supporting RNA stability and translational efficiency is encompassed.

In some aspects, the poly-A sequence contained in an RNA polynucleotide described herein essentially consists of adenosine nucleotides, but is interrupted by a random sequence of the four nucleotides (A, C, G, U). Such a random sequence may be at least, at most, exactly, or between any two of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some aspects, no nucleotides other than adenosine nucleotides flank a poly-A sequence at its 3′-end, e.g., the poly-A sequence, is not masked or followed at its 3′-end by a nucleotide other than adenosine.

In some aspects, the RNA molecule may further include an endonuclease recognition site sequence immediately downstream of the poly-A tail sequence. The RNA molecule may further include a poly-A polymerase recognition sequence (e.g. AAUAAA) near its 3′ end.

The poly-A sequence may be of any length. In some aspects, the poly-A tail may include 5 to 300 nucleotides in length. In some aspects, the RNA molecule includes a poly-A tail that comprises, essentially consists of, or consists of a sequence of about 25 to about 400 adenosine nucleotides, a sequence of about 50 to about 400 adenosine nucleotides, a sequence of about 50 to about 300 adenosine nucleotides, a sequence of about 50 to about 250 adenosine nucleotides, a sequence of about 60 to about 250 adenosine nucleotides, or a sequence of about 40 to about 100 adenosine nucleotides. In some aspects, the poly-A tail comprises, essentially consists of, or consists of at least, at most, exactly, or between any two of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 adenosine nucleotides. In this context, “essentially consists of” means that most nucleotides in the poly-A sequence, typically at least 75%, at least 80%, at least 85%, at least 90%, at least 95%, at least 96%, at least 97%, at least 98%, or at least 99% by number of nucleotides in the poly-A sequence are adenosine nucleotides, but permits that remaining nucleotides are nucleotides other than adenosine nucleotides, such as uridine, guanosine, or cytosine. In this context, “consists of” means that all nucleotides in the poly-A sequence, e.g., 100% by number of nucleotides in the poly-A sequence, are adenosine nucleotides.

In some aspects, the RNA molecule includes a poly-A tail that includes a sequence of greater than 30 adenosine nucleotides. In some aspects, the RNA molecule includes a poly-A tail that includes about 40 adenosine nucleotides. In some aspects, the RNA molecule includes a poly-A tail that includes about 80 adenosine nucleotides. In some aspects, the 3′ poly-A tail has a stretch of at least 10 consecutive adenosine residues and at most 300 consecutive adenosine residues. In some specific aspects, the RNA molecule includes about 40 consecutive adenosine residues. In some aspects, the RNA molecule includes about 80 consecutive adenosine residues. Poly-A tails may play key regulatory roles in enhancing translation efficiency and regulating the efficiency of mRNA quality control and degradation. Short sequences or hyperpolyadenylation may signal for RNA degradation. Some designs include a poly-A tails of about 40 adenosine nucleotides, about adenosine nucleotides.

In some aspects, a poly-A tail may be located within an RNA molecule or other nucleic acid molecule, such as, e.g., in a vector, for example, in a vector serving as template for the generation of an RNA, e.g. an mRNA, e.g., by transcription of the vector. In some aspects, the RNA molecule may not include a poly-A tail.

In one aspect, an RNA disclosed herein comprises a poly-A tail comprising a sequence having at least, at most, exactly, or between any two of 99%, 98%, 97%, 96%, 95%, 90%, 85%, or 80% identity to SEQ ID NO: 26. In one aspects, the poly-A tail comprises a sequence of SEQ ID NO: 26.

### 5. Self-Amplifying RNA (SaRNA)

In some aspects, the RNA molecule may be an saRNA. “Self-amplifying RNA,” “self-amplifying RNA,” “self-replicating” and “replicon” may all be used interchangeably, and refer to RNA with the ability to replicate itself. Self-amplifying RNA molecules may be produced by using replication elements derived from, e.g. alphaviruses, and substituting the structural viral polypeptides with a nucleotide sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNA molecules. These daughter RNA molecules, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the antigen. The overall result of this sequence of transcriptions is an amplification in the number of the introduced saRNA molecules and so the encoded gene of interest, e.g., a viral antigen, becomes a major polypeptide product of the cells.

In some aspects, the self-amplifying RNA includes at least one or more genes including any one of viral replicases, viral proteases, viral helicases and other nonstructural viral proteins, or combination thereof. In some aspects, the self-amplifying RNA may also include 5′- and 3′-end tractive replication sequences, and optionally a heterologous sequence that encodes a desired amino acid sequence (e.g., an antigen of interest). A subgenomic promoter that directs expression of the heterologous sequence may be included in the self-amplifying RNA. Optionally, the heterologous sequence (e.g., an antigen of interest) may be fused in frame to other coding regions in the self-amplifying RNA and/or may be under the control of an internal ribosome entry site (IRES).

In one aspect, a self-amplifying RNA disclosed herein comprises a subgenomic promoter comprising a sequence having at least, at most, exactly, or between any two of 99%, 98%, 97%, 96%, 95%, 90%, 85%, or 80% identity to SEQ ID NO: 49. In one aspect, the subgenomic promoter comprises a sequence of SEQ ID NO: 49.
SEQ ID NO: 49 (RNA) is set forth as:

CCUGAAUGGACUACGACAUAGUCUAGUCCGCCAAG

In some aspects, a self-amplifying RNA molecule described herein encodes (i) an RNA-dependent RNA polymerase that may transcribe RNA from the self-amplifying RNA molecule and (ii) a polypeptide of interest, e.g., a viral antigen. In some aspects, the polymerase may be an alphavirus replicase, e.g., including any one of alphavirus protein nsP1, nsP2, nsP3, nsP4, and any combination thereof.

In one aspect, a self-amplifying RNA disclosed herein comprises an alphavirus replicase, e.g., including any one of alphavirus protein nsP1, nsP2, nsP3, nsP4, and any combination thereof, comprising a sequence having at least, at most, exactly, or between any two of 99%, 98%, 97%, 96%, 95%, 90%, 85%, or 80% identity to SEQ ID NO: 50-53, respectively. In one aspect, the alphavirus protein nsP1, nsP2, nsP3 and nsP4 each comprise a sequence of SEQ ID NO: 50-53, respectively.
SEQ ID NO: 50 (nsP1 RNA); SEQ ID NO: 51 (NSP2 RNA); SEQ ID NO: 52 (NSP3 RNA); SEQ ID NO: 53 (NSP4 RNA)

In some aspects, the self-amplifying RNA molecule may have two open reading frames. The first (5′) open reading frame may encode a replicase; the second (3′) open reading frame may encode a polypeptide comprising an antigen of interest. In some aspects the RNA may have additional (e.g., downstream) open reading frames, e.g., to encode further antigens or to encode accessory polypeptides.

In some aspects, the saRNA molecule further includes (1) an alphavirus 5′ replication recognition sequence, and (2) an alphavirus 3′ replication recognition sequence. In some aspects, the 5′ sequence of the self-amplifying RNA molecule is selected to ensure compatibility with the encoded replicase.

In some aspects, the self-amplifying RNA molecule may encode a single polypeptide antigen or, optionally, two or more of polypeptide antigens linked together in a way that each of the sequences retains its identity (e.g., linked in series) when expressed as an amino acid sequence. The polypeptides generated from the self-amplifying RNA may then be produced as a fusion polypeptide or engineered in such a manner to result in separate polypeptide or peptide sequences.

In some aspects, the self-amplifying RNA described herein may encode one or more polypeptide antigens that include a range of epitopes. In some aspects, the self-amplifying RNA described herein may encode epitopes capable of eliciting either a helper T-cell response or a cytotoxic T-cell response or both.

### IV. RNA Transcription

In some aspects, the RNA disclosed herein is produced by in vitro transcription or chemical synthesis. In the context of the present disclosure, the term “transcription” relates to a process, wherein the genetic code in a DNA sequence is transcribed into RNA. Subsequently, the RNA may be translated into peptide or protein.

According to the present disclosure, “transcription” comprises “in vitro transcription” or “IVT,” which refers to the process whereby transcription occurs in vitro in a non-cellular system to produce a synthetic RNA product for use in various applications, including, e.g., production of protein or polypeptides. Cloning vectors may be applied for the generation of transcripts. These cloning vectors are generally designated as transcription vectors and are according to the present invention encompassed by the term “vector.” According to specific aspects, the RNA used is in vitro transcribed RNA (IVT-RNA) and may be obtained by in vitro transcription of an appropriate DNA template. The promoter for controlling transcription may be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

Synthetic IVT RNA products may be translated in vitro or introduced directly into cells, where they may be translated. With respect to RNA, the term “expression” or “translation” relates to the process in the ribosomes of a cell by which a strand of mRNA directs the assembly of a sequence of amino acids to make a peptide or protein. Such synthetic RNA products include, e.g., but are not limited to mRNA molecules, saRNA molecules, antisense RNA molecules, shRNA molecules, long non-coding RNA molecules, ribozymes, aptamers, guide RNA molecules (e.g., for CRISPR), ribosomal RNA molecules, small nuclear RNA molecules, small nucleolar RNA molecules, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In some aspects, an mRNA is produced by in vitro transcription using a DNA template where DNA refers to a nucleic acid that contains deoxyribonucleotides. In some aspects, an RNA disclosed herein is in vitro transcribed RNA (IVT-RNA) and may be obtained by in vitro transcription of an appropriate DNA template. The promoter for controlling transcription may be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In some aspects, starting material for IVT may include linearized DNA template, nucleotides, RNase inhibitor, pyrophosphatase, and/or T7 RNA polymerase. In some aspects, the IVT process is conducted in a bioreactor. The bioreactor may comprise a mixer. In some aspects, nucleotides may be added into the bioreactor throughout the IVT process.

In some aspects, one or more post-IVT agents are added into the IVT mixture comprising RNA in the bioreactor after the IVT process. Exemplary post-IVT agents may include DNAse I configured to digest the linearized DNA template, and proteinase K configured to digest DNAse I and T7 RNA polymerase. In some aspects, the post-IVT agents are incubated with the mixture in the bioreactor after IVT. In some aspects, the bioreactor may contain at least, at most, exactly, or between any two of 60, 70, 80, 90, 100, 110, 120, 130, 140, 150,160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 or more liters IVT mixture. The IVT mixture may have an RNA concentration at least, at most, exactly, or between any two of 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 7.0, 8.0, 9.0, 10, 11, 12, 13, 14, 15, 16, 17, 18, 19, 20, 30, 40, 50, 60, 70, 80, 90, and 100 mg/mL or more RNA.

In some aspects, the IVT mixture may include residual spermidine, residual DNA, residual proteins, peptides, HEPES, EDTA, ammonium sulfate, cations (e.g., Mg2+, Na+, Ca2+), RNA fragments, residual nucleotides, free phosphates, or any combinations thereof.

In some aspects, at least a portion of the IVT mixture is filtered. The IVT mixture may be filtered via ultrafiltration and/or diafiltration to remove at least some impurities from the IVT mixture and/or to change buffer solution for the at least a portion of IVT mixture to produce a concentrated RNA solution as a retentate.

In some aspects, both “ultrafiltration” and “diafiltration” refer to a membrane filtration process. Ultrafiltration typically uses membranes having pore sizes of at least, at most, exactly, or between any two of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1 μm. In some aspects, ultrafiltration membranes are typically classified by molecular weight cutoff (MWCO) rather than pore size. For example, the MWCO may be at least, at most, exactly, or between any two of 30 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 500 kDa, 600 kDa, 700 kDa, 800 kDa, 900 kDa, 1000 kDa, 2000 kDa, 3000 kDa, 4000 kDa, 5000 kDa, 6000 kDa, 7000 kDa, 8000 kDa, 9000 kDa, and 10000 kDa. A skilled artisan will understand that filtration membranes may be of different suitable materials, including, e.g., polymeric, cellulose, ceramic, etc., depending upon the application. In some aspects, membrane filtration may be more desirable for large volume purification process.

In some aspects, ultrafiltration and diafiltration of the IVT mixture for purifying RNA may include (1) Direct Flow Filtration (DFF), also known as “dead-end” filtration, that applies a feed stream perpendicular to the membrane face and attempts to pass 100% of the fluid through the membrane, and/or (2) Tangential Flow Filtration (TFF), also known as crossflow filtration, where a feed stream passes parallel to the membrane face as one portion passes through the membrane (permeate) while the remainder (retentate) is retained and/or recirculated back to the feed tank.

In some aspects, the filtering of the IVT mixture is conducted via TFF that comprises an ultrafiltration step, a first diafiltration step, and a second diafiltration step. In some aspects, the first diafiltration step is conducted in the presence of ammonium sulfate. The first diafiltration step may be configured to remove a majority of impurities from the IVT mixture. In some aspects, the second diafiltration step is conducted without ammonium sulfate. The second diafiltration step may be configured to transfer the RNA into a DS buffer formulation.

A filtration membrane with an appropriate MWCO may be selected for the ultrafiltration in the TFF process. The MWCO of a TFF membrane determines which solutes may pass through the membrane into the filtrate and which are retained in the retentate. The MWCO of a TFF membrane may be selected such that substantially all of the solutes of interest (e.g., desired synthesized RNA species) remains in the retentate, whereas undesired components (e.g., excess ribonucleotides, small nucleic acid fragments such as digested or hydrolyzed DNA template, peptide fragments such as digested proteins and/or other impurities) pass into the filtrate. In some aspects, the retentate comprising desired synthesized RNA species may be re-circulated to a feed reservoir to be re-filtered in additional cycles. In some aspects, a TFF membrane may have a MWCO equal to at least, at most, exactly, or between any two of 30 kDa, 40 kDa, 50 kDa, 60 kDa, 70 kDa, 80 kDa, 90 kDa, or more. In some aspects, a TFF membrane may have a MWCO equal to at least, at most, exactly, or between any two of 100 kDa, 150 kDa, 200 kDa, 250 kDa, 300 kDa, 350 kDa, 400 kDa, or more. In some aspects, a TFF membrane may have a MWCO of about 250-350 kDa. In some aspects, a TFF membrane (e.g., a cellulose-based membrane) may have a MWCO of about 30-300 kDa; in some aspects about 50-300 kDa, about 100-300 kDa, or about 200-300 kDa.

Diafiltration may be performed either discontinuously, or alternatively, continuously. For example, in continuous diafiltration, a diafiltration solution may be added to a sample feed reservoir at the same rate as filtrate is generated. In this way, the volume in the sample reservoir remains constant but small molecules (e.g., salts, solvents, etc.) that may freely permeate through a membrane are removed. Using solvent removal as an example, each additional diafiltration volume (DV) reduces the solvent concentration further. In discontinuous diafiltration, a solution is first diluted and then concentrated back to the starting volume. This process is then repeated until the desired concentration of small molecules (e.g. salts, solvents, etc.) remaining in the reservoir is reached. Each additional diafiltration volume (DV) reduces the small molecule (e.g., solvent) concentration further. Continuous diafiltration typically requires a minimum volume for a given reduction of molecules to be filtered. Discontinuous diafiltration, on the other hand, permits fast changes of the retentate condition, such as pH, salt content, and the like. In some aspects, the first diafiltration step is conducted with diavolumes equal to at least, at most, exactly, or between any two of 2, 3, 4, 5, 6, 7, 8, 9, 10, or more. In some aspects, the second diafiltration step is conducted with diavolumes equal to at least, at most, exactly, or between any two of 5, 6, 7, 8, 9, 10, 11, 12, 13, 14, 15, 16, 17, 18, 19, 20, or more. In some aspects, the first diafiltration step is conducted with 5 diavolumes, and second diafiltration step is conducted with 10 diavolumes.

In some aspects, for the ultrafiltration and/or diafiltration, the IVT mixture is filtered at a rate equal to at least, at most, exactly, or between any two of 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 600, 700, 800, 900, or 1000 L/m2 of filter area per hour, or more. The concentrated RNA solution may comprise at least, at most, exactly, or between any two of 2.0, 2.1, 2.2, 2.3, 2.4, or 2.5 mg/mL single stranded RNA.

The bioburden of the concentrated RNA solution via filtration to obtain an RNA product solution may also be reduced, in some aspects. The filtration for reducing bioburden may be conducted using one or more filters. The one or more filters may include a filter with a pore size of at least, at most, exactly, or between any two of 0.2 μm, 0.45 μm, 0.65 μm, 0.8 μm, or any other pore size configured to remove bioburdens.

As one example, reducing the bioburden may include draining a retentate tank containing retentate obtained from the ultrafiltration and/or diafiltration to obtain the retentate. Reducing the bioburden may include flushing a filtration system for ultrafiltration and/or diafiltration using a wash buffer solution to obtain a wash pool solution comprising residue RNA remaining in the filtration system. The retentate may be filtered to obtain a filtered retentate. The wash pool solution may be filtered using a first 0.2 μm filter to obtain a filtered wash pool solution. The retentate may be filtered using the first 0.2 μm filter or another 0.2 μm filter.

The filtered wash pool solution and the filtered retentate may be combined to form a combined pool solution. The combined pool solution may be filtered using a second 0.2 μm filter to obtain a filtered combined pool solution, which is further filtered using a third 0.2 μm filter to produce an RNA product solution.

### V. RNA Encapsulation

The RNA in an RNA product solution may be encapsulated, and the RNA solution may further comprise at least one encapsulating agent. In one aspect, the encapsulating agent comprises a lipid, a lipid nanoparticle (LNP), lipoplexes, polymeric particles, polyplexes, and monolithic delivery systems, and a combination thereof.

In one aspect, the encapsulating agent is a lipid, and produced is lipid nanoparticle (LNP)-encapsulated RNA. Without intending to be bound by any theory, it is believed that the cationic or cationically ionizable lipid or lipid-like material and/or the cationic polymer combine together with the nucleic acid to form aggregates, and this aggregation results in colloidally stable particles. A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof. A lipid is a substance that is insoluble in water and extractable with an organic solvent. Compounds other than those specifically described herein are understood by one of skill in the art as lipids, and are encompassed by the compositions and methods of the present disclosure. A lipid component and a non-lipid may be attached to one another, either covalently or non-covalently.

In some aspects, LNPs may be designed to protect RNA molecules (e.g., saRNA, mRNA) from extracellular RNases and/or may be engineered for systemic delivery of the RNA to target cells. In some aspects, such LNPs may be particularly useful to deliver RNA molecules (e.g., mRNA, saRNA, modRNA) when RNA molecules are intravenously administered to a subject in need thereof. In some aspects, such LNPs may be particularly useful to deliver RNA molecules (e.g., saRNA, mRNA) when RNA molecules are intramuscularly administered to a subject in need thereof.

In one aspect, the RNA in the RNA solution is at a concentration of <1 mg/mL. In another aspect, the RNA is at a concentration of at least about 0.05 mg/mL. In another aspect, the RNA is at a concentration of at least about 0.5 mg/mL. In another aspect, the RNA is at a concentration of at least about 1 mg/mL. In another aspect, the RNA concentration is from about 0.05 mg/mL to about 0.5 mg/mL. In another aspect, the RNA is at a concentration of at least 10 mg/mL. In another aspect, the RNA is at a concentration of at least 50 mg/mL. In some aspects, the RNA is at a concentration of at least, at most, exactly, or between any two of about 0.05 mg/mL, 0.5 mg/mL, 1 mg/mL, 10 mg/mL, 50 mg/mL, 75 mg/mL, 100 mg/mL, 150 mg/mL, 200 mg/mL, 250 mg/mL, 300 mg/mL, 400 mg/mL, or more.

The present disclosure provides for an RNA solution and lipid preparation mixture or compositions thereof comprising at least one RNA encoding, e.g., an antigen (e.g., an RSV prefusion F protein and/or an antigen derived from influenza, e.g., HA and/or NA) complexed with, encapsulated in, and/or formulated with one or more lipids, and forming lipid nanoparticles (LNPs), liposomes, lipoplexes and/or nanoliposomes. In some aspects, the composition comprises a lipid nanoparticle.

A lipid nanoparticle or LNP refers to particles of any morphology generated when a cationic lipid and optionally one or more further lipids are combined, e.g. in an aqueous environment and/or in the presence of RNA. In some aspects, lipid nanoparticles are included in a formulation that may be used to deliver an active agent or therapeutic agent, such as a nucleic acid (e.g., mRNA, saRNA, modRNA) to a target site of interest (e.g., cell, tissue, organ, tumor, and the like). In some aspects, the lipid nanoparticles of the present disclosure comprise a nucleic acid. Such lipid nanoparticles typically comprise a cationic lipid and one or more excipients, e.g., one or more neutral lipids, charged lipids, steroids, polymer conjugated lipids, or combinations thereof. In some aspects, the active agent or therapeutic agent, such as a nucleic acid (e.g., mRNA, saRNA, modRNA), may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response. The nucleic acid (e.g., mRNA, saRNA, modRNA) or a portion thereof may also be associated and complexed with the lipid nanoparticle. A lipid nanoparticle may comprise any lipid capable of forming a particle to which the nucleic acids are attached, or in which the one or more nucleic acids are encapsulated.

In some aspects, provided RNA molecules (e.g., mRNA, saRNA, modRNA) may be formulated with LNPs. In some aspects, the lipid nanoparticles may have a mean diameter of about 1 to 500 nm. In some aspects, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or at least, at most, exactly, or between any two of 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PDI), which is dimensionless (Koppel, D., J. Chem. Phys. 57, 1972, pp 4814-4820, ISO 13321). Here, “mean diameter,” “diameter,” or “size” for particles is used synonymously with this value of the Z-average.

LNPs described herein may exhibit a polydispersity index less than about 0.5, less than about 0.4, less than about 0.3, or about 0.2 or less. By way of example, the LNPs may exhibit a polydispersity index of at least, at most, exactly, or between any two of 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5. The polydispersity index is, in some aspects, calculated based on dynamic light scattering measurements by the so-called cumulant analysis as mentioned in the definition of the “average diameter.” Under certain prerequisites, it may be taken as a measure of the size distribution of an ensemble of nanoparticles.

In certain aspects, nucleic acids (e.g., RNA molecules), when present in provided LNPs, are resistant in aqueous solution to degradation with a nuclease. In some aspects, LNPs are liver-targeting lipid nanoparticles. In some aspects, LNPs are cationic lipid nanoparticles comprising one or more cationic lipids (e.g., ones described herein). In some aspects, cationic LNPs may comprise at least one cationic lipid, at least one polymer conjugated lipid, and at least one helper lipid (e.g., at least one neutral lipid).

In certain aspects, the RNA solution and lipid preparation mixture or compositions thereof may have, have at least, or have at least, at most, exactly, or between any two of about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of a particular lipid, lipid type, or non-lipid component such as lipid-like materials and/or cationic polymers or an adjuvant, antigen, peptide, polypeptide, sugar, nucleic acid or other material disclosed herein or as would be known to one of skill in the art.

LNPs described herein may be prepared using a wide range of methods that may involve obtaining a colloid from at least one cationic or cationically ionizable lipid or lipid-like material and/or at least one cationic polymer and mixing the colloid with nucleic acid to obtain nucleic acid particles. The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media). In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in some aspects, formed as follows: an ethanol solution comprising lipids, such as cationic lipids and additional lipids, is injected into an aqueous solution under stirring. In some aspects, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

In some aspects, LNP-encapsulated RNA may be produced by rapid mixing of an RNA solution described herein (e.g., the RNA product solution) and a lipid preparation described herein (comprising, e.g., at least one cationic lipid and optionally one or more other lipid components, in an organic solvent) under conditions such that a sudden change in solubility of lipid component(s) is triggered, which drives the lipids towards self-assembly in the form of LNPs. In some aspects, suitable buffering agents comprise tris, histidine, citrate, acetate, phosphate, or succinate. The pH of a liquid formulation relates to the pKa of the encapsulating agent (e.g. cationic lipid). The pH of the acidifying buffer may be at least half a pH scale less than the pKa of the encapsulating agent (e.g. cationic lipid), and the pH of the final buffer may be at least half a pH scale greater than the pKa of the encapsulating agent (e.g. cationic lipid). In some aspects, properties of a cationic lipid are chosen such that nascent formation of particles occurs by association with an oppositely charged backbone of a nucleic acid (e.g., RNA). In this way, particles are formed around the nucleic acid, which, for example, in some aspects, may result in much higher encapsulation efficiency than it is achieved in the absence of interactions between nucleic acids and at least one of the lipid components.

In certain aspects, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

Some aspects described herein relate to compositions, methods and uses involving more than one, e.g., 2, 3, 4, 5, 6 or even more nucleic acid species such as RNA species. In an LNP formulation, it is possible that each nucleic acid species is separately formulated as an individual LNP formulation. In that case, each individual LNP formulation will comprise one nucleic acid species. The individual LNP formulations may be present as separate entities, e.g. in separate containers. Such formulations are obtainable by providing each nucleic acid species separately (typically each in the form of a nucleic acid-containing solution) together with suitable cationic or cationically ionizable lipids or lipid-like materials and cationic polymers that allow the formation of LNPs. Respective particles will contain exclusively the specific nucleic acid species that is being provided when the particles are formed (individual particulate formulations).

In some aspects, a composition such as a pharmaceutical composition comprises more than one individual LNP formulation. Respective pharmaceutical compositions are referred to as mixed LNP formulations. Mixed LNP formulations according to the invention are obtainable by forming, separately, individual LNP formulations, as described above, followed by a step of mixing of the individual LNP formulations. By the step of mixing, a formulation comprising a mixed population of nucleic acid-containing LNPs is obtainable. Individual LNP populations may be together in one container, comprising a mixed population of individual LNP formulations.

Alternatively, it is possible that different nucleic acid species are formulated together as a combined LNP formulation. Such formulations are obtainable by providing a combined formulation (typically combined solution) of different RNA species together with suitable cationic or cationically ionizable lipids or lipid-like materials and cationic polymers that allow the formation of LNPs. As opposed to a mixed LNP formulation, a combined LNP formulation will typically comprise LNPs that comprise more than one RNA species. In a combined LNP composition, different RNA species are typically present together in a single particle.

### 1. Cationic Polymeric Materials

Given their high degree of chemical flexibility, polymeric materials are commonly used for nanoparticle-based delivery. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic materials useful in some aspects herein. In addition, some investigators have synthesized polymeric materials specifically for nucleic acid delivery. Poly(P-amino esters), in particular, have gained widespread use in nucleic acid delivery owing to their ease of synthesis and biodegradability. In some aspects, such synthetic materials may be suitable for use as cationic materials herein.

A “polymeric material,” as used herein, is given its ordinary meaning, e.g., a molecular structure comprising one or more repeat units (monomers), connected by covalent bonds. In some aspects, such repeat units may all be identical; alternatively, in some cases, there may be more than one type of repeat unit present within the polymeric material. In some cases, a polymeric material is biologically derived, e.g., a biopolymer such as a protein. In some cases, additional moieties may also be present in the polymeric material, for example targeting moieties such as those described herein.

Those skilled in the art are aware that, when more than one type of repeat unit is present within a polymer (or polymeric moiety), then the polymer (or polymeric moiety) is said to be a “copolymer.” In some aspects, a polymer (or polymeric moiety) utilized in accordance with the present disclosure may be a copolymer. Repeat units forming the copolymer may be arranged in any fashion. For example, in some aspects, repeat units may be arranged in a random order; alternatively or additionally, in some aspects, repeat units may be arranged in an alternating order, or as a “block” copolymer, e.g., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

In certain aspects, a polymeric material for use in accordance with the present disclosure is biocompatible. Biocompatible materials are those that typically do not result in significant cell death at moderate concentrations. In certain aspects, a biocompatible material is biodegradable, e.g., is able to degrade, chemically and/or biologically, within a physiological environment, such as within the body. In certain aspects, a polymeric material may be or comprise protamine or polyalkyleneimine, in particular protamine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some aspects, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some aspects, a polyalkyleneimine comprises polyethylenimine and/or polypropylenimine. In some aspects, the polyalkyleneimine is polyethyleneimine (PEI). In some aspects, the polyalkyleneimine is a linear polyalkyleneimine, e.g., linear polyethyleneimine (PEI).

Cationic materials (e.g., polymeric materials, including polycationic polymers) contemplated for use herein include those which are able to electrostatically bind nucleic acid. In some aspects, cationic polymeric materials contemplated for use herein include any cationic polymeric materials with which nucleic acid may be associated, e.g. by forming complexes with the nucleic acid or forming vesicles in which the nucleic acid is enclosed or encapsulated.

In some aspects, particles described herein may comprise polymers other than cationic polymers, e.g., non-cationic polymeric materials and/or anionic polymeric materials. Collectively, anionic and neutral polymeric materials are referred to herein as non-cationic polymeric materials.

### 2. Lipids & Lipid-Like Materials

The terms “lipid” and “lipid-like material” are used herein to refer to molecules which comprise one or more hydrophobic moieties or groups and optionally also one or more hydrophilic moieties or groups. According to the disclosure, lipids and lipid-like materials may be cationic, anionic or neutral. Neutral lipids or lipid-like materials exist in an uncharged or neutral zwitterionic form at a selected pH.

The term “lipid” refers to a group of organic compounds that are characterized by being insoluble in water but soluble in many organic solvents. Generally, lipids may be divided into eight categories: fatty acids and their derivatives (including tri-, di-, monoglycerides, and phospholipids), glycerolipids, glycerophospholipids, sphingolipids, saccharolipids, polyketides, sterol lipids as well as sterol-containing metabolites such as cholesterol, and prenol lipids. Examples of fatty acids include, but are not limited to, fatty esters and fatty amides. Examples of glycerolipids include, but are not limited to, glycosylglycerols and glycerophospholipids (e.g., phosphatidylcholine, phosphatidylethanolamine, phosphatidylserine). Examples of sphingolipids include, but are not limited to, ceramides phosphosphingolipids (e.g., sphingomyelins, phosphocholine), and glycosphingolipids (e.g., cerebrosides, gangliosides). Examples of sterol lipids include, but are not limited to, cholesterol and its derivatives and tocopherol and its derivatives.

The term “lipid-like material,” “lipid-like compound,” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

In some aspects, the RNA solution and lipid preparation mixture or compositions thereof may comprise cationic lipids, neutral lipids, cholesterol, and/or polymer (e.g., polyethylene glycol) conjugated lipids which form lipid nanoparticles that encompass the RNA molecules. Therefore, in some aspects, the LNP may comprise a cationic lipid and one or more excipients, e.g., one or more neutral lipids, charged lipids, steroids or steroid analogs (e.g., cholesterol), polymer conjugated lipids (e.g. PEG-lipid), or combinations thereof. In some aspects, the LNPs encompass, or encapsulate, the nucleic acid molecules.

### 3. Cationic Lipids

Cationic or cationically ionizable lipids or lipid-like materials refer to a lipid or lipid-like material capable of being positively charged and able to electrostatically bind nucleic acid. As used herein, a “cationic lipid” or “cationic lipid-like material” refers to a lipid or lipid like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge. Exemplary cationic lipids include one or more amine group(s) which bear the positive charge. Cationic lipids may encapsulate negatively charged RNA.

In some aspects, cationic lipids are ionizable such that they may exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “cationically ionizable” lipids or lipid-like materials are comprised by the term “cationic lipid” or “cationic lipid-like material” unless contradicted by the circumstances.

In some aspects, a cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

In some aspects, a cationic lipid may be at least, at most, exactly, or between any two of 10 mol %, 20 mol %, 30 mol %, 40 mol %, 50 mol %, 60 mol %, 70 mol %, 80 mol %, 90 mol %, or 100 mol %, or any range or value derivable therein, of the total lipid present in the particle.

Examples of cationic lipids include, but are not limited to: ((4-hydroxybutyl)azanediyl)bis(hexane-6,1-diyl)bis(2-hexyldecanoate); 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-I-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DM A), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy)propan-1-aminium (DOBAQ), 2-({8-[(3b)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid 02-200); or heptadecan-9-yl 8-((2-hydroxyethyl) (6-oxo-6-(undecyloxy)hexyl) amino) octanoate (SM-102).

In some aspects, the lipid nanoparticles comprise one or more cationic lipids. In one aspect, the lipid nanoparticles comprise (4-hydroxybutyl)azanediyl)bis(hexane-6,1-diyl)bis(2-hexyldecanoate) (ALC-0315), having the formula:

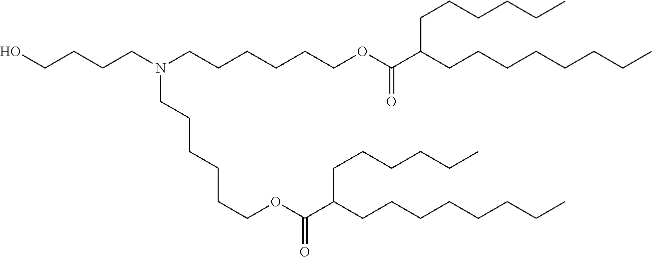 [SMILES: CCCCCCCCC(CCCCCC)C(=O)OCCCCCCN(CCCCO)CCCCCCOC(=O)C(CCCCCC)CCCCCCCC]

Cationic lipids are disclosed in, e.g., U.S. Pat. No. 10,166,298, the full disclosures of which are herein incorporated by reference in their entirety for all purposes. Representative cationic lipids include:

| No. | Structure |
|---|---|
| 1 | 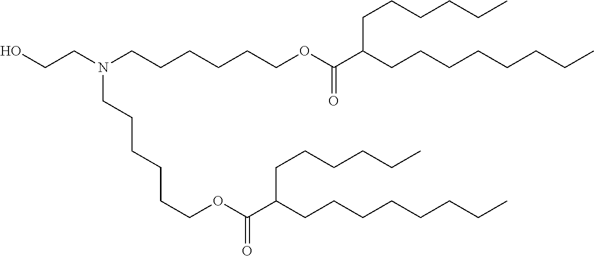 [SMILES: CCCCCCCCC(CCCCCC)C(=O)OCCCCCCN(CCO)CCCCCCOC(=O)C(CCCCCC)CCCCCCCC] |
| 2 | 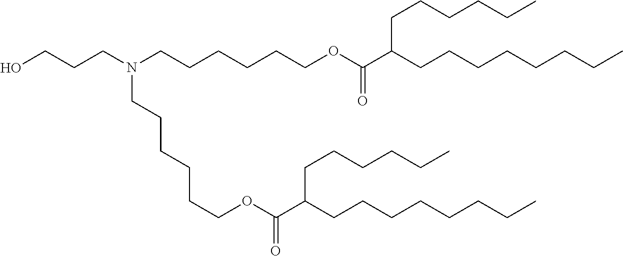 [SMILES: CCCCCCCCC(CCCCCC)C(=O)OCCCCCCN(CCCO)CCCCCCOC(=O)C(CCCCCC)CCCCCCCC] |
| 3 | 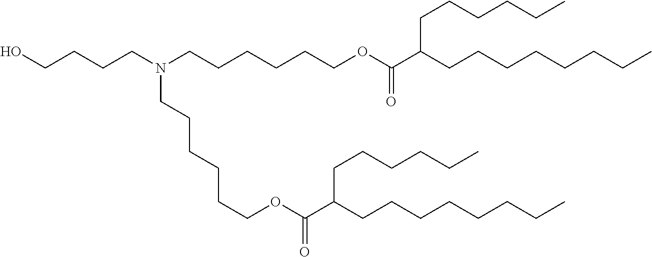 [SMILES: CCCCCCCCC(CCCCCC)C(=O)OCCCCCCN(CCCCO)CCCCCCOC(=O)C(CCCCCC)CCCCCCCC] |
| 4 | 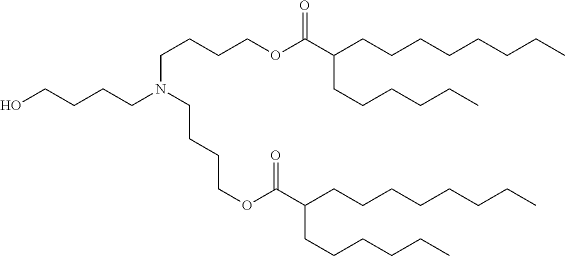 [SMILES: CCCCCCCCC(CCCCCC)C(=O)OCCCCN(CCCCO)CCCCOC(=O)C(CCCCCC)CCCCCCCC] |
| 5 | 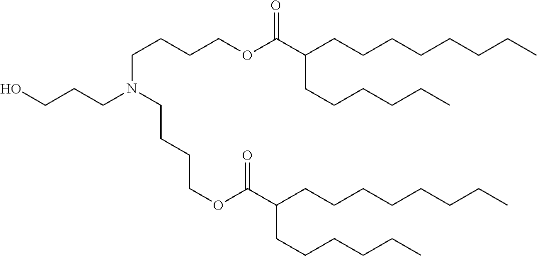 [SMILES: CCCCCCCCC(CCCCCC)C(=O)OCCCCN(CCCO)CCCCOC(=O)C(CCCCCC)CCCCCCCC] |
| 6 | 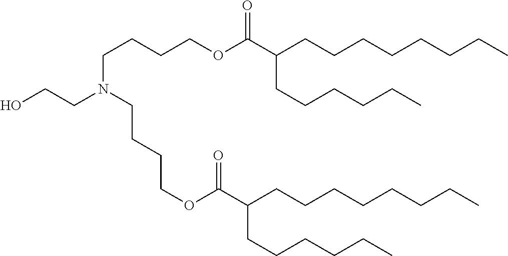 [SMILES: CCCCCCCCC(CCCCCC)C(=O)OCCCCN(CCO)CCCCOC(=O)C(CCCCCC)CCCCCCCC] |
| 7 | 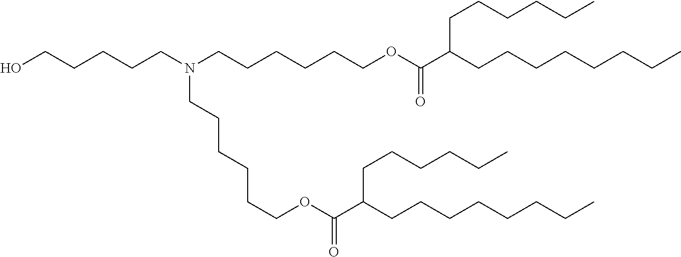 [SMILES: CCCCCCCCC(CCCCCC)C(=O)OCCCCCCN(CCCCCO)CCCCCCOC(=O)C(CCCCCC)CCCCCCCC] |
| 8 | 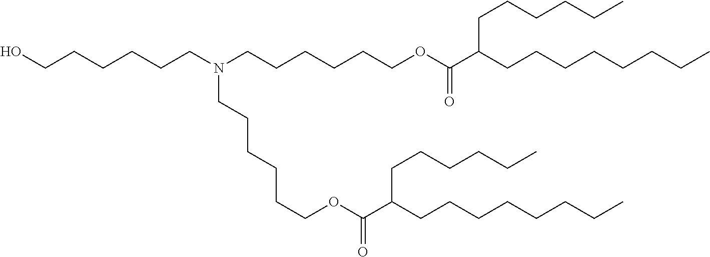 [SMILES: CCCCCCCCC(CCCCCC)C(=O)OCCCCCCN(CCCCCCO)CCCCCCOC(=O)C(CCCCCC)CCCCCCCC] |
| 9 | 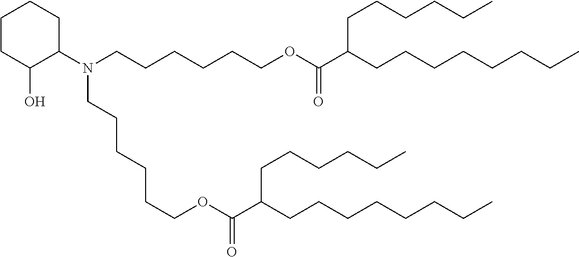 [SMILES: CCCCCCCCC(CCCCCC)C(=O)OCCCCCCN(CCCCCCOC(=O)C(CCCCCC)CCCCCCCC)C1CCCCC1O] |
| 10 | 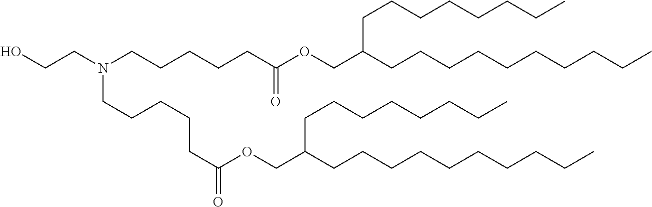 [SMILES: CCCCCCCCCCC(CCCCCCCC)COC(=O)CCCCCN(CCO)CCCCCC(=O)OCC(CCCCCCCC)CCCCCCCCCC] |
| 11 | 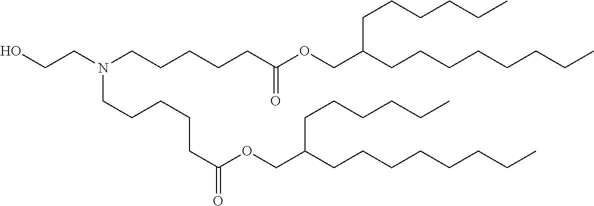 [SMILES: CCCCCCCCC(CCCCCC)COC(=O)CCCCCN(CCO)CCCCCC(=O)OCC(CCCCCC)CCCCCCCC] |
| 12 | 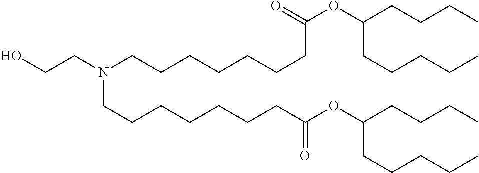 [SMILES: CCCCCC(CCCC)OC(=O)CCCCCCCN(CCO)CCCCCCCC(=O)OC(CCCC)CCCCC] |
| 13 | 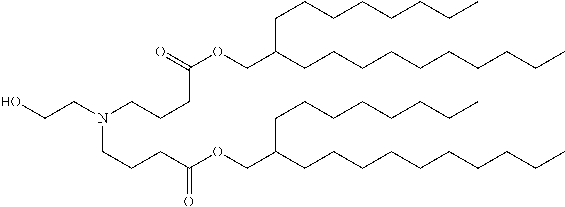 [SMILES: CCCCCCCCCCC(CCCCCCCC)COC(=O)CCCN(CCO)CCCC(=O)OCC(CCCCCCCC)CCCCCCCCCC] |
| 14 | 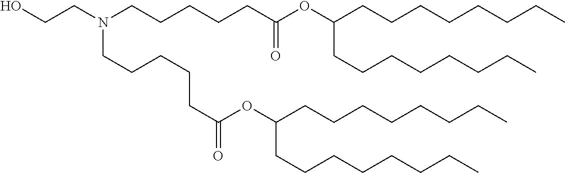 [SMILES: CCCCCCCCC(CCCCCCCC)OC(=O)CCCCCN(CCO)CCCCCC(=O)OC(CCCCCCCC)CCCCCCCC] |
| 15 | 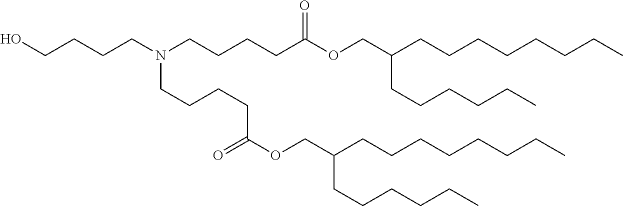 [SMILES: CCCCCCCCC(CCCCCC)COC(=O)CCCCN(CCCCO)CCCCC(=O)OCC(CCCCCC)CCCCCCCC] |
| 16 | 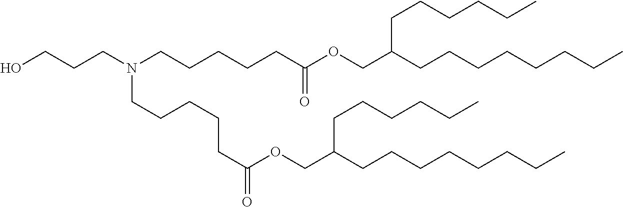 [SMILES: CCCCCCCCC(CCCCCC)COC(=O)CCCCCN(CCCO)CCCCCC(=O)OCC(CCCCCC)CCCCCCCC] |
| 17 | 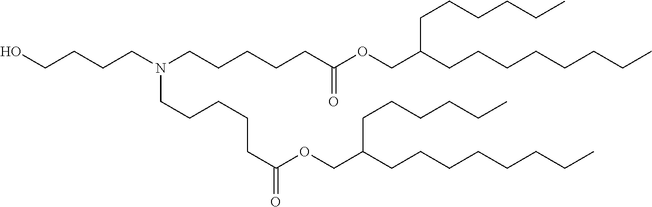 [SMILES: CCCCCCCCC(CCCCCC)COC(=O)CCCCCN(CCCCO)CCCCCC(=O)OCC(CCCCCC)CCCCCCCC] |
| 18 | 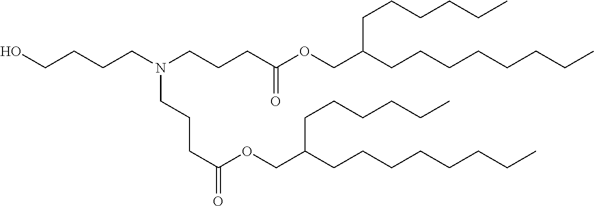 [SMILES: CCCCCCCCC(CCCCCC)COC(=O)CCCN(CCCCO)CCCC(=O)OCC(CCCCCC)CCCCCCCC] |
| 19 | 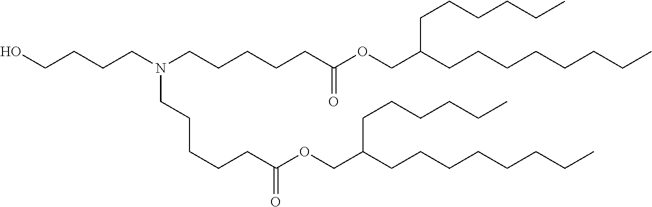 [SMILES: CCCCCCCCC(CCCCCC)COC(=O)CCCCCN(CCCCO)CCCCCC(=O)OCC(CCCCCC)CCCCCCCC] |
| 20 | 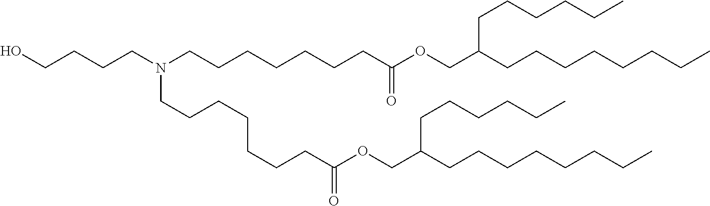 [SMILES: CCCCCCCCC(CCCCCC)COC(=O)CCCCCCCN(CCCCO)CCCCCCCC(=O)OCC(CCCCCC)CCCCCCCC] |
| 21 | 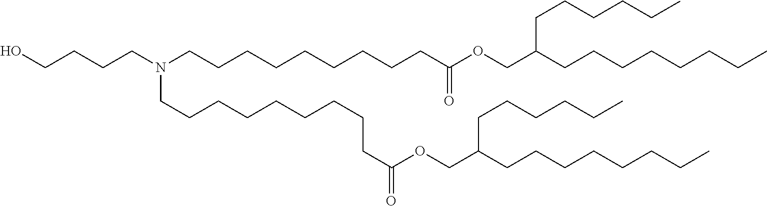 [SMILES: CCCCCCCCC(CCCCCC)COC(=O)CCCCCCCCCN(CCCCO)CCCCCCCCCC(=O)OCC(CCCCCC)CCCCCCCC] |
| 22 | 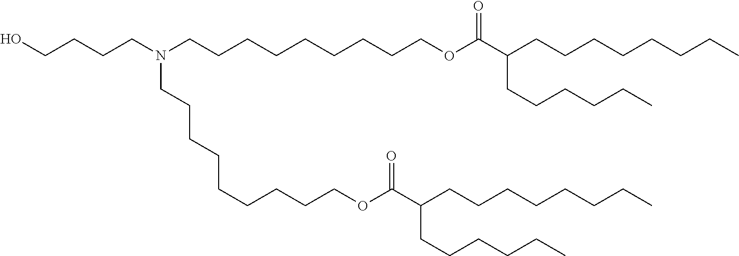 [SMILES: CCCCCCCCC(CCCCCC)C(=O)OCCCCCCCCCN(CCCCO)CCCCCCCCCOC(=O)C(CCCCCC)CCCCCCCC] |
| 23 | 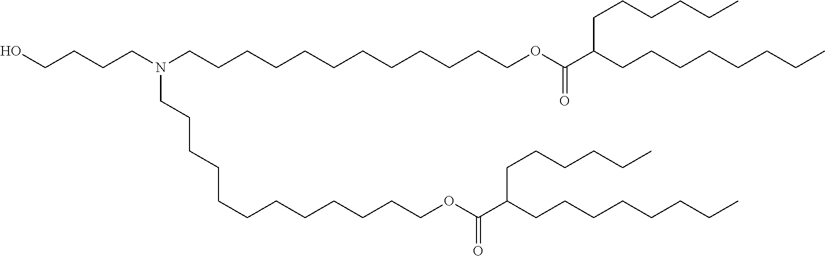 [SMILES: CCCCCCCCC(CCCCCC)C(=O)OCCCCCCCCCCCCN(CCCCO)CCCCCCCCCCCCOC(=O)C(CCCCCC)CCCCCCCC] |
| 24 | 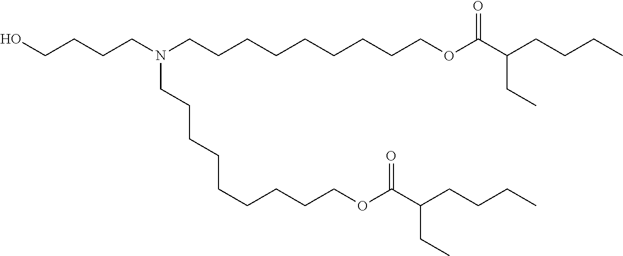 [SMILES: CCCCC(CC)C(=O)OCCCCCCCCCN(CCCCO)CCCCCCCCCOC(=O)C(CC)CCCC] |
| 25 | 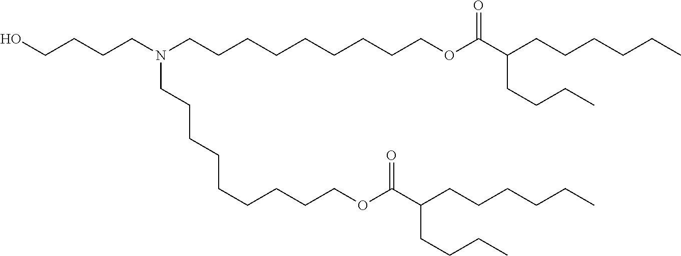 [SMILES: CCCCCCC(CCCC)C(=O)OCCCCCCCCCN(CCCCO)CCCCCCCCCOC(=O)C(CCCC)CCCCCC] |
| 26 | 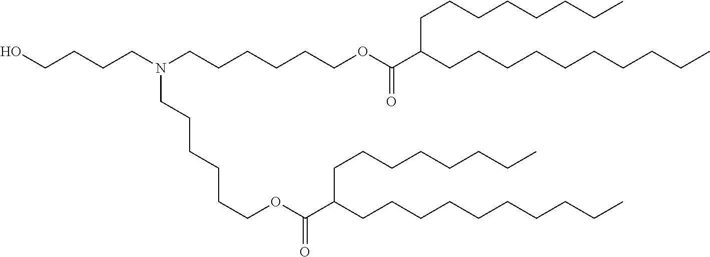 [SMILES: CCCCCCCCCCC(CCCCCCCC)C(=O)OCCCCCCN(CCCCO)CCCCCCOC(=O)C(CCCCCCCC)CCCCCCCCCC] |
| 27 | 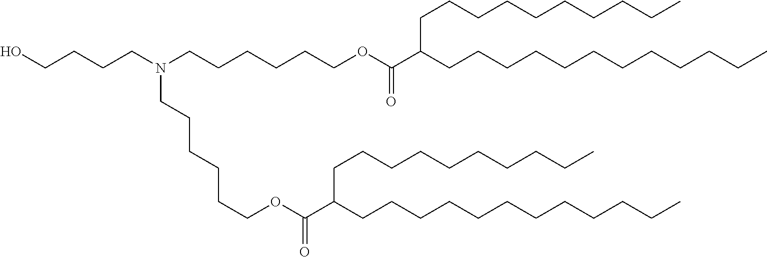 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| 28 | 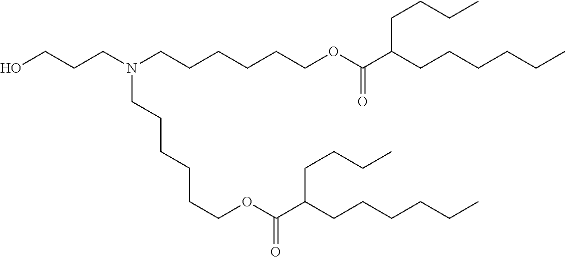 [SMILES: CCCCCCC(CCCC)C(=O)OCCCCCCN(CCCO)CCCCCCOC(=O)C(CCCC)CCCCCC] |
| 29 | 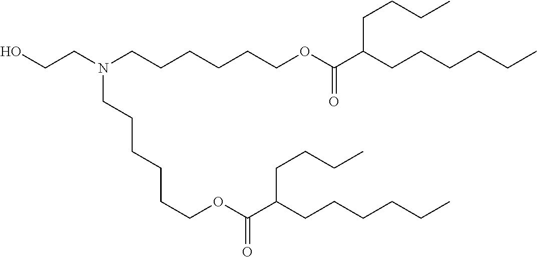 [SMILES: CCCCCCC(CCCC)C(=O)OCCCCCCN(CCO)CCCCCCOC(=O)C(CCCC)CCCCCC] |
| 30 | 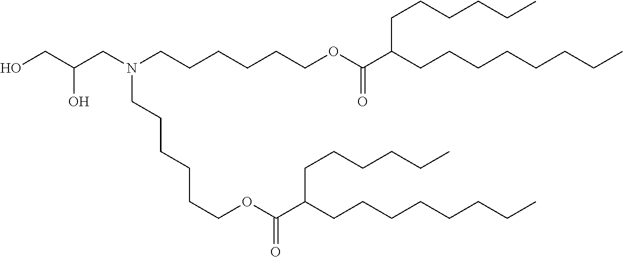 [SMILES: CCCCCCCCC(CCCCCC)C(=O)OCCCCCCN(CCCCCCOC(=O)C(CCCCCC)CCCCCCCC)CC(O)CO] |
| 31 | 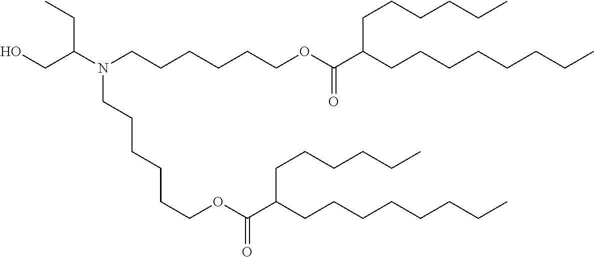 [SMILES: CCCCCCCCC(CCCCCC)C(=O)OCCCCCCN(CCCCCCOC(=O)C(CCCCCC)CCCCCCCC)C(CC)CO] |
| 32 | 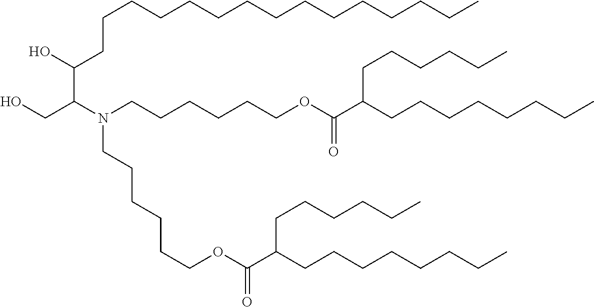 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| 33 | 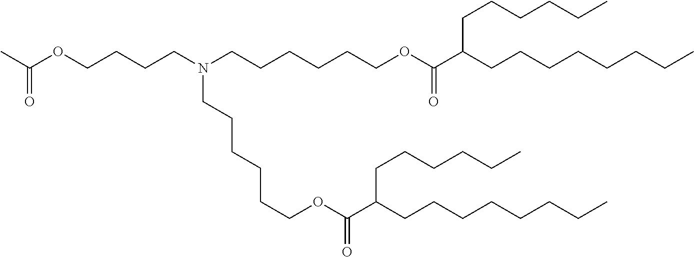 [SMILES: CCCCCCCCC(CCCCCC)C(=O)OCCCCCCN(CCCCCCOC(=O)C(CCCCCC)CCCCCCCC)CCCCOC(C)=O] |
| 34 | 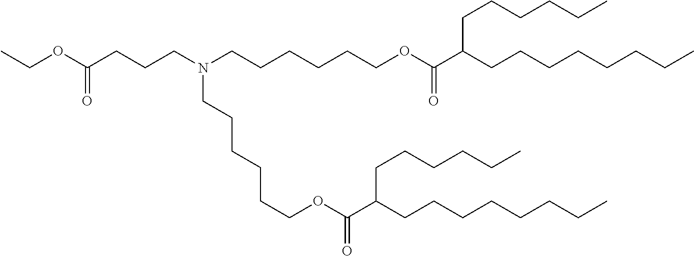 [SMILES: CCCCCCCCC(CCCCCC)C(=O)OCCCCCCN(CCCCCCOC(=O)C(CCCCCC)CCCCCCCC)CCCC(=O)OCC] |
| 35 | 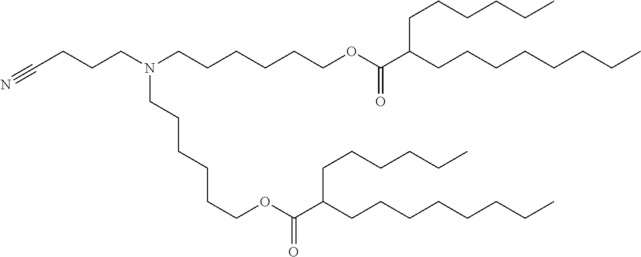 [SMILES: CCCCCCCCC(CCCCCC)C(=O)OCCCCCCN(CCCC#N)CCCCCCOC(=O)C(CCCCCC)CCCCCCCC] |
| 36 | 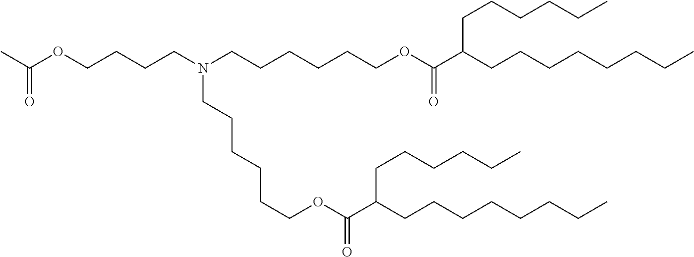 [SMILES: CCCCCCCCC(CCCCCC)C(=O)OCCCCCCN(CCCCCCOC(=O)C(CCCCCC)CCCCCCCC)CCCCOC(C)=O] |

In some aspects, the RNA-LNPs comprise a cationic lipid, a RNA molecule as described herein and one or more of neutral lipids, steroids, pegylated lipids, or combinations thereof. If more than one cationic lipid is incorporated within the LNP, such percentages apply to the combined cationic lipids. In one aspect, the cationic lipid is present in the LNP in an amount such as at least, at most, exactly, or between any two of about 40, 41, 42, 43, 44, 45, 46, 47, 48, 49, 50, 51, 52, 53, 54, 55, 56, 57, 58, 59 or 60 mole percent, respectively.

In some aspects of the disclosure the LNP comprises a combination or mixture of any the lipids described above.

### 4. Polymer Conjugated Lipid

In some aspects, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), 2-[(polyethylene glycol)-2000]-N,N-ditetradecylacetamide, and the like.

In certain aspects, the LNP comprises an additional, stabilizing-lipid which is a polyethylene glycol-lipid (pegylated lipid). A polymer conjugated lipid (e.g. PEG-lipid) refers to a molecule comprising both a lipid portion and a polymer portion. An example of a polymer conjugated lipid is a PEG-lipid. A PEG-lipid refers to a molecule comprising both a lipid portion and a polyethylene glycol portion. PEG-lipids include, but are not limited to, PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one aspect, the polyethylene glycol-lipid is N-[(methoxy polyethylene glycol)2000)carbamyl]-1,2-dimyristyloxlpropyl-3-amine (PEG-c-DMA). In one aspect, the polyethylene glycol-lipid is PEG-2000-DMG. In one aspect, the polyethylene glycol-lipid is PEG-c-DOMG). In other aspects, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl)carbamate or 2,3-di(tetradecanoxy)propyl-N-(u>-methoxy(polyethoxy)ethyl)carbamate. PEG-lipids are disclosed in, e.g., U.S. Pat. No. 9,737,619, the full disclosures of which are herein incorporated by reference in their entirety for all purposes.

In some aspects, the lipid nanoparticles comprise a polymer conjugated lipid. In one aspect, the lipid nanoparticle comprises 2-[(polyethylene glycol)-2000]-N,N-ditetradecylacetamide (ALC-0159), having the formula:

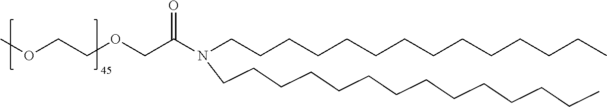

In various aspects, the molar ratio of the cationic lipid to the pegylated lipid ranges from about 100:1 to about 20:1, e.g., from about 20:1, 25:1, 30:1, 35:1, 40:1, 45:1, 50:1, 55:1, 60:1, 65:1, 70:1, 75:1, 80:1, 85:1, 90:1, 95:1, or 100:1, or any range or value derivable therein.

In certain aspects, the PEG-lipid is present in the LNP in an amount from about 1 to about 10 mole percent (mol %) (e.g., at least, at most, exactly, or between any two of 1, 2, 3, 4, 5, 6, 7, 8, 9, or 10 mol %), relative to the total lipid content of the nanoparticle.

### 5. Additional Lipids

In certain aspects, the LNP comprises one or more additional lipids or lipid-like materials that stabilize the formation of particles during their formation. Suitable stabilizing or structural lipids include non-cationic lipids, e.g., neutral lipids and anionic lipids. Without being bound by any theory, optimizing the formulation of LNPs by addition of other hydrophobic moieties, such as cholesterol and lipids, in addition to an ionizable/cationic lipid or lipid-like material may enhance particle stability and efficacy of nucleic acid delivery.

As used herein, an “anionic lipid” refers to any lipid that is negatively charged at a selected pH. The term “neutral lipid” refers to any one of a number of lipid species that exist in either an uncharged or neutral zwitterionic form at physiological pH. In some aspects, additional lipids comprise one of the following neutral lipid components: (1) a phospholipid, (2) cholesterol or a derivative thereof; or (3) a mixture of a phospholipid and cholesterol or a derivative thereof.

Representative neutral lipids include phosphatidylcholines, phosphatidylethanolamines, phosphatidylglycerols, phosphatidic acids, phosphatidylserines, ceramides, sphingomyelins, dihydro-sphingomyelins, cephalins, and cerebrosides. Exemplary phospholipids include, for example, phosphatidylcholines, e.g., diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), dioleoylphosphatidylglycerol (DOPG), dipalmitoylphosphatidylglycerol (DPPG), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), I-oleoyl-2-cholesterylhemisuccinoyl-sn-glycero-3-phosphocholine (OChemsPC), and 1-hexadecyl-sn-glycero-3-phosphocholine (C16 Lyso PC); and phosphatidylethanolamines, e.g., diacylphosphatidylethanolamines, such as dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-Icarboxylate (DOPE-mal), dipalmitoyl phosphatidyl ethanolamine (DPPE), dimyristoylphosphoethanolamine (DMPE), dilauroyl-phosphatidylethanolamine (DLPE), distearoyl-phosphatidylethanolamine (DSPE), iphytanoyl-phosphatidylethanolamine (DpyPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one aspect, the neutral lipid is 1,2-distearoyl-sn-glycero-3phosphocholine (DSPC), having the formula:

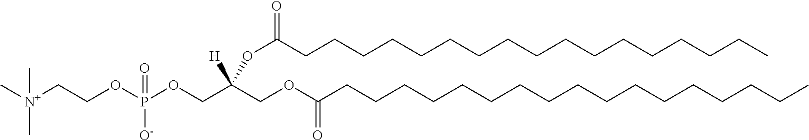 [SMILES: CCCCCCCCCCCCCCCCCC(=O)OC[C@H](COP(=O)([O-])OCC[N+](C)(C)C)OC(=O)CCCCCCCCCCCCCCCCC]

In some aspects, the LNPs comprise a neutral lipid, and the neutral lipid comprises one or more of DSPC, DPPC, DMPC, DOPC, POPC, DOPE, or SM.

In various aspects, the LNPs further comprise a steroid or steroid analogue. A “steroid” is a compound comprising the following carbon skeleton:

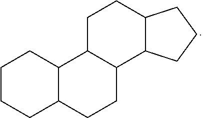 [SMILES: C1CCC2C(C1)CCC1C3CCCC3CCC21]

In certain aspects, the steroid or steroid analogue is cholesterol. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof. In one aspect, the cholesterol has the formula:

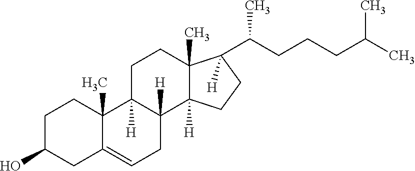

Without being bound by any theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some aspects, the molar ratio of the cationic lipid to the neutral lipid ranges from about 2:1 to about 8:1, or from about 10:0 to about 1:9, about 4:1 to about 1:2, or about 3:1 to about 1:1.

In some aspects, the non-cationic lipid, e.g., neutral lipid (e.g., one or more phospholipids and/or cholesterol), may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle. In some aspects, the non-cationic lipid, e.g., neutral lipid (e.g., one or more phospholipids and/or cholesterol), may be at least, at most, exactly, or between any two of 0 mol %, 10 mol %, 20 mol %, 30 mol %, 40 mol %, 50 mol %, 60 mol %, 70 mol %, 80 mol %, or 90 mol % of the total lipid present in the particle.

### VI. Characterization and Analysis of RNA Molecule

The RNA molecule described herein may be analyzed and characterized using various methods. Analysis may be performed before or after capping. Alternatively, analysis may be performed before or after poly-A capture-based affinity purification. In another aspect, analysis may be performed before or after additional purification steps, e.g., anion exchange chromatography and the like. For example, RNA template quality may be determined using Bioanalyzer chip based electrophoresis system. In other aspects, RNA template purity is analyzed using analytical reverse phase HPLC respectively. Capping efficiency may be analyzed using, e.g., total nuclease digestion followed by MS/MS quantitation of the dinucleotide cap species vs. uncapped GTP species. In vitro efficacy may be analyzed by, e.g., transfecting RNA molecule into a human cell line. Protein expression of the polypeptide of interest may be quantified using methods such as ELISA or flow cytometry. Immunogenicity may be analyzed by, e.g., transfecting RNA molecules into cell lines that indicate innate immune stimulation, e.g., PBMCs. Cytokine induction may be analyzed using, e.g., methods such as ELISA to quantify a cytokine, e.g., Interferon-α. Biodistribution may be analyzed, e.g. by bioluminescence measurements.

In some aspects, an RNA polynucleotide disclosed herein is characterized in that, when assessed in an organism administered a composition or medical preparation comprising an RNA polynucleotide, elevated expression of a gene of interest (e.g., an antigen); increased duration of expression (e.g., prolonged expression) of a gene of interest (e.g., an antigen); elevated expression and increased duration of expression (e.g., prolonged expression) of a gene of interest (e.g., an antigen); decreased interaction with IFIT1 of an RNA polynucleotide; increased translation of an RNA polynucleotide; is observed relative to an appropriate reference.

In some aspects, a reference comprises an organism administered an otherwise similar RNA polynucleotide without a m7(3′OMeG)(5′)ppp(5′)(2′OMeAi)pG2 cap. In some aspects, a reference comprises an organism administered an otherwise similar RNA polynucleotide without a cap proximal sequence disclosed herein. In some aspects, a reference comprises an organism administered an otherwise similar RNA polynucleotide with a self-hybridizing sequence.

In some aspects, elevated expression is determined at least 24 hours, at least 48 hours at least 72 hours, at least 96 hours, or at least 120 hours after administration of a composition or medical preparation comprising an RNA polynucleotide. In some aspects, elevated expression is determined at least 24 hours after administration of a composition or medical preparation comprising an RNA polynucleotide. In some aspects, elevated expression is determined at least 48 hours after administration of a composition or medical preparation comprising an RNA polynucleotide. In some aspects, elevated expression is determined at least 72 hours after administration of a composition or medical preparation comprising an RNA polynucleotide. In some aspects, elevated expression is determined at least 96 hours after administration of a composition or medical preparation comprising an RNA polynucleotide. In some aspects, elevated expression is determined at least 120 hours after administration of a composition or medical preparation comprising an RNA polynucleotide.

In some aspects, elevated expression is determined at about 24-120 hours after administration of a composition or medical preparation comprising an RNA polynucleotide. In some aspects, elevated expression is determined at about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide.

In some aspects, elevated expression of a gene of interest (e.g., an antigen) is at least 2-fold to at least 10-fold. In some aspects, elevated expression of a gene of interest (e.g., an antigen) is at least 2-fold. In some aspects, elevated expression of a gene of interest (e.g., an antigen) is at least 3-fold. In some aspects, elevated expression of a gene of interest (e.g., an antigen) is at least 4-fold. In some aspects, elevated expression of a gene of interest (e.g., an antigen) is at least 6-fold. In some aspects, elevated expression of a gene of interest (e.g., an antigen) is at least 8-fold. In some aspects, elevated expression of a gene of interest (e.g., an antigen) is at least 10-fold.

In some aspects, elevated expression of a gene of interest (e.g., an antigen) is about 2-fold to about 50-fold. In some aspects, elevated expression of a gene of interest (e.g., an antigen) is about 2-fold to about 45-fold, about 2-fold to about 40-fold, about 2-fold to about 30-fold, about 2-fold to about 25-fold, about 2-fold to about 20-fold, about 2-fold to about 15-fold, about 2-fold to about 10-fold, about 2-fold to about 8-fold, about 2-fold to about 5-fold, about 5-fold to about 50-fold, about 10-fold to about 50-fold, about 15-fold to about 50-fold, about 20-fold to about 50-fold, about 25-fold to about 50-fold, about 30-fold to about 50-fold, about 40-fold to about 50-fold, or about 45-fold to about 50-fold. In some aspects, elevated expression of a gene of interest (e.g., an antigen) is at least, at most, exactly, or between any two of 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, or any range or value derivable therein.

In some aspects, elevated expression (e.g., increased duration of expression) of a gene of interest (e.g., an antigen) persists for at least, at most, exactly, or between any two of 24 hours, 48 hours, 72 hours, 96 hours, or 120 hours after administration of a composition or a medical preparation comprising an RNA polynucleotide. In some aspects, elevated expression of a gene of interest (e.g., an antigen) persists for at least 24 hours after administration. In some aspects, elevated expression of a gene of interest (e.g., an antigen) persists for at least 48 hours after administration. In some aspects, elevated expression of a gene of interest (e.g., an antigen) persists for at least 72 hours after administration. In some aspects, elevated expression of a gene of interest (e.g., an antigen) persists for at least 96 hours after administration. In some aspects, elevated expression of a gene of interest (e.g., an antigen) persists for at least 120 hours after administration of a composition or medical preparation comprising an RNA polynucleotide.

In some aspects, elevated expression of a gene of interest (e.g., an antigen) persists for about 24-120 hours after administration of a composition or medical preparation comprising an RNA polynucleotide. In some aspects, elevated expression persists for about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide. In some aspects, elevated expression of a gene of interest (e.g., an antigen) persists for at least, at most, exactly, or between any two of 24 hours, 36 hours, 48 hours, 60 hours, 72 hours, 84 hours, 96 hours, 108 hours, or 120 hours, or any range or value derivable therein.

### VII. Immune Response and Assays

As discussed herein, the disclosure concerns evoking or inducing an immune response in a subject against an RSV protein, e.g., a wild type or variant RSV F protein, and an influenza protein, e.g., HA and/or NA. In one aspect, the immune response may protect against or treat a subject having, suspected of having, or at risk of developing an infection or related disease, particularly those related to RSV and influenza. One use of the immunogenic compositions of the disclosure is to prevent RSV infections and influenza infections by inoculating or vaccination of a subject.

### 1. Immunoassays

The present disclosure includes the implementation of serological assays to evaluate whether and to what extent an immune response is induced or evoked by compositions of the disclosure. There are many types of immunoassays that may be implemented. Immunoassays encompassed by the present disclosure include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. In some aspects, the immunoassays are the various types of enzyme linked immunosorbent assays (ELISAs) and radioimmunoassays (RIA) known in the art. Immunohistochemical detection using tissue sections is also particularly useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

### 2. Diagnosis of Rsv Infection

The present disclosure contemplates the use of RSV polypeptides, proteins, and/or peptides in a variety of ways, including the detection of the presence of RSV to diagnose an infection. In accordance with the disclosure, a method of detecting the presence of infections involves the steps of obtaining a sample suspected of being infected by one or more RSV strains, such as a sample taken from an individual, for example, from one's blood, saliva, tissues, bone, muscle, cartilage, or skin. Following isolation of the sample, diagnostic assays utilizing the polypeptides, proteins, and/or peptides of the present disclosure may be carried out to detect the presence of RSV, and such assay techniques for determining such presence in a sample are well known to those skilled in the art and include methods such as radioimmunoassay, western blot analysis and ELISA assays.

In general, in accordance with the disclosure, a method of diagnosing an infection is contemplated wherein a sample suspected of being infected with RSV has added to it the polypeptide, protein, or peptide, in accordance with the present disclosure, and RSV is indicated by antibody binding to the polypeptides, proteins, and/or peptides, or polypeptides, proteins, and/or peptides binding to the antibodies in the sample.

Accordingly, RNA molecules encoding RSV polypeptides, proteins, and/or peptides in accordance with the disclosure may be used for to treat, prevent, or reduce the severity of illness due to RSV infection (e.g., active or passive immunization) or for use as research tools.

Any of the above described polypeptides, proteins, and/or peptides may be labeled directly with a detectable label for identification and quantification of RSV. Labels for use in immunoassays are generally known to those skilled in the art and include enzymes, radioisotopes, and fluorescent, luminescent and chromogenic substances, including colored particles such as colloidal gold or latex beads. Suitable immunoassays include enzyme-linked immunosorbent assays (ELISA).

### 3. Protective Immunity

In some aspects of the disclosure, RNA molecules encoding RSV F protein and/or an antigen derived from influenza, e.g., HA and/or NA, RNA-LNPs and compositions thereof, confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein the phrase “immune response” or its equivalent “immunological response” refers to the development of a humoral (antibody mediated), cellular (mediated by antigen-specific T cells or their secretion products) or both humoral and cellular response directed against an antigen. Such a response may be an active response or a passive response. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject from the production of antibodies in response to the presence of an of an antigen, e.g. an RSV F protein encoded by an RNA molecule of the present disclosure and/or an antigen derived from influenza, e.g., HA and/or NA encoded by an RNA molecule of the present disclosure.

As used herein “passive immunity” includes, but is not limited to, administration of activated immune effectors including cellular mediators or protein mediators (e.g., monoclonal and/or polyclonal antibodies) of an immune response. A monoclonal or polyclonal antibody composition may be used in passive immunization to treat, prevent, or reduce the severity of illness caused by infection by organisms that carry the antigen recognized by the antibody. An antibody composition may include antibodies that bind to a variety of antigens that may in turn be associated with various organisms. The antibody component may be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as viruses, including but not limited to RSV and/or influenza.

Passive immunity may be imparted to a patient or subject by administering to the patient immunoglobulins (Ig) and/or other immune factors obtained from a donor or other non-patient source having a known immunoreactivity. In other aspects, an immunogenic composition of the present disclosure may be administered to a subject who then acts as a source or donor for globulin, produced in response to challenge with the immunogenic composition (“hyperimmune globulin”), that contains antibodies directed against a RSV and influenza and/or other organism. A subject thus treated would donate plasma from which hyperimmune globulin would then be obtained, via conventional plasma-fractionation methodology, and administered to another subject in order to impart resistance against or to treat RSV infection and influenza infection.

For purposes of this specification and the accompanying claims the terms “epitope” and “antigenic determinant” are used interchangeably to refer to a site on an antigen to which B and/or T cells respond or recognize. B-cell epitopes may be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope may be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope may be identified by in vitro assays that measure antigen-dependent proliferation, as determined by ³H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response may be determined by proliferation assays (CD4 (+) T cells) or CTL (cytotoxic T lymphocyte) assays. The relative contributions of humoral and cellular responses to the protective or therapeutic effect of an immunogenic composition may be distinguished by separately isolating IgG and T-cells from an immunized syngeneic animal and measuring protective or therapeutic effect in a second subject.

As used herein, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins. Under normal physiological conditions antibodies are found in plasma and other body fluids and in the membrane of certain cells and are produced by lymphocytes of the type denoted B cells or their functional equivalent.

As used herein the terms “immunogenic agent” or “immunogen” or “antigen” are used interchangeably to describe a molecule capable of inducing an immunological response against itself on administration to a recipient, either alone, in conjunction with an adjuvant, or presented on a display vehicle.

### VIII. Compositions

In some aspects, an RNA molecules and/or RNA-LNPs disclosed herein may be administered in a pharmaceutical composition or a medicament and may be administered in the form of any suitable pharmaceutical composition. In some aspects, a pharmaceutical composition is for therapeutic or prophylactic treatments. In one aspect, the disclosure relates to a composition for administration to a host. In some aspects, the host is a human. In other aspects, the host is a non-human.

In some aspects, an RNA molecules and/or RNA-LNPs disclosed herein may be administered in a pharmaceutical composition which may be formulated into preparations in solid, semi-solid, liquid, lyophilized, frozen, or gaseous forms. In some aspects, an RNA molecule and/or RNA-LNPs disclosed herein may be administered in a pharmaceutical composition which may comprise a pharmaceutically acceptable carrier and may optionally comprise one or more adjuvants, stabilizers, salts, buffers, preservatives, and optionally other therapeutic agents. In some aspects, a pharmaceutical composition disclosed herein comprises one or more pharmaceutically acceptable carriers, diluents and/or excipients. In some aspects, pharmaceutical compositions do not include an adjuvant (e.g., they are adjuvant free).

Suitable preservatives for use in a pharmaceutical compositions of the present disclosure include, without limitation, benzalkonium chloride, chlorobutanol, paraben and thimerosal. The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some aspects, the pharmaceutical composition of the present disclosure includes sodium chloride.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents may be selected with regard to the intended route of administration and standard pharmaceutical practice.

In some aspects, the composition comprises an RNA molecule comprising an open reading frame encoding an immunogenic polypeptide. In some aspects, the immunogenic polypeptide comprises a RSV antigen and influenza antigen. In some aspects, the RSV antigen is a RSV F protein and the influenza antigen is HA and/or NA.

In some aspects, the composition comprises an RNA molecule comprising an open reading frame encoding a full-length RSV F protein. In some aspects, the encoded immunogenic polypeptide is a truncated RSV F protein. In some aspects, the encoded immunogenic polypeptide is a variant of a RSV F protein. In some aspects, the encoded immunogenic polypeptide is a fragment of a RSV F protein.

In some aspects, the composition comprises an RNA molecule comprising an open reading frame encoding a full-length influenza HA protein. In some aspects, the encoded immunogenic polypeptide is a truncated influenza HA protein. In some aspects, the encoded immunogenic polypeptide is a variant of a influenza HA protein. In some aspects, the encoded immunogenic polypeptide is a fragment of a influenza HA protein.

In some aspects, the composition comprises an RNA molecule comprising an open reading frame encoding a full-length influenza NA protein. In some aspects, the encoded immunogenic polypeptide is a truncated influenza NA protein. In some aspects, the encoded immunogenic polypeptide is a variant of a influenza NA protein. In some aspects, the encoded immunogenic polypeptide is a fragment of a influenza NA protein.

In some aspects, the composition comprises a RSV subunit and an influenza RNA-LNP. In preferred embodiments, the RSV subunit comprises a mutant of a wild-type RSV F protein and are immunogenic against the wild-type RSV F protein or against a virus comprising the wild-type F protein, as explained above.

In one aspect, the disclosure relates to an article of manufacture comprising a vial having (a) a first chamber that comprises a first composition comprising an RNA polynucleotide; (b) a second chamber that comprises a lyophilized composition comprising a polypeptide; and (c) a septum separating the first and second chambers and impermeable to the gaseous medium; and (d) actuating means effective to bring the first composition and the second composition into contact by breach of the septum.

In one aspect, the disclosure relates to an article of manufacture comprising a vial having (a) a first chamber that is substantially filled with an aqueous suspension that comprises (i) an aqueous medium; (ii) a drug in solid particulate form in a therapeutically effective amount suspended in the medium; (b) a second chamber; (c) a septum separating the first and second chambers and impermeable to the gaseous medium; and (d) actuating means effective to bring the aqueous suspension and the gaseous medium into contact by breach of the septum such that the gaseous medium acts as an effective headspace for agitation of the formulation. In some embodiments, the vial comprises a polypeptide antigen or fragment thereof in the first chamber and RNA-encapsulated LNP composition in the second chamber. In some embodiments, the polypeptide antigen or fragment thereof is lyophilized. In some embodiments, the polypeptide antigen or fragment thereof is RSVpreF. In some embodiments, the polypeptide antigen or fragment thereof is a lyophilized RSVpreF antigen. In some embodiments, the vial comprises a two-chamber vial as described in U.S. Pat. No. 7,387,623, which is incorporated by reference in its entirety herein.

A mixing vial as described herein is in commercial use. for example as a packaging system under the name Act-O-Vial® of Pharmacia Corporation Such a vial may be used with a lyophilized powder formulation of a drug in the lower compartment and an aqueous diluent in the upper compartment. In some embodiments, the mixing via: has an aqueous suspension formulation in the upper compartment and only a gaseous medium, typically air, in the lower compartment, wherein the lower compartment may function as a reservoir for air or other gaseous medium to provide a headspace for effective agitation following actuation of the vial, but, by virtue of its lack of contact with the upper compartment prior to actuation, minimizes or prevents exposure to oxygen of ingredients of the formulation that are susceptible to oxidative degradation.

In some embodiments, the vial has a lower compartment comprising a lyophilized powder formulation comprising a polypeptide and an upper compartment comprising an mRNA-encapsulated LNP. In some embodiments, the vial has an upper compartment comprising a lyophilized powder formulation comprising a polypeptide and a lower compartment comprising an mRNA-encapsulated LNP. In some embodiments, the compartment comprising the mRnNA-encapsuated LNP, whether lower or upper, includes minimal amount of gaseous medium or air.

In some embodiments, an article of manufacture disclosed herein comprises an Act-C-Vial® or substantially similar mixing vial containing, in an upper compartment thereof, a formulation that comprises (a) an aqueous medium having dispersed therein in a therapeutically effective amount and: in a lower compartment thereof, a lyophilized composition comprising a polypeptide. In some embodiments, an article of manufacture disclosed herein comprises an Act-C-Vial® or substantially similar mixing vial containing, in an upper compartment thereof, a formulation that comprises (a) a lyophilized composition comprising a polypeptide and; in a lower compartment thereof, an aqueous medium having dispersed therein in a therapeutically effective amount. In some embodiments, the lyophilized composition comprises RSVpreF. In some embodiments, the aqueous medium comprises an RNA polynucleotide. In some embodiments, the aqueous medium comprises an mRNA-encapsulated LNP.

It will be apparent to those of skill in the art that many modifications can be made to the article of manufacture described immediately above without taking the article outside the scope of the present invention. For example, the actuating means can comprise, in place of a means for applying hydraulic pressure to the contents of the upper compartment, a substantially rigid member that, when a downward force is applied to the cap assembly or a portion thereof, transmits the force directly to the septum or plug separating the upper and lower compartments.

Other two-chamber devices that can be substituted include those described, for example, in the patents individually listed: U.S. Pat. Nos. 3,464,414; 4,614,267 to Larkin.; U.S. Pat. No. 4,871,354 to Conn et al.; U.S. Pat. No. 5,335,773 to Haber et al.; U.S. Pat. No. 5,336,180 to Kriesel & Thompson; U.S. Pat. No. 5,350,372 to Ikeda et al.; U.S. Pat. No. 5,385,546 to Kriesel & Thompson. Other than the Act-C-Vial® system of Pharmacia Corporation, two-chamber systems in commercial use for mixing a lyophilized powder with a diluent include those sold under the names Univial™ and ADD-Vantage™ of Abbott Laboratories and Piggyback™ of SmithKline Beecham An article of the invention comprises, in the formulation that substantially fills the first chamber of the vial, a drug in a therapeutically effective amount. Typically the drug is one of low solubility in water, for example having a solubility of less than about 10 mg/ml, illustratively less than about 1 mg/ml or even less than about 1 mg/ml.

A composition of the invention can be prepared by a process comprising a first step of formulating a suspension of the drug in particulate form in an aqueous medium that comprises one or more wetting and/or suspending agents, at least one of which is susceptible to oxidative degradation as defined herein. Any suitable formulation method can be used that brings the ingredients of the composition together in a way that results in an aqueous suspension exhibiting controlled flocculation. Such methods are well known in the art. An antioxidant can optionally be added at any suitable point in the formulating step.

Although it is not generally necessary to use an oxygen-depleted atmosphere in the second chamber of the vial, if desired an oxygen-depleted gaseous medium can be provided in a number of ways. An especially convenient way is to prepare the vial, before insertion of the septum or plug, under a blanket of a nonreactive gas such as nitrogen or a noble gas (e.g., argon or helium). An oxygen-depleted gaseous medium preferably consists essentially of nitrogen and oxygen in a weight ratio not less than about 10:1, more preferably not less than about 20:1 and still more preferably not less than about 40:1. Other gases such as carbon dioxide, water vapor and noble gases can be present in such a medium, for example at concentrations in which they occur in ambient air. The gaseous medium can consist essentially of nitrogen.

### 1. Immunogenic Compositions Including LNPS

In some aspects, a pharmaceutical composition comprises an RNA molecule (e.g., polynucleotide) disclosed herein formulated with a lipid-based delivery system. Thus, some aspects, the composition includes a lipid-based delivery system (e.g., LNPs) (e.g., a lipid-based vaccine), which delivers a nucleic acid molecule to the interior of a cell, where it may then replicate, inhibit protein expression of interest, and/or express the encoded polypeptide of interest. The delivery system may have adjuvant effects which enhance the immunogenicity of an encoded antigen. In some aspects, the composition comprises at least one RNA molecule encoding a RSV polypeptide complexed with, encapsulated in, and/or formulated with one or more lipids, and forming lipid nanoparticles (LNPs), liposomes, lipoplexes and/or nanoliposomes. In some aspects, the composition comprises a lipid nanoparticle. Thus, in certain aspects, the present disclosure concerns compositions comprising one or more lipids associated with a nucleic acid or a polypeptide/peptide (e.g., RSV RNA-LNPs). In some aspects, the composition comprises at least one RNA molecule encoding an influenza polypeptide complexed with, encapsulated in, and/or formulated with one or more lipids, and forming lipid nanoparticles (LNPs), liposomes, lipoplexes and/or nanoliposomes. In some aspects, the composition comprises a lipid nanoparticle. Thus, in certain aspects, the present disclosure concerns compositions comprising one or more lipids associated with a nucleic acid or a polypeptide/peptide (e.g., influenza HA and/or NA RNA-LNPs).

The immunogenic composition including a lipid-based delivery system may further include one or more salts and/or one or more pharmaceutically acceptable surfactants, preservatives, carriers, diluents, and/or excipients, in some cases. In some aspects, the immunogenic composition including a lipid-based delivery system further include a pharmaceutically acceptable vehicle. In some aspects, each of a buffer, stabilizing agent, and optionally a salt, may be included in the immunogenic composition including a lipid-based delivery system. In other aspects, any one or more of a buffer, stabilizing agent, salt, surfactant, preservative, and excipient may be excluded from the immunogenic composition including a lipid-based delivery system.

In a further aspect, the immunogenic composition including a lipid-based delivery system further comprises a stabilizing agent. In some aspects, the stabilizing agent comprises sucrose, mannose, sorbitol, raffinose, trehalose, mannitol, inositol, sodium chloride, arginine, lactose, hydroxyethyl starch, dextran, polyvinylpyrolidone, glycine, or a combination thereof. In some aspects, the stabilizing agent is a disaccharide, or sugar. In one aspect, the stabilizing agent is sucrose. In another aspect, the stabilizing agent is trehalose. In a further aspect, the stabilizing agent is a combination of sucrose and trehalose. In some aspects, the total concentration of the stabilizing agent(s) in the composition is about 5% to about 10% w/v. For example, the total concentration of the stabilizing agent may be equal to at least, at most, exactly, or between any two of 1%, 2%, 3%, 4%, 5%, 6%, 7%, 8%, 9%, 10%, 11%, 12%, 13%, 14%, 15%, 16%, 17%, 18%, 19%, or 20% w/v or any range or value derivable therein. In some aspects, the stabilizing agent concentration includes, but is not limited to, a concentration of about 10 mg/mL to about 400 mg/mL, about 100 mg/mL to about 200 mg/mL, about 100 mg/mL to about 150 mg/mL, about 100 mg/mL to about 140 mg/mL, about 100 mg/mL to about 130 mg/mL, about 100 mg/mL to about 120 mg/mL, about 100 mg/mL to about 110 mg/mL, or about 100 mg/mL to about 105 mg/mL. In some aspects, the concentration of the stabilizing agent is equal to at least, at most, exactly, or between any two of 10 mg/mL, 20 mg/mL, 50 mg/mL, 100 mg/mL, 101 mg/mL, 102 mg/mL, 103 mg/mL, 104 mg/mL, 105 mg/mL, 106 mg/mL, 107 mg/mL, 108 mg/mL, 109 mg/mL, 110 mg/mL, 150 mg/mL, 200 mg/mL, 300 mg/mL, 400 mg/mL, or more.

In a further aspect, the mass amount of the stabilizing agent and the mass amount of the RNA are in a specific ratio. In one aspect, the ratio of the mass amount of the stabilizing agent and the RNA is no greater than 5000. In another aspect, the ratio of the mass amount of the stabilizing agent and the RNA is no greater than 2000. In another aspect, the ratio of the mass amount of the stabilizing agent and the RNA is no greater than 1000. In another aspect, the ratio of the mass amount of the stabilizing agent and the RNA is no greater than 500. In another aspect, the ratio of the mass amount of the stabilizing agent and the RNA is no greater than 100. In another aspect, the ratio of the mass amount of the stabilizing agent and the pharmaceutical substance is no greater than 50. In another aspect, the ratio of the mass amount of the stabilizing agent and the RNA is no greater than 10. In another aspect, the ratio of the mass amount of the stabilizing agent and the RNA is no greater than 1. In another aspect, the ratio of the mass amount of the stabilizing agent and the RNA is no greater than 0.5. In another aspect, the ratio of the mass amount of the stabilizing agent and the RNA is no greater than 0.1. In another aspect, the stabilizing agent and RNA comprise a mass ratio of about 200-2000 of the stabilizing agent: 1 of the RNA.

In some aspects, the immunogenic composition including a lipid-based delivery system further comprises a buffer. Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. In some aspects, the buffer is a HEPES buffer, a Tris buffer, or a PBS buffer. Accordingly, in some embodiments, the composition further includes an adjuvant, e.g., aluminum-containing compounds, such as, for example, any of the adjuvants listed herein, including aluminum hydroxide and AlPO₄. In one aspect, the buffer is Tris buffer. In another aspect, the buffer is a HEPES buffer. In a further aspect, the buffer is a PBS buffer. For example, the buffer concentration may be equal to at least, at most, exactly, or between any two of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM, or any range or value derivable therein. The buffer may be at a neutral pH, pH 6.5 to 8.5, pH 7.0 to pH 8.0, or pH 7.2 to pH 7.6. For example, the buffer may be at least, at most, exactly, or between any two of pH 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein. In specific aspects, the buffer is at pH 7.4.

In some aspects, the immunogenic composition including a lipid-based delivery system may further comprise a salt. Examples of salts include but not limited to sodium salts and/or potassium salts. In one aspect, the salt is a sodium salt. In a specific aspect, the sodium salt is sodium chloride. In one aspect, the salt is a potassium salt. In some aspects, the potassium salt comprises potassium chloride. The concentration of the salts in the composition may be about 70 mM to about 140 mM. For example, the salt concentration may be equal to at least, at most, exactly, or between any two of 50 mM, 60 mM, 70 mM, 80 mM, 90 mM, 100 mM, 120 mM, 130 mM, 140 mM, 150 mM, 160 mM, 170 mM, 180 mM, 190 mM, or 200 mM.

In some aspects, the salt concentration includes, but is not limited to, a concentration of about 1 mg/mL to about 100 mg/mL, about 1 mg/mL to about 50 mg/mL, about 1 mg/mL to about 40 mg/mL, about 1 mg/mL to about 30 mg/mL, about 1 mg/mL to about 20 mg/mL, about 1 mg/mL to about 10 mg/mL, or about 1 mg/mL to about 15 mg/mL. In some aspects, the concentration of the salt is equal to at least, at most, exactly, or between any two of 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, or more. The salt may be at a neutral pH, pH 6.5 to 8.5, pH 7.0 to pH 8.0, or pH 7.2 to pH 7.6. For example, the salt may be at a pH equal to at least, at most, exactly, or between any two of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5.

In some aspects, the immunogenic composition including a lipid-based delivery system further comprises a surfactant, a preservative, any other excipient, or a combination thereof. As used herein, “any other excipient” includes, but is not limited to, antioxidants, glutathione, EDTA, methionine, desferal, antioxidants, metal scavengers, or free radical scavengers. In one aspect, the surfactant, preservative, excipient or combination thereof is sterile water for injection (sWFI), bacteriostatic water for injection (BWFI), saline, dextrose solution, polysorbates, poloxamers, Triton, divalent cations, Ringer's lactate, amino acids, sugars, polyols, polymers, or cyclodextrins.

Examples of excipients, which refer to ingredients in the immunogenic compositions that are not active ingredients, include but are not limited to carriers, binders, diluents, lubricants, thickeners, surface active agents, preservatives, stabilizers, emulsifiers, buffers, flavoring agents, disintegrants, coatings, plasticizers, compression agents, wet granulation agents, or colorants. Preservatives for use in the compositions disclosed herein include but are not limited to benzalkonium chloride, chlorobutanol, paraben and thimerosal. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Diluents, or diluting or thinning agents, include but are not limited to ethanol, glycerol, water, sugars such as lactose, sucrose, mannitol, and sorbitol, and starches derived from wheat, corn rice, and potato; and celluloses such as microcrystalline cellulose. The amount of diluent in the composition may range from about 10% to about 90% by weight of the total composition, about 25% to about 75%, about 30% to about 60% by weight, or about 12% to about 60%.

The pH and exact concentration of the various components in the immunogenic composition including a lipid-based delivery system are adjusted according to well-known parameters. The use of such media and agents for pharmaceutical active substances is well known in the art. Except insofar as any conventional media or agent is incompatible with the active ingredients, its use in immunogenic, prophylactic and/or therapeutic compositions is contemplated.

In one aspect, a pharmaceutical composition comprises an RSV RNA molecule encoding a RSV polypeptide as disclosed herein that is complexed with, encapsulated in, and/or formulated with one or more lipids to form RSV RNA-LNPs. In some aspects, the RSV RNA-LNP composition is a liquid. In some aspects, the RSV RNA-LNP composition is frozen. In some aspects, the RSV RNA-LNP composition is lyophilized. In some aspects, a RSV RNA-LNP composition comprises a RSV RNA polynucleotide molecule encoding a RSV polypeptide as disclosed herein, encapsulated in LNPs with a lipid composition of a cationic lipid, a PEGylated lipid (i.e. PEG-lipid), and one or more structural lipids (e.g., a neutral lipid).

In one aspect, a pharmaceutical composition comprises an influenza RNA molecule encoding an influenza polypeptide as disclosed herein that is complexed with, encapsulated in, and/or formulated with one or more lipids to form influenza RNA-LNPs. In some aspects, the influenza RNA-LNP composition is a liquid. In some aspects, the influenza RNA-LNP composition is frozen. In some aspects, the influenza RNA-LNP composition is lyophilized. In some aspects, a influenza RNA-LNP composition comprises an influenza RNA polynucleotide molecule encoding a influenza polypeptide as disclosed herein, encapsulated in LNPs with a lipid composition of a cationic lipid, a PEGylated lipid (i.e. PEG-lipid), and one or more structural lipids (e.g., a neutral lipid).

In some aspects, a RSV RNA-LNP composition comprises an cationic lipid. In some aspects, an influenza RNA-LNP composition comprises an cationic lipid. The cationic lipid may comprise any one or more cationic lipids disclosed herein. In specific aspects, the cationic lipid comprises ((4-hydroxybutyl)azanediyl)bis(hexane-6,1-diyl)bis(2-hexyldecanoate) (ALC-0315). In some aspects, the cationic lipid (e.g., ALC-0315) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the cationic lipid (e.g., ALC-0315) is included in the composition at a concentration of at least, at most, between any two of, or exactly 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the cationic lipid (e.g., ALC-0315) is included in the composition at a concentration of at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the cationic lipid (e.g., ALC-0315) is included in the composition at a concentration of between 0.4 and 0.5, between 0.5 and 0.6, between 0.6 and 0.7, between 0.7 and 0.8, between 0.8 and 0.9, or between 0.9 and 1. In some aspects, the cationic lipid (e.g., ALC-0315) is included in the composition at a concentration of between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95, or between 0.95 and 1 mg/mL.

In specific aspects, the cationic lipid (e.g., ALC-0315) is included in the composition at a concentration of 0.8 to 0.95 mg/mL. In specific aspects, the cationic lipid (e.g., ALC-0315) is included in the composition at a concentration of 0.8 to 0.9 mg/mL. In specific aspects, the cationic lipid (e.g., ALC-0315) is included in the composition at a concentration of 0.85 to 0.9 mg/mL. In specific aspects, the cationic lipid (e.g., ALC-0315) is included in the composition at a concentration of 0.8, 0.81, 0.82, 0.83, 0.84, 0.85, 0.86, 0.87, 0.88, 0.89, 0.9, 0.91, 0.92, 0.93, 0.94, or 0.95 mg/mL. Concentrations for lyophilized compositions are determined post-reconstitution.

In some aspects, a RSV RNA-LNP composition further comprises a PEGylated lipid (i.e., PEG-lipid). In some aspects, an influenza RNA-LNP composition further comprises a PEGylated lipid (i.e., PEG-lipid). The PEGylated lipid may comprise any one or more PEGylated lipids disclosed herein. In specific aspects, the PEGylated lipid comprises 2-[(polyethylene glycol)-2000]-N,N-ditetradecylacetamide (ALC-0159). In some aspects, the PEGylated lipid (e.g., ALC-0159) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the PEGylated lipid (e.g., ALC-0159) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the PEGylated lipid (e.g., ALC-0159) is included in the composition at a concentration of at least 0.01, at least 0.05, at least 0.1, at least 0.15, at least 0.2, at least 0.25 mg/mL, at least 0.3 mg/mL, at least 0.35 mg/mL, at least 0.4 mg/mL, at least 0.45 mg/mL or at least 0.5 mg/mL. In some aspects, the PEGylated lipid (e.g., ALC-0159) is included in the composition at a concentration of between 0.01 and 0.05, between 0.05 and 0.1, between 0.1 and 0.15, between 0.15 and 0.2, or between 0.2 and 0.25 mg/mL.

In specific aspects, the PEGylated lipid (e.g., ALC-0159) is included in the composition at a concentration of 0.05 to 0.15 mg/mL. In specific aspects, the PEGylated lipid (e.g., ALC-0159) is included in the composition at a concentration of 0.10 to 0.15 mg/mL. In specific aspects, the PEGylated lipid (e.g., ALC-0159) is included in the composition at a concentration of 0.05, 0.06, 0.07, 0.08, 0.09, 0.1, 0.11, 0.12, 0.13, 0.14 or 0.15 mg/mL. Concentrations for lyophilized compositions are determined post-reconstitution.

In some aspects, a RSV RNA-LNP composition further comprises one or more structural lipids. In some aspects, an influenza RNA-LNP composition further comprises one or more structural lipids. The one or more structural lipids may comprise any one or more structural lipids disclosed herein. In specific aspects, the one or more structural lipids comprise a neutral lipid and a steroid or steroid analog. In specific aspects, the one or more structural lipids comprise 1,2-Distearoyl-sn-glycero-3-phosphocholine (DSPC) and cholesterol. In some aspects, the one or more structural lipids (e.g., DSPC and cholesterol) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the one or more structural lipids (e.g., DSPC and cholesterol) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the one or more structural lipids (e.g., DSPC and cholesterol) are included in the composition at a concentration of at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the one or more structural lipids (e.g., DSPC and cholesterol) are included in the composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95 or between 0.95 and 1 mg/mL.

In specific aspects, the one or more structural lipids include DSPC, and the DSPC is included in the composition at a concentration of 0.1 to 0.25 mg/mL. In specific aspects, the one or more structural lipids include DSPC, and the DSPC is included in the composition at a concentration of 0.15 to 0.25 mg/mL. In specific aspects, the one or more structural lipids include DSPC, and the DSPC is included in the composition at a concentration of 0.1, 0.11, 0.12, 0.13, 0.14, 0.15, 0.16, 0.17, 0.18, 0.19, 0.2, 0.21, 0.22, 0.23, 0.24 or 0.25 mg/mL.

In specific aspects, the one or more structural lipids include cholesterol, and the cholesterol is included in the composition at a concentration of 0.3 to 0.45 mg/mL. In specific aspects, the one or more structural lipids include cholesterol, and the cholesterol is included in the composition at a concentration of 0.3 to 0.4. In specific aspects, the one or more structural lipids include cholesterol, and the cholesterol is included in the composition at a concentration of 0.35 to 0.45. In specific aspects, the one or more structural lipids include cholesterol, and the cholesterol is included in the composition at a concentration of 0.30, 0.31, 0.32, 0.33, 0.34, 0.35, 0.36, 0.37, 0.38, 0.39, 0.40, 0.41, 0.42, 0.43, 0.44, or 0.45 mg/mL. Concentrations for lyophilized compositions are determined post-reconstitution.

In some aspects, the RSV RNA-LNP composition and/or influenza RNA-LNP composition further comprises one or more buffers and stabilizing agents, and optionally, salts. Thus, in some aspects, the RSV RNA-LNP composition and/or influenza RNA-LNP composition comprises an cationic lipid, a PEGylated lipid, one or more structural lipids, one or more buffers, a stabilizing agent, and optionally, a salt. In some aspects, the composition comprising a RSV subunit and an influenza RNA-LNP comprises one or more buffers and stabilizing agents, and optionally, salts.

Thus, in some aspects, the composition comprising a RSV subunit and an influenza RNA-LNP comprises an cationic lipid, a PEGylated lipid, one or more structural lipids, one or more buffers, a stabilizing agent, and optionally, a salt.

In some aspects, a RSV RNA-LNP composition and/or influenza RNA-LNP composition comprises one or more buffers. In some aspects, the composition comprising a RSV subunit and an influenza RNA-LNP comprises one or more buffers. The one or more buffers may comprise any one or more buffering agents disclosed herein. In specific aspects, the composition comprises a Tris buffer comprising at least a first buffer and a second buffer. In some aspects, the first buffer is tromethamine. In some aspects, the second buffer is Tris hydrochloride (HCl). In some aspects, the first buffer and second buffer of the Tris buffer (e.g., tromethamine and Tris HCl) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. Concentrations for lyophilized compositions are determined post-reconstitution.

In some aspects, the RSV RNA-LNP composition and/or influenza RNA-LNP composition is a liquid composition comprising a Tris buffer. In some aspects, the composition comprising a RSV subunit and an influenza RNA-LNP comprises a Tris buffer. In some aspects, the Tris buffer comprises a first buffer. In some aspects, the first buffer is tromethamine. In some aspects, the first buffer (e.g., tromethamine) is included in the liquid composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the liquid composition at a concentration of at least 0.1, at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the first buffer (e.g., tromethamine) is included in the liquid composition at a concentration of between 0.05 and 0.15, between 0.15 and 0.25, between 0.25 and 0.35, between 0.35 and 0.45, between 0.45 and 0.55, between 0.55 and 0.65, between 0.65 and 0.75, between 0.75 and 0.85, or between 0.85 and 0.95. In some aspects, the first buffer (e.g., tromethamine) is included in the liquid composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95 or between 0.95 and 1 mg/mL.

In specific aspects, the first buffer (e.g., tromethamine) is included in the liquid composition at a concentration of 0.1 to 0.3 mg/mL. In specific aspects, the first buffer (e.g., tromethamine) is included in the liquid composition at a concentration of 0.15 to 0.25 mg/mL. In specific aspects, the first buffer (e.g., tromethamine) is included in the liquid composition at a concentration of 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29 or 0.3 mg/mL.

In some aspects, the RSV RNA-LNP composition and/or influenza RNA-LNP composition is a liquid composition comprising a Tris buffer comprising a second buffer. In some aspects, the composition comprising a RSV subunit and an influenza RNA-LNP comprises a Tris buffer and a second buffer. In some aspects, the second buffer comprises Tris HCl. In some aspects, the second buffer (e.g., Tris HCl) is included in the liquid composition at a concentration of at least, at most, between any two of, or exactly 0.5, 0.55, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or 1.5 mg/mL. In some aspects, the second buffer (e.g., Tris HCl) is included in the liquid composition at a concentration of at least 0.5, at least 0.55, at least 0.6, at least 0.65, at least 0.7, at least 0.75, at least 0.8, at least 0.85, at least 0.9, at least 0.95, at least 1, at least 1.05, at least 1.10, at least 1.15, at least 1.20, at least 1.25, at least 1.30, at least 1.35, at least 1.40, at least 1.45, or at least 1.50 mg/mL. In some aspects, the second buffer (e.g., Tris HCl) is included in the liquid composition at a concentration of between 0.5 and 0.6, between 0.6 and 0.7, between 0.7 and 0.8, between 0.8 and 0.9, between 0.9 and 1, between 1 and 1.10, between 1.10 and 1.20, between 1.20 and 1.30, between 1.30 and 1.40, or between 1.40 and 1.50 mg/mL.

In specific aspects, the second buffer (e.g., Tris HCl) is included in the liquid composition at a concentration of 1.25 to 1.40 mg/mL. In specific aspects, the second buffer (e.g., Tris HCl) is included in the liquid composition at a concentration of 1.30 to 1.40 mg/mL. In specific aspects, the second buffer (e.g., Tris HCl) is included in the liquid composition at a concentration of 1.25, 1.26, 1.27, 1.28, 1.29, 1.30, 1.31, 1.32, 1.33, 1.34, or 1.35, 1.36, 1.37, 1.38, 1.39, or 1.40 mg/mL.

In some aspects, the RSV RNA-LNP composition and/or influenza RNA-LNP composition is a lyophilized composition comprising a Tris buffer. In some aspects, the composition comprising a RSV subunit and an influenza RNA-LNP comprises a Tris buffer. In some aspects, the Tris buffer comprises a first buffer. In some aspects, the first buffer is tromethamine. In some aspects, the first buffer (e.g., tromethamine) is included in the lyophilized composition at a concentration, after reconstitution, of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the lyophilized composition at a concentration, after reconstitution, of at least 0.01, of at least 0.05, of at least 0.1, of at least 0.15, of at least 0.2, of at least 0.25, of at least 0.3, of at least 0.35, of at least 0.4, of at least 0.45, or of at least 0.5 mg/mL. In some aspects, the first buffer (e.g., tromethamine (Tris base)) is included in the lyophilized composition at a concentration, after reconstitution, of between 0.01 and 0.05, between 0.05 and 0.1, between 0.1 and 0.15, between 0.15 and 0.2, between 0.2 and 0.25 mg/mL, between 0.25 and 0.3 mg/mL, between 0.3 and 0.35 mg/mL, between 0.35 and 0.4 mg/mL, between 0.4 and 0.45 mg/mL, or between 0.45 and 0.5 mg/mL.

In specific aspects, the first buffer (e.g., tromethamine) is included in the lyophilized composition at a concentration, after reconstitution, of 0.01 and 0.15 mg/mL. In specific aspects, the first buffer (e.g., tromethamine) is included in the lyophilized composition at a concentration, after reconstitution, of 0.01 and 0.10 mg/mL. In specific aspects, the first buffer (e.g., tromethamine) is included in the lyophilized composition at a concentration, after reconstitution, of 0.05 and 0.15 mg/mL. In specific aspects, the first buffer (e.g., tromethamine) is included in the lyophilized composition at a concentration, after reconstitution, of 0.01, 0.02, 0.03, 0.04, 0.05, 0.06, 0.07, 0.08, 0.09, 0.1, 0.11, 0.12, 0.13, 0.14, or 0.15 mg/mL.

In some aspects, the RSV RNA-LNP composition and/or influenza RNA-LNP composition is a lyophilized composition comprising a Tris buffer comprising a second buffer. In some aspects, the composition comprising a RSV subunit and an influenza RNA-LNP comprises a Tris buffer and a second buffer. In some aspects, the second buffer comprises Tris HCl. In some aspects, the second buffer (e.g., Tris HCl) is included in the lyophilized composition at a concentration, after reconstitution, of at least, at most, between any two of, or exactly 0.1, 0.15, 0.2, 0.25, 0.3, 0.35, 0.4, 0.45, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the second buffer (e.g., Tris HCl) is included in the lyophilized composition at a concentration, after reconstitution, of at least 0.1, at least 0.2, at least 0.3, at least 0.4, at least 0.5, at least 0.6, at least 0.7, at least 0.8, at least 0.9, or at least 1 mg/mL. In some aspects, the second buffer (e.g., Tris HCl) is included in the lyophilized composition at a concentration, after reconstitution, of between 0.1 and 0.2, between 0.2 and 0.3, between 0.3 and 0.4, between 0.4 and 0.5, between 0.5 and 0.6, between 0.6 and 0.7, between 0.7 and 0.8, between 0.8 and 0.9, or between 0.9 and 1 mg/mL.

In specific aspects, the second buffer (e.g., Tris HCl) is included in the lyophilized composition at a concentration, after reconstitution, of 0.5 and 0.65 mg/mL. In specific aspects, the second buffer (e.g., Tris HCl) is included in the lyophilized composition at a concentration, after reconstitution, of 0.5 and 0.6 mg/mL. In specific aspects, the second buffer (e.g., Tris HCl) is included in the lyophilized composition at a concentration, after reconstitution, of 0.55 and 0.65 mg/mL. In specific aspects, the second buffer (e.g., Tris HCl) is included in the lyophilized composition at a concentration, after reconstitution, of 0.5, 0.51, 0.52, 0.53, 0.54, 0.55, 0.56, 0.57, 0.58, 0.59, 0.6, 0.61, 0.62, 0.63, 0.64, or 0.65 mg/mL.

In some aspects, a RSV RNA-LNP composition and/or influenza RNA-LNP composition comprises a stabilizing agent. In some aspects, the composition comprising a RSV subunit and an influenza RNA-LNP comprises a stabilizing agent. The stabilizing agent may comprise any one or more stabilizing agents disclosed herein. In some aspects, the stabilizing agent also functions as a cryoprotectant. In specific aspects, the stabilizing agent comprises sucrose. In some aspects, the stabilizing agent (e.g., sucrose) is included in the composition at a concentration of at least, at most, between any two of, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 ng/μg/mg per mL.

In some aspects, the RSV RNA-LNP composition and/or influenza RNA-LNP composition is a liquid composition. In some aspects, the RSV subunit composition and influenza RNA-LNP composition is a liquid composition. In such liquid compositions, the stabilizing agent (e.g., sucrose) is included in the liquid composition at a concentration of at least, at most, between any two of, or exactly 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121,122, 123, 124, 125, 126, 127, 128, 129 or 130 mg/mL. In some aspects, the stabilizing agent (e.g., sucrose) is included in the liquid composition at a concentration of at least 70, at least 75, at least 80, at least 85, at least 90, at least 95, at least 100, at least 105, at least 110, at least 115, at least 120, at least 125 or at least 130 mg/mL. In some aspects, the stabilizing agent (e.g., sucrose) is included in the liquid composition at a concentration of between 70 and 80, between 80 and 90, between 90 and 100, between 100 and 110, between 110 and 120, or between 120 and 130 mg/mL.

In specific aspects, the stabilizing agent (e.g., sucrose) is included in the liquid composition at a concentration of 95 to 110 mg/mL. In specific aspects, the stabilizing agent (e.g., sucrose) is included in the liquid composition at a concentration of 95 to 105 mg/mL. In specific aspects, the stabilizing agent (e.g., sucrose) is included in the liquid composition at a concentration of 100 to 110 mg/mL. In specific aspects, the stabilizing agent (e.g., sucrose) is included in the liquid composition at a concentration of 95, 96, 97, 98, 99, 100, 101, 102, 103, 104, 105, 106, 107, 108, 109, or 110 mg/mL.

In some aspects, the RSV RNA-LNP composition and/or influenza RNA-LNP composition is a lyophilized composition. In some aspects, the RSV subunit composition and influenza RNA-LNP composition is a lyophilized composition. In such compositions, the stabilizing agent (e.g., sucrose) is included in the lyophilized composition at a concentration, after reconstitution, of at least, at most, between any two of, or exactly 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 mg/mL.

In some aspects, the stabilizing agent (e.g., sucrose) is included in the lyophilized composition at a concentration, after reconstitution, of at least 20, at least 25, at least 30, at least 35, at least 40, at least 45, at least 50, at least 55, at least 60, at least 65, at least 70, at least 75 or at least 80 mg/mL. In some aspects, the stabilizing agent (e.g., sucrose) is included in the lyophilized composition at a concentration, after reconstitution, of between 20 to 30, between 30 to 40, between 40 to 50, between 50 to 60, between 60 to 70 or between 70 to 80 mg/mL.

In specific aspects, the stabilizing agent (e.g., sucrose) is included in the lyophilized composition at a concentration, after reconstitution, of 35 to 50 mg/mL. In specific aspects, the stabilizing agent (e.g., sucrose) is included in the lyophilized composition at a concentration, after reconstitution, of 35 to 45 mg/mL. In specific aspects, the stabilizing agent (e.g., sucrose) is included in the lyophilized composition at a concentration, after reconstitution, of 40 to 50 mg/mL. In specific aspects, the stabilizing agent (e.g., sucrose) is included in the lyophilized composition at a concentration, after reconstitution, of 35, 36, 37, 38, 39, 40, 41, 42, 43, 44, 45, 46, 47, 48, 49 or 50 mg/mL.

In some aspects, the RSV RNA-LNP composition and/or influenza RNA-LNP composition is a lyophilized composition, and the lyophilized RSV RNA-LNP composition and/or influenza RNA-LNP composition further comprises a salt. In some aspects, the composition comprising a RSV subunit and influenza RNA-LNP is a lyophilized composition, and the lyophilized composition further comprises a salt. The salt may comprise any one or more salts disclosed herein. In specific aspects, the salt comprises sodium chloride (NaCl). In some aspects, the salt (e.g., NaCl) is included in the lyophilized composition at a concentration, after reconstitution, of at least, at most, between any two of, or exactly 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or 50 ng/μg/mg per mL. In some aspects, the salt (e.g., NaCl) is included in the lyophilized composition at a concentration, after reconstitution, of in at least, at most, between any two of, or exactly 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 mg/mL.

In some aspects, the salt (e.g., NaCl) is included in the lyophilized composition at a concentration, after reconstitution, of at least 1, at least 2, at least 3, at least 4, at least 5, at least 6, at least 7, at least 8, at least 9, at least 10, at least 11, at least 12, at least 13, at least 14, at least 15, or at least 20 mg/mL.

In specific aspects, the salt (e.g., NaCl) is included in the lyophilized composition at a concentration, after reconstitution, of between 5 and 15 mg/mL. In some aspects, the salt (e.g., NaCl) is included in the lyophilized composition at a concentration, after reconstitution, of between 5 and 10 mg/mL. In specific aspects, the salt (e.g., NaCl) is included in the lyophilized composition at a concentration, after reconstitution, of 5, 6, 7, 8, 9, 10, 11, 12, 13, 14, or 15 mg/mL.

In some aspects, lyophilized compositions are reconstituted in a suitable carrier or diluent. The carrier or diluent may comprise any one or more carriers or diluents disclosed herein. In specific aspects, the carrier or diluent comprises saline, e.g., physiological saline. The saline may comprise 0.9% saline for injection. In some aspects, the lyophilized compositions are reconstituted in at least, at most, between any two of, or exactly 0.1, 0.2, 0.3, 0.4, 0.5, 0.6, 0.7, 0.8, 0.9, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1 mL of saline. In some aspects, the lyophilized compositions are reconstituted in at least 0.1, at least 0.2, at least 0.3, at least 0.4, at least 0.5, at least 0.6, at least 0.7, at least 0.8, at least 0.9, or at least 1 mL of saline.

In specific aspects, the lyophilized compositions are reconstituted in 0.6 to 0.75 mL of saline. In specific aspects, the lyophilized compositions are reconstituted in 0.65 to 0.75 mL of saline. In specific aspects, the lyophilized compositions are reconstituted in 0.6, 0.61, 0.62, 0.63, 0.64, 0.65, 0.66, 0.67, 0.68, 0.69, 0.7, 0.71, 0.72, 0.73, 0.74 or 0.75 mL of saline. The pH of the RSV RNA-LNP composition and/or influenza RNA-LNP composition may be at least, at most, exactly, or between any two of pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5, or any range or value derivable therein.

In some aspects, the RSV RNA-LNP composition and/or influenza RNA-LNP composition is at a pH of at least 6.5, at least 7.0, at least 7.5, at least 8.0, or at least 8.5. In specific aspects, the RSV RNA-LNP composition and/or influenza RNA-LNP composition is at a pH between 6.0 and 7.5, between 6.5 and 7.5, between 7.0 and 8.0, between and 7.5 and 8.5. In specific aspects, the RSV RNA-LNP composition and/or influenza RNA-LNP composition is between 7.0 and 8.0. In specific aspects, the RSV RNA-LNP composition and/or influenza RNA-LNP composition is at pH 7.0, 7.1, 7.2, 7.3, 7.4, 7.5, 7.6, 7.7, 7.8, 7.9 or 8.0.

The pH of the composition comprising a RSV subunit and an influenza RNA-LNP may be at least, at most, exactly, or between any two of pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5, or any range or value derivable therein. In some aspects, the composition comprising a RSV subunit and an influenza RNA-LNP is at a pH of at least 6.5, at least 7.0, at least 7.5, at least 8.0, or at least 8.5. In specific aspects, the composition comprising a RSV subunit and an influenza RNA-LNP is at a pH between 6.0 and 7.5, between 6.5 and 7.5, between 7.0 and 8.0, between and 7.5 and 8.5. In specific aspects, the composition comprising a RSV subunit and an influenza RNA-LNP is between 7.0 and 8.0. In specific aspects, the composition comprising a RSV subunit and an influenza RNA-LNP is at pH 7.0, 7.1, 7.2, 7.3, 7.4, 7.5, 7.6, 7.7, 7.8, 7.9 or 8.0.

In specific aspects, a RSV RNA-LNP composition comprises a RSV RNA polynucleotide encoding a RSV polypeptide as disclosed herein, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL.

In specific aspects, a RSV RNA-LNP composition comprises a RSV RNA polynucleotide encoding a RSV polypeptide as disclosed herein, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL.

In specific aspects, the RSV RNA-LNP composition is a liquid RSV RNA-LNP composition, and the liquid RSV RNA-LNP composition further comprises a buffer composition comprising a first buffer at a concentration of 0.15 to 0.3 mg/mL, a second buffer at a concentration of 1.25 to 1.4 mg/mL, and a stabilizing agent at a concentration of 95 to 110 mg/mL.

In specific aspects, the RSV RNA-LNP composition is a liquid RSV RNA-LNP composition, and the liquid RSV RNA-LNP composition further comprises a Tris buffer composition comprising tromethamine at a concentration of 0.1 to 0.3 mg/mL, Tris HCl at a concentration of 1.25 to 1.4 mg/mL, and sucrose at a concentration of 95 to 110 mg/mL.

Thus, in specific aspects, a liquid RSV RNA-LNP composition comprises an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, a second structural lipid at a concentration of 0.3 to 0.45 mg/mL, and further comprises a first buffer at a concentration of 0.1 to 0.3 mg/mL, a second buffer at a concentration of 1.25 to 1.4 mg/mL, and a stabilizing agent at a concentration of 95 to 110 mg/mL.

Thus, in specific aspects, a liquid RSV RNA-LNP composition comprises ALC-0315 at a concentration of 0.8 to 0.95 mg/mL, ALC-0159 at a concentration of 0.05 to 0.15 mg/mL, DSPC at a concentration of 0.1 to 0.25 mg/mL, cholesterol at a concentration of 0.3 to 0.45 mg/mL, and further comprises tromethamine at a concentration of 0.1 to 0.3 mg/mL, Tris HCl at a concentration of 1.25 to 1.4 mg/mL, and sucrose at a concentration of 95 to 110 mg/mL.

In specific aspects, the RSV RNA-LNP composition is a lyophilized RSV RNA-LNP composition, and the lyophilized RSV RNA-LNP composition further comprises a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL.

In specific aspects, the RSV RNA-LNP composition is a lyophilized RSV RNA-LNP composition, and the lyophilized RSV RNA-LNP composition further comprises a Tris buffer composition comprising tromethamine at a concentration of 0.01 and 0.15 mg/mL, Tris HCl at a concentration of 0.5 and 0.65 mg/mL, sucrose at a concentration of 35 to 50 mg/mL, and a sodium chloride (NaCl) at a concentration of 5 to 15 mg/mL.

Thus, in specific aspects, a lyophilized RSV RNA-LNP composition comprises a cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, a second structural lipid at a concentration of 0.3 to 0.45 mg/mL, and further comprises a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL. In specific aspects, the lyophilized compositions are reconstituted in 0.6 to 0.75 mL of carrier or diluent.

Thus, in some aspects, a lyophilized RSV RNA-LNP composition comprises ALC-0315 at a concentration of 0.8 to 0.95 mg/mL, ALC-0159 at a concentration of 0.05 to 0.15 mg/mL, DSPC at a concentration of 0.1 to 0.25 mg/mL, cholesterol at a concentration of 0.3 to 0.45 mg/mL, and further comprises tromethamine at a concentration of 0.01 and 0.15 mg/mL, Tris HCl at a concentration of 0.5 and 0.65 mg/mL, sucrose at a concentration of 35 to 50 mg/mL, and NaCl at a concentration of 5 to 15 mg/mL. In specific aspects, the lyophilized compositions are reconstituted in 0.6 to 0.75 mL of saline.

Concentrations in the lyophilized RSV RNA-LNP composition are determined post-reconstitution.

The RSV RNA-LNP compositions further comprise RSV RNA described herein encapsulated in LNPs, see section D. ADMINISTRATION.

In specific aspects, a RSV RNA-LNP composition is a liquid RSV RNA-LNP composition comprising a RSV RNA polynucleotide encoding a RSV polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL, and further comprising a buffer composition comprising a first buffer at a concentration of 0.15 to 0.3 mg/mL, a second buffer at a concentration of 1.25 to 1.4 mg/mL, and a stabilizing agent at a concentration of 95 to 110 mg/mL.

In specific aspects, a liquid RSV RNA-LNP composition comprises a RSV RNA polynucleotide encoding a RSV polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL, and further comprising a Tris buffer composition comprising tromethamine at a concentration of 0.1 to 0.3 mg/mL, Tris HCl at a concentration of 1.25 to 1.4 mg/mL, and sucrose at a concentration of 95 to 110 mg/mL.

In specific aspects, the RSV RNA-LNP composition is a lyophilized RSV RNA-LNP composition comprising a RSV RNA polynucleotide encoding a RSV polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL, and further comprising a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL. In specific aspects, the lyophilized compositions are reconstituted in 0.6 to 0.75 mL of carrier or diluent. Concentrations in the lyophilized RSV RNA-LNP composition are determined post-reconstitution.

In specific aspects, a lyophilized RSV RNA-LNP composition comprises a RSV RNA polynucleotide encoding a RSV polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL, and further comprising tromethamine at a concentration of 0.01 and 0.15 mg/mL, Tris HCl at a concentration of 0.5 and 0.65 mg/mL, sucrose at a concentration of 35 to 50 mg/mL, and NaCl at a concentration of 5 to 15 mg/mL. In specific aspects, the lyophilized compositions are reconstituted in 0.6 to 0.75 mL of saline. Concentrations in the lyophilized RSV RNA-LNP composition are determined post-reconstitution.

In specific aspects, an influenza RNA-LNP composition comprises an influenza RNA polynucleotide encoding an influenza polypeptide as disclosed herein, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL.

In specific aspects, an influenza RNA-LNP composition comprises an influenza RNA polynucleotide encoding an influenza polypeptide as disclosed herein, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL.

In specific aspects, the influenza RNA-LNP composition is a liquid influenza RNA-LNP composition, and the liquid influenza RNA-LNP composition further comprises a buffer composition comprising a first buffer at a concentration of 0.15 to 0.3 mg/mL, a second buffer at a concentration of 1.25 to 1.4 mg/mL, and a stabilizing agent at a concentration of 95 to 110 mg/mL. In specific aspects, the influenza RNA-LNP composition is a liquid influenza RNA-LNP composition, and the liquid influenza RNA-LNP composition further comprises a Tris buffer composition comprising tromethamine at a concentration of 0.1 to 0.3 mg/mL, Tris HCl at a concentration of 1.25 to 1.4 mg/mL, and sucrose at a concentration of 95 to 110 mg/mL.

Thus, in specific aspects, a liquid influenza RNA-LNP composition comprises an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, a second structural lipid at a concentration of 0.3 to 0.45 mg/mL, and further comprises a first buffer at a concentration of 0.1 to 0.3 mg/mL, a second buffer at a concentration of 1.25 to 1.4 mg/mL, and a stabilizing agent at a concentration of 95 to 110 mg/mL.

Thus, in specific aspects, a liquid influenza RNA-LNP composition comprises ALC-0315 at a concentration of 0.8 to 0.95 mg/mL, ALC-0159 at a concentration of 0.05 to 0.15 mg/mL, DSPC at a concentration of 0.1 to 0.25 mg/mL, cholesterol at a concentration of 0.3 to 0.45 mg/mL, and further comprises tromethamine at a concentration of 0.1 to 0.3 mg/mL, Tris HCl at a concentration of 1.25 to 1.4 mg/mL, and sucrose at a concentration of 95 to 110 mg/mL.

In specific aspects, the influenza RNA-LNP composition is a lyophilized influenza RNA-LNP composition, and the lyophilized influenza RNA-LNP composition further comprises a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL.

In specific aspects, the influenza RNA-LNP composition is a lyophilized influenza RNA-LNP composition, and the lyophilized influenza RNA-LNP composition further comprises a Tris buffer composition comprising tromethamine at a concentration of 0.01 and 0.15 mg/mL, Tris HCl at a concentration of 0.5 and 0.65 mg/mL, sucrose at a concentration of 35 to 50 mg/mL, and a sodium chloride (NaCl) at a concentration of 5 to 15 mg/mL.

Thus, in specific aspects, a lyophilized influenza RNA-LNP composition comprises a cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, a second structural lipid at a concentration of 0.3 to 0.45 mg/mL, and further comprises a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL. In specific aspects, the lyophilized compositions are reconstituted in 0.6 to 0.75 mL of carrier or diluent.

Thus, in some aspects, a lyophilized influenza RNA-LNP composition comprises ALC-0315 at a concentration of 0.8 to 0.95 mg/mL, ALC-0159 at a concentration of 0.05 to 0.15 mg/mL, DSPC at a concentration of 0.1 to 0.25 mg/mL, cholesterol at a concentration of 0.3 to 0.45 mg/mL, and further comprises tromethamine at a concentration of 0.01 and 0.15 mg/mL, Tris HCl at a concentration of 0.5 and 0.65 mg/mL, sucrose at a concentration of 35 to 50 mg/mL, and NaCl at a concentration of 5 to 15 mg/mL. In specific aspects, the lyophilized compositions are reconstituted in 0.6 to 0.75 mL of saline.

Concentrations in the lyophilized influenza RNA-LNP composition are determined post-reconstitution.

The influenza RNA-LNP compositions further comprise influenza RNA described herein encapsulated in LNPs, see section D. ADMINISTRATION.

In specific aspects, an influenza RNA-LNP composition is a liquid influenza RNA-LNP composition comprising an influenza RNA polynucleotide encoding an influenza polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL, and further comprising a buffer composition comprising a first buffer at a concentration of 0.15 to 0.3 mg/mL, a second buffer at a concentration of 1.25 to 1.4 mg/mL, and a stabilizing agent at a concentration of 95 to 110 mg/mL.

In specific aspects, a liquid influenza RNA-LNP composition comprises an influenza RNA polynucleotide encoding an influenza polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL, and further comprising a Tris buffer composition comprising tromethamine at a concentration of 0.1 to 0.3 mg/mL, Tris HCl at a concentration of 1.25 to 1.4 mg/mL, and sucrose at a concentration of 95 to 110 mg/mL.

In specific aspects, the influenza RNA-LNP composition is a lyophilized influenza RNA-LNP composition comprising an influenza RNA polynucleotide encoding an influenza polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL, and further comprising a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL. In specific aspects, the lyophilized compositions are reconstituted in 0.6 to 0.75 mL of carrier or diluent. Concentrations in the lyophilized influenza RNA-LNP composition are determined post-reconstitution.

In specific aspects, a lyophilized influenza RNA-LNP composition comprises an influenza RNA polynucleotide encoding an influenza polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL, and further comprising tromethamine at a concentration of 0.01 and 0.15 mg/mL, Tris HCl at a concentration of 0.5 and 0.65 mg/mL, sucrose at a concentration of 35 to 50 mg/mL, and NaCl at a concentration of 5 to 15 mg/mL. In specific aspects, the lyophilized compositions are reconstituted in 0.6 to 0.75 mL of saline. Concentrations in the lyophilized influenza RNA-LNP composition are determined post-reconstitution.

Some embodiments of lyophilized pharmaceutical compositions and/or compositions made by lyophilization methods disclosed herein comprise low moisture contents. Moisture contents may be measured by methods known in the art, such as gas chromatography. In some embodiments, the composition has a moisture content of less than or equal to 6.0% w/w. In some embodiments, the composition has a moisture content of less than or equal to 5.0% w/w. In some embodiments, the composition has a moisture content of less than or equal to 4.0% w/w. In some embodiments, the composition has a moisture content of less than or equal to 3.5% w/w. In some embodiments, the composition has a moisture content of less than or equal to 3.0% w/w. In some embodiments, the composition has a moisture content of less than or equal to 2.5% w/w. In some embodiments, the composition has a moisture content of less than or equal to 2.0% w/w. In some embodiments, the composition has a moisture content of less than or equal to 1.0% w/w. In some embodiments, the composition has a moisture content of less than or equal to 0.5% w/w. In some embodiments, the composition has a moisture content between 0.01% and 6.0% w/w, between 0.01% and 5.0% w/w, between 0.01% and 4.0% w/w, between 0.01% and 3.0% w/w, between 0.01% and 2.0% w/w, or between 0.01% and 1.0% w/w. In some embodiments, the composition has a moisture content between 0.01% w/w and 2.0% w/w. In some embodiments, the composition has a moisture content between 0.01% w/w and 1.0% w/w. In some embodiments, the composition has a moisture content between 0.01% w/w and 0.5% w/w. In some embodiments, moisture content refers to the amount of moisture in a lyophilized composition after 1 or more (e.g., 3, 6, 9, 12, 24, 36, 48, 60, 72, 84, 96, 108, 120, or more) months of storage.

In some embodiments, upon reconstitution the lipid nanoparticle has a diameter (or a composition has a mean lipid particle diameter) of 120 nm or less, such as 80 nm or less, 70 nm or less, 60 nm or less, 50 nm or less, 40 nm or less, 30 nm or less, or 20 nm or less. In some embodiments, upon reconstitution the lipid nanoparticle (or a composition has a mean lipid particle diameter) has a diameter of at most 30 nm. In some embodiments, the lipid nanoparticle has a diameter from 5-120 nm, 5-80 nm, 5-70 nm, 5-60 nm, 5-50 nm, 5-40 nm, 5-30 nm, or 5-20 nm. In some embodiments, the lyophilization increases the lipid nanoparticle size (or a mean lipid particle diameter) (e.g., as determined by DLS) by about 30 nm or less, such as about 25 nm or less, about 20 nm or less, about 15 nm or less, about 10 nm or less, or about 5 nm or less. In some embodiments, the lyophilization does not measurably increase the lipid nanoparticle size (or mean lipid particle diameter).

In some embodiments, a coefficient of degradation at 5° C. of the mRNA in the lyophilized composition is at most 0.05 month-1, at most 0.04 month-1, at most 0.03 month-1, at most 0.02 month-1, or at most 0.01 month-1. In some embodiments, the coefficient of degradation is at most 0.02 month-1. In some embodiments, the coefficient of degradation is at most 0.01 month-1. In some embodiments, the coefficient of degradation is between 0.0001 and 0.05 month-1, 0.0001 and 0.04 month-1, 0.0001 and 0.03 month-1, 0.001 and 0.02 month-1, or 0.001 and 0.01 month-1. In some embodiments, the coefficient of degradation is between 0.0001 and 0.02 month-1. In some embodiments, the coefficient of degradation is between 0.001 and 0.01 month-1. In some embodiments, the mRNA in the lyophilized composition is at least 50% pure after at least 12 months, at least 18 months, at least 21 months, at least 24 months, at least 27 months, at least 30 months, at least 33 months, or at least 36 months of storage. In some embodiments, the mRNA in the lyophilized composition is at least 50% pure after 12 to 120 months, 12 to 108 months, 12 to 96 months, 12 to 84 months, 12 to 72 months, 12 to 60 months, 12 to 54 months, 12 to 48 months, 12 to 42 months, 12 to 36 months, 12 to 30 months, 12 to 24 months, or 12 to 18 months of storage at a temperature between about 2° C. and about 8° C.

In some embodiments, the storage is conducted at a temperature of about 4° C. In some embodiments, the storage is conducted at about 5° C. In some embodiments, the mRNA in the lyophilized composition is at least 50% pure after at least 24 months of storage. In some embodiments, the mRNA in the lyophilized composition is at least 50% pure after 24 to 120 months, 24 to 108 months, 24 to 96 months, 24 to 84 months, 24 to 72 months, 24 to 60 months, 24 to 54 months, 24 to 48 months, 24 to 42 months, 24 to 36 months, or 24 to 30 months of storage at a temperature between about 2° C. and about 8° C. In some embodiments, the storage is conducted at a temperature of about 4° C. In some embodiments, the storage is conducted at a temperature between about 2° C. and about 8° C. In some embodiments, the storage is conducted at a temperature of about 4° C. In some embodiments, the storage is conducted at about 5° C. In some embodiments, the moisture content of a lyophilized composition remains below 6.0% (w/w), 5.0%, 4.0%, 3.0%, 2.0%, 1.5%, 1.0%, or 0.5% after 3-12 months of storage. For example, in some embodiments, the moisture content of a composition after 12 months of storage at 2-8° C. is less than or equal to 6.0% w/w. In some embodiments, the moisture content of a composition after 3-12 months of storage at 2-8° C. is less than or equal to 5.0% w/w. In some embodiments, the moisture content of a composition after 3-12 months of storage at 2-8° C. is less than or equal to 4.0% w/w. In some embodiments, the moisture content of a composition after 3-12 months of storage at 2-8° C. is less than or equal to 3.5% w/w. In some embodiments, the moisture content of a composition after 3-12 months of storage at 2-8° C. is less than or equal to 3.0% w/w. In some embodiments, the moisture content of a composition after 3-12 months of storage at 2-8° C. is less than or equal to 2.5% w/w. In some embodiments, the moisture content of a composition after 3-12 months of storage at 2-8° C. is less than or equal to 2.0% w/w. In some embodiments, the moisture content of a composition after 3-12 months of storage at 2-8° C. is less than or equal to 2.0% w/w. In some embodiments, the moisture content of a composition after 3-12 months of storage at 2-8° C. is less than or equal to 1.5% w/w. In some embodiments, the moisture content of a composition after 3-12 months of storage at 2-8° C. is less than or equal to 1.0%. In some embodiments, the moisture content of a composition after 3-12 months of storage at 2-8° C. is less than or equal to 0.5%. In some embodiments, the moisture content of a lyophilized composition is less than or equal to 2.0% w/w after 3 or more, 4 or more, 5 or more, 6 or more, 9 or more, or 12 or more months of storage at 2-8° C. In some embodiments, the moisture content of a lyophilized composition remains below 6.0% w/w after 3-12 months of storage at a given temperature and/or relative humidity. For example, in some embodiments, the moisture content of a composition after 3-12 months of storage at 20-30° C. is less than or equal to 6.0% w/w. In some embodiments, the moisture content of a composition after 3-12 months of storage at 20-30° C. is less than or equal to 5.0% w/w. In some embodiments, the moisture content of a composition after 3-12 months of storage at 20-30° C. is less than or equal to 4.0% w/w. In some embodiments, the moisture content of a composition after 3-12 months of storage at 20-30° C. is less than or equal to 3.5% w/w. In some embodiments, the moisture content of a composition after 3-12 months of storage at 20-30° C. is less than or equal to 3.0% w/w. In some embodiments, the moisture content of a composition after 3-12 months of storage at 20-30° C. is less than or equal to 2.5% w/w. In some embodiments, the moisture content of a composition after 3-12 months of storage at 20-30° C. is less than or equal to 2.0% w/w. In some embodiments, the moisture content of a composition after 3-12 months of storage at 20-30° C. is less than or equal to 2.0% w/w. In some embodiments, the moisture content of a composition after 3-12 months of storage at 20-30° C. is less than or equal to 1.5% w/w. In some embodiments, the moisture content of a composition after 3-12 months of storage at 20-30° C. is less than or equal to 1.0%. In some embodiments, the moisture content of a composition after 3-12 months of storage at 20-30° C. is less than or equal to 0.5%. In some embodiments, the moisture content of a lyophilized composition is less than or equal to 2.0% w/w after 3 or more, 4 or more, 5 or more, 6 or more, 9 or more, or 12 or more months of storage at 20-30° C.

In some embodiments, the storage is conducted at 50-100%, 60-100%, 70-100%, 75%-100%, 80-100%, 90-100% or 95-100% relative humidity.

In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 6 months of storage. In some embodiments, the storage is conducted at a temperature between 2-8° C. In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 9 months of storage. In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 12 months of storage. In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 24 months of storage. In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 36 months of storage.

In some embodiments, the storage is conducted at a temperature between 10-20° C. In some embodiments, the storage is conducted at a temperature between 20-30° C. In some embodiments, the storage is conducted at a temperature between 40-50° C. In some embodiments, the storage is conducted at a temperature between 50-60° C. In some embodiments, the storage is conducted for 3-120 months, 3-96 months, 3-72 months, 3-60 months. 3-48 months, 3-36 months, 3-24 months, 3-12 months, 12-120 months, 12-96 months, 12-72 months, 12-60 months, 12-48 months, 12-36 months, 12-24 months, 24-120 months, 24-96 months, 24-72 months, or 24-48 months. In some embodiments, the storage is conducted at 50-100%, 60-100%, 70-100%, 75%-100%, 80-100%, 90-100% or 95-100% relative humidity.

In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 3, 6, 9, 12, 18, 24, 36, 48, 60, or more months of storage above 30° C. (e.g., from about 40° C. to about 50° C.). In some embodiments, the lyophilized compositions comprise mRNA with at least 96% relative purity after 1 month of storage between 30-60° C. In some embodiments, the lyophilized compositions comprise mRNA with at least 93% relative purity after 2 months of storage between 30-60° C. In some embodiments, the lyophilized compositions comprise mRNA with at least 90% relative purity after 3 months of storage between 30-60° C. In some embodiments, the lyophilized compositions comprise mRNA with at least 85% relative purity after 4 months of storage between 30-60° C. In some embodiments, the lyophilized compositions comprise mRNA with at least 85% relative purity after 5 months of storage between 30-60° C. In some embodiments, the lyophilized compositions comprise mRNA with at least 80% relative purity after 6 months of storage between 30-60° C. In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 6 months of storage at a relative humidity above 50% (e.g., 50-100%, 50-75%, 50-80%, 80-100%, or 60-75%). In some embodiments, the lyophilized compositions comprise mRNA with at least 93% relative purity after 3 months of storage at relative humidity between 70-100%. In some embodiments, the lyophilized compositions comprise mRNA with at least 87% relative purity after 6 months of storage at relative humidity between 70-100%. In some embodiments, the lyophilized compositions comprise mRNA with at least 85% relative purity after 9 months of storage at relative humidity between 70-100%. In some embodiments, the lyophilized compositions comprise mRNA with at least 80% relative purity after 12 months of storage at relative humidity between 70-100%. In some embodiments, the lyophilized compositions comprise mRNA with at least 80% relative purity after 24 months of storage at relative humidity between 70-100%. In some embodiments, the lyophilized compositions comprise mRNA with at least 80% relative purity after 36 months of storage at relative humidity between 70-100%. In some embodiments, the lyophilized compositions comprise mRNA with at least 80% relative purity after 48 months of storage at relative humidity between 70-100%. In some embodiments, the lyophilized compositions comprise mRNA with at least 80% relative purity after 60 months of storage at relative humidity between 70-100%. In some embodiments, the lyophilized compositions comprise mRNA with at least 90%, at least 91%, at least 92%, at least 93%, at least 94%, at least 96%, at least 97%, at least 98%, or at least 99% relative purity, compared to the purity of mRNA immediately after the conclusion of lyophilization, after 3, 6, 9, 12, 18, 24, 36, 48, 60, or more months of storage between 2-8° C. In some embodiments, the lyophilized compositions comprise mRNA with at least 90% relative purity after 12 months of storage between 2-8° C. In some embodiments, the lyophilized compositions comprise mRNA with at least 90% relative purity after 24 months of storage between 2-8° C. In some embodiments, the lyophilized compositions comprise mRNA with at least 90% relative purity after 36 months of storage between 2-8° C. In some embodiments, the lyophilized compositions comprise mRNA with at least 90% relative purity after 48 months of storage between 2-8° C. In some embodiments, the lyophilized compositions comprise mRNA with at least 90% relative purity after 60 months of storage between 2-8° C. In some embodiments, the lyophilized compositions comprise mRNA with at least 90% relative purity after 72, 96, 108, 120, or more, months of storage between 2-8° C. In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 3, 6, 9, 12, 18, 24, 36, 48, 60, or more months of storage between 20-30° C. In some embodiments, the lyophilized compositions comprise mRNA with at least 80% relative purity after 12 months of storage between 20-30° C. In some embodiments, the lyophilized compositions comprise mRNA with at least 80% relative purity after 24 months of storage between 20-30° C. In some embodiments, the lyophilized compositions comprise mRNA with at least 80% relative purity after 36 months of storage between 20-30° C. In some embodiments, the lyophilized compositions comprise mRNA with at least 80% relative purity after 48 months of storage between 20-30° C. In some embodiments, the lyophilized compositions comprise mRNA with at least 80% relative purity after 60 months of storage between 20-30° C. In some embodiments, the lyophilized compositions comprise mRNA with at least 80% relative purity after 72, 96, 108, 120, or more, months of storage between 20-30° C.

In some embodiments, lipid nanoparticles of the lyophilized compositions comprise lipid nanoparticles comprising mRNA with an encapsulation efficiency of 70%. As used herein, “encapsulation efficiency” refers to the percentage of nucleic acid (e.g., mRNA) in a composition that is comprised within lipid nanoparticles. mRNA encapsulated within a lipid nanoparticle is not exposed to the environment outside the lipid nanoparticle, and is thus protected from the action of environmental factors such as nucleases, which can cleave free mRNA. Encapsulation efficiency can be measured by any one of multiple methods known in the art, such as a RiboGreen assay. RiboGreen is a fluorescent dye that is fluorescent only when bound to a nucleic acid. In a RiboGreen assay, one sample of a composition containing mRNA in lipid nanoparticles is subjected to a treatment that disrupts lipid nanoparticle integrity to release encapsulated mRNA, such as exposure to a detergent, while another sample is not disrupted. RiboGreen is then added to both samples, and the fluorescence emitted from both samples is measured. Encapsulation efficiency (E.E.) is calculated by known equations. In some embodiments, the encapsulation efficiency is at least 70%, at least 75%, at least 80%, at least 85%, at least 90%, at least 91%, at least 92%, at least 93%, at least 94%, at least 95%, at least 96%, at least 97%, at least 98%, at least 99%, or up to 100%. In some embodiments, RNA encapsulation decreases by about 10% or less, such as about 9% or less, about 8% or less, about 7% or less, about 6% or less, about 5% or less, about 4% or less, about 3% or less, about 2% or less, or about 1% or less immediately after lyophilization and/or after storage following lyophilization.

In some embodiments, the amount of encapsulated RNA does not substantially decrease during lyophilization and/or storage of a lyophilized LNP. In some embodiments, the lyophilized compositions comprise mRNA with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, up to 100%, or greater than 100% in vitro potency, relative to the in vitro potency of the mRNA that was present in the composition prior to lyophilization. As used herein, “in vitro potency” of an mRNA refers to the capacity of an mRNA to be translated into a protein encoded by the mRNA during an in vitro translation reaction. In in vitro translation, an mRNA encoding a protein is incubated in the presence of ribosomes and aminoacyl-tRNAs, which allow the encoded protein to be produced in the absence of cells. Calculating the in vitro potency of a first mRNA relative to a second mRNA encoding the same protein may be calculated by known methods.

Some embodiments comprise reconstituting a lyophilized lipid nanoparticle composition, e.g., in a reconstitution buffer suitable for pharmaceutical administration. In some embodiments, the lyophilized lipid nanoparticle composition is reconstituted in water. In some embodiments, the lyophilized lipid nanoparticle composition is reconstituted in a salt solution (e.g., a sodium chloride solution), such as an about 0.5%, about 0.9%, about 1%, about 1.5%, about 2%, about 5%, about 10%, about 30%, about 40%, about 50%, about 60%, about 70%, about 80%, or about 90% salt solution.

In some embodiments, the reconstitution buffer is at a physiological pH (e.g, pH 7-7.5, such as 7.4).

Some embodiments comprise a reconstituted lipid nanoparticle composition. In some embodiments, the lyophilized composition is reconstituted (e.g., via agitation, swirling, shaking, and/or repeated pipette aspirating and dispensing) at a temperature of from about 1° C. to about 75° C., such as about 4° C., about 5° C., about 10° C., about 15° C., about 20° C., about 25° C. about 30° C., about 35° C., about 40° C., about 45° C., about 50° C., about 55° C., about 60° C., about 65° C., about 70° C., or about 75° C.

### 2. Vaccines

In some aspects, a pharmaceutical composition described herein is an immunogenic composition for inducing an immune response. For example, in some aspects, an immunogenic composition is a vaccine. In some aspects, the compositions described herein include at least one isolated nucleic acid or polypeptide molecule as described herein. In specific aspects, the immunogenic compositions comprise nucleic acids, and the immunogenic compositions are nucleic acid vaccines. In some aspects, the immunogenic compositions comprise RNA (e.g. mRNA, saRNA), and vaccines are RNA vaccines. In other aspects, the immunogenic compositions comprise DNA, and vaccines are DNA vaccines. In yet other aspects, the immunogenic compositions comprise a polypeptide, and vaccines are polypeptide vaccines. Conditions and/or diseases that may be treated with the nucleic acid and/or peptide or polypeptide compositions include, but are not limited to, those caused and/or impacted by infection, cancer, rare diseases, and other diseases or conditions caused by overproduction, underproduction, or improper production of protein or nucleic acids.

In some aspects, the composition is substantially free of one or more impurities or contaminants and, for instance, includes nucleic acid or polypeptide molecules that are equal to at least, at most, exactly, or between any two of 90%, 91%, 92%, 93%, 94%, 95%, 96%, 97%, 98%, or 99% pure; at least 98% pure, or at least 99% pure.

The present disclosure includes methods for preventing, treating or ameliorating an infection, disease or condition in a subject, including administering to a subject an effective amount of an RNA molecule that includes at least one open reading frame encoding a polypeptide or composition described herein. As such, the disclosure contemplates vaccines for use in both active and passive immunization aspects. Immunogenic compositions, proposed to be suitable for use as a vaccine, may be prepared from RNA molecules encoding polypeptide(s), such as RSV glycoproteins and/or polypeptides derived from influenza, e.g., HA and/or NA. In certain aspects, immunogenic compositions are lyophilized for more ready formulation into a desired vehicle.

The preparation of vaccines that contain nucleic acid and/or peptide or polypeptide as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference in their entireties. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific aspects, vaccines are formulated with a combination of substances, as described in U.S. Pat. Nos. 6,793,923 and 6,733,754, which are incorporated herein by reference in their entireties.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%. In some aspects, suppositories may be formed from mixtures containing the active ingredient in the range of about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient.

The polypeptide-encoding nucleic acid constructs and polypeptides may be formulated into a vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner.

However, suitable dosage ranges are of the order of several hundred micrograms of active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain aspects, it will be desirable to have one administration of the vaccine. In some aspects, it will be desirable to have multiple administrations of the vaccine, e.g., 2, 3, 4, 5, 6 or more administrations. The vaccinations may be at 1, 2, 3, 4, 5, 6, 7, 8, to 5, 6, 7, 8, 9, 10, 11, 12 twelve week intervals, including all ranges there between. In some aspects, vaccinations may be at 1, 2, 3, 4, 5, 6, 7, 8, 9, 10, 11, 12 month intervals, including all ranges there between. Periodic boosters at intervals of 1-5 years may be desirable to maintain protective levels of the antibodies.

### 3. Carriers

A pharmaceutically acceptable carrier may include the liquid or non-liquid basis of a composition. If a composition is provided in liquid form, the carrier may be water, such as pyrogen-free water; isotonic saline or buffered (aqueous) solutions, e.g. phosphate, citrate buffered solutions. Water or a buffer, such as an aqueous buffer, may be used, containing a sodium salt, a calcium salt, and and/or a potassium salt. The sodium, calcium and/or potassium salts may occur in the form of their halogenides, e.g. chlorides, iodides, or bromides, in the form of their hydroxides, carbonates, hydrogen carbonates, or sulfates, etc. Examples of sodium salts include, but are not limited to, NaCl, Nal, NaBr, Na₂CO₃, NaHCO₃, Na₂SO₄, Na₂HPO₄, Na₂HPO₄·₂H₂O, examples of potassium salts include, but are not limited to, KCl, KI, KBr, K₂CO₃, KHCO₃, K₂SO₄, KH₂PO₄, and examples of calcium salts include, but are not limited to, CaCl₂, CaI₂, CaBr₂, CaCO₃, CaSO₄, Ca(OH)₂. Examples of further carriers may include sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid. Examples of further carriers may include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

### 4. Adjuvants

In some embodiments, the composition further includes an adjuvant. Suitable adjuvants include all acceptable immunostimulatory compounds, such as cytokines, toxins, or synthetic compositions. A number of adjuvants may be used to enhance an antibody response. Adjuvants include, but are not limited to, oil-in-water emulsions, water-in-oil emulsions, mineral salts, polynucleotides, and natural substances. Specific adjuvants that may be used include Freund's adjuvant, oil such as MONTANIDE® ISA51, IL1, IL2, IL3, IL4, IL5, IL6, IL7, IL8, IL9, IL10, IL12, alpha-interferon, PTNGg, GM-CSF, GMCSP, BCG, LT-a, aluminum salts, such as aluminum hydroxide or other aluminum compound, MDP compounds, such as thur-MDP and nor-MDP, CGP (MTP-PE), lipid A, monophosphoryl lipid A (MPL), lipopeptides (e.g., Pam3Cys). RIBI, which contains three components extracted from bacteria, MPL, trehalose dimycolate (TDM), and cell wall skeleton (CWS) in a 2% squalene/Tween 80 emulsion. MHC antigens may even be used. In some embodiments, the composition further includes any one of the following adjuvants: a cytokine-inducing agent, or benzonaphthyridine compounds, CpG sequences, and 3dMPL (also known as 3 de-O-acylated monophosphoryl lipid A or 3-O-desacyl-4′-monophosphoryl lipid A).

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (CARBOPOL®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (FLUOSOL-DA®) used as a block substitute may also be employed to produce an adjuvant effect.

In addition to adjuvants, it may be desirable to co-administer biologic response modifiers (BRM) to enhance immune responses. BRMs have been shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m²) (Johnson/Mead, NJ) and cytokines such as y-interferon, IL-2, or IL-12 or genes encoding proteins involved in immune helper functions, such as B-7.

### 5. Combination Therapy

The compositions and related methods of the present disclosure, particularly administration of a RNA molecule encoding a RSV polypeptide and/or polypeptide derived from influenza, e.g., HA and/or NA, may also be used in combination with the administration of traditional therapies. These include, but are not limited to, the administration of antiviral therapies such as acyclovir, valacyclovir, and famciclovir, or various combinations of antivirals. Also included are the administration of one or more therapies to treat one or more symptoms of RSV infection and/or influenza infection, including, but not limited to, steroids including corticosteroids, anti-inflammatories including acetaminophen or ibuprofen, pain-relief agents, creams or lotions to relieve itching, cool compresses, or various combinations thereof.

In one aspect, it is contemplated that a vaccine and/or therapy is used in conjunction with antiviral treatment. Alternatively, the therapy may precede or follow the other agent treatment by intervals ranging from minutes to weeks. In aspects where the other agents and/or vaccines are administered separately, one would generally ensure that a significant period of time did not expire between the time of each delivery, such that the agent and immunogenic composition would still be able to exert an advantageously combined effect on the subject. In such aspects, it is contemplated that one may administer both modalities within about 12-24 h of each other or within about 6-12 h of each other. In some situations, it may be desirable to extend the time period for administration significantly, where several days (2, 3, 4, 5, 6 or 7) to several weeks (1, 2, 3, 4, 5, 6, 7 or 8) lapse between the respective administrations.

Various combinations may be employed, for example antiviral therapy “A” and immunogenic polypeptide given as part of an immune therapy regime “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the immunogenic compositions of the present disclosure to a patient/subject will follow general protocols for the administration of such compounds, taking into account the toxicity, if any, of the RSV RNA vaccine composition and/or influenza RNA vaccine composition and/or RSV subunit vaccine composition, or other compositions described herein. It is expected that the treatment cycles would be repeated as necessary. It also is contemplated that various standard therapies, such as hydration, may be applied in combination with the described therapy.

### 6. Administration

Administration of the compositions described herein may be carried out via any of the accepted modes of administration of agents for serving similar utilities. In some aspects, a pharmaceutical composition described herein may be administered intravenously, intraarterially, subcutaneously, intradermally or intramuscularly. In specific aspects, the RSV RNA molecules and/or influenza RNA molecules and/or respective RNA-LNP compositions are administered intramuscularly. In certain aspects, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In one aspect, the pharmaceutical composition is formulated for intramuscular administration. In another aspect, the pharmaceutical composition is formulated for systemic administration, e.g., for intravenous administration.

Pharmaceutical compositions may be formulated into preparations in solid, semi-solid, liquid, lyophilized, frozen, or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection, or infusion techniques. Pharmaceutical compositions described herein are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound in aerosol form may hold a plurality of dosage units. The composition to be administered will, in any event, contain a therapeutically and/or prophylactically effective amount of a compound within the scope of this disclosure, or a pharmaceutically acceptable salt thereof, for treatment of a disease or condition of interest in accordance with the teachings described herein.

A pharmaceutical composition within the scope of this disclosure may be in the form of a solid or liquid and may be frozen or lyophilized. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalatory administration. In some aspects, when intended for oral administration, the pharmaceutical composition is in either solid or liquid form, where semi-solid, semi-liquid, suspension, and gel forms are included within the forms considered herein as either solid or liquid. As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present or exclude: binders such as carboxymethylcellulose, ethyl cellulose, microcrystalline cellulose, gum tragacanth, or gelatin; excipients such as starch, lactose, or dextrins; disintegrating agents such as alginic acid, sodium alginate, PRIMOJEL®, corn starch and the like; lubricants such as magnesium stearate or STEROTEX®; glidants such as colloidal silicon dioxide; sweetening agents such as sucrose or saccharin; a flavoring agent such as peppermint, methyl salicylate, or orange flavoring; and a coloring agent. When the pharmaceutical composition is in the form of a capsule, for example, a gelatin capsule, it may contain, in addition to materials of the above type, a liquid carrier such as polyethylene glycol or oil. The pharmaceutical composition may be in the form of a liquid, for example, an elixir, syrup, solution, emulsion or suspension. The liquid may be for oral administration or for delivery by injection, as two examples. In some aspects, when intended for oral administration, compositions contain, in addition to the present compounds, one or more of a sweetening agent, preservatives, dye/colorant, and flavor enhancer. In a composition intended to be administered by injection, one or more of a surfactant, preservative, wetting agent, dispersing agent, suspending agent, buffer, stabilizer, and isotonic agent may be included or excluded.

A liquid pharmaceutical composition, whether they be solutions, suspensions, or other like form, may include or exclude one or more of the following adjuvants: sterile diluents such as water for injection, saline solution, e.g., physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose; agents to act as cryoprotectants such as sucrose or trehalose. The parenteral preparation may be enclosed in ampoules, disposable syringes, or multiple dose vials made of glass or plastic. In one aspect, physiological saline is the adjuvant. In one aspect, an injectable pharmaceutical composition is sterile. A liquid pharmaceutical composition intended for either parenteral or oral administration should contain an amount of a compound such that a suitable dosage will be obtained.

The pharmaceutical compositions may be prepared by methodology well known in the pharmaceutical art. For example, a pharmaceutical composition intended to be administered by injection may be prepared by combining the nucleic acid or polypeptide with sterile, distilled water or other carrier so as to form a solution. A surfactant may be added to facilitate the formation of a homogeneous solution or suspension. Surfactants are compounds that non-covalently interact with a compound consistent with the teachings herein so as to facilitate dissolution or homogeneous suspension of the compound in the aqueous delivery system.

The pharmaceutical compositions according to the present disclosure, or their pharmaceutically acceptable salts, are generally applied in a “therapeutically effective amount” or a “prophylactically effective amount” and in “a pharmaceutically acceptable preparation.” The term “pharmaceutically acceptable” refers to the non-toxicity of a material which does not interact with the action of the active component of the pharmaceutical composition. The terms “therapeutically effective amount” and “prophylactically effective amount” refer to the amount which achieves a desired reaction or a desired effect alone or together with further doses. In the case of the treatment of a particular disease, in one aspect, the desired reaction relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition.

The compositions within the scope of the disclosure are administered in a therapeutically and/or prophylactically effective amount, which will vary depending upon a variety of factors including the activity of the specific therapeutic and/or prophylactic agent employed; the metabolic stability and length of action of the therapeutic and/or prophylactic agent; the individual parameters of the patient, including the age, body weight, general health, gender, and diet of the patient; the mode, time, and/or duration of administration; the rate of excretion; the drug combination; the severity of the particular disorder or condition; and the subject undergoing therapy. Accordingly, the doses administered of the compositions described herein may depend on various of such parameters. In the case that a reaction in a patient is insufficient with an initial dose, higher doses (or effectively higher doses achieved by a different, more localized route of administration) may be used. In some aspects, compositions (e.g., RSV RNA-LNP compositions and/or influenza RNA-LNP compositions) may be administered at dosage levels sufficient to deliver 0.0001 ng/μg/mg per kg to 100 ng/μg/mg per kg, 0.001 ng/μg/mg per kg to 0.05 ng/μg/mg per kg, 0.005 ng/μg/mg per kg to 0.05 ng/μg/mg per kg, 0.001 ng/μg/mg per kg to 0.005 ng/μg/mg per kg, 0.05 ng/μg/mg per kg to 0.5 ng/μg/mg per kg, 0.01 ng/μg/mg per kg to 50 ng/μg/mg per kg, 0.1 ng/μg/mg per kg to 40 ng/μg/mg per kg, 0.5 ng/μg/mg per kg to 30 ng/μg/mg per kg, 0.01 ng/μg/mg per kg to 10 ng/μg/mg per kg, 0.1 ng/μg/mg per kg to 10 ng/μg/mg per kg, or 1 ng/μg/mg per kg to 25 ng/μg/mg per kg, of subject body weight per day, one or more times a day, per week, per month, etc. to obtain the desired therapeutic, diagnostic, prophylactic, or imaging effect (see e.g., the range of unit doses described in International Publication No. WO2013/078199, herein incorporated by reference in its entirety). In some aspects, compositions (e.g., RSV RNA-LNP compositions and/or influenza RNA-LNP compositions) may be administered at dosage levels sufficient to deliver at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg per kg, of subject body weight per day, one or more times a day, per week, per month, etc. to obtain the desired therapeutic, diagnostic, prophylactic, or imaging effect.

In some aspects, compositions (e.g., RSV RNA-LNP compositions and/or influenza RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg per day, one or more times a day, per week, per month, etc. to obtain the desired therapeutic, diagnostic, prophylactic, or imaging effect.

In specific aspects, compositions (e.g., RSV RNA-LNP compositions and/or influenza RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL RSV RNA encapsulated in LNP and/or influenza RNA encapsulated in LNP.

In exemplary aspects, compositions (e.g., RSV RNA-LNP compositions and/or influenza RNA-LNP compositions) may be administered at dose levels of at least, at most, exactly, or between any two of 0.01, 0.15, 0.30, 0.45, 0.60, 0.75, or 0.90 mg/mL RSV RNA encapsulated in LNP and/or influenza RNA encapsulated in LNP. In exemplary aspects, compositions (e.g., RSV RNA-LNP compositions) may be administered at dose levels of at least, at most, exactly, or between any two of 0.01, 0.15, 0.30, 0.45, 0.60, 0.75, or 0.90 mg RSV RNA encapsulated in LNP and/or influenza RNA encapsulated in LNP.

In specific aspects, compositions (e.g., RSV RNA-LNP compositions and/or influenza RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL RSV RNA encapsulated in LNP and/or influenza RNA encapsulated in LNP.

In exemplary aspects, compositions (e.g., RSV RNA-LNP compositions and/or influenza RNA-LNP compositions) may be administered at dose levels of at least, at most, exactly, or between any two of 1, 15, 30, 45, 60, 75, or 90 μg/mL RSV RNA encapsulated in LNP and/or influenza RNA encapsulated in LNP. In exemplary aspects, compositions (e.g., RSV RNA-LNP compositions and/or influenza RNA-LNP compositions) may be administered at dose levels of at least, at most, exactly, or between any two of 1, 15, 30, 45, 60, 75, or 90 μg RSV RNA encapsulated in LNP and/or influenza RNA encapsulated in LNP.

The desired dosage may be delivered multiple times a day (e.g., 1, 2, 3, 4, 5, or more times a day), every other day, every third day, every week, every two weeks, every three weeks, every four weeks, every 2 months, every three months, every 6 months, etc. In certain aspects, the desired dosage may be delivered using a single-dose administration. In certain aspects, the desired dosage may be delivered using multiple administrations (e.g., two, three, four, five, six, seven, eight, nine, ten, eleven, twelve, thirteen, fourteen, or more administrations). When multiple administrations are employed, split dosing regimens may be used. The time of administration between the initial administration of the composition and a subsequent administration of the composition may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In some aspects, compositions (e.g., RSV RNA-LNP compositions and/or influenza RNA-LNP compositions) may be administered in a single dose. In some aspects, compositions (e.g., RSV RNA-LNP compositions and/or influenza RNA-LNP compositions) may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 1 month later, Day 0 and 2 months later, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later), with each administration at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg RSV RNA encapsulated in LNP and/or influenza RNA encapsulated in LNP. Higher and lower dosages and frequency of administration are encompassed by the present disclosure. For example, compositions (e.g., RSV RNA-LNP compositions and/or influenza RNA-LNP compositions) may be administered three or four times. Periodic boosters at intervals of 1-5 years may be desirable to maintain protective levels of the antibodies.

In some aspects, the compositions (e.g., RSV RNA-LNP compositions and/or influenza RNA-LNP compositions) are administered to a subject as a single dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg of RSV RNA encapsulated in LNP and/or influenza RNA encapsulated in LNP. In some aspects, the compositions (e.g., RSV RNA-LNP compositions and/or influenza RNA-LNP compositions) are administered the subject as a single dose of at least, at most, exactly, or between any two of 1 μg, 15 μg, 30 μg, 45 μg, 60 μg, 75 μg, or 90 μg of RSV RNA encapsulated in LNP and/or influenza RNA encapsulated in LNP.

In some aspects, the compositions (e.g., RSV RNA-LNP compositions and/or influenza RNA-LNP compositions) are administered to a subject as two doses of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg of RSV RNA encapsulated in LNP and/or influenza RNA encapsulated in LNP. In some aspects, the compositions (e.g., RSV RNA-LNP compositions and/or influenza RNA-LNP compositions) are administered the subject as two doses of at least, at most, exactly, or between any two of 1 μg, 15 μg, 30 μg, 45 μg, 60 μg, 75 μg, or 90 μg of RSV RNA encapsulated in LNP and/or influenza RNA encapsulated in LNP.

In specific aspects, compositions (e.g., RSV RNA-LNP compositions and/or influenza RNA-LNP compositions) may be administered twice (e.g., Day 0 and Day 28, Day 0 and Day 60, Day 0 and Day 180, Day 0 and 2 months later, Day 0 and 6 months later), with each administration at a total dose of or at dosage levels sufficient to deliver a total dose of at least, at most, exactly, or between any two of 1 μg, 15 μg, 30 μg, 45 μg, 60 μg, 75 μg, or 90 μg RSV RNA encapsulated in LNP and/or influenza RNA encapsulated in LNP.

### IX. Methods of Use

Provided herein are compositions (e.g., pharmaceutical compositions comprising RSV RNA molecules and/or RSV RNA-LNPs and/or influenza RNA and/or influenza RNA-LNPs), methods, kits and reagents for prevention and/or treatment of RSV in humans and other mammals.

RSV RNA compositions and/or influenza RNA compositions (e.g., RSV RNA-LNP compositions and/or influenza RNA-LNP compositions) may be used as prophylactic agents. They may be used in medicine to prevent and/or treat infectious disease. In exemplary aspects, the RSV RNA compositions and/or influenza RNA compositions are used to provide prophylactic protection from acute lower respiratory infection (ALRI). The RSV vaccines of the present disclosure may be used to prevent RSV and/or influenza (infection-associated illness, including pneumonia and bronchitis) and may be particularly useful for prevention and/or treatment of immunocompromised and elderly patients to prevent or to reduce the severity and/or duration of RSV infection and/or influenza infection.

In some aspects, the RSV RNA compositions (e.g., RSV RNA-LNP compositions) and/or influenza RNA compositions (e.g., influenza RNA-LNP compositions) of the disclosure are administered to a subject (e.g., a mammalian subject, such as a human subject), and the RNA polynucleotides are translated in vivo to produce an antigenic polypeptide.

In some aspects, the RSV RNA compositions and/or influenza RNA compositions of the disclosure may be used to prime immune effector cells, for example, to activate peripheral blood mononuclear cells (PBMCs) ex vivo, which are then infused (re-infused) into a subject.

In some aspects, after administration of a RSV RNA molecule and/or influenza RNA molecule described herein, e.g., formulated as RNA-LNPs, at least a portion of the RNA is delivered to a target cell. In some aspects, at least a portion of the RNA is delivered to the cytosol of the target cell. In some aspects, the RNA is translated by the target cell to produce the polypeptide or protein it encodes. In some aspects, the target cell is a spleen cell. In some aspects, the target cell is an antigen presenting cell such as a professional antigen presenting cell in the spleen. In some aspects, the target cell is a dendritic cell or macrophage. RNA molecules such as RNA-LNPs described herein may be used for delivering RNA to such target cell. Accordingly, the present disclosure also relates to a method for delivering RNA to a target cell in a subject comprising the administration of the RNA-particles described herein to the subject.

In some aspects, the RNA is delivered to the cytosol of the target cell. In some aspects, the RNA is translated by the target cell to produce the polypeptide or protein encoded by the RNA. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, may be referred to as encoding the protein or other product of that gene or cDNA.

In some aspects, nucleic acid compositions described herein, e.g., compositions comprising a RSV RNA-LNP are characterized by (e.g., when administered to a subject) sustained expression of an encoded polypeptide. In some aspects, nucleic acid compositions described herein, e.g., compositions comprising an influenza RNA-LNP are characterized by (e.g., when administered to a subject) sustained expression of an encoded polypeptide. For example, in some aspects, such compositions are characterized in that, when administered to a human, they achieve detectable polypeptide expression in a biological sample (e.g., serum) from such human and, in some aspects, such expression persists for a period of time that is at least at least 36 hours or longer, including, e.g., at least 48 hours, at least 60 hours, at least 72 hours, at least 96 hours, at least 120 hours, at least 148 hours, or longer.

In some aspects, the disclosure relates to a method of inducing an immune response in a subject. The method includes administering to the subject an effective amount of an RNA molecule, RNA-LNP and/or composition as described herein.

In another aspect, the disclosure relates to a method of vaccinating a subject. The method includes administering to the subject in need thereof an effective amount of an RNA molecule, RNA-LNP and/or composition described herein.

In another aspect, the disclosure relates to a method of treating or preventing an infectious disease. The method includes administering to the subject an effective amount of an RNA molecule RNA-LNP and/or composition as described herein.

In another aspect, the disclosure relates to a method of treating or preventing or reducing the severity of an RSV infection and/or illness caused by RSV, and/or an influenza infection and/or illness caused by influenza. The method includes administering to the subject an effective amount of an RNA molecule, RNA-LNP and/or composition as described herein.

In another aspect, the disclosure relates to a method of treating or preventing or reducing the severity of an infectious disease in a subject by, for example, inducing an immune response to an infectious disease in the subject. In some aspects, the method includes administering a priming composition that includes an effective amount of an RNA molecule, RNA-LNP and/or composition described herein, and administering a booster composition including an effective amount of an RNA molecule, RNA-LNP and/or composition. In some aspects, the composition elicits an immune response including an antibody response. In some aspects, the composition elicits an immune response including a T cell response.

In another aspect, the disclosure relates to a method of treating or preventing or reducing the severity of an RSV infection and/or illness caused by RSV and/or an influenza infection and/or illness caused by influenza, in a subject by, for example, inducing a respective immune response to RSV and/or influenza in the subject. In some aspects, the method includes administering a priming composition that includes an effective amount of an RNA molecule, RNA-LNP and/or composition described herein, and administering a booster composition including an effective amount of an RNA molecule RNA-LNP and/or composition as described herein. In some aspects, the composition elicits an immune response including an antibody response. In some aspects, the composition elicits an immune response including a T cell response.

The methods disclosed herein may involve administering to the subject a RSV RNA-LNP composition comprising at least one RSV RNA molecule having an open reading frame encoding at least one RSV antigenic polypeptide, thereby inducing in the subject an immune response specific to RSV antigenic polypeptide, wherein anti-antigenic polypeptide antibody titer in the subject is increased following vaccination relative to anti-antigenic polypeptide antibody titer in a subject vaccinated with a prophylactically effective dose (e.g., a therapeutically effective dose that prevents infection with the virus at a clinically acceptable level) of a traditional vaccine against the RSV. An “anti-antigenic polypeptide antibody” is a serum antibody the binds specifically to the antigenic polypeptide. In some aspects, the anti-antigenic polypeptide antibody titer in the subject is increased at least, at most, between any two of, or exactly 1, 2, 3, 4, 5, 6, 7, 8, 9, or 10 log following administration of the RSV RNA-LNP composition relative to anti-antigenic polypeptide antibody titer in a subject administered a prophylactically effective dose of a traditional composition against RSV.

The methods disclosed herein may involve administering to the subject a RSV RNA-LNP composition comprising at least one RSV RNA molecule having an open reading frame encoding at least one RSV antigenic polypeptide, thereby inducing in the subject an immune response specific to RSV antigenic polypeptide, wherein the immune response in the subject is equivalent to an immune response in a subject administered with a traditional composition against the RSV at least, at most, in between any two of, or exactly 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 times the dosage level relative to the RNA composition.

The methods disclosed herein may involve administering to the subject an influenza RNA-LNP composition comprising at least one influenza RNA molecule having an open reading frame encoding at least one influenza antigenic polypeptide, thereby inducing in the subject an immune response specific to influenza antigenic polypeptide, wherein anti-antigenic polypeptide antibody titer in the subject is increased following vaccination relative to anti-antigenic polypeptide antibody titer in a subject vaccinated with a prophylactically effective dose (e.g., a therapeutically effective dose that prevents infection with the virus at a clinically acceptable level) of a traditional vaccine against influenza. An “anti-antigenic polypeptide antibody” is a serum antibody the binds specifically to the antigenic polypeptide. In some aspects, the anti-antigenic polypeptide antibody titer in the subject is increased at least, at most, between any two of, or exactly 1, 2, 3, 4, 5, 6, 7, 8, 9, or 10 log following administration of the influenza RNA-LNP composition relative to anti-antigenic polypeptide antibody titer in a subject administered a prophylactically effective dose of a traditional composition against influenza.

The methods disclosed herein may involve administering to the subject an influenza RNA-LNP composition comprising at least one influenza RNA molecule having an open reading frame encoding at least one influenza antigenic polypeptide, thereby inducing in the subject an immune response specific to influenza antigenic polypeptide, wherein the immune response in the subject is equivalent to an immune response in a subject administered with a traditional composition against influenza at least, at most, in between any two of, or exactly 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 times the dosage level relative to the RNA composition.

In some aspects, the RNA molecule, RNA-LNP and/or composition is used as a vaccine. In some aspects, the RNA molecule, RNA-LNP and/or composition may be used in various therapeutic or prophylactic methods for preventing, treating or ameliorating of acute lower respiratory infection (ALRI), or a disorder related to respiratory illness, including pneumonia and bronchitis.

In some aspects, the RNA molecule, RNA-LNP and/or composition may be used in various therapeutic or prophylactic methods for preventing, treating or ameliorating of acute lower respiratory infection (ALRI), including pneumonia and bronchitis.

RSV RNA compositions and/or influenza RNA compositions may be administered prophylactically to healthy subjects or early in infection during the incubation phase or during active infection after onset of symptoms. In some aspects, the subject is immunocompetent. In some aspects, the subject is immunocompromised.

In some aspects, the RNA molecule, RNA-LNP and/or composition is administered in a single dose. In some aspects, a second, third or fourth dose may be given. In some aspects, the RNA molecule, RNA-LNP and/or composition is administered in multiple doses.

In some aspects, the RNA molecule, RNA-LNP and/or composition is administered intramuscularly (IM) or intradermally (ID).

The present disclosure further provides a kit comprising the RNA molecule, RNA-LNP, and/or composition.

In some aspects, the RNA molecule, RNA-LNP and/or composition described herein is administered to a subject that is less than about 1 years old, or about 1 years old to about 10 years old, or about 10 years old to about 20 years old, or about 20 years old to about 50 years old, or about 60 years old to about 70 years old, or older.

In some aspects the subject is at least, at most, exactly, or between any two of less than 1 year of age, greater than 1 year of age, greater than 5 years of age, greater than 10 years of age, greater than 20 years of age, greater than 30 years of age, greater than 40 years of age, greater than 50 years of age, greater than 60 years of age, greater than 70 years of age, or older.

In some aspects, the subject is greater than 50 years of age.

In some aspects the subject is at least, at most, exactly, or between any two of about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older. In some aspects, the subject may be about 50 years of age or older.

In some aspects the subject is at least, at most, exactly, or between any two of 1 year of age or older, 5 years of age or older, 10 years of age or older, 20 years of age or older, 30 years of age or older, 40 years of age or older, 50 years of age or older, 60 years of age or older, 70 years of age or older, or older. In some aspects the subject may be 50 years of age or older.

### Further Embodiments

In some embodiments, the present disclosure demonstrates that an improved immune response can be produced by increasing the relative amount of RNA encoding an antigen of a type B influenza virus as compared to RNA encoding an influenza type A virus (e.g., an immune response that comprises higher neutralization titers against an influenza type B virus (e.g., higher neutralization titers as compared to a composition comprising equal amounts of RNA encoding an influenza type A antigen and RNA encoding an influenza type B antigen (e.g., as determined by a pseudovirus neutralization assay described herein))). The present disclosure also provides exemplary doses of RNA that can produce strong immune responses against both types of influenza viruses (e.g., neutralizing titers and/or seroconversion rates that are at clinically relevalent levels (e.g., (i) neutralizing titers that are comparable or superior to those previously shown to prevent influenza symptoms, and/or (ii) neutralizing titers and/or seroconversion rates that are comparable or superior to those induced by a relevant comparator (e.g., a commercially approved influenza vaccine or an influenza RNA vaccine))).

In some embodiments, a composition comprising a greater amount of RNA encoding influenza B antigens as compared to RNA encoding influenza A antigens produces an immune response against each of an influenza type B virus and influenza type A virus that is comparable or superior to that induced by a non-RNA influenza vaccine (e.g., an approved vaccine) and/or an RNA vaccine comprising equal amounts of RNA encoding influenza A antigens and RNA encoding influenza B antigens.

In some embodiments, the concentration of RNA in a pharmaceutical RNA preparation is about 0.1-0.2 mg/ml. In some embodiments, the concentration of RNA in a pharmaceutical RNA preparation is about 0.1 mg/ml. In some embodiments, the concentration of RNA in a pharmaceutical RNA preparation is about 0.12 mg/ml. In some embodiments, the concentration of RNA in a pharmaceutical RNA preparation is about 0.14 mg/ml. In some embodiments, the concentration of RNA in a pharmaceutical RNA preparation is about 0.16 mg/ml. In some embodiments, the concentration of RNA in a pharmaceutical RNA preparation is about 0.18 mg/ml. In some embodiments about 30 ug of RNA is administered by administering about 200 μL of RNA preparation. In some embodiments, the RNA in a pharmaceutical RNA preparation is diluted prior to administration (e.g., diluted to a concentration of about 0.05 mg/ml). In some embodiments, administration volumes are between about 200 μl and about 300 μl. In some embodiments, the RNA in a pharmaceutical RNA preparation is formulated in about 10 mM Tris buffer, and about 10% sucrose.

In some embodiments, a pharmaceutical RNA preparation comprises RNA in a concentration of about 0.1 mg/ml, and is formulated in about 10 mM Tris buffer, and about 10% sucrose. In some embodiments, a pharmaceutical RNA preparation comprises RNA in a concentration of about 0.12 mg/ml, and is formulated in about 10 mM Tris buffer, and about 10% sucrose. In some embodiments, a pharmaceutical RNA preparation comprises RNA in a concentration of about 0.14 mg/ml, and is formulated in about 10 mM Tris buffer, and about 10% sucrose. In some embodiments, a pharmaceutical RNA preparation comprises RNA in a concentration of about 0.16 mg/ml, and is formulated in about 10 mM Tris buffer, and about 10% sucrose. In some embodiments, a pharmaceutical RNA preparation comprises RNA in a concentration of about 0.18 mg/ml, and is formulated in about 10 mM Tris buffer, and about 10% sucrose. Such a formulation can be diluted as needed prior to administration to administer different doses of RNA while keeping total injection volume relatively constant. For example, a dose of RNA of about 10 μg can be administered by diluting such a pharmaceutical RNA preparation by about 1:1 and administering about 200 μl of diluted pharmaceutical RNA preparation.

In some embodiments, a vaccine is formulated in a vial (e.g., a glass vial). In some embodiments, a glass vial is sealed with a bromobutyl elastomeric stopper and an aluminum seal with flip-off plastic cap.

In some embodiments, a composition comprises an RNA encoding an antigen (e.g., an HA protein) of an influenza virus that is recommended by a relevant health authority for inclusion in a seasonally-adapted vaccine (e.g., a cell-based, recombinant, or live attenuated virus). In some embodiments a composition comprises a plurality of RNAs, encoding antigens (e.g., HA proteins) of each influenza virus recommended by a relevant health authority for inclusion in a seasonally-adapted vaccine (e.g., a cell-based, recombinant, or live attenuated virus).

In some embodiments, the influenza virus is an influenza A, influenza B, or influenza C virus. In some embodiments, the influenza A virus is an H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, H10N7, or H10N8 virus. In some embodiments, the influenza A virus is an H1N1, H3N2, H5N1, or H5N8 virus. In some embodiments, the influenza A virus is an H1N1 virus (e.g., A/Wisconsin/588/2019 or A/Sydney/5/2021). In some embodiments the influenza A virus is an H3N2 virus. In some embodiments the H3N2 virus is A/Cambodia/e0826360/2020 or A/Darwin/6/2021. In some embodiments, the influenza B virus is of a B/Yamagata or BNictoria lineage. In some embodiments, the BNictoria lineage influenza virus is B/Washington/02/2019. In some embodiments, the BNictoria lineage virus is B/Austria/1359417/2021. In some embodiments, the B/Yamagata lineage influenza virus is B/Phuket/3073/2013.

In some embodiments, a composition described herein comprises a multivalent influenza vaccine. In some embodiments, a multivalent influenza vaccine comprises 2 to 50 RNA distinct molecules (e.g., 2 to 40, 2 to 30, or 2 to 20 RNA molecules), each of which, in some embodiments, may encode a different antigenic polypeptide (or a different version of a particular antigenic polypeptide) associated with influenza, e.g., as described in Arevalo, Claudia P., et al. “A multivalent nucleoside-modified mRNA vaccine against all known influenza virus subtypes.” Science 378.6622 (2022): 899-904. In some embodiments, a composition described herein comprises a trivalent influenza vaccine. In some embodiments, a trivalent influenza vaccine comprises RNAs encoding an antigenic polypeptide associated with two type A viruses and one type B virus that are predicted to be prevalent in a relevant jurisdiction. In some embodiments, the trivalent composition includes a modRNA suspension for injection, e.g., 0.09 mg/mL, having a ratio of 1:1:4 of influenza HA A:A:B strain, respectively. In some embodiments, the trivalent composition comprises 0.015 mg/ml modRNA encoding HA from an influenza A strain, 0.015 mg/ml modRNA encoding HA from an influenza A strain, and 0.060 mg/ml of an influenza B strain. The composition may further comprise a cationic lipid, a pegylated lipid, phospholipid, and sterol, sucrose, tromethamine, and Tris-HCl. A trivalent modRNA HA (B/Austria, A/Wisconsin, A/Darwin) 0.6 ug composition elicited an immune response in mice, wherein the composition included 0.2 ug of each of the 3 HA. The dose volume and immunization route was 50 ul/IM, administered on day 0 and 28. Bleed occurred on day 21 and 42.

In some embodiments, the trivalent composition comprises an Influenza modRNA-LNP suspension for Injection, 0.09 mg/ml at a ratio of 1:1:4 A:A:B.

In some embodiments, a composition described herein comprises a tetravalent influenza vaccine. In some embodiments, a tetravalent influenza vaccine comprises RNAs encoding an antigenic polypeptide associated with two type A viruses and two type B viruses that are predicted to be prevalent in a relevant jurisdiction. In some embodiments, a composition described herein comprises an octavalent influenza vaccine. In some embodiments, an octavalent influenza vaccine comprises RNAs encoding two antigenic polypeptides associated with each of two type A viruses and two type B viruses that are predicted to be prevalent in a relevant jurisdiction (e.g., an HA protein and an NA protein associated with each virus, or immunogenic fragments thereof). In some embodiments, a composition disclosed herein comprises a tetravalent influenza vaccine comprising an RNA comprising a nucleotide sequence encoding an HA protein associated with an H1N1 virus (e.g., A/Wisconsin/588/2019), an RNA comprising a nucleotide sequence encoding an HA protein associated with an H3N2 virus (e.g., A/Cambodia/e0826360/2020), an RNA comprising a nucleotide sequence encoding an HA protein associated with a B/Victoria lineage influenza virus (e.g., B/Washington/02/2019), and an HA protein associated with a B/Yamagata lineage influenza virus (e.g., B/Phuket/3073/2013).

In some embodiments, a composition comprises a tetravalent influenza vaccine comprises RNA encoding an antigenic polypeptide associated with two type A viruses and two type B viruses that are predicted to be prevalent in a relevant jurisdiction. In some embodiments, a tetravalent influenza vaccine comprises RNA encoding an antigenic polypeptide associated with an H1N1 influenza virus, RNA encoding an antigenic polypeptide associated with an H3N2 influenza virus, RNA encoding an antigenic polypeptide associated with a Victoria lineage influenza virus, and RNA encoding an antigenic polypeptide associated with a Yamagata lineage influenza virus. In some embodiments, the tetravalent influenza vaccine comprises RNA associated with influenza types that are predicted to be prevalent in a relevant jurisdiction (e.g., HA polypeptides associated with the H1N1, H3N2, BNictoria, and B/Yamagata influenza viruses that are predicted to be prevalent in a relevant jurisdiction).

In some embodiments, each of the RNAs in a composition disclosed herein encodes an antigenic polypeptide associated with an infectious agent that is predicted to be prevalent in a relevant jurisdiction. Such compositions can reduce the number of vaccinations needed.

In some embodiments, a nucleic acid containing particle comprises two or more RNA molecules, each comprising a nucleotide sequence encoding an antigen (e.g., an HA protein) associated with a different influenza virus. In some embodiments, a nucleic acid containing particle comprises three or more RNA molecules, each comprising a nucleotide sequence encoding an antigen (e.g., an HA protein) associated with a different influenza virus. In some embodiments, a nucleic acid containing particle comprises four or more RNA molecules, each comprising a nucleotide sequence encoding an antigen (e.g., an HA protein) associated with a different influenza virus. In some embodiments, a nucleic acid containing particle comprises an RNA molecule comprising a nucleotide sequence encoding an antigenic polypeptide associated with an H1N1 influenza virus, an RNA molecule comprising a nucleotide sequence encoding an antigenic polypeptide associated with an H3N2 influenza virus, an RNA molecule comprising a nucleotide sequence encoding an antigenic polypeptide associated with a BNictoria lineage influenza virus, and an RNA molecule comprising a nucleotide sequence encoding an antigenic polypeptide associated with a B/Yamagata influenza virus. In some embodiments, each RNA in a composition comprising a nucleotide sequence encoding an antigenic polypeptide associated with an influenza virus is formulated in the same nucleic acid containing particle. In some embodiments, each RNA in a composition comprising a nucleotide sequence encoding an antigenic polypeptide associated with an influenza virus is formulated in separate nucleic acid containing particles.

In some embodiments, a nucleic acid containing particle (e.g., in some embodiments an LNP as described herein) comprising two or more RNA molecules, comprises each RNA molecule in the same amount (i.e., at a 1:1 ratio).

In some embodiments, a nucleic acid containing particle (e.g., in some embodiments an LNP as described herein) comprising two or more RNA molecules, comprises a different amount of each RNA molecule. For example, in some embodiments, a nucleic acid containing particle comprises a first RNA molecule and a second RNA molecule, where the first RNA molecule is present in an amount that is 0.01 to 100 times that of the second RNA molecule (e.g., wherein the amount of the first RNA molecule is 0.01 to 50, 0.01 to 4, 0.01 to 30, 0.01 to 25, 0.01 to 20, 0.01 to 15, 0.01 to 10, 0.01 to 9, 0.01 to 8, 0.01 to 7, 0.01 to 6, 0.01 to 5, 0.01 to 4, 0.01 to 3, 0.01 to 2, 0.01 to 1.5, 1 to 50, 1 to 4, 1 to 30, 1 to 25, 1 to 20, 1 to 15, 1 to 10, 1 to 9, 1 to 8, 1 to 7, 1 to 6, 1 to 5, 1 to 4, 1 to 3, 1 to 2, or 1 to 1.5 times higher than the second RNA molecule). In some embodiments, a nucleic acid containing particle comprises a first RNA molecule and a second RNA molecule, wherein the concentration of the first RNA molecule is 1 to 10 times that of the second RNA molecule. In some embodiments, a nucleic acid containing particle comprises a first RNA molecule and a second RNA molecule, wherein the concentration of the first RNA molecule is 1 to 5 times that of the second RNA molecule. In some embodiments, a nucleic acid containing particle comprises a first RNA molecule and a second RNA molecule, wherein the concentration of the first RNA molecule is 1 to 3 times that of the second RNA molecule. In some embodiments, a nucleic acid containing particle comprises a first RNA molecule and a second RNA molecule, wherein the concentration of the first RNA molecule is 2 times that of the second RNA molecule. In some embodiments, a nucleic acid containing particle comprises a first RNA molecule and a second RNA molecule, wherein the concentration of the first RNA molecule is 3 times that of the second RNA molecule.

In some embodiments, a nucleic acid containing particle (e.g., in some embodiments an LNP as described herein) comprising three RNA molecules, comprises each RNA molecule in the same amount (i.e., at a 1:1:1 ratio).

In some embodiments, a nucleic acid containing particle (e.g., in some embodiments an LNP as described herein) comprising three RNA molecules, comprises a different amount of each RNA molecule. For example, in some embodiments, the ratio of first RNA molecule: second RNA molecule: third RNA molecule is 1:0.01-100:0.01-100 (e.g., 1:0.01-50:0.01-50; 1:0.01-40:0.01-40; 1:0.01-30:0.01-25; 1:0.01-25:0.01-25; 1:0.01-20:0.01-20; 1:0.01-15:0.01-15; 1:0.01-10:0.01-9; 1:0.01-9:0.01-9; 1:0.01-8:0.01-8; 1:0.01-7:0.01-7; 1:0.01-6:0.01-6; 1:0.01-5:0.01-5; 1:0.01-4:0.01-4; 1:0.01-3:0.01-3; 1:0.01-2:0.01-2; or 1:0.01-1.5:0.01-1.5). In some embodiments, the ratio of first RNA molecule: second RNA molecule: third RNA molecule is 1:1:3. In some embodiments, the ratio of first RNA molecule: second RNA molecule: third RNA molecule is 1:3:3.

The term “dose” as used herein refers in general to a “dose amount” which relates to the amount of RNA administered per administration, i.e., per dosing.

In some embodiments, administration of an immunogenic composition or vaccine of the present disclosure may be performed by single administration or boosted by multiple administrations.

In some embodiments, a regimen described herein includes at least one dose. In some embodiments, a regimen includes a first dose and at least one subsequent dose. In some embodiments, the first dose is the same amount as at least one subsequent dose. In some embodiments, the first dose is the same amount as all subsequent doses. In some embodiments, the first dose is a different amount as at least one subsequent dose. In some embodiments, the first dose is a different amount than all subsequent doses. In some embodiments, a regimen comprises two doses. In some embodiments, a provided regimen consists of two doses. In some embodiments, a regimen comprises three doses.

In one embodiment, the disclosure envisions administration of a single dose. In one embodiment, the disclosure envisions administration of a priming dose followed by one or more booster doses. The booster dose or the first booster dose may be administered 7 to 28 days or 14 to 24 days following administration of the priming dose. In some embodiments, a first booster dose may be administered 1 week to 3 months (e.g., 1 week, 2 weeks, 3 weeks, 4 weeks, 5 weeks, 6 weeks, 7 weeks, 8 weeks, 9 weeks, 10 weeks, 11 weeks, 12 weeks) following administration of a priming dose. In some embodiments, a subsequent booster dose may be administered at least 1 week or longer, including, e.g., at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, or longer, following a preceding booster dose. In some embodiments, subsequent booster doses may be administered about 5-9 weeks or 6-8 weeks apart. In some embodiments, at least one subsequent booster dose (e.g., after a first booster dose) may be administered at least 3 months or longer, including, e.g., at least 4 months, at least 5 months, at least 6 months, at least 7 months, at least 8 months, at least 9 months, at least 10 months, or longer, following a preceding dose.

In some embodiments, a dose comprises a total amount of RNA of 0.1 μg to 300 μg, 0.5 μg to 200 μg, or 1 μg to 100 μg, such as about 1 μg, about 2 μg, about 3 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg. In some embodiments, a dose comprises a total amount of RNA (e.g., modRNA) of up to about 100 μg. In some embodiments, a dose comprises 0.1 μg to 100 μg of one or more first RNAs and 0.1 μg to 100 μg of one or more second RNAs, wherein the one or more first RNAs each comprise a nucleotide sequence encoding an antigenic polypeptide associated with a first infectious agent (e.g., a coronavirus), and the one or more second RNAs each comprise a nucleotide sequence encoding an antigenic polypeptide associated with a second infectious agent (e.g., influenza). In some embodiments, a dose comprises 3 to 60 μg of one or more first RNAs and 3 to 90 μg of one or more second RNAs. In some embodiments, a dose comprises 3 to 60 μg of one or more first RNAs and 3 to 90 μg of one or more second RNAs, wherein the dose comprises up to 100 μg of RNA total. In some embodiments, a dose comprises 3 to 30 μg of one or more first RNAs and 3 to 60 μg of one or more second RNAs, wherein the dose comprises up to 100 μg of RNA total. In some embodiments, a dose comprises 3 μg of one or more first RNAs and 3 μg of one or more second RNAs. In some embodiments, a dose comprises 3 μg of one or more first RNAs and 6 μg of one or more second RNAs. In some embodiments, a dose comprises 10 μg of one or more first RNAs and 10 μg of one or more second RNAs. In some embodiments, a dose comprises 10 μg of one or more first RNAs and 20 μg of one or more second RNAs. In some embodiments, a dose comprises 30 μg of one or more first RNAs and 30 μg of one or more second RNAs. In some embodiments, a dose comprises 30 μg of one or more first RNAs and 60 μg of one or more second RNAs. In some embodiments, a dose comprises 60 μg of one or more first RNAs and 30 μg of one or more second RNAs.

In some embodiments, a subsequent dose given to an individual (e.g., as part of a primary regimen or booster regimen) can have the same amount of RNA as previously given to the individual. In some embodiments, a subsequent dose given to an individual (e.g., as part of a primary regimen or booster regimen) can differ in the amount of RNA, as compared to the amount previously given to the individual. For example, in some embodiments, a subsequent dose can be higher or lower than the prior dose, for example, based on consideration of various factors, including, e.g., immunogenicity and/or reactogenicity induced by the prior dose, prevalence of the disease, etc. In some embodiments, a subsequent dose can be higher than a prior dose by at least 30%, at least 40%, at least 50%, at least 60%, at least 70%, at least 80%, at least 90%, or higher. In some embodiments, a subsequent dose can be higher than a prior dose by at least 1.5-fold, at least 2-fold, at least 2.5 fold, at least 3-fold, or higher. In some embodiments, a subsequent dose can be higher than a prior dose by at least 30%, at least 40%, at least 50%, at least 60%, at least 70%, at least 80%, at least 90%, or higher. In some embodiments, a subsequent dose can be lower than a prior dose by at least 10%, at least 20%, at least 30%, at least 40%, at least 50%, at least 60%, at least 70% or lower. In some embodiments, an amount the RNA described herein from 0.1 μg to 300 μg, 0.5 μg to 200 μg, or 1 μg to 100 μg, such as about 1 μg, about 2 μg, about 3 μg, about 10 μg, about 15 μg, about 20 μg, about 25 μg, about 30 μg, about 35 μg, about 40 μg, about 45 μg, about 50 μg, about 55 μg, about 60 μg, about 70 μg, about 80 μg, about 90 μg, or about 100 μg may be administered per dose (e.g., in a given dose).

In some embodiments, an amount of the RNA described herein of 60 μg or lower, 55 μg or lower, 50 μg or lower, 45 μg or lower, 40 μg or lower, 35 μg or lower, 30 μg or lower, 25 μg or lower, 20 μg or lower, 15 μg or lower, 10 μg or lower, 5 μg or lower, 3 μg or lower, 2.5 μg or lower, or 1 μg or lower may be administered per dose (e.g., in a given dose).

In some embodiments, an amount of the RNA described herein of at least 0.25 μg, at least 0.5 μg, at least 1 μg, at least 2 μg, at least 3 μg, at least 4 μg, at least 5 μg, at least 10 μg, at least 15 μg, at least 20 μg, at least 25 μg, at least 30 μg, at least 40 μg, at least 50 μg, or at least 60 μg may be administered per dose (e.g., in a given dose). In some embodiments, an amount of the RNA described herein of at least 3 ug may be administered in at least one of given doses.

In some embodiments, an amount of the RNA described herein of at least 10 ug may be administered in at least one of given doses. In some embodiments, an amount of the RNA described herein of at least 15 ug may be administered in at least one of given doses. In some embodiments, an amount of the RNA described herein of at least 20 ug may be administered in at least one of given doses. In some embodiments, an amount of the RNA described herein of at least 25 ug may be administered in at least one of given doses. In some embodiments, an amount of the RNA described herein of at least 30 ug may be administered in at least one of given doses. In some embodiments, an amount of the RNA described herein of at least 50 ug may be administered in at least one of given doses. In some embodiments, an amount of the RNA described herein of at least 60 ug may be administered in at least one of given doses. In some embodiments, combinations of aforementioned amounts may be administered in a regimen comprising two or more doses (e.g., a prior dose and a subsequent dose can be of different amounts as described herein). In some embodiments, combinations of aforementioned amounts may be administered in a primary regimen and a booster regimen (e.g., different doses can be given in a primary regimen and a booster regimen).

In some embodiments, an amount of an RNA described herein of 0.25 μg to 60 μg, 0.5 μg to 55 μg, 1 μg to 50 μg, 5 μg to 40 μg, or 10 μg to 30 μg may be administered per dose. In some embodiments, an amount of the RNA described herein of 3 μg to 30 μg may be administered in at least one of given doses. In some embodiments, an amount of the RNA described herein of 3 μg to 20 μg may be administered in at least one of given doses. In some embodiments, an amount of the RNA described herein of 3 μg to 15 μg may be administered in at least one of given doses. In some embodiments, an amount of the RNA described herein of 3 μg to 10 μg may be administered in at least one of given doses. In some embodiments, an amount of the RNA described herein of 10 μg to 30 μg may be administered in at least one of given doses.

In some embodiments, a regimen administered to a subject may comprise a plurality of doses (e.g., at least two doses, at least three doses, or more). In some embodiments, a regimen administered to a subject may comprise a first dose and a second dose, which are given at least 2 weeks apart, at least 3 weeks apart, at least 4 weeks apart, or more. In some embodiments, such doses may be at least 1 month, at least 2 months, at least 3 months, at least 4 months, at least 5 months, at least 6 months, at least 7 months, at least 8 months, at least 9 months, at least 10 months, at least 11 months, at least 12 months, or more apart. In some embodiments, doses may be administered days apart, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more days apart. In some embodiments, doses may be administered about 1 to about 3 weeks apart, or about 1 to about 4 weeks apart, or about 1 to about 5 weeks apart, or about 1 to about 6 weeks apart, or about 1 to more than 6 weeks apart. In some embodiments, doses may be separated by a period of about 7 to about 60 days, such as for example about 14 to about 48 days, etc. In some embodiments, a minimum number of days between doses may be about 1, 2, 3, 4, 5, 6, 7, 8, 9, 10, 11, 12, 13, 14, 15, 16, 17, 18, 19, 20, 21 or more. In some embodiments, a maximum number of days between doses may be about 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or fewer. In some embodiments, doses may be about 21 to about 28 days apart. In some embodiments, doses may be about 19 to about 42 days apart. In some embodiments, doses may be about 7 to about 28 days apart. In some embodiments, doses may be about 14 to about 24 days. In some embodiments, doses may be about 21 to about 42 days.

In some embodiments, a vaccination regimen comprises a first dose and a second dose. In some embodiments, a first dose and a second dose are administered by at least 21 days apart.

In some embodiments, a first dose and a second dose are administered by at least 28 days apart.

In some embodiments, a vaccination regimen comprises a first dose and a second dose, wherein the amount of RNA administered in the first dose is the same as the amount of RNA administered in the second dose. In some embodiments, a vaccination regimen comprises a first dose and a second dose wherein the amount of RNA administered in the first dose differs from that administered in the second dose.

In some embodiments, a vaccination regimen comprises a first dose and a second dose, wherein the amount of RNA administered in the first dose is less than that administered in the second dose. In some embodiments, the amount of RNA administered in the first dose is 10%-90% of the second dose. In some embodiments, the amount of RNA administered in the first dose is 10%-50% of the second dose. In some embodiments, the amount of RNA administered in the first dose is 10%-20% of the second dose. In some embodiments, the first dose and the second dose are administered at least 2 weeks apart, including, at least 3 weeks apart, at least 4 weeks apart, at least 5 weeks apart, at least 6 weeks apart or longer. In some embodiments, the first dose and the second dose are administered at least 3 weeks apart.

In some embodiments, a first dose comprises less than about 30 ug of RNA and a second dose comprises at least about 30 ug of RNA. In some embodiments, a first dose comprises about 1 to less than about 30 ug of RNA (e.g., about 0.1, about 1, about 3, about 5, about 10, about 15, about 20, about 25, or less than about 30 ug of RNA) and a second dose comprises about 30 to about 100 ug of RNA (e.g., about 30, about 40, about 50, or about 60 ug of RNA). In some embodiments, a first dose comprises about 1 to about 20 ug of RNA, about 1 to about 10 ug of RNA, or about 1 to about 5 ug of RNA and a second dose comprises about 30 to about 60 ug of RNA.

In some embodiments, a first dose comprises about 1 to about 10 ug of RNA (e.g., about 1, about 2, about 3, about 4, about 5, about 6, about 7, about 8, about 9, or about 10 ug of RNA) and a second dose comprises about 30 to about 60 ug of RNA (e.g., about 30, about 35, about 40, about 45, about 50, about 55, or about 60 ug of RNA).

In some embodiments, a first dose comprises about 1 ug of RNA and a second dose comprises about 30 ug of RNA. In some embodiments, a first dose comprises about 3 ug of RNA and a second dose comprises about 30 ug of RNA. In some embodiments, a first dose comprises about 5 ug of RNA and a second dose comprises about 30 ug of RNA. In some embodiments, a first dose comprises about 10 ug of RNA and a second dose comprises about 30 ug of RNA. In some embodiments, a first dose comprises about 15 ug of RNA and a second dose comprises about 30 ug of RNA.

In some embodiments, a first dose comprises about 1 ug of RNA and a second dose comprises about 60 ug of RNA. In some embodiments, a first dose comprises about 3 ug of RNA and a second dose comprises about 60 ug of RNA. In some embodiments, a first dose comprises about 5 ug of RNA and a second dose comprises about 60 ug of RNA. In some embodiments, a first dose comprises about 6 ug of RNA and a second dose comprises about 60 ug of RNA. In some embodiments, a first dose comprises about 10 ug of RNA and a second dose comprises about 60 ug of RNA. In some embodiments, a first dose comprises about 15 ug of RNA and a second dose comprises about 60 ug of RNA. In some embodiments, a first dose comprises about 20 ug of RNA and a second dose comprises about 60 ug of RNA. In some embodiments, a first dose comprises about 25 ug of RNA and a second dose comprises about 60 ug of RNA.

In some embodiments, a first dose comprises about 30 ug of RNA and a second dose comprises about 60 ug of RNA.

In some embodiments, a first dose comprises less than about 10 ug of RNA and a second dose comprises at least about 10 ug of RNA. In some embodiments, a first dose comprises about 0.1 to less than about 10 ug of RNA (e.g., about 0.1, about 0.5, about 1, about 2, about 3, about 4, about 5, about 6, about 7, about 8, or less than about 10 ug of RNA) and a second dose comprises about 10 to about 30 ug of RNA (e.g., about 10, about 15, about 20, about 25, or about 30 ug of RNA). In some embodiments, a first dose comprises about 0.1 to about 10 ug of RNA, about 1 to about 5 ug of RNA, or about 0.1 to about 3 ug of RNA and a second dose comprises about 10 to about 30 ug of RNA.

In some embodiments, a first dose comprises about 0.1 to about 5 ug of RNA (e.g., about 0.1, about 0.5, about 1, about 2, about 3, about 4, about 5 ug of RNA) and a second dose comprises about 10 to about 20 ug of RNA (e.g., about 10, about 12, about 14, about 16, about 18, about 20 ug of RNA).

In some embodiments, a first dose comprises about 0.1 ug of RNA and a second dose comprises about 10 ug of RNA. In some embodiments, a first dose comprises about 0.3 ug of RNA and a second dose comprises about 10 ug of RNA. In some embodiments, a first dose comprises about 1 ug of RNA and a second dose comprises about 10 ug of RNA. In some embodiments, a first dose comprises about 3 ug of RNA and a second dose comprises about 10 ug of RNA.

In some embodiments, a first dose comprises less than about 3 ug of RNA and a second dose comprises at least about 3 ug of RNA. In some embodiments, a first dose comprises about 0.1 to less than about 3 ug of RNA (e.g., about 0.1, about 0.2, about 0.3, about 0.5, about 0.6, about 0.7, about 0.8, about 0.9, about 1.0, about 1.5, about 2.0, or about 2.5 ug of RNA) and a second dose comprises about 3 to about 10 ug of RNA (e.g., about 3, about 4, about 5, about 6, or about 7, about 8, about 9, or about 10 ug of RNA). In some embodiments, a first dose comprises about 0.1 to about 3 ug of RNA, about 0.1 to about 1 ug of RNA, or about 0.1 to about 0.5 ug of RNA and a second dose comprises about 3 to about 10 ug of RNA.

In some embodiments, a first dose comprises about 0.1 to about 1.0 ug of RNA (e.g., about 0.1, about 0.2, about 0.3, about 0.4, about 0.5, about 0.6, about 0.7, about 0.8, about 0.9, or about 1.0 ug of RNA) and a second dose comprises about 1 to about 3 ug of RNA (e.g., about 1.0, about 1.5, about 2.0, about 2.5, or about 3.0 ug of RNA).

In some embodiments, a first dose comprises about 0.1 ug of RNA and a second dose comprises about 3 ug of RNA. In some embodiments, a first dose comprises about 0.3 ug of RNA and a second dose comprises about 3 ug of RNA. In some embodiments, a first dose comprises about 0.5 ug of RNA and a second dose comprises about 3 ug of RNA. In some embodiments, a first dose comprises about 1 ug of RNA and a second dose comprises about 3 ug of RNA.

In some embodiments, a vaccination regimen comprises a first dose and a second dose, wherein the amount of RNA administered in the first dose is greater than that administered in the second dose. In some embodiments, the amount of RNA administered in the second dose is 10%-90% of the first dose. In some embodiments, the amount of RNA administered in the second dose is 10%-50% of the first dose. In some embodiments, the amount of RNA administered in the second dose is 10%-20% of the first dose. In some embodiments, the first dose and the second dose are administered at least 2 weeks apart, including, at least 3 weeks apart, at least 4 weeks apart, at least 5 weeks apart, at least 6 weeks apart or longer. In some embodiments, the first dose and the second dose are administered at least 3 weeks apart In some embodiments, a first dose comprises at least about 30 ug of RNA and a second dose comprises less than about 30 ug of RNA. In some embodiments, a first dose comprises about 30 to about 100 ug of RNA (e.g., about 30, about 40, about 50, or about 60 ug of RNA) and a second dose comprises about 1 to about 30 ug of RNA (e.g., about 0.1, about 1, about 3, about 5, about 10, about 15, about 20, about 25, or about 30 ug of RNA). In some embodiments, a second dose comprises about 1 to about 20 ug of RNA, about 1 to about 10 ug of RNA, or about 1 to 5 ug of RNA. In some embodiments, a first dose comprises about 30 to about 60 ug of RNA and a second dose comprises about 1 to about 20 ug of RNA, about 1 to about 10 ug of RNA, or about 0.1 to about 3 ug of RNA.

In some embodiments, a first dose comprises about 30 to about 60 ug of RNA (e.g., about 30, about 35, about 40, about 45, about 50, about 55, or about 60 ug of RNA) and a second dose comprises about 1 to about 10 ug of RNA (e.g., about 1, about 2, about 3, about 4, about 5, about 6, about 7, about 8, about 9, or about 10 ug of RNA).

In some embodiments, a first dose comprises about 30 ug of RNA and a second dose comprises about 1 ug of RNA. In some embodiments, a first dose comprises about 30 ug of RNA and a second dose comprises about 3 ug of RNA. In some embodiments, a first dose comprises about 30 ug of RNA and a second dose comprises about 5 ug of RNA. In some embodiments, a first dose comprises about 30 ug of RNA and a second dose comprises about 10 ug of RNA. In some embodiments, a first dose comprises about 30 ug of RNA and a second dose comprises about 15 ug of RNA.

In some embodiments, a first dose comprises about 60 ug of RNA and a second dose comprises about 1 ug of RNA. In some embodiments, a first dose comprises about 60 ug of RNA and a second dose comprises about 3 ug of RNA. In some embodiments, a first dose comprises about 60 ug of RNA and a second dose comprises about 5 ug of RNA. In some embodiments, a first dose comprises about 60 ug of RNA and a second dose comprises about 6 ug of RNA. In some embodiments, a first dose comprises about 60 ug of RNA and a second dose comprises about 10 ug of RNA. In some embodiments, a first dose comprises about 60 ug of RNA and a second dose comprises about 15 ug of RNA. In some embodiments, a first dose comprises about 60 ug of RNA and a second dose comprises about 20 ug of RNA. In some embodiments, a first dose comprises about 60 ug of RNA and a second dose comprises about 25 ug of RNA. In some embodiments, a first dose comprises about 60 ug of RNA and a second dose comprises about 30 ug of RNA.

In some embodiments, a first dose comprises at least about 10 ug of RNA and a second dose comprises less than about 10 ug of RNA. In some embodiments, a first dose comprises about 10 to about 30 ug of RNA (e.g., about 10, about 15, about 20, about 25, or about 30 ug of RNA) and a second dose comprises about 0.1 to less than about 10 ug of RNA (e.g., about 0.1, about 0.5, about 1, about 2, about 3, about 4, about 5, about 6, about 7, about 8, or less than about 10 ug of RNA). In some embodiments, a first dose comprises about 10 to about 30 ug of RNA, or about 0.1 to about 3 ug of RNA and a second dose comprises about 1 to about 10 ug of RNA, or about 1 to about 5 ug of RNA.

In some embodiments, a first dose comprises about 10 to about 20 ug of RNA (e.g., about 10, about 12, about 14, about 16, about 18, about 20 ug of RNA) and a second dose comprises about 0.1 to about 5 ug of RNA (e.g., about 0.1, about 0.5, about 1, about 2, about 3, about 4, or about 5 ug of RNA).

In some embodiments, a first dose comprises about 10 ug of RNA and a second dose comprises about 0.1 ug of RNA. In some embodiments, a first dose comprises about 10 ug of RNA and a second dose comprises about 0.3 ug of RNA. In some embodiments, a first dose comprises about 10 ug of RNA and a second dose comprises about 1 ug of RNA. In some embodiments, a first dose comprises about 10 ug of RNA and a second dose comprises about 3 ug of RNA.

In some embodiments, a first dose comprises at least about 3 ug of RNA and a second dose comprises less than about 3 ug of RNA. In some embodiments, a first dose comprises about 3 to about 10 ug of RNA (e.g., about 3, about 4, about 5, about 6, or about 7, about 8, about 9, or about 10 ug of RNA) and a second dose comprises 0.1 to less than about 3 ug of RNA (e.g., about 0.1, about 0.2, about 0.3, about 0.5, about 0.6, about 0.7, about 0.8, about 0.9, about 1.0, about 1.5 about 2.0, or about 2.5 ug of RNA). In some embodiments, a first dose comprises about 3 to about 10 ug of RNA and a second dose comprises about 0.1 to about 3 ug of RNA, about 0.1 to about 1 ug of RNA, or about 0.1 to about 0.5 ug of RNA.

In some embodiments, a first dose comprises about 1 to about 3 ug of RNA (e.g., about 1, about 1.5, about 2.0, about 2.5, or about 3.0 ug of RNA) and a second dose comprises about 0.1 to 0.3 ug of RNA (e.g., about 0.1, about 0.2, about 0.3, about 0.4, about 0.5, about 0.6, about 0.7, about 0.8, about 0.9, or about 1.0 ug of RNA).

In some embodiments, a first dose comprises about 3 ug of RNA and a second dose comprises about 0.1 ug of RNA. In some embodiments, a first dose comprises about 3 ug of RNA and a second dose comprises about 0.3 ug of RNA. In some embodiments, a first dose comprises about 3 ug of RNA and a second dose comprises about 0.6 ug of RNA. In some embodiments, a first dose comprises about 3 ug of RNA and a second dose comprises about 1 ug of RNA.

In some embodiments, a vaccination regimen comprises at least two doses, including, e.g., at least three doses, at least four doses or more. In some embodiments, a vaccination regimen comprises three doses. In some embodiments, the time interval between the first dose and the second dose can be the same as the time interval between the second dose and the third dose.

In some embodiments, the time interval between the first dose and the second dose can be longer than the time interval between the second dose and the third dose, e.g., by days or weeks (including, e.g., at least 3 days, at least 4 days, at least 5 days, at least 6 days, at least 1 week, at least 2 weeks, at least 3 weeks, at least 4 weeks, at least 5 weeks, at least 6 weeks, or longer). In some embodiments, the time interval between the first dose and the second dose can be shorter than the time interval between the second dose and the third dose, e.g., by days or weeks (including, e.g., at least 3 days, at least 4 days, at least 5 days, at least 6 days, at least 1 week, at least 2 weeks, at least 3 weeks, at least 4 weeks, at least 5 weeks, at least 6 weeks, or longer). In some embodiments, the time interval between the first dose and the second dose can be shorter than the time interval between the second dose and the third dose, e.g., by at least 1 month (including, e.g., at least 2 months, at least 3 months, at least 4 months, at least 5 months, at least 6 months, at least 7 months, at least 8 months, at least 9 months, at least 10 months, at least 11 months, at least 12 months, or longer).

In some embodiments, a last dose of a primary regimen and a first dose of a booster regimen are given at least 2 months, at least 3 months, at least 4 months, at least 5 months, at least 6 months, at least 7 months, at least 8 months, at least 9 months, at least 10 months, at least 11 months, at least 12 months, or more apart. In some embodiments, a primary regimen may comprises two doses. In some embodiments, a primary regimen may comprises three doses.

In some embodiments, a first dose and a second dose (and/or other subsequent dose) may be administered by intramuscular injection. In some embodiments, a first dose and a second dose (and/or other subsequent dose) may be administered in the deltoid muscle. In some embodiments, a first dose and a second dose (and/or other subsequent dose) may be administered in the same arm.

In some embodiments, an mRNA composition described herein is administered (e.g., by intramuscular injection) as a series of two doses (e.g., 0.3 mL each) 21 days apart. In some embodiments, an mRNA composition described herein is administered (e.g., by intramuscular injection) as a series of two doses (e.g., 0.2 mL each) 21 days apart. In some embodiments, an mRNA composition described herein is administered (e.g., by intramuscular injection) as a series of three doses (e.g., 0.3 mL or lower including, e.g., 0.2 mL), wherein doses are given at least 3 weeks apart. In some embodiments, the first and second doses may be administered 3 weeks apart, while the second and third doses may be administered at a longer time interval than that between the first and the second doses, e.g., at least 4 weeks apart or longer (including, at least 5 weeks, at least 6 weeks, at least 7 weeks, at least 8 weeks, at least 9 weeks, or longer). In some embodiments, each dose is about 60 ug. In some embodiments, each dose is about 50 ug. In some embodiments, each dose is about 30 ug. In some embodiments, each dose is about 25 ug. In some embodiments, each dose is about 20 ug. In some embodiments, each dose is about 15 ug. In some embodiments, each dose is about 10 ug. In some embodiments, each dose is about 3 ug.

In some embodiments, at least one dose given in a vaccination regimen (e.g., a primary vaccination regimen and/or a booster vaccination regimen) is about 60 ug. In some embodiments, at least one dose given in a vaccination regimen (e.g., a primary vaccination regimen and/or a booster vaccination regimen) is about 50 ug. In some embodiments, at least one dose given in a vaccination regimen (e.g., a primary vaccination regimen and/or a booster vaccination regimen) is about 30 ug. In some embodiments, at least one dose given in a vaccination regimen (e.g., a primary vaccination regimen and/or a booster vaccination regimen) is about 25 ug. In some embodiments, at least one dose given in a vaccination regimen (e.g., a primary vaccination regimen and/or a booster vaccination regimen) is about 20 ug. In some embodiments, at least one dose given in a vaccination regimen (e.g., a primary vaccination regimen and/or a booster vaccination regimen) is about 15 ug. In some embodiments, at least one dose given in a vaccination regimen (e.g., a primary vaccination regimen and/or a booster vaccination regimen) is about 10 ug. In some embodiments, at least one dose given in a vaccination regimen (e.g., a primary vaccination regimen and/or a booster vaccination regimen) is about 3 ug.

In one embodiment, an amount of the RNA described herein of about 60 μg is administered per dose. In one embodiment, an amount of the RNA described herein of about 50 μg is administered per dose. In one embodiment, an amount of the RNA described herein of about 30 μg is administered per dose. In one embodiment, an amount of the RNA described herein of about 25 μg is administered per dose. In one embodiment, an amount of the RNA described herein of about 20 μg is administered per dose. In one embodiment, an amount of the RNA described herein of about 15 μg is administered per dose. In one embodiment, an amount of the RNA described herein of about 10 μg is administered per dose. In one embodiment, an amount of the RNA described herein of about 5 μg is administered per dose. In one embodiment, an amount of the RNA described herein of about 3 μg is administered per dose. In one embodiment, at least two of such doses are administered. For example, a second dose may be administered about 21 days following administration of the first dose.

In some embodiments, the efficacy of the RNA vaccine described herein (e.g., administered in two doses, wherein a second dose may be administered about 21 days following administration of the first dose, and administered, for example, in an amount of about 30 μg per dose) is at least 70%, at least 80%, at least 90, or at least 95% beginning 7 days after administration of the second dose (e.g., beginning 28 days after administration of the first dose if a second dose is administered 21 days following administration of the first dose). In some embodiments, such efficacy is observed in populations of age of at least 50, at least 55, at least 60, at least 65, at least 70, or older. In some embodiments, the efficacy of the RNA vaccine described herein (e.g., administered in two doses, wherein a second dose may be administered about 21 days following administration of the first dose, and administered, for example, in an amount of about 30 μg per dose) beginning 7 days after administration of the second dose (e.g., beginning 28 days after administration of the first dose if a second dose is administered 21 days following administration of the first dose) in populations of age of at least 65, such as 65 to 80, 65 to 75, or 65 to 70, is at least 90%, at least 91%, at least 92%, at least 93%, at least 94%, or at least 95%. Such efficacy may be observed over time periods of up to 1 month, 2 months, 3 months, 6 months or even longer.

In one embodiment, vaccine efficacy is defined as the percent reduction in the number of subjects with evidence of infection (vaccinated subjects vs. non-vaccinated subjects).

In one embodiment, methods and agents described herein are administered to a paediatric population. In various embodiments, the paediatric population comprises or consists of subjects under 18 years, e.g., 5 to less than 18 years of age, 12 to less than 18 years of age, 16 to less than 18 years of age, 12 to less than 16 years of age, 5 to less than 12 years of age, or 6 months to less than 12 years of age. In various embodiments, the paediatric population comprises or consists of subjects under 5 years, e.g., 2 to less than 5 years of age, 12 to less than 24 months of age, 7 to less than 12 months of age, or less than 6 months of age. In some such embodiments, an mRNA composition described herein is administered to subjects of less than 2 years old, for example, 6 months to less than 2 years old. In some such embodiments, an mRNA composition described herein is administered to subjects of less than 6 months old, for example, 1 month to less than 4 months old. In some embodiments, a dosing regimen (e.g., doses and/or dosing schedule) for a paediatric population may vary for different age groups. For example, in some embodiments, a subject 6 months through 4 years of age may be administered according to a primary regimen comprising at least three doses, in which the initial two doses are administered at least 3 weeks (including, e.g., at least 4 weeks, at least 5 weeks, at least 6 weeks, or longer) apart followed by a third dose administered at least 8 weeks (including, e.g., at least 9 weeks, at least 10 weeks, at least 11 weeks, at least 12 weeks, or longer) after the second dose. In some such embodiments, at least one dose administered is 3 ug RNA described herein. In some embodiments, a subject 5 years of age and older may be administered according to a primary regimen comprising at least two doses, in which the two doses are administered at least 3 weeks (including, e.g., at least 3 weeks, at least 4 weeks, at least 5 weeks, at least 6 weeks, or longer) apart. In some such embodiments, at least one dose administered is 10 ug RNA described herein. In some embodiments, a subject 5 years of age and older who are immunocompromised (e.g., in some embodiments subjects who have undergone solid organ transplantation, or who are diagnosed with conditions that are considered to have an equivalent of immunocompromise) may be administered according to a primary regimen comprising at least three doses, in which the initial two doses are administered at least 3 weeks (including, e.g., at least 3 weeks, at least 4 weeks, at least 5 weeks, at least 6 weeks, or longer) apart, followed by a third dose administered at least 4 weeks (including, e.g., at least 5 weeks, at least 6 weeks, at least 7 weeks, at least 8 weeks, at least 9 weeks, at least 10 weeks, at least 11 weeks, at least 12 weeks, or longer) after the second dose.

In some embodiments, an mRNA composition described herein is administered to subjects of age 12 or older and each dose is about 30 ug. In some embodiments, an mRNA composition described herein is administered to subjects of age 12 or older (including, e.g., age 18 or older) and each dose is higher than 30 ug, including, e.g., 35 ug, 40 ug, 45 ug, 50 ug, 55 ug, 60 ug, 65 ug, 70 ug, or higher. In some such embodiments, an mRNA composition described herein is administered to subjects of age 12 or older and each dose is about 60 ug. In some such embodiments, an mRNA composition described herein is administered to subjects of age 12 or older and each dose is about 50 ug. In one embodiment, the paediatric population comprises or consists of subjects 12 to less than 18 years of age including subjects 16 to less than 18 years of age and/or subjects 12 to less than 16 years of age. In this embodiment, treatments may comprise 2 vaccinations 21 days apart, wherein, in one embodiment, the vaccine is administered in an amount of 30 μg RNA per dose, e.g., by intramuscular administration. In some embodiments, higher doses are administered to older pediatric patients and adults, e.g., to patients 12 years or older, compared to younger children or infants, e.g. 2 to less than 5 years old, 6 months to less than 2 years old, or less than 6 months old. In some embodiments, higher doses are administered to children who are 2 to less than 5 years old, as compared to toddlers and/or infants, e.g., who are 6 months to less than 2 years old, or less than 6 months old.

In one embodiment, the paediatric population comprises or consists of subjects 5 to less than 18 years of age including subjects 12 to less than 18 years of age and/or subjects 5 to less than 12 years of age. In this embodiment, treatments may comprise 2 vaccinations 21 days apart, wherein, in various embodiments, the vaccine is administered in an amount of 10 μg, 20 μg, or 30 μg RNA per dose, e.g., by intramuscular administration. In some such embodiments, an mRNA composition described herein is administered to subjects of age 5 to 11 and each dose is about 10 ug.

In one embodiment, the paediatric population comprises or consists of subjects less than 5 years of age including subjects 2 to less than 5 years of age, subjects 12 to less than 24 months of age, subjects 7 to less than 12 months of age, subjects 6 to less than 12 months of age and/or subjects less than 6 months of age. In this embodiment, treatments may comprise 2 vaccinations, e.g., 21 to 42 days apart, e.g., 21 days apart, wherein, in various embodiments, the vaccine is administered in an amount of 3 μg, 10 μg, 20 μg, or 30 μg RNA per dose, e.g., by intramuscular administration. In some such embodiments, an mRNA composition described herein is administered to subjects of age 2 to less than 5 and each dose is about 3 ug. In some such embodiments, an mRNA composition described herein is administered to subjects of about 6 months to less than about 5 years and each dose is about 3 ug.

In some embodiments, an mRNA composition described herein is administered to subjects of age 12 or older and at least one dose given in a vaccination regimen (e.g., a primary vaccination regimen and/or a booster vaccination regimen) is about 60 ug. In some embodiments, an mRNA composition described herein is administered to subjects of age 12 or older and at least one dose given in a vaccination regimen (e.g., a primary vaccination regimen and/or a booster vaccination regimen) is about 30 ug. In some embodiments, an mRNA composition described herein is administered to subjects of age 12 or older and at least one dose given in a vaccination regimen (e.g., a primary vaccination regimen and/or a booster vaccination regimen) is about 15 ug. In some embodiments, an mRNA composition described herein is administered to subjects of age 5 to less than 12 years of age and at least one dose given in a vaccination regimen (e.g., a primary vaccination regimen and/or a booster vaccination regimen) is about 10 ug. In some embodiments, an mRNA composition described herein is administered to subjects of age 2 to less than 5 and at least one dose given in a vaccination regimen (e.g., a primary vaccination regimen and/or a booster vaccination regimen) is about 3 ug. In some embodiments, an mRNA composition described herein is administered to subjects of 6 months to less than age 2 and at least one dose given in a vaccination regimen (e.g., a primary vaccination regimen and/or a booster vaccination regimen) is about 3 ug or lower, including, e.g., 2 ug, 1 ug, or lower). In some embodiments, an mRNA composition described herein is administered to infants of less than 6 months and at least one dose given in a vaccination regimen (e.g., a primary vaccination regimen and/or a booster vaccination regimen) is about 3 ug or lower, including, e.g., 2 ug, 1 ug, 0.5 ug, or lower).

In some embodiments, a dose administered to subjects in need thereof may comprise administration of a single mRNA composition described herein.

In some embodiments, a dose administered to subjects in need thereof may comprise administration of at least two or more (including, e.g., at least three or more) different drug products/formulations. For example, in some embodiments, at least two or more different drug products/formulations may comprise at least two different mRNA compositions described herein (e.g., in some embodiments each comprising a different RNA construct).

In some embodiments, a subject is administered two or more RNAs (e.g., as part of either a primary regimen or a booster regimen), wherein the two or more RNAs are administered on the same day or same visit. In some embodiments, the two or more RNAs are administered in separate compositions, e.g., by administering each RNA to a separate part of the subject (e.g., by intramuscular administration to different arms of the subject or to different sites of the same arm of the subject). In some embodiments, the two or more RNAs are mixed prior to administration (e.g., mixed immediately prior to administration, e.g., by the administering practitioner). In some embodiments, the two or more RNAs are formulated together (e.g., by (a) mixing separate populations of LNPs, each population comprising a different RNA; or (b) by mixing two or more RNAs prior to LNP formulation, so that each LNP comprises two or more RNAs).

In some embodiments, a subject is administered or a composition comprises one or more first RNAs and one or more second RNAs, each in the same amount (i.e., at a 1:1 ratio).

In some embodiments, a subject is administered or a composition comprises one or more first RNAs and one or more second RNAs, each in a different amount. For example, in some embodiments, a subject is administered or a composition comprises one or more first RNAs in an amount that is 0.01 to 100 times that of one or more second RNAs (e.g., wherein the amount of the one or more first RNAs is 0.01 to 50, 0.01 to 4, 0.01 to 30, 0.01 to 25, 0.01 to 20, 0.01 to 15, 0.01 to 10, 0.01 to 9, 0.01 to 8, 0.01 to 7, 0.01 to 6, 0.01 to 5, 0.01 to 4, 0.01 to 3, 0.01 to 2, 0.01 to 1.5, 1 to 50, 1 to 4, 1 to 30, 1 to 25, 1 to 20, 1 to 15, 1 to 10, 1 to 9, 1 to 8, 1 to 7, 1 to 6, 1 to 5, 1 to 4, 1 to 3, 1 to 2, or 1 to 1.5 times that of the one or more second RNAs). In some embodiments, a subject is administered or a composition comprises one or more first RNAs and one or more second RNAs, wherein the concentration of the one or more first RNAs is 1 to 10 times that of the one or more second RNAs. In some embodiments, a subject is administered or a composition comprises one or more first RNAs and one or more second RNAs, wherein the amount of the one or more first RNAs is 1 to 5 times that of the one or more second RNAs. In some embodiments, a subject is administered or a composition comprises one or more first RNAs and one or more second RNAs, wherein the concentration of the one or more first RNAs is 1 to 3 times that of the one or more second RNAs. In some embodiments, a subject is administered or a composition comprises one or more first RNAs and one or more second RNAs, wherein the amount of the one or more first RNAs is 2 times that of the one or more second RNAs. In some embodiments, a subject is administered or a composition comprises one or more first RNAs and one or more second RNAs, wherein the concentration of the one or more first RNAs is 3 times that of the one or more second RNAs.

In some embodiments, a subject is administered or a composition comprises two first RNAs, each encoding an antigen derived from an influenza strain or variant, wherein the amount of each RNA is not the same. For example, in some embodiments, the ratio of the two first RNAs is 1:0.01-100 (e.g., 1:0.01-50; 1:0.01-40; 1:0.01-30; 1:0.01-25; 1:0.01-20; 1:0.01-15; 1:0.01-10; 1:0.01-9; 1:0.01-8; 1:0.01-7; 1:0.01-6; 1:0.01-5; 1:0.01-4; 1:0.01-3; 1:0.01-2; 1:0.01-1.5, 1:0.1-10, 1:0.1-5, 1:0.1-3, 1:2-10, 1:2-5, or 1: 2-3). In some embodiments, a subject is administered or a composition comprises two first RNAs at a ratio of 1:3. In some embodiments, a subject is administered or a composition comprises two first RNAs at a ratio of 1:2.

For example, in some embodiments, the ratio of the three first RNAs is 1:0.01-100:0.01-100 (e.g., 1:0.01-50:0.01-50; 1:0.01-40:0.01-40; 1:0.01-30:0.01-30; 1:0.01-25:0.01-25; 1:0.01-20:0.01-20; 1:0.01-15:0.01-15; 1:0.01-10:0.01-10; 1:0.01-9:0.01-9; 1:0.01-8:0.01-8; 1: 0.01-7:0.01-7; 1:0.01-6:0.01-6; 1:0.01-5:0.01-5; 1:0.01-4:0.01-4; 1:0.01-3:0.01-3; 1:0.01-2:0.01-2; 1:0.01-1.5:0.01-1.5; 1:0.1-10:0.1-10, 1:0.1-5:0.1-5, 1:0.1-3:0.1-3, 1:2-10: 2-10, 1:2-5: 2-5, or 1: 2-3:2-3). In some embodiments, a subject is administered or a composition comprises three first RNAs at a ratio of 1:1:3. In some embodiments, a subject is administered or a composition comprises three first RNAs at a ratio of 1:3:3.

In some embodiments, a subject is administered or a composition comprises two or more second RNAs, one or more of which encode an HA protein of a Type A influenza virus, and one or more of which encode an HA protein of a Type B influenza virus. In some embodiments, the one or more second RNAs that encode an HA protein of a Type A influenza virus and the one or more second RNAs that encode an HA protein of a Type B influenza virus are present or are administered in the same amount (i.e., at a ratio of 1:1). In some embodiments, the one more second RNAs that encode an HA protein of a Type A influenza virus and the one or more second RNAs that encode an HA protein of a Type B influenza virus are administered in different amounts (e.g., in a ratio of between 1:10 and 10:1, or in a ratio of 1:2, 1:3, 1:4, 1:5, 2:1, 3:1, 4:1, or 5:1 (total RNA encoding an A antigen:total RNA encoding a B antigen).

In some embodiments, a subject is administered or a composition comprises two second RNAs, each encoding an HA protein of a different influenza virus type (e.g., a second RNA encoding an HA protein of a Type A influenza virus and a second RNA encoding an HA protein of a Type B influenza virus). In some embodiments, the second RNAs are administered or are present in the same amount (i.e., at a 1:1 ratio). In some embodiments, the second RNAs are administered or are present in different amounts (e.g., in a ratio of between 1:10 and 10:1, or in a ratio of 1:2, 1:3, 1:4, 1:5, 2:1, 3:1, 4:1, or 5:1 (A:B)).

In some embodiments, a subject is administered or a composition comprises three second RNAs, each encoding an HA protein of a different influenza virus subtype (e.g., an HA protein of an A/Wisconsin (H1N1) virus, an A/Darwin (H3N2) virus, and a B/Austria (Victoria) virus). In some embodiments, a subject is administered or a composition comprises each of the three second RNAs in the same amount (i.e., at a 1:1:1 ratio). In some embodiments, a subject is administered or a composition comprises a different amount of one or more of the three second RNAs (e.g., in a ratio of between 1:1:2 and 1:1:10 (e.g., in a ratio of 1:1:2, 1:1:3, 1:1:4, or 1:1:5), or in a ratio of between 2:2:1 and 2:2:10, (e.g., in a ratio of 2:2:1, 3:3:1, 4:4:1, or 5:5:1). In some embodiments, a subject is administered or a composition comprises three second RNAs, two of which encode HA proteins of different influenza type A virus, and one of which encodes an HA protein of an influenza type B virus. In some such embodiments, the second RNA encoding an HA protein of an influenza type B virus is present or is administered in a higher amount as compared to either second RNA encoding an HA protein from a type A virus (e.g., in some embodiments, the ratios of the two second RNAs encoding HA proteins from type A influenza viruses relative to the second RNA encoding an HA protein from a type B influenza virus is 1:1:1-10, 1:1:2, 1:1:3, 1:1:4, or 1:1:5 (A:A:B)). In some embodiments, a subject is administered or a composition comprises three second RNAs, two encoding an HA protein of an influenza type A virus and one encoding an HA protein of an influenza type B virus, wherein the ratio of the three second RNAs 1:1:4 (A:A:B). In some embodiments, the two second RNAs encoding an HA protein of an influenza type A viruses are each present or are each administered in a higher amount as compared to the second RNA encoding an HA protein from a type B virus (e.g., in some embodiments, the ratios of the two second RNAs encoding HA proteins from type A influenza viruses relative to the second RNA encoding an HA protein from a type B influenza virus is 1-10:1-10:1, 2:2:1, 3:3:1, 4:4:1, or 5:5:1 (A:A:B)).

In some embodiments, a subject is administered or a composition comprises four second RNAs, each encoding an HA protein of a different influenza virus subtype. In some such embodiments, the four second RNAs comprise two second RNAs encoding HA proteins of different influenza type A viruses and two second RNAs encoding HA proteins of different influenza type B virus (e.g., an HA protein of an H1N1 virus, an HA protein of an H3N2 virus, an HA protein of a BNictoria lineage virus, and an HA protein of a B/Yamagata lineage virus). In some embodiments, each of the two second RNAs encoding an HA protein of an influenza type A virus and each of the two second RNAs encoding an HA protein of an influenza type B virus are present in the same amount (i.e., the ratio of the four second RNAs is 1:1:1:1). In some embodiments, the two second RNAs encoding an HA protein of an influenza type B virus are each administered or are each present in a higher amount as compared to either second RNA encoding an HA protein from a type A virus (e.g., in some embodiments, the ratios of the two second RNAs encoding HA proteins from type A influenza viruses relative to the two second RNAs encoding an HA protein from a type B influenza virus is 1:1:2-10:2-10, 1:1:2-5:2-5, 1:1:2:2, 1:1:3:3, 1:1:4:4, 1:1:5:5, 1:1:6:6, 1:1:7:7, 1:1:8:8, 1:1:9:9, 1:1:10:10 (A:A:B:B)). In some embodiments, a subject is administered or a composition comprises four second RNAs, two encoding an HA protein of an influenza type A virus and two encoding an HA protein of an influenza type B virus, wherein the ratio of the four second RNAs 1:1:5:5 (A:A:B:B). In some embodiments, the two second RNAs encoding an HA protein of an influenza type A virus are each administered or are each present in a higher amount as compared to either second RNA encoding an HA protein from a type B virus (e.g., in some embodiments, the ratios of the two second RNAs encoding HA proteins from type A influenza viruses relative to the two second RNAs encoding an HA protein from a type B influenza virus is 2-10:2-10:1:1, 2-5:2-5:1:1, 2:2:1:1, 3:3:1:1, 4:4:1:1, 5:5:1:1, 6:6:1:1, 7:7:1:1, 8:8:1:1, 9:9:1:1, 10:10:1:1 (A:A:B:B)).

In some embodiments, a composition comprises or a subject is administered four second RNAs, comprising three second RNAs that encode HA proteins of different influenza type A viruses and one second RNA encoding an HA protein of an influenza type B virus (e.g., A/Wisconsin (H1N1), A/Darwin (H3N2), A/Cambodia (H3N2), and B/Austria (Victoria)). In some such embodiments, each of the four second RNAs is administered or is present in the same amount (i.e., at a 1:1:1:1 ratio). In some embodiments, the amount of second RNA encoding an HA protein of an influenza type B virus is higher than any one of the second RNAs encoding an HA protein of an influenza type A virus (e.g., in some embodiments, the ratio of second RNAs is 1:1:1:1-10, 1:1:1:1-5, 1:1:1:2, 1:1:1:3, 1:1:1:4, or 1:1:1:5 (A:A:A:B)). In some embodiments, the ratio of second RNAs administered or in a composition is 1:1:1:5 (A:A:A:B). In some embodiments, the amount of each of the second RNAs encoding an HA protein of an influenza type A virus is higher than that of the second RNA encoding an HA protein of an influenza type B virus (e.g., in some embodiments, the ratio of second RNAs is 1-10:1-10:1-10:1, 1-5:1-5:1-5:1, 2:2:2:1, 3:3:3:1, 4:4:4:1, or 5:5:5:1 (A:A:A:B)).

In some embodiments, a subject is administered or a composition comprises one or more second RNAs encoding an HA protein of an influenza virus (e.g., two second RNAs, three second RNAs, or four second RNAs, each encoding an HA protein of a different influenza virus) in a total amount of 0.1 to 100 μg (e.g., 1 to 90 μg, 3 to 90 μg, 1 to 60 μg, 3 to 60 μg, 5 to 60 μg, 10 to 60 μg, 30 to 60 μg, 3 to 30 μg). In some embodiments, a subject is administered or a composition comprises one or more second RNAs encoding an HA protein of an influenza virus in a total amount of 3 μg, 5 μg, 6 μg, 10 μg, 15 μg, 20 μg, 25 μg, 30 μg, 45 μg, 60 μg, 75 μg, or 90 μg.

In some embodiments, a subject is administered or a composition comprises three or four second RNAs, each encoding an HA antigen of a different influenza strain, in one of the amounts listed in the below Table C (each “Influenza Component” corresponding to a second RNA encoding an HA antige (e.g., a second RNA as described herein).

TABLE C
Exemplary Amounts of Second RNAs Encoding HA Antigens

| Combination | Influenza | Influenza | Influenza | Influenza | |
|---|---|---|---|---|---|
| # | Component 1 | Component 2 | Component 3 | Component 4 | Total |
| 1 | 7.5 μg (A type) | 7.5 μg (A type) | 7.5 μg (B type) | 7.5 μg (B type) | 30 μg |
| 2 | 15 μg (A type) | 15 μg (A type) | 15 μg (B type) | 15 μg (B type) | 60 μg |
| 3 | 11.25 μg (A type) | 11.25 μg (A type) | 11.25 μg (B type) | 11.25 μg (B type) | 45 μg |
| 4 | 5 μg (A type) | 5 μg (A type) | 25 μg (B type) | 25 μg (B type) | 60 μg |
| 5 | 2.5 μg (A type) | 2.5 μg (A type) | 12.5 μg (B type) | 12.5 μg (B type) | 30 μg |
| 6 | 7.5 μg (A type) | 7.5 μg (A type) | 30 μg (B type) | — | 45 μg |
| 7 | 7.5 μg (A type) | 7.5 μg (A type) | 7.5 μg (A type) | 7.5 μg (B type) | 30 μg |

In some embodiments, a composition described herein is characterized in that it produces influenza neutralizing antibody titers that are within at least two fold of those produced by a reference vaccine for each influenza virus that it encodes antigens of (e.g., wherein the reference vaccine is a quadrivalent influenza RNA vaccine administered alone, or an approved (non-RNA) influenza vaccine).

In some embodiments, the influenza vaccine is an alphainfluenza virus, a betainfluenza virus, a gammainfluenza virus or a deltainfluenza virus vaccine. In some embodiments the vaccine is an Influenza A virus, an Influenza B virus, an Influenza C virus, or an Influenza D virus vaccine. In some embodiments, the influenza A virus vaccine comprises a hemagglutinin selected from H1, H2, H3, H4, H5, H6, H7, H8, H9, H10, H11, H12, H13, H14, H15, H16, H17, and H18, or an immunogenic fragment or variant of the same, or a nucleic acid (e.g., RNA) encoding any one of the same. In some embodiments the influenza A vaccine comprises or encodes a neuraminidase (NA) selected from N1, N2, N3, N4, N5, N6, N7, N8, N9, N10, and N11, or an immunogenic fragment or variant of the same, or a nucleic acid (e.g., RNA) encoding any one of the same. In some embodiments, the influenza vaccine comprises at least one Influenza virus hemagglutinin (HA), neuraminidase (NA), nucleoprotein (NP), matrix protein 1 (M1), matrix protein 2 (M2), non-structural protein 1 (NS1), non-structural protein 2 (NS2), nuclear export protein (NEP), polymerase acidic protein (PA), polymerase basic protein PB1, PB1-F2, and/or polymerase basic protein 2 (PB2), or an immunogenic fragment or variant thereof, or a nucleic acid (e.g., RNA) encoding any of one of the same.

### EXAMPLES

Below are examples of specific aspects for carrying out the present disclosure. The following examples are included to demonstrate aspects of the disclosure. The examples are offered for illustrative purposes only and are not intended to limit the scope of the present disclosure in any way. It should be appreciated by those of skill in the art that the techniques disclosed in the examples which follow represent techniques discovered by the inventor to function well in the practice of the disclosure. However, those of skill in the art should, in light of the present disclosure, appreciate that many changes may be made in the specific aspects which are disclosed and still obtain a like or similar result without departing from the spirit and scope of the disclosure. Efforts have been made to ensure accuracy with respect to numbers used (e.g., amounts, temperatures, etc.), but some experimental error and deviation should, of course, be allowed for.

### Example 1: Influenza modRNA Drug Product Composition

The drug product composition is an influenza modRNA drug substance targeting the Wisconsin 2021/2022 hemagglutinin.

TABLE 1
Exemplary formulation composition of the ready-to-
use (RTU) presentation of Flu vaccine drug product

| Components | Function | Concentration, mg/mL |
|---|---|---|
| PF-07829855 Drug substance (mRNA) | Active | 0.1 |
| ALC-0315 | Functional lipid | 1.43 |
| ALC-0159 | Functional lipid | 0.18 |
| DSPC | Structural lipid | 0.31 |
| Cholesterol | Structural lipid | 0.62 |
| Sucrose | Cryoprotectant/Tonicifier | 1.3 |
| Tris/Tromethamine | Buffer, pH 7.4 | 0.18 |
| Tris HCl | | 1.34 |
| Water for injection | Solvent/Vehicle | q.s. |

Unless otherwise stated, as used herein, the Flu modRNA molecule encoding HA (also referred to herein as the flu mRNA drug substance) includes the following elements listed in Table 2:

TABLE 2
Table of elements

| Element | Description | Position |
|---|---|---|
| cap | A modified 5′-cap1 structure | 1-2 (GA) |
| | (m⁷G⁺m^{3′}-5′-ppp-5′-Am) | |
| 5′-UTR | 5′-untranslated region derived from | 3-54 |
| | human alpha-globin RNA with an | |
| | optimized Kozak sequence | |
| | SEQ ID NO: 54 | |
| 3′-UTR | The 3′ untranslated region comprises two sequence | 3880-4174 |
| | elements derived from the amino-terminal | |
| | enhancer of split (AES) mRNA and the | |
| | mitochondrial encoded 12S | |
| | ribosomal RNA to confer RNA stability and high | |
| | total protein expression. SEQ ID NO: 55 | |
| poly(A) | A 110-nucleotide poly(A)-tail consisting | 4175-4284 |
| | of a stretch of 30 adenosine residues, | |
| | followed by a 10-nucleotide linker | |
| | sequence and another 70 adenosine residues. | |
| | SEQ ID NO: 56 | |

Sequences

GA

| | |
|---|---|
| GAAΨAAAC ΨAGΨAΨΨCΨΨ CΨGGΨCCCCA CAGACΨCAGA GAGAACCCGC | 50 |
| CACC (SEQ ID NO: 54) | 54 |
| C ΨCGAGCΨGGΨ ACΨGCAΨGCA | 3900 |
| CGCAAΨGCΨA GCΨGCCCCΨΨ ΨCCCGΨCCΨG GGΨACCCCGA GΨCΨCCCCCG | 3950 |
| ACCΨCGGGΨC CCAGGΨAΨGC ΨCCCACCΨCC ACCΨGCCCCA CΨCACCACCΨ | 4000 |
| CΨGCΨAGΨΨC CAGACACCΨC CCAAGCACGC AGCAAΨGCAG CΨCAAAACGC | 4050 |
| ΨΨAGCCΨAGC CACACCCCCA CGGGAAACAG CAGΨGAΨΨAA CCΨΨΨAGCAA | 4100 |
| ΨAAACGAAAG ΨΨΨAACΨAAG CΨAΨACΨAAC CCCAGGGΨΨG GΨCAAΨΨΨCG | 4150 |
| ΨGCCAGCCAC ACCCΨGGAGC ΨAGC (SEQ ID NO: 55) | |
| AAAAAA AAAAAAAAAA AAAAAAAAAA | 4200 |
| AAAAGCAΨAΨ GACΨAAAAAA AAAAAAAAAA AAAAAAAAAA AAAAAAAAAA | 4250 |
| AAAAAAAAAA AAAAAAAAAA AAAAAAAAAA AAAA 4284 (SEQ ID NO: 56) | |

Ψ=1-methyl-3′-pseudouridylyl

In some embodiments, the immunogenic composition comprising one lipid nanoparticle encapsulated mRNA molecule encoding HA is monovalent and has a dose selected from any one of 1 μg mRNA, 2 μg RNA, 5 μg RNA, and 20 μg RNA.

In some embodiments, the immunogenic composition comprising one lipid nanoparticle encapsulated mRNA molecule encoding HA, a second lipid nanoparticle encapsulated mRNA molecule encoding HA, a third lipid nanoparticle encapsulated mRNA molecule encoding NA, and a fourth lipid nanoparticle encapsulated mRNA molecule encoding NA, wherein the total dose is up to 20 μg RNA.

In some embodiments, the subject is aged 30-50 years.

### Example 2: DESCRIPTION OF QUADRIVALENT DRUG PRODUCT

The quadrivalent drug product is a preservative-free, sterile dispersion of liquid nanoparticles (LNP) in aqueous cryoprotectant buffer for intramuscular administration. The drug product is formulated at 0.1 mg/mL RNA in 10 mM Tris buffer, 300 mM sucrose, pH 7.4.

The drug product is supplied in a 2 mL glass vial sealed with a chlorobutyl rubber stopper and an aluminum seal with flip-off plastic cap (maximum nominal volume of 0.3 mL).

TABLE 3
Composition of Quadrivalent Drug Product

| | | | | | Nominal |
|---|---|---|---|---|---|
| | | | | | Amount or |
| | | | | Filled | Net |
| | | | Unit | Amount | Quantity |
| | Grade/Quality | | Formula | (Total | (Net |
| Name of Ingredient | Standard | Function | (mg/mL) | mg/vial) | mg/vial) |
| PF-07829855 Drug | In-house | Active | 0.025 | 0.013 | 0.008 |
| Substance (Wisconsin) | specification | ingredient | | | |
| PF-07836258 Drug | In-house | Active | 0.025 | 0.013 | 0.008 |
| Substance (Cambodia) | specification | ingredient | | | |
| PF-07836259 Drug | In-house | Active | 0.025 | 0.013 | 0.008 |
| Substance (Phuket) | specification | ingredient | | | |
| PF-07836261 Drug | In-house | Active | 0.025 | 0.013 | 0.008 |
| Substance | specification | ingredient | | | |
| (Washington) | | | | | |
| ALC-0315^{a} | In-house | Functional | 1.43 | 0.72 | 0.43 |
| | specification | lipid | | | |
| ALC-0159^{b} | In-house | Functional | 0.18 | 0.09 | 0.05 |
| | specification | lipid | | | |
| DSPC^{c} | In-house | Structural | 0.31 | 0.16 | 0.09 |
| | specification | lipid | | | |
| Cholesterol | Ph. Eur., NF | Structural | 0.62 | 0.31 | 0.2 |
| | | lipid | | | |
| Sucrose | Ph. Eur., NF | Cryoprotecta | 102.69 | 51.35 | 30.81 |
| | | nt | | | |
| Tromethamine (Tris | Ph. Eur., USP | Buffer | 0.20 | 0.10 | 0.06 |
| base) | | component | | | |
| Tris (hydroxymethyl) | In-house | Buffer | 1.32 | 0.66 | 0.40 |
| aminomethane | specification | component | | | |
| hydrochloride (Tris HCl) | | | | | |
| Water for Injection | Ph. Eur., USP, | Solvent | q.s. ^{d} to | q.s. ^{d} to | q.s. ^{d} to |
| | JP | | 1.00 mL | 0.50 mL | 0.30 mL |

^{a}ALC-0315 = ((4-hydroxybutyl)azanediyl)bis(hexane-6,1-diyl)bis(2-hexyldecanoate)
^{b}ALC-0159 = 2-[(polyethylene glycol)-2000]-N,N-ditetradecylacetamide
^{c}DSPC = 1,2-Distearoyl-sn-glycero-3-phosphocholine
^{d} q.s. is an abbreviation for quantum satis meaning as much as is sufficient.

The recommended storage temperature of the FIH drug substance is −20±5° C.

The recommended long term storage temperature of the FIH drug product is −60 to −90° C.

The drug product may be stored at 2-8° C. at Point of Use.

### Example 3: RSV F Protein Composition

Unless stated otherwise, the RSV subunit component of the combination RSV subunit and influenza mRNA-LNP composition as used in the present Examples is described as follows: RSVpreF, an RSV bivalent stabilized prefusion F subunit vaccine (project code PF-06928316), is intended to prevent RSV-associated lower respiratory tract disease in older adults via active immunization or in infants through administration to pregnant women. RSVpreF contains two engineered prefusion F antigens, 847 A (a modified F from RSV subgroup A [strain Ontario]) and 847 B (a modified F from subgroup B [strain Buenos Aires]), each structurally engineered for stability in the prefusion conformation. The vaccine is comprised of co-lyophilized antigens reconstituted with sterile water. The selected commercial dose for RSVpreF is 120 μg (60 μg of 847A and 60 μg of 847B) in 0.5 mL administered as a single IM dose for both older adults and maternal immunization.

The investigational RSV vaccine is comprised of equal quantities of two recombinant RSV F antigens, 847A from subgroup A and 847B from subgroup B, each structurally engineered for enhanced stability in the prefusion conformation. The RSVpreF drug product is presented as a sterile Iyophilized powder at the target strength of 240 pig/mL upon reconstitution and is filled in a 2 mL Type 1 glass vial. Each vial of the lyophilized vaccine is reconstituted with 0.65 mL of sterile water diluent. The bulk drug product formulation contains 20 mM Tris (tromethamine), 50 mM sodium chloride, 0.2 mg/mL polysorbate 80, 30 mg/mL sucrose and 60 mg/mL mannitol.

### Example 4: Optimization of Aqueous Compositions Comprising RSV F Protein Trimer in Prefusion Conformation to Reduce Duration of Lyophilization while Limiting Loss of Prefusion Content and Increase of Aggregates

To optimize the choice and ratio of stabilizer/bulking agents to be used in the composition, 10 different matrices shown in Table 4 were selected and lyophilized. Developmental lyophilization cycles were applied to produce lyophilized compositions containing sucrose/mannitol or sucrose/glycine. All test formulations were monitored for aggregation (% HMMS), and prefusion content by AM14 ELISA and AM22 Fab titration before and after lyophilization.

TABLE 4
RSV F protein composition

| | Common | Sucrose | Mannitol | Glycine | NaCl |
|---|---|---|---|---|---|
| Formulation # | components | %(w/v) | %(w/v) | %(w/v) | (mM) |
| 1 | 240 μg/mL | 3 | 6 | 0 | 50 |
| 2 | RSV A and 240 | 3 | 6 | 0 | 0 |
| 3 | μg/mL RSV B, | 2 | 5.5 | 0 | 50 |
| 4 | 20 mM Tris at | 2 | 5.5 | 0 | 0 |
| 5 | pH 7.4, 0.02% | 1.5 | 0 | 3 | 25 |
| 6 | PS80 | 1.5 | 0 | 3 | 0 |
| 7 | | 1 | 0 | 2.5 | 25 |
| 8 | | 1 | 0 | 2.5 | 0 |
| 9 | | 2 | 4 | 0 | 50 |
| 10 (Gen 1 | | 9 | 0 | 0 | 50 |
| matrix, control) | | | | | |

Compositions #1 to #9 could all be lyophilized in about 24 hours. For the compositions containing sucrose and mannitol, two compositions containing 3% sucrose/6% mannitol with or without NaCl (#1 and #2 in Table 4) demonstrated the best overall stability among all tested compositions upon lyophilization. Composition #4 (2% sucrose/5.5% mannitol) and composition #9 (2% sucrose/4% mannitol with NaCl) showed the largest drop in prefusion content and the largest increase in aggregation upon lyophilization indicating non-ideal sucrose/mannitol levels and/or ratios. In addition, data suggest that the presence of NaCl in the composition can potentially reduce the rate of aggregation in most composition upon lyophilization.

For composition containing sucrose and glycine, the one containing 1% sucrose/2.5% glycine with or without NaCl (#7 and #8 in Table 4) showed marginally better stability than the one containing 1.5% sucrose/3% glycine with or without NaCl (#5 and #6 in Table 4).

In view of the above, inclusion of sucrose and mannitol or sucrose and glycine in composition comprising RSV F protein trimer in the prefusion conformation is particularly advantageous to obtain compositions which can be lyophilized and where the loss of prefusion content and the increase of aggregation is limited. In particular, compositions where the ratio of sucrose to mannitol or sucrose to glycine is between 1 to 1 and 1 to 4, preferably 1 to 2 to 1 to 4, more preferably 1 to 2 provide compositions that can be lyophilized in 24H and where the loss of prefusion content and the increase of aggregation is acceptable.

### Example 5: Development of a Lyophilization Process for RSV F Protein Trimer in the Prefusion Conformation

The objective of the lyophilization development was to develop a target lyophilization cycle by optimizing the conditions for each stage of the lyophilization cycle including freezing, annealing, and drying to produce a visually elegant cake with acceptable product quality. After initial development to obtain the target lyophilization cycle, the robustness of the lyophilization cycle was assessed by challenging the target cycle parameters and evaluating the impact to product quality. The critical quality attributes evaluated during lyophilization process development included residual moisture content, HMMS and prefusion content. Additionally, lyophilization of a mannitol-sucrose formulation can result in the presence of multiple polymorphic forms of mannitol, therefore presence of Mannitol Hemihydrate (MHH) was also monitored, as this phase can have a negative impact on product stability over time.

The major steps in the lyophilization process include the freezing, annealing, drying via sublimation, and drying via desorption of residual moisture. Several parameters must be considered throughout each of the steps including shelf temperature, ramp and hold times, chamber pressure during drying, and product temperature throughout the cycle. Freezing hold temperatures affect the structural characteristics of the frozen matrix. Inclusion of an annealing step reduces vial to vial heterogeneity typically induced during freezing by promoting crystallization and homogeneity of ice crystal size, and maximizing the crystallization of mannitol, which can ultimately minimize differences in cake appearance and reduce drying time. The cycle time, shelf temperature and chamber pressure used during the first ramp of drying directly affect the temperature of the frozen matrix, the rate of sublimation and the structure and appearance of the dried cake. The overall cycle time and shelf temperature utilized during the second ramp of drying primarily impacts the residual moisture level in the dried cake. Selection of the appropriate parameter values for each step collectively enables a lyophilization cycle that produces a dried cake in vial having a desirable appearance of an elegant cake with a low level of residual moisture to support the stabilization of the drug product.

Following thermal analysis of the drug product formulation, initial parameters for the lab-scale lyophilization cycle were selected based on historical process development experience with semi-crystalline formulations (see Table 5 below).

TABLE 5
Target Lyophilization Cycle

| | | Ramp | Ramp | Hold | Chamber |
|---|---|---|---|---|---|
| | Temperature | Rate | Time | Time | Pressure |
| Segment | (° C.) | (° C./min) | (min) | (min) | (mTorr/μbar) |
| Freezing | −50 | 1.0 | 70 | 60 | NA |
| Annealing | −10 | 0.5 | 80 | 210 | NA |
| Refreezing | −50 | 0.5 | 80 | 60 | NA |
| Vacuum Initiation | −50 | NA | NA | NA | 200 mTorr (267 μbar) |
| First Dry Step | 0 | 0.5 | 100 | 0 | 200 mTorr (267 μbar) |
| Second Dry Step | 50 | 0.2 | 250 | 515 | 200 mTorr (267 μbar) |
| Stoppering | NA | NA | NA | NA | 600,000 mTorr (800 mbar) |

Abbreviations: NA = not applicable

Aggressive cycle conditions were then created to confirm the critical process parameters and define the upper and lower boundaries to be challenged to assess process robustness. The RSV F protein composition (see Table 6) was lyophilized using the five aggressive cycles described in Table 7 to confirm the critical process parameters and define the upper and lower boundaries to be challenged in the process robustness studies. The cycles assessed the lyophilization parameters of freezing ramp rate, annealing ramp rate and temperature, and drying ramp rate and temperature by challenging the parameter values of the target lyophilization cycle. The cumulative aggressive cycle combined the slow freezing and annealing ramp rates with a high freezing temperature, annealing temperature and drying temperature. Individual cycles were executed as listed in Table 7 and varied either the freezing ramp rate, annealing temperature, annealing ramp rate, or drying temperature and ramp rate while keeping all other parameters consistent with the target cycle values.

TABLE 6
RSV A and B composition pre- and post- lyophilisation

| | RSV F protein composition to | Composition Post- |
|---|---|---|
| | be lyophilised | Reconstitution with Sterile Water |
| Ingredient | (mg/mL or g/L) | Diluent (mg/mL) |
| RSV A | 0.32 | 0.24 |
| RSV B | 0.32 | 0.24 |
| Tromethamine^{b} | 0.30^{d} | 0.23^{e} |
| Tris-HCl^{c} | 2.76^{d} | 2.07^{e} |
| Sucrose | 30 | 22.5 |
| Mannitol | 60 | 45.0 |
| Polysorbate 80 | 0.20 | 0.15 |
| NaCl | 2.92^{f} | 2.19^{g} |
| Water for Injection | q.s to 1 mL or L | q.s to 1 mL |

^{b}Also known as Trometamol and Tris Base
^{c}Also known as Tris (hydroxymethyl) aminomethane hydrochloride (Tris-HCl), Tromethamine HCl, and Trometamol HCl
^{d}Tromethamine + Tris-Hydrochloride composition is equivalent to 20 mM Tris
^{e}Tromethamine + Tris-Hydrochloride composition is equivalent to 15 mM Tris
^{f}Equivalent to 50 mM NaCl
^{g}Equivalent to 37.5 mM NaCl

TABLE 7
Critical Process Parameter Assessment Cycles

| | | Freezing | | Annealing | Drying | 2nd |
|---|---|---|---|---|---|---|
| | Freezing | Ramp | Annealing | Ramp | Ramp | Drying |
| Cycle | Temp. | Rate | Temp | Rate | Rate | Temp. |
| Description | (° C.) | (° C./min) | (° C.) | (° C./min) | (° C./min) | (° C.) |
| Target | −50 | 1 | −10 | 0.5 | 0.5 (1^{st} Drying); | 50 |
| | | | | | 0.2 (2^{nd} Drying) | |
| Cumulative | −45 | 0.25 | −5 | 0.2 | 0.1 (1^{st} Drying); | 55 |
| Aggressive | | | | | 0.1 (2^{nd} Drying) | |
| Freezing | −50 | 0.25 | −10 | 0.5 | 0.5 (1^{st} Drying); | 50 |
| Ramp | | | | | 0.2 (2^{nd} Drying) | |
| Aggressive | | | | | | |
| Annealing | −50 | 1 | −5 | 0.5 | 0.5 (1^{st} Drying); | 50 |
| Temp | | | | | 0.2 (2^{nd} Drying) | |
| Aggressive | | | | | | |
| Annealing | −50 | 1 | −10 | 0.2 | 0.5 (1^{st} Drying); | 50 |
| Ramp | | | | | 0.2 (2^{nd} Drying) | |
| Aggressive | | | | | | |
| Drying | −50 | 1 | −10 | 0.5 | 0.5 (1^{st} Drying); | 55 |
| Temp + Ramp | | | | | 0.1 (2^{nd} Drying) | |
| Aggressive | | | | | | |

All experiments were conducted with a LyoStar3 Lyophilizer a from FTS System Inc. The aggressive cycles assessed the impact of freezing ramp rate, annealing ramp rate and temperature, and drying ramp rate and temperature on moisture content, prefusion content, HMMS and presence of MHH in comparison to the lyophilized composition produced using the target cycle.

The impact of the cumulative aggressive cycle to the critical quality attributes indicated a minor impact while HMMS is within typical method variability for SE-HPLC, a drop in prefusion content relative to the pre-lyophilized composition and the lyophilized composition obtained using the target cycle is observed.

### Example 6: Quadrivalent Influenza and RSV Bivalent Combination Composition

The quadrivalent influenza mRNA vaccine & RSV bivalent vaccine materials used in the Phase I Dosage and Administration Instructions (DAI) verification study is listed in Table 8

TABLE 8

| Use | Material/Component | Description |
|---|---|---|
| Active DP | See also Table 3 | 0.12 mg/ml Flu mRNA in 10 mM Tris buffer, 300 |
| (Quadrivalent | | mM sucrose, pH 7.4 |
| Influenza) | | |
| Active DP (RSV | | 120 μg of RSV bivalent antigen, 1.15 mg Tris, |
| Bivalent) | | 11.3 mg sucrose, 22.5 mg mannitol, 1.1 mg |
| | | NaCl, 0.08 mg polysorbate 80 (PS80), pH 7.4 |
| Diluent (RSV | | Sterile Water as Diluent |
| Bivalent) | | |
| Vial adapter | 13 mm APE Medical vial | 13 mm APE Medical vial adapter |
| | adapter | |
| Syringes | 1 mL polycarbonate | Disposable sterile polycarbonate; 1 mL |
| | syringe | |
| Syringes | 3 mL polypropylene | Disposable sterile polypropylene; 3 mL |
| | syringe | |
| Syringes | 10 mL polypropylene | Disposable sterile polypropylene; 10 mL |
| | syringe | |
| Use | Material/Component | Description |
| Stainless Steel | 21G × 1″ stainless steel | 21G 1″ |
| Needles (for sample | needle | |
| drawing) | | |
| Stainless Steel | 25G × 1.5″ stainless steel | 25G 1.5″ |
| Needles | needle | |
| (for capping) | | |
| fluid dispensing | Fluid Dispensing | Fluid Dispensing Connector |
| connector | Connector | |
| Sample Collection | RNAse free 1.2 mL | Vials for sample storage |
| | centrifuge tubes | |

### Example 7: Compatibility of Combination of Influenza mRNA & RSV Subunit Vaccine

This study was designed to demonstrate that:
a) Combination of Influenza mRNA & RSV subunit Vaccine (“Influenza mRNA & RSV Combination Vaccine”) dosing suspension is compatible with the administration component(s) required for dosage preparation.
b) Combination of Influenza mRNA & RSV subunit Vaccine dosing suspension is stable for a period of time adequate for the operation specified in the DAI.

The planned Phase I presentation for the Influenza mRNA & RSV Combination Vaccine includes:
Quadrivalent Influenza mod mRNA Vaccine:
Quadrivalent Influenza mod mRNA drug product, Suspension for Injection (60 mcg/vial)
   In addition to the 60 mcg/vial described herein, the DAI study also investigated a Quadrivalent Influenza mod mRNA drug product, Suspension for Injection (90 mcg/vial).
RSV Bivalent Vaccine Kit:
   RSV Bivalent drug product, Powder for Solution for Injection (120 mcg/vial)
   Sterile Water as Diluent Prefilled Syringe (SW PFS)
   13 mm vial adapter

This study evaluated the stability of prepared quadrivalent influenza and RSV bivalent combination vaccine when held in contact with vials, dosing syringes, and stainless-steel needles. The evaluated concentrations are based on a bracket of lowest and highest final clinical doses of 180 mcg (1:2 ratio of Influenza:RSV) and 210 mcg (1:1.33 of Influenza:RSV). Table 9 provides information on the dose configuration. To evaluate the bracket, the dosage mixing configuration for this study was designed to prepare 180 mcg and 210 mcg doses as bulk preparations, as summarized in Table 10.

TABLE 9
Dose Calculations for Dosage Form Configurations of
PF-07941314 Combination Vaccine for IM Injection

| | 180 mcg dose (Group 1 - | | 210 mcg dose (Group 2 - | |
|---|---|---|---|---|
| | 1:2 ratio) | | 1:1.33 ratio) | |
| | Final Dose | Dosing Volume | Final Dose | Dosing Volume |
| Active DP | (mcg) | (mL) | (mcg) | (mL) |
| Quadrivalent | 60 | 0.5 | 90 | 0.75 |
| Influenza | | | | |
| (0.12 mg/mL) | | | | |
| RSV Bivalent | 120 | 0.5 | 120 | 0.5 |
| (0.24 mg/mL) | | | | |
| Total | 180 | 1.0 | 210 | 1.25 |

TABLE 10
Bulk Prep Calculations Per Sample

| | | Bulk Prep | | Bulk Prep | |
|---|---|---|---|---|---|
| | | Group 1 | | Group 2 | |
| | | (1:2) 180 | | (1:1.33) 210 | |
| Active DP | Metrics | mcg dose | | mcg dose | |
| Quadrivalent | Volume | 3 | mL | 3.6 | mL |
| Influenza | Number of Vials | 6 | vials | 8 | vials |
| (0.12 mg/mL) | | | | | |
| RSV Bivalent | Volume (mL) | 3 | mL | 2.4 | mL |
| (0.24 mg/mL) | Number of Vials | 6 | vials | 5 | vials |
| Combined Vaccine | Volume (mL) | 6 | mL | 6 | mL |

Quadrivalent influenza vials were transferred directly from −80° C. freezer and thawed at room temperature under ambient light. Upon complete thaw, the vial contents were mixed by inversion for 10 times. RSV bivalent vaccine preparation is performed using the sterile water diluent and vial adapter in alignment with the proposed instructions for use.

Samples prepared using syringe-to-syringe mixing support syringe contact samples. Samples prepared using an in-vial dilution will support the glass vial contact samples. The prepared solutions in polycarbonate (PC) syringes, polypropylene (PP) syringes, and glass vials will be held at 30° C. for up to 4 hours, which supports the commercial RSV vaccine temperature hold guidance. After the incubation, the preparations were observed for particulates and ejected through 25-gauge 1.5″ needles. The sample aliquots were submitted in real-time for immediate analysis by the assays listed in Table 11 below.

The composition of the “180 mcg dose” final drug product is 120 mcg of RSV and 60 mcg of Flu modRNA OP. The matrices of the pre-combination OP are: RSV OP Composition post-reconstitution with WFI includes: 240 mcg/ml RSV (120 mcg RSV-A+120 mcg RSV-B) in 15 mM Tris, 2.25% Sucrose, 4.5% Mannitol, 37.5 mM NaCl, 0.015% PS-80, pH 7.4, and does not include an adjuvant. The quadrivalent modRNA Flu DP Composition includes: 120 mg/ml Flu modRNA in 10 mM Tris and 300 mM Sucrose, pH 7.4. Accordingly, the RSV Control Post Mix Composition includes: 120 mcg/ml (60 mcg RSV-A+60 mcg RSV-B) in 12.5 mM Tris, 6.26% Sucrose, 2.25% Mannitol, 18.75 mM NaCl, 0.0075% PS-80, pH 7.4. The Flu Control Post Mix Composition includes: 60 mcg/ml Flu modRNA in 12.5 mM Tris, 6.26% Sucrose, 2.25% Mannitol, 18.75 mM NaCl, 0.0075% PS-80, pH 7.4

The composition of the “210 mcg dose” final drug product dose is 120 mcg of RSV and 90 mcg of Flu modRNA DP. The matrices of the pre-combination drug product (DP) are:

RSV subunit DP composition post-reconstitution with WFI includes: 240 mcg/ml RSV (120 mcg RSV-A+120 mcg RSV-B) in 15 mM Tris, 2.25% Sucrose, 4.5% Mannitol, 37.5 mM NaCl, 0.015% PS-80, pH 7.4, and does not include an adjuvant. The quadrivalent modRNA Flu DP Composition includes 120 mg/ml Flu modRNA in 10 mM Tris and 300 mM Sucrose, pH 7.4. Accordingly, the RSV Control Post Mix Composition includes: 96 mcg/ml (48 mcg RSV-A+48 mcg RSV-B) in 12 mM Tris, 7.06% Sucrose, 1.8% Mannitol, 15 mM NaCl, 0.006% PS-80, pH 7.4. The Flu Control Post Mix Composition includes: 72 mcg/ml Flu modRNA in 12 mM Tris, 7.06% Sucrose, 1.8% Mannitol, 15 mM NaCl, 0.006% PS-80, pH 7.4.

Exemplary In Vial Dilutions are described as follows: RSV DP Control Vials of RSV DP were reconstituted with a pre-filled syringe containing WFI using the necessary vial adaptor. 3 mL polypropylene syringes were filled with 1.0 mL of Flu DP Matrix using a vial adaptor. Flu modRNA DP Matrix containing syringe was dispensed into the RSV DP vial. This was inverted to mix. This was repeated and each vial was capped. Flu DP Control Vials of RSV Post-Recon Matrix (0.7 mL fill) was pierced using a vial adaptor. 3 mL polypropylene syringes were filled with 1.05 mL of Flu Quad modRNA LNP DP. Using a vial adaptor, Flu DP containing syringe was dispensed into the RSV Post-Recon Matrix containing vial. This was inverted to mix. This was repeated and each vial was capped. The in-vial dilutions were performed at the 4, 2, and 0-hour timepoints. The 4 and 2-hour samples were staged at 30° C.

TABLE 11
Analytical Methods

| | | | Method | |
|---|---|---|---|---|
| Assay | | | Number and | Volume |
| Key | Assay | Study Criteria | Version | Needed (mL) |
| A | Appearance | White to off- | Research | Share with pH |
| | | white | Method | |
| | | suspension | | |
| | Appearance | Essentially Free | | |
| | (Particles) | from Visible | | |
| | | Particles | | |
| | pH | 7.4 ± 0.5 | M00125 | 1 |
| | RNA Content (RG) | T0 ± 20% | Research | 0.1 |
| | | | Method | |
| | RNA Integrity (CGE | ≥50% Intact | Research | 0.2 |
| | by fragment | RNA | Method | |
| | analyzer) | | | |
| | RNA Encapsulation | ≥80% | Research | Share with RNA |
| | (RG) | | Method | Content |
| | LNP Size (DLS) | 40-120 nm | Research | 0.1 |
| | | | Method | |
| | LNP PDI (DLS) | ≤0.3 | Research | Share with DLS |
| | | | Method | |
| | In vitro Expression | Report Result | Research | 0.5 |
| | | | Method | |
| | Protein | T0 ± 20% | Research | 1 |
| | Concentration | | Method | |
| | HMMS (SEC) | ≤25% | Research | 1 |
| | LMMS (SEC) | ≤5% | Method | |
| | Trimer (SEC) | ≥70% | | |
| | Relative Prefusion | 50-150% | Research | 0.5 |
| | Content (Prefusion F | | Method | |
| | ELISA)^{a} | | | |

| | |
|---|---|
| Contingency | 1.6 |
| Total Volume | 6 |

^{a}Prefusion F ELISA was tested in duplicate from the same sample. Lot release result for Protein Concentration will be used for dilution calculation.
Abbreviations:
RG = RiboGreen;
LNP = Lipid Nano Particle;
CGE = capillary gel electrophoresis;
DLS = Dynamic Light Scattering;
HMMS = high molecular weight species;
LMMS = low molecular weight species;
SEC = size exclusion chromatograph

6.1. Dose Form Stability Data for the mRNA LNP

The data generated from this study show comparable results for Appearance, Visible Particulates, pH, RNA integrity, In Vitro Expression (IVE), and LNP size and PDI between the T4 hour samples and their respective TO sample for both 180 μg and 210 μg dose forms. All data there met the study criteria.

All stability results for both 180 ug and 210 ug samples are shown in Table 12 and Table 13, respectively.

### 6.2. Dose Form Stability Data for the RSV Protein

The data generated from this study show comparable results for Appearance, Visible Particulates, pH, Protein Concentration, and Relative Prefusion Content between the T4 hour samples and their respective TO sample for both 180 μg and 210 μg dose forms. All data there met the study criteria. All stability results for both 180 ug and 210 ug samples are shown in

TABLE 12
Test Results for PF-07941314 Influenza mod mRNA & RSV Combination Vaccine (180 μg dose [1:2] Preparation)

| | | | 30° C. | | | | | |
|---|---|---|---|---|---|---|---|---|
| | Study | | | | | | T2H | T4H |
| Test | Criteria | T0 | T2H PP | T4H PP | T2H PC | T4H PC | Vial | Vial |
| Appearance | White to | WOWS | WOWS | WOWS | WOWS | WOWS | WOWS | WOWS |
| | off-white | | | | | | | |
| | suspension | | | | | | | |
| Appearance | Essentially | EFVP | EFVP | EFVP | EFVP | EFVP | EFVP | EFVP |
| (Visible | Free | | | | | | | |
| Particles) | from | | | | | | | |
| | Visible | | | | | | | |
| | Particles | | | | | | | |
| pH | 7.4 ± 0.5 | 7.3 | 7.3 | 7.2 | 7.2 | 7.3 | 7.3 | 7.3 |
| RNA | T0 ± 20% | 62 | 59 | 61 | 59 | 54 | 58 | 53 |
| Content | | | | | | | | |
| (RiboGreen) | | | | | | | | |
| RNA | ≥50% | 77 | 79 | 77 | 78 | 77 | 79 | 78 |
| Integrity | Intact | | | | | | | |
| (CGE by | RNA | | | | | | | |
| fragment | | | | | | | | |
| analyzer) | | | | | | | | |
| RNA | ≥80% | 93 | 90 | 86 | 88 | 83 | 86 | 83 |
| Encapsulation | | | | | | | | |
| (RiboGreen) | | | | | | | | |
| LNP Size | 40-120 | 68 | 69 | 69 | 68 | 69 | 68 | 70 |
| (DLS) | nm | | | | | | | |
| LNP PDI | ≤0.3 | 0.15 | 0.15 | 0.16 | 0.15 | 0.16 | 0.15 | 0.18 |
| (DLS) | | | | | | | | |
| In vitro | Report | A/Wisc: 93% | A/Wisc: 92% | A/Wisc: 92% | A/Wisc: 91% | A/Wisc: 91% | A/Wisc: 92% | A/Wisc: 98% |
| Expression | Result | [22 ng/well], | [22 ng/well], | [22 ng/well], | [22 ng/well], | [22 ng/well], | [22 ng/well], | [22 ng/well], |
| | | A/Darwin: 95% | A/Darwin: 95% | A/Darwin: 95% | A/Darwin: 94% | A/Darwin: 94% | A/Darwin: 96% | A/Darwin: 97% |
| | | [7 ng/well], | [7 ng/well], | [7 ng/well], | [7 ng/well], | [7 ng/well], | [7 ng/well], | [7 ng/well], |
| | | B/Austria: 91% | B/Austria: 90% | B/Austria: 89% | B/Austria: 89% | B/Austria: 85% | B/Austria: 85% | B/Austria: 86% |
| | | [22 ng/well], | [22 ng/well], | [22 ng/well], | [22 ng/well], | [22 ng/well], | [22 ng/well], | [22 ng/well], |
| | | B/Phuket: 85% | B/Phuket: 86% | B/Phuket: 86% | B/Phuket: 85% | B/Phuket: 83% | B/Phuket: 87% | B/Phuket: 95% |
| | | [67 ng/well] | [67 ng/well] | [67 ng/well] | [67 ng/well] | [67 ng/well] | [67 ng/well] | [67 ng/well] |
| Protein | T0 ± 20% | 115 | 113 | 113 | 103 | 107 | 102 | 104 |
| Concentration | (mcg/mL) | | | | | | | |
| HMMS | ≤25% | 2.4 | 5.3 | 4.5 | 6.1 | 5.9 | 4.0 | 5.7 |
| (SEC) | | | | | | | | |
| LMMS (SEC) | ≤5% | 1.6 | 2.1 | 1.9 | 1.7 | 2.0 | 1.9 | 1.9 |
| Trimer | ≥70% | 96.0 | 92.6 | 93.5 | 92.2 | 92.1 | 94.1 | 92.4 |
| (SEC) | | | | | | | | |
| Relative | 50-150% | 81 | 94 | 80 | 88 | 89 | 83 | 105 |
| Prefusion | | | | | | | | |
| Content | | | | | | | | |
| (Prefusion F | | | | | | | | |
| ELISA)^{a} | | | | | | | | |

Abbreviations:
PP = polypropylene;
PC = polycarbonate;
WOWS = White to off-white suspension;
EFVP = Essentially Free of Visible Particles;
LNP = Lipid Nano Particle;
CGE = capillary gel electrophoresis;
DLS = Dynamic Light Scattering;
HMMS = high molecular weight species;
LMMS = low molecular weight species;
SEC = size exclusion chromatograph

TABLE 13
Test Results for PF-07941314 Influenza mod mRNA & RSV Combination Vaccine (210 μg dose (1:1.33) Preparation)

| | | | 30° C. | | | | | |
|---|---|---|---|---|---|---|---|---|
| | Expected | | | | | | T2H | T4H |
| Test | Results | T0 | T2H PP | T4H PP | T2H PC | T4H PC | Vial | Vial |
| Appearance | White to | WOWS | WOWS | WOWS | WOWS | WOWS | WOWS | WOWS |
| | off-white | | | | | | | |
| | suspension | | | | | | | |
| Appearance | Essentially | EFVP | EFVP | EFVP | EFVP | EFVP | EFVP | EFVP |
| (Visible | Free from | | | | | | | |
| Particles) | Visible | | | | | | | |
| | Particles | | | | | | | |
| | (EFVP) | | | | | | | |
| pH | 7.4 ± 0.5 | 7.2 | 7.2 | 7.3 | 7.2 | 7.2 | 7.2 | 7.2 |
| RNA | T0 ± 20% | 75 | 71 | 70 | 81 | 71 | 73 | 76 |
| Content | | | | | | | | |
| (RiboGreen) | | | | | | | | |
| RNA | ≥50% | 81 | 83 | 80 | 81 | 81 | 81 | 81 |
| Integrity | Intact | | | | | | | |
| (CGE by | RNA | | | | | | | |
| fragment | | | | | | | | |
| analyzer) | | | | | | | | |
| RNA | ≥80% | 90 | 89 | 89 | 85 | 87 | 89 | 88 |
| Encapsulation | | | | | | | | |
| (RiboGreen) | | | | | | | | |
| LNP Size | 40-120 | 67 | 68 | 67 | 68 | 68 | 69 | 69 |
| (DLS) | nm | | | | | | | |
| LNP PDI | ≤0.3 | 0.16 | 0.16 | 0.14 | 0.15 | 0.16 | 0.17 | 0.18 |
| (DLS) | | | | | | | | |
| In vitro | Report | A/Wisc: 89% | A/Wisc: 87% | A/Wisc: 87% | A/Wisc: 90% | A/Wisc: 87% | A/Wisc: 88% | A/Wisc: 90% |
| Expression | Result | [22 ng/well], | [22 ng/well], | [22 ng/well], | [22 ng/well], | [22 ng/well], | [22 ng/well], | [22 ng/well], |
| | | A/Darwin: 86% | A/Darwin: 86% | A/Darwin: 83% | A/Darwin: 87% | A/Darwin: 83% | A/Darwin: 85% | A/Darwin: 85% |
| | | [7 ng/well], | [7 ng/well], | [7 ng/well], | [7 ng/well], | [7 ng/well], | [7 ng/well], | [7 ng/well], |
| | | B/Austria: 88% | B/Austria: 87% | B/Austria: 88% | B/Austria: 87% | B/Austria: 83% | B/Austria: 82% | B/Austria: 80% |
| | | [22 ng/well], | [22 ng/well], | [22 ng/well], | [22 ng/well], | [22 ng/well], | [22 ng/well], | [22 ng/well], |
| | | B/Phuket: 80% | B/Phuket: 79% | B/Phuket: 77% | B/Phuket: 74% | B/Phuket: 71% | B/Phuket: 74% | B/Phuket: 71% |
| | | [67 ng/well] | [67 ng/well] | [67 ng/well] | [67 ng/well] | [67 ng/well] | [67 ng/well] | [67 ng/well] |
| Protein | T0 ± 20% | 85 | 85 | 84 | 78 | 81 | 85 | 84 |
| Concentration | (mcg/mL) | | | | | | | |
| HMMS | ≤25% | 1.7 | 4.6 | 6.1 | 11.7 | 7.7 | 4.2 | 3.1 |
| (SEC) | | | | | | | | |
| LMMS | ≤5% | 1.9 | 2.1 | 1.7 | 1.8 | 1.7 | 1.6 | 1.9 |
| (SEC) | | | | | | | | |
| Trimer | ≥70% | 96.4 | 93.3 | 92.2 | 86.5 | 90.6 | 94.2 | 95.0 |
| (SEC) | | | | | | | | |
| Relative | 50-150% | 90 | 96 | 95 | 90 | 100 | 107 | 96 |
| Prefusion | | | | | | | | |
| Content | | | | | | | | |
| (Prefusion | | | | | | | | |
| F ELISA)^{a} | | | | | | | | |

Abbreviations:
PP = polypropylene;
PC = polycarbonate;
WOWS = White to off-white suspension;
EFVP = Essentially Free of Visible Particles;
LNP = Lipid Nano Particle;
CGE = capillary gel electrophoresis;
DLS = Dynamic Light Scattering;
HMMS = high molecular weight species;
LMMS = low molecular weight species;
SEC = size exclusion chromatograph

### 7. Conclusion

The DAI verification study was conducted at temperature of 30° C. for up to 4 hours. The data generated from this study show comparable in-use stability results of investigated RSV bivalent protein subunit antigens and Quadrivalent Influenza mod RNA antigens for up to 4 hours in the combined dosing formulation presentation in three types of container/closure systems. The results from this study support physicochemical stability of the dosing solution in administration component for the proposed in-use guidance for immediate use a prepared quadrivalent influenza and RSV bivalent combination vaccine for clinical studies.

### Example 8: Flu mRNA+RSV Subunit Compatibility

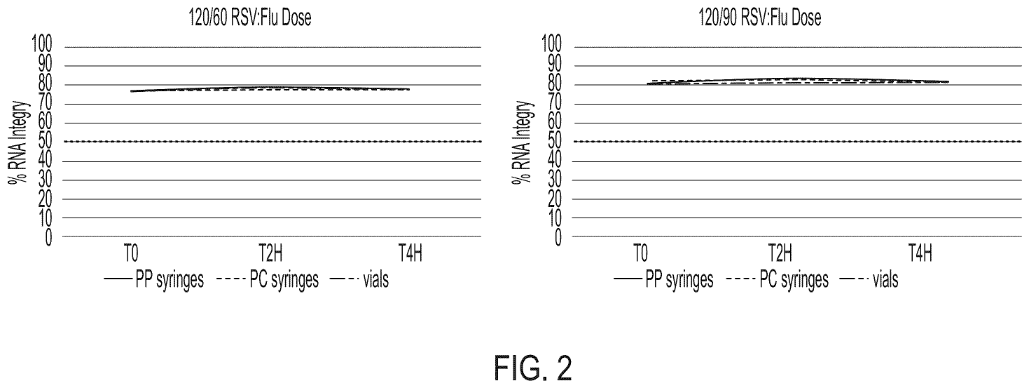

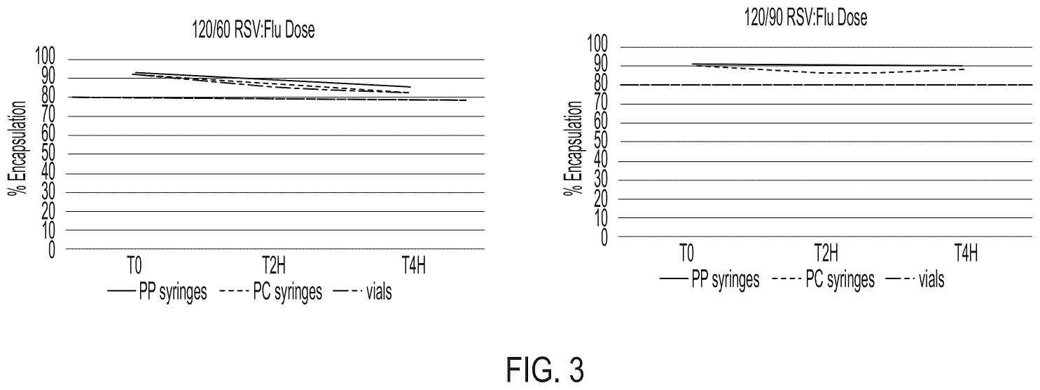

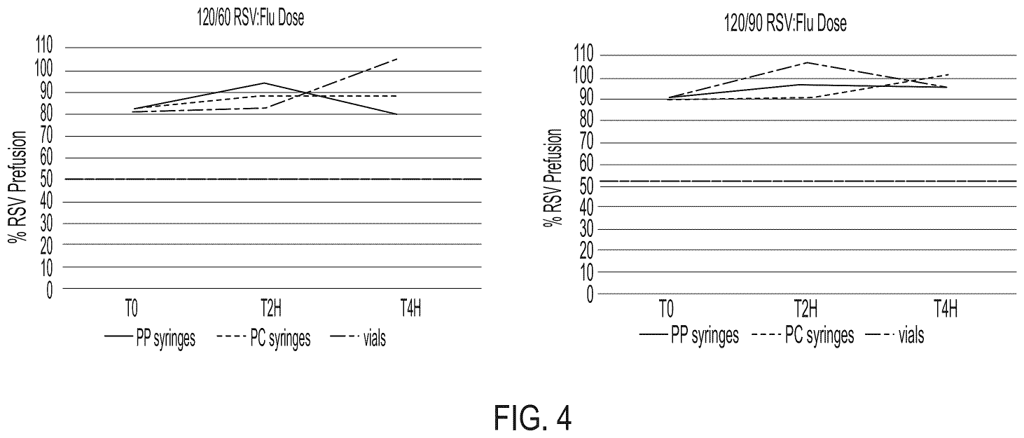

The clinical DAI experimental design tested 2 arms: (i) 120 ug dose (total RSV protein)+60 ug dose (total RNA) in 1 mL dose; and (ii) 120 ug dose (total RSV protein)+90 ug dose (total RNA) in 1.25 mL dose. The doses were assessed at 30° initially, for 2 hrs, and 4 hrs: (a) In vials; (b) Poly Propylene syringes; and (c) Poly Carbonate syringes. Conclusion: all pre-specified criteria were met which supports compatibility and stability of the mixture for up to 4 hours. See FIG. 2 and FIG. 3.

In addition, no Significant Changes in RNA expression were observed in in vitro expression (IVE) analyses. See Table 14 and Table 15, wherein All values reported are % positive cells for A/Wisc: [22 ng/well], A/Darwin: [7 ng/well], By/Austria: [22 ng/well], By/Phuket: [67 ng/well]. Target: Report Results.

TABLE 14

| 120 ug RSV + | | | |
|---|---|---|---|
| 60 ug Flu Dose | T0 | T2 H | T4 H |
| PP | A/Wisc: 93% | A/Wisc: 92% | A/Wisc: 92% |
| | A/Darwin: 95% | A/Darwin: 95% | A/Darwin: 95% |
| | Bv/Austria: 91% | Bv/Austria: 90% | Bv/Austria: 89% |
| | By/Phuket: 85% | By/Phuket: 86% | By/Phuket: 86% |
| PC | | A/Wisc: 91% | A/Wisc: 91% |
| | | A/Darwin: 94% | A/Darwin: 94% |
| | | Bv/Austria: 89% | Bv/Austria: 85% |
| | | By/Phuket: 85% | By/Phuket: 83% |
| Vial | | A/Wisc: 92% | A/Wisc: 98% |
| | | A/Darwin: 96% | A/Darwin: 97% |
| | | Bv/Austria: 85% | Bv/Austria: 86% |
| | | By/Phuket: 87% | By/Phuket: 95% |

TABLE 15

| 120 ug RSV + | | | |
|---|---|---|---|
| 90 ug Flu Dose | T0 | T2 H | T4 H |
| PP | A/Wisc: 89% | A/Wisc: 87% | A/Wisc: 87% |
| | A/Darwin: 86% | A/Darwin: 86% | A/Darwin: 83% |
| | Bv/Austria: 88% | Bv/Austria: 87% | Bv/Austria: 88% |
| | By/Phuket: 80% | By/Phuket: 79% | By/Phuket: 77% |
| PC | | A/Wisc: 90% | A/Wisc: 87% |
| | | A/Darwin: 87% | A/Darwin: 83% |
| | | Bv/Austria: 87% | Bv/Austria: 83% |
| | | By/Phuket: 74% | By/Phuket: 71% |
| Vial | | A/Wisc: 88% | A/Wisc: 90% |
| | | A/Darwin: 85% | A/Darwin: 85% |
| | | Bv/Austria: 82% | Bv/Austria: 80% |
| | | By/Phuket: 74% | By/Phuket: 71% |

Moreover, there were no Significant Changes in LNP Size and polydispersity index by DLS. See Table 16 and

Table 17, wherein the Targets for Size: 40-120 nm and PDI: ≤0.3.

TABLE 16

| 120 ug RSV + | T0 | T2 H | T4 H |
|---|---|---|---|
| 60 ug Flu Dose | (Size/PDI) | (Size/PDI) | (Size/PDI) |
| PP | 68 nm/0.15 | 69 nm/0.15 | 69 nm/0.16 |
| PC | | 68 nm/0.15 | 69 nm/0.16 |
| Vial | | 68 nm/0.15 | 70 nm/0.18 |

TABLE 17

| 120 ug RSV + | T0 | T2 H | T4 H |
|---|---|---|---|
| 90 ug Flu Dose | (Size/PDI) | (Size/PDI) | (Size/PDI) |
| PP | 67 nm/0.16 | 68 nm/0.16 | 67 nm/0.14 |
| PC | | 68 nm/0.15 | 68 nm/0.16 |
| Vial | | 69 nm/0.17 | 69 nm/0.18 |

Furthermore, there were no Significant Changes in RSV Concentration, wherein the target was T0±20% (ug/mL). See Table 18.

TABLE 18

| | T0 | T2 H | T4 H |
|---|---|---|---|
| 120 ug RSV + | | | |
| 60 ug Flu Dose | | | |
| PP | 115 | 113 | 113 |
| PC | 115 | 103 | 107 |
| Vial | 115 | 102 | 104 |
| 120 ug RSV + | | | |
| 90 ug Flu Dose | | | |
| PP | 85 | 85 | 84 |
| PC | 85 | 78 | 81 |
| Vial | 85 | 85 | 84 |

### Example 9: In Vitro Expression (IVE) Assay Using 293F Suspension Cells

DAI Study T0 T2h T41h; testing a combination composition comprising RSV protein reconstituted with WFI and then mixed with quadrivalent modFlu-encapsulated LNPs in vial Samples tested shown in All Samples: 200-0.1 ng/mL

Table 19.

All Samples: 200-0.1 ng/mL

TABLE 19

| | Day 1 | Day 2 |
|---|---|---|
| | (120 ugRSV + | (120 ugRSV + |
| Sample Name | 60 ugFlu, G1) | 90 ugFlu, G2) |
| PPSyringe_30C | T0_G1 | T0_G2 |
| PPSyringe_30C | T2 H_G1_PP | T2 H_G2_PP |
| PPSyringe_30C | T4 H_G1_PP | T4 H_G2_PP |
| PCSyringe_30C | T2 H_G1_PC | T2 H_G2_PC |
| PCSyringe_30C | T4 H_G1_PC | T4 H_G2_PC |
| Vial_30C | T2 H_G1_Vial | T2 H_G2_Vial |
| Vial_30C | T4 H_G1_Vial | T4 H_G2_Vial |
| RSV protein reconstituted | T0_G1_RSV | T0_G2_RSV |
| with WFI and then to mix with | T0_G1_Flu | T0_G2_Flu |
| modRNA LNP matrix in Vial | T2 H_G1_RSV | T2 H_G2_RSV |
| modRNA LNP mixed with | T2 H_G1_Flu | T2 H_G2_Flu |
| RSV matrix in Vial | T4 H_G1_RSV | T4 H_G2_RSV |
| | T4 H_G1_Flu | T4 H_G2_Flu |

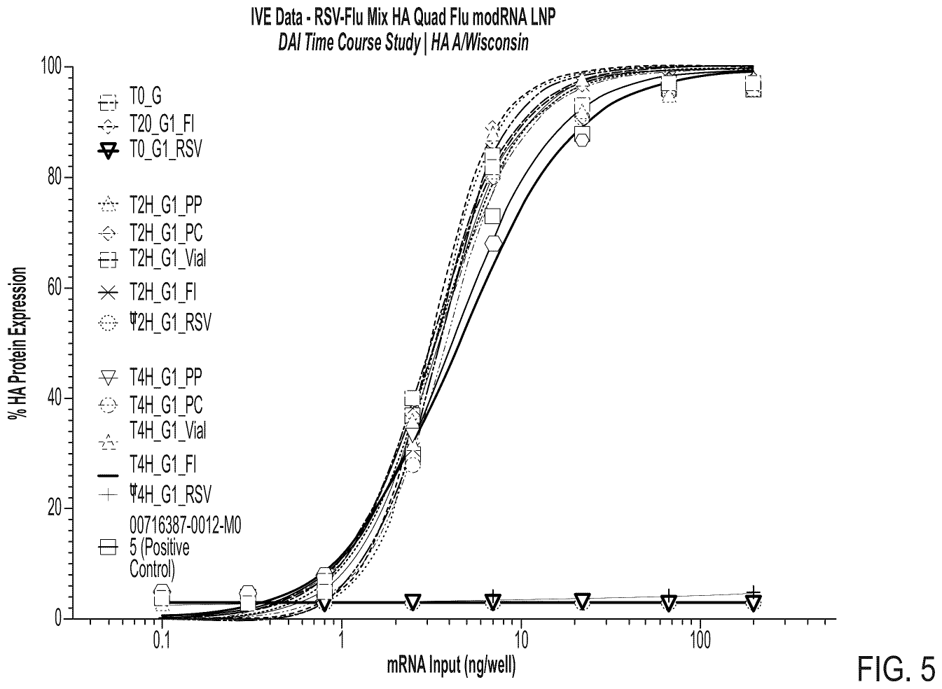

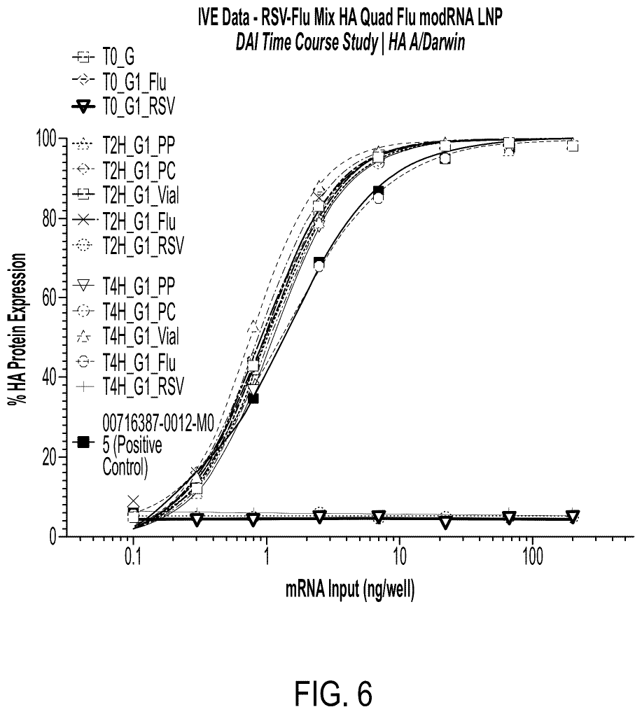

IVE Titration Curves of HA A/Wisconsin and A/Darwin Strains Show Similar Performance. See FIG. 5 and FIG. 6.

Results also shown in Table 20.

TABLE 20
Day 1 (120 ugRSV + 60 ugFlu, G1)

| | | Staining for HA A/Wisconsin | | Staining for HA A/Darwin | |
|---|---|---|---|---|---|
| | | % Protein | EC50 | % Protein | EC50 |
| | | Expression | (ng/well) | Expression | (ng/well) |
| Sample Lot # | Analyte of Interest | (22 ng/well) | constrained | (7 ng/well) | constrained |
| T0_G1 | RSV + modRNA HA | 93 | 3 | 95 | 1 |
| | Wisconsin/Darwin/ | | | | |
| | Austria/Phuket | | | | |
| T2 H_G1_PP | RSV + modRNA HA | 92 | 3 | 95 | 1 |
| | Wisconsin/Darwin/ | | | | |
| | Austria/Phuket | | | | |
| T4 H_G1_PP | RSV + modRNA HA | 92 | 3 | 95 | 1 |
| | Wisconsin/Darwin/ | | | | |
| | Austria/Phuket | | | | |
| T2 H_G1_PC | RSV + modRNA HA | 91 | 3 | 94 | 1 |
| | Wisconsin/Darwin/ | | | | |
| | Austria/Phuket | | | | |
| T4 H_G1_PC | RSV + modRNA HA | 91 | 4 | 94 | 1 |
| | Wisconsin/Darwin/ | | | | |
| | Austria/Phuket | | | | |
| T2 H_G1_Vial | RSV + modRNA HA | 92 | 3 | 96 | 1 |
| | Wisconsin/Darwin/ | | | | |
| | Austria/Phuket | | | | |
| T4 H_G1_Vial | RSV + modRNA HA | 98 | 3 | 97 | 1 |
| | Wisconsin/Darwin/ | | | | |
| | Austria/Phuket | | | | |
| T0_G1_RSV | RSV | 3 | Greater than top | 5 | Greater than top |
| | | | dose of curve | | dose of curve |
| T0_G1_Flu | modRNA HA | 97 | 3 | 96 | 1 |
| | Wisconsin/Darwin/ | | | | |
| | Austria/Phuket | | | | |
| T2 H_G1_RSV | RSV | 3 | Greater than top | 5 | Greater than top |
| | | | dose of curve | | dose of curve |
| T2 H_G1_Flu | modRNA HA | 97 | 3 | 96 | 1 |
| | Wisconsin/Darwin/ | | | | |
| | Austria/Phuket | | | | |
| T4 H_G1_RSV | RSV | 3 | Greater than top | 5 | Greater than top |
| | | | dose of curve | | dose of curve |
| T4 H_G1_Flu | modRNA HA | 87 | 5 | 85 | 1 |
| | Wisconsin/Darwin/ | | | | |
| | Austria/Phuket | | | | |
| 00716387- | modRNA HA | 88 | 4 | 87 | 1 |
| 0012-M05 | Wisconsin/Darwin/ | | | | |
| (Positive | Austria/Phuket | | | | |
| Control) | | | | | |

All Formulations Appear Stable within TO-T4 Stability Window. No expression with RSV Only Material in the IVE assay, as expected.

IVE Titration Curves of HA A/Wisconsin and A/Darwin Strains Show Similar Performance;

No significant differences were observed between Flu-RSV formulations tested

All Flu-RSV Formulations Appear Stable within TO-T4 Stability Window

### Example 10. Generation of RSV RNA Constructs

RNA constructs generated herein encode RSV F protein wild-type (WT) and RSV F protein variants/mutants (i.e. RSV pre-fusion F protein). Table 21 shows WT F proteins (WT F) and variant RSV F proteins (RSV A 847 F and RSV B 847 F).

TABLE 21
RSV F proteins and description

| RSV Protein | RSV Protein Description |
|---|---|
| SEQ ID NO: 1 | Full Length F0 of Native RSV A2 (GenBank GI: |
| | 138251; Swiss Prot P03420) (574 aa) |
| SEQ ID NO: 2 | Full Length F0 of Native RSV B (18537 strain; |
| | GenBank GI: 138250; Swiss Prot P13843) (574 aa) |
| RSV A WT F | Full length WT RSV subtype A, F protein (574 aa) |
| SEQ ID NO: 3 | |
| RSV A 847 F | RSV subtype A 847 mutant F protein with |
| SEQ ID NO: 4 | substitutions A103C, I148C, S190I and D486S (574 aa) |
| RSV B WT F | Full length WT RSV subtype B, F protein (574 aa) |
| SEQ ID NO: 5 | |
| RSV B 847 F | RSV subtype B 847 mutant F protein with |
| SEQ ID NO: 6 | substitutions A103C, I148C, S190I and D486S (574 aa) |

DNA sequences encoding RSV F proteins were prepared and utilized for in vitro transcription reactions to generate RNA. In vitro transcription of RNA is known in the art and is described herein. DNA templates were cloned into a plasmid vector with backbone sequence elements (T7 promoter, 5′ and 3′ UTR, poly-A tail) for improved RNA stability and translational efficiency. The DNA was purified, spectrophotometrically quantified and in vitro-transcribed by T7 RNA polymerase in the presence of a trinucleotide cap S analogue ((m₂^{7,3′-O})Gppp(m^{2′-O})ApG) (TriLink) and with N1-methylpseudouridine (T) replacing uridine (modified RNA (modRNA)).

The RSV RNA was generated from codon-optimized (00) DNA for stabilization and superior protein expression. Table 22 shows RNA constructs of the present disclosure, and corresponding sequences, comprising a 5′ UTR, an open reading frame encoding a respiratory syncytial virus (RSV) polypeptide, a 3′ UTR and a poly-A tail.

TABLE 22
RSV F modRNA constructs

| | | | | | RSV F |
|---|---|---|---|---|---|
| | | RSV | | Poly-A | Protein/ |
| | 5′-UTR | [ORF] | 3′-UTR | tail* | DNA |
| RNA | SEQ | SEQ | SEQ | SEQ | SEQ |
| Construct | ID NO | ID NO | ID NO | ID NO | ID NO |
| RSV F 847A | 18 | 11 | 21 | 26 | 4/8 |
| modRNA | | | | | |
| RSV F 847B | 18 | 12 | 21 | 26 | 6/9 |
| modRNA | | | | | |

*Poly-A tail length may contain +1/−1 A.

### Example 11. RSV 847A and 847B F Expression

This example serves to capture the in vitro expression (IVE) results generated for the respiratory syncytial virus (RSV) modRNA lipid nanoparticle (LNP) drug product described herein. The constructs encapsulated in the LNP encode for the full length RSV trimeric fusion glycoprotein (F-protein) from the A-strain and B-strain viruses, respectively.

TABLE 23
List of Antibodies

| | | In-Assay |
|---|---|---|
| Item | Source | Concentration |
| Human mAb anti-RSV mAb1 | In-house | 500 ng/mL |
| Mouse mAb anti-RSV L4-6 | In-house | 500 ng/mL |
| Goat Anti-Mouse IgG AF647 | Invitrogen | 500 ng/mL |
| Goat Anti-Human Kappa-PE | Southern Biotech | 500 ng/mL |

TABLE 24
List of Test Articles

| Item | Test Article |
|---|---|
| 1 | LNP containing RSV F modRNA_847A_{—} |
| 2 | LNP containing RSV F modRNA_847B_{—} |

The description of the protocol below is for total staining (surface+intracellular proteins), for surface only staining protocol the fixation and wash buffers are changed to remove the permeabilization agent all other steps and reagents are the same. In brief, 96-well culture plates were seeded with HEK293F cells at a density of 2.5×105 cells/well and were placed in a shaking incubator (350 RPM, 37° C., humidified, 5% CO2) while the samples titations were prepared. The LNP drug product was diluted in DPBS to a concentration of 80 ng/uL and serially diluted 8-pts with a dilution factor of 4. Then the 96-well culture plate was removed from the incubator and 50 uL of each step of the diluted LNP were added to duplicate wells of the 96-well culture plate to generate a titration curve ranging from 8,000 ng/well-1.95 ng/well. The 96-well culture plate was placed back into the shaking incubator overnight. After incubation, 250 uL of cells are transferred to a 96-well u-bottom polystyrene plate and pelleted using a swinging bucket centrifuge (500 rcf, 5 min at RT). The supernatant is removed and cells resuspended in 100 uL solution of Aqua405 live/dead stain. The plate is incubated 30 min at room temperature, protected from light. After incubation cells are washed with wash buffer and pelleted using centrifugation (500 rcf, 5 min at RT). The supernatant is removed and cells are resuspended in 100 uL fixation/permeabilization buffer and the plate is incubated 30 minutes at 2-8° C., protected from light. Once incubation is complete the cells are pelleted using a swinging bucket centrifuge (500 rcf, 5 min at RT). The supernatant is removed and cells resuspended with 250 uL wash buffer, this is repeated for a total of 2 washes. After the final wash step the cells pelleted, supernatant removed, and resuspended in 50 uL of primary antibody solution. The plates are sealed and incubated for 45 minutes at 2-8° C., protected from light. Once completed the cells are pelleted using a swinging bucket centrifuge (500 rcf, 5 min at RT). The supernatant is removed and cells resuspended with 250 uL wash buffer, this is repeated for a total of 2 washes. After the final wash step the cells are pelleted, supernatant removed, and resuspended in 50 uL of secondary antibody solution. The plates are sealed and incubated for 45 minutes at 2-8° C., protected from light. Once completed the cells are pelleted using a swinging bucket centrifuge (500 rcf, 5 min at RT). The supernatant is removed and cells resuspended with 250 uL wash buffer, this is repeated for a total of 2 washes. After the final wash step cells are pelleted, supernatant removed, and resuspended in 200 uL of wash buffer and data acquired by flow cytometry.

In vitro Expression (IVE) for the modRNA LNP drug products was assessed by transfecting HEK293F cells with a dose titration curves and staining for antibodies; RSVmab specific for the trimeric RSV F-protein and L4-6 specific for total RSV F-protein. These antibodies have been shown to recognize both the A-strain and B-strain RSV F-protein and were used in the assay with either permeabilizing or non-permeabilizing conditions to asses the total cell vs cell surface content of the RSV F-protein. The measured % positive cells (2,000 ng/well input) and EC50 of the dose response curves of the drug product lots are shown in Table 25.

TABLE 25
Drug Product IVE Results

| | | RSVF-Protein | | | |
|---|---|---|---|---|---|
| | | Cellular | | EC50 | % Positive Cells |
| Item | Test Article | Localization | Antibody | (ng/well) | (2000 ng/well) |
| 1 | LNP containing | Cell | RSV mab1 | 131 | 89 |
| | modRNA_847A_{—} | Surface/TGN | L4-6 | 33 | 99 |
| 2 | LNP containing | Cell | RSV mab1 | 187 | 89 |
| | modRNA_847B_{—} | Surface/TGN | L4-6 | 95 | 94 |

### Example 13. Preparation of RSV F modRNA Formulated in LNP

The LNP formulation contains 2 functional lipids, ALC-0315 and ALC-0159, and 2 structural lipids DSPC (1,2distearoyl-sn-glycero-3-phosphocholine) and cholesterol. The physicochemical properties and the structures of the 4 lipids are shown in the Table 26 below.

Lipid nanoparticles were prepared and tested according to the general procedures described in U.S. Pat. No. 9,737,619 (PCT Pub. No. WO2015/199952) and U.S. patent Ser. No. 10/166,298 (WO 2017/075531) and WO2020/146805, each of which is hereby incorporated by reference in its entirety. Briefly, cationic lipid, DSPC, cholesterol and PEG-lipid were solubilized in ethanol at a molar ratio of about 47.5:10:40.7:1.8.

TABLE 26
Lipids in the LNP Formulation

| | Molecular | Molecular Formula | |
|---|---|---|---|
| Lipid (CAS | Weight | Physical state and | |
| number) | [Da] | Storage condition | Chemical name and structure |
| Cationic Lipid ALC-0315 (Not applicable) | 766 | C₄₈H₉₅NO₅ Liquid (oil) −20° C. | 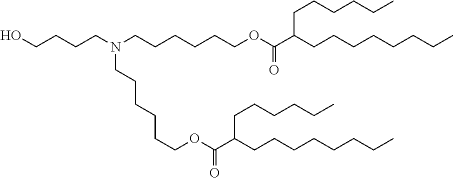 [SMILES: CCCCCCCCC(CCCCCC)C(=O)OCCCCCCN(CCCCO)CCCCCCOC(=O)C(CCCCCC)CCCCCCCC] |
| | | | (4-hydroxybutyl)azanediyl)bis(hexane-6,1-diyl)bis(2-hexyldecanoate) |
| PEG-Lipid ALC-0159 (1849616- 42-7) | ~2400-2600 | C₃₀H₆₀NO(C₂H₄O)nOCH₃ n = 45-50 Solid −20° C. | 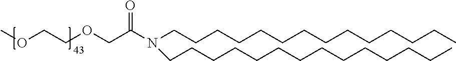 |
| | | | 2-[(polyethylene glycol)-2000]-N,N-ditetradecyclacetamide |
| DSPC (816-94-4) | 790 | C₃₀H₈₈NO₈P Solid −20° C. | 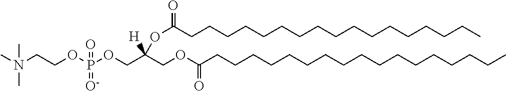 |
| | | | 1,2-Distearoyl-sn-glycero-3-phosphcholine |
| Cholesterol (57-88-5) | 387 | C₂₇H₄₆O Solid −20° C. | 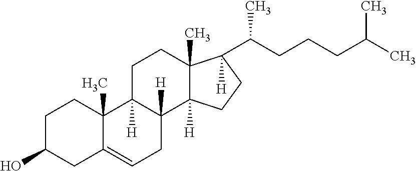 |

CAS = Chemical Abstract Service; DSPC = 1,2-disteroyl-sn-glycero-3-phosphocholine

### Example 14. Immune Responses (In Vivo Experiments)

Female BALB/c mice were immunized with RSV prefusion F (874) in bivalent protein subunit version (RSV 847A+847B) as described in WO2017/109629 or modRNA-LNP formulation described herein either as monovalent (RSV 847A) or bivalent (RSV 847A+847B) at different doses on day 0 and day 21. Immunogenicity was evaluated by measuring RSV neutralizing antibody response and RSV F-specific T-cell response. Serum was collected on day 21 and day 35 (2 weeks post dose 2, PD2) for RSV neutralizing assay and spleen was collected on day 35 (2 weeks PD2) for T-cell assays (ELISpot and Intracellular Cytokine Staining, ICS assays).

### Neutralization Assay

The RSV microneutralization assay is a 3-day assay done using A₅₄₉ cells (human alveolar basal epithelial cells) to measure functional antibodies in serum that neutralize RSV activity, preventing infection of a host cell monolayer. On Day 0, A₅₄₉ cells (human alveolar basal epithelial cells; ATCC, cat #CCL-185) are seeded in 96-well tissue-culture treated plates at 2.5×10⁴ cells per well and incubated for at least 20 hours to form a confluent monolayer. On Day 1, diluted virus (RSV A, M37; RSV B, B18537; 500 FFU/well) is added to 3-fold serial dilutions of heat inactivated test serum prepared in duplicate and incubated for 1 hour to allow antibodies to bind to the virus. The neutralization reaction is then transferred onto the prepared A549 cell monolayers and incubated for 2 hours. Additional media is supplemented onto the plates prior to an overnight incubation (at least 16 hours). On Day 2, the plates are fixed with methanol and stained with a mouse anti-RSV F (Pfizer, L4-6) primary antibody followed by an Alexa 488 fluorescently labeled secondary antibody to detect viral foci. A 50% neutralization titer is calculated as the last reciprocal serum dilution at which 50% of the virus is neutralized compared to wells containing virus only. A titer is reported as the geometric mean titer (GMT) of the two replicate titers of each sample. The assay titer range is 20 to 43,740. Any samples with a titer >43,740 are prediluted and repeated to extend the upper titer limit. Any samples below the lower limit of detection (LLOD) are reported at LLOD of 20.

### T-Cell Response Measurement

Vaccine-induced T-cell response to RSV F is assessed by ex vivo stimulation of splenocytes in the presence of RSV F (A+B) peptide pool to activate production of various cytokine such as IFN-γ in antigen-specific T cells. The cytokines secreted outside the cells can be measured by ELISpot (expressed as spot forming cells, SFC per million cells) or cytokine secretion can be blocked within the cells to be measured by ICS (expressed as percentage of cytokine expressing CD4+ T cells and CD8+ T cells).

In case of ELISpot, the cytokine IFN-γ secreted by activated T cells is captured by an antilFN-γ antibody coated onto the polyvinylidene fluoride (PVDF) membrane of the well bottom on a microplate. The captured IFN-γ is developed into a spot by another noncompeting biotinylated anti-IFN-γ secondary antibody followed by an enzymatic color reaction using streptavidin-alkaline phosphatase (ALP) conjugate and the substrate solution, nitro-blue tetrazolium and 5-bromo-4-chloro-3′-indolyphosphate (BCIP/NBT-plus) that yield a dark purple precipitate or spot. T-cell IFN-γ response is measured using Mabtech Mouse IFN-γ ELISpot PLUS kit (ALP) and expressed as spot forming cells (SFC) per million cells.

ICS staining can detect multiple cytokines, including IFN-γ, produced in both CD4+ and CD8+ T cells following antigen peptide stimulation. Single cell suspensions of splenocytes (2×10⁶ cells/well) were cultured ex vivo in cRPMI with media-DMSO (unstimulated) or specific peptide pool (15aa, 11 aa overlap, 2 μg/mL/peptide) representing RSV F A+B for 5 hours at 37° C. in the presence of anti-CD107a APC antibody and protein transport inhibitors, GolgiPlug and GolgiStop. Following stimulation, splenocytes were incubated with fluorescently conjugated antibodies to the surface proteins CD19, CD3, CD4, CD8, CD44 (25±5 minutes at 18-25° C.) followed by fixation and permeabilization and staining for intracellular proteins IFN-γ, TNF-α, IL-2 and CD40L/CD154 (25±5 minutes at 18-25° C.). After staining, the cells are washed and resuspended in FC buffer. Cells were acquired on LSR Fortessa and data analyzed by FlowJo (10.7.1). Results are background (media-DMSO) subtracted and shown as percentage of cytokine-expressing CD4+ T cells and CD8+ T cells.

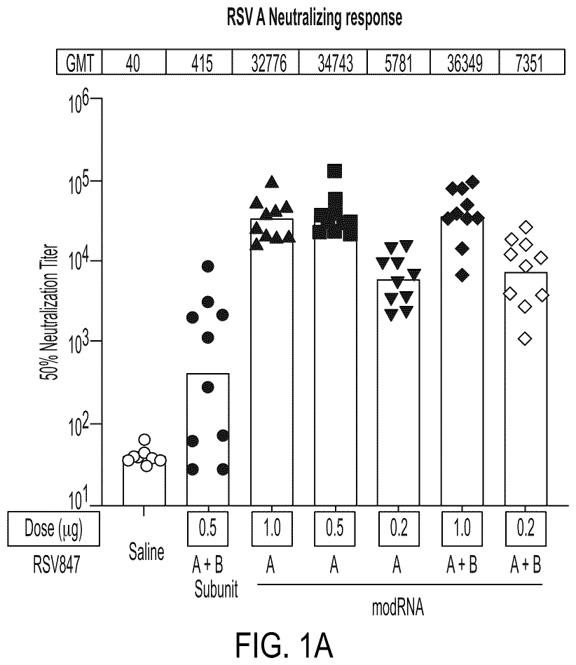

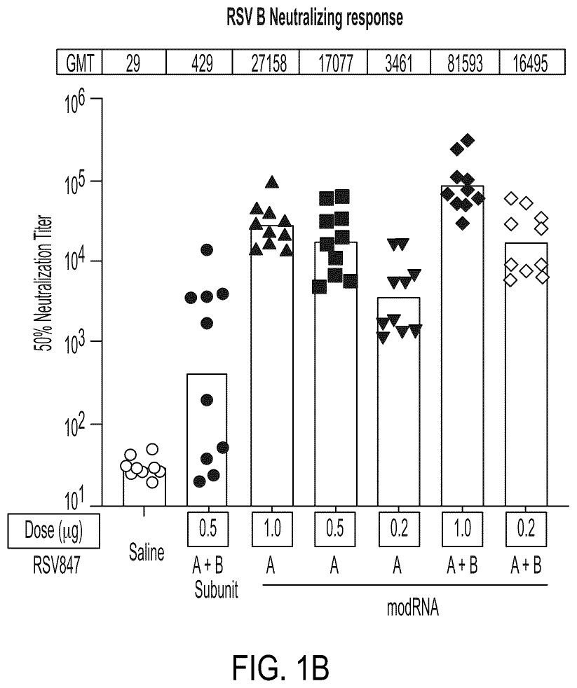

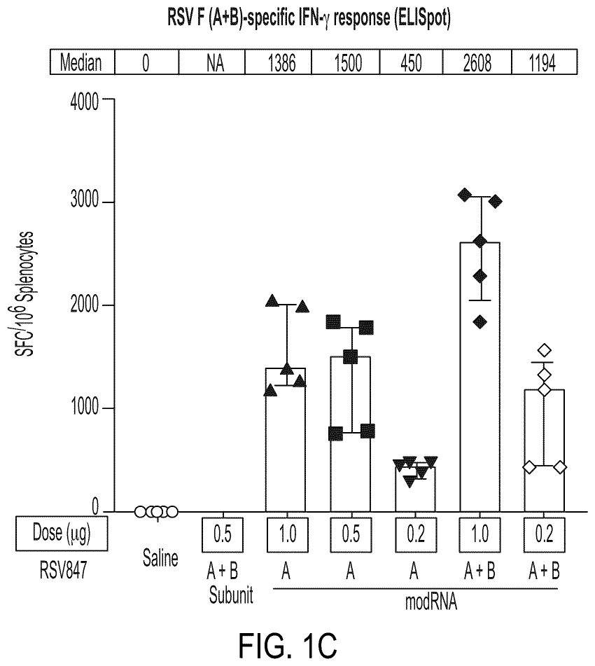

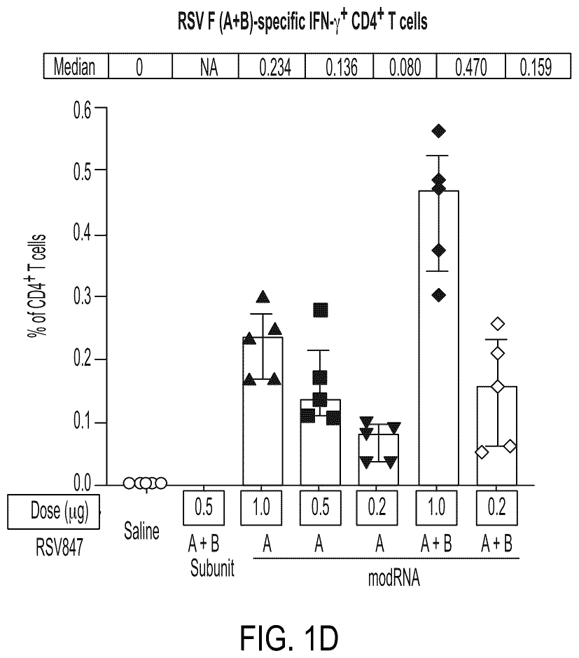

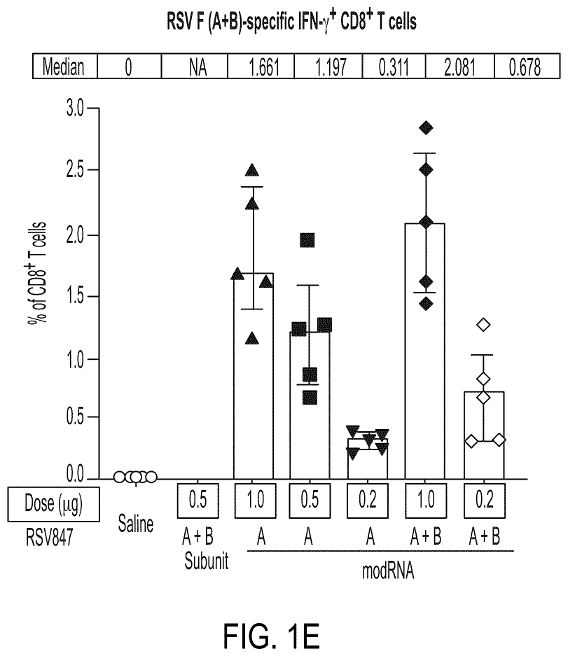

Results of neutralization assay at day 35 (2 weeks PD2) revealed that immunization of mice with modRNA-LNP formulations of RSV 847 as either monovalent RSV 847A or bivalent form RSV 847A and 847B induce a strong neutralizing antibody response to both RSV A and RSV B, which is higher compared to bivalent protein subunit version (FIGS. 1A and 1B, Table 27). Furthermore, the RSV B neutralizing response induced by bivalent formulation RSV847A+847B of modRNA-LNP is higher response than monovalent formulation RSV847A (FIG. 1B, Table 27), supporting the need for a bivalent version of RSV 847 modRNA vaccine for an optimal immune response in humans.

We next evaluated the T-cell response induced by these modRNA-LNP formulations of RSV 847 in mice. IFN-γ ELISpot results showed that the RSV 847 modRNA vaccines induce a strong T-cell response with a trend similar that of RSV neutralizing response (FIG. 1C, Table 27). Further, the ICS assay results revealed that the RSV 847 modRNA vaccines induce a high frequency of F-specific IFN-γ-expressing CD4+ T cells as well as CD8+ T cells in a dose-dependent manner (FIGS. 1D and 1E, Table 27). Notably, RSV A+B F-specific T-cell response induced by bivalent formulation (RSV 847A+847B) is higher than that of monovalent formulation (RSV 847A), further supporting that a bivalent version of RSV 847 induces higher magnitude of T-cell response, similar to that of neutralizing response.

In summary, mouse study results demonstrate a higher immunogenicity of modRNA-LNP formulation of RSV prefusion F constructs compared to that of protein subunit vaccine.

TABLE 27
Immune response induced by protein subunit and modRNA-LNP formulations of RSV prefusion F (847) in mice

| | | | RSV 847 | | | | RSV 847 | RSV 847 |
|---|---|---|---|---|---|---|---|---|
| | | | A + B | RSV 847 A | RSV 847 A | RSV 847 A | A + B | A + B |
| Assay Results | | | Subunit @ | modRNA @ | modRNA @ | modRNA @ | modRNA @ | modRNA @ |
| (Units) | Group | Saline | 0.5 μg | 1.0 μg | 0.5 μg | 0.2 μg | 1.0 μg | 0.2 μg |
| RSV A | Geomean | 40 | 415 | 32776 | 34743 | 5781 | 36349 | 7351 |
| 50% | STDEV | 8 | 2554 | 22871 | 30468 | 4498 | 27605 | 7629 |
| neutralizing | | | | | | | | |
| titer | | | | | | | | |
| RSV B | Geomean | 29 | 429 | 27158 | 17077 | 3461 | 81593 | 16495 |
| 50% | STDEV | 8 | 4045 | 22737 | 19634 | 5246 | 85120 | 18874 |
| neutralizing | | | | | | | | |
| titer | | | | | | | | |
| IFN-γ ELISpot | Median | 0 NA | | 1386 | 1500 | 450 | 2608 | 1194 |
| (SFC/10⁶ cells) | STDEV | 2 NA | | 368 | 468 | 67 | 469 | 464 |
| IFN-γ+ | Median | 0.00 NA | | 0.23 | 0.14 | 0.08 | 0.47 | 0.16 |
| CD4+ T cells | STDEV | 0.00 NA | | 0.05 | 0.06 | 0.03 | 0.09 | 0.08 |
| (%) | | | | | | | | |
| IFN-γ+ | Median | 0.00 NA | | 1.66 | 1.20 | 0.31 | 2.08 | 0.68 |
| CD8+ T cells | STDEV | 0.00 NA | | 0.49 | 0.43 | 0.06 | 0.52 | 0.35 |
| (%) | | | | | | | | |

NA: not analyzed

### Example 15. RSV F saRNA

saRNA synthesis occurs via in vitro transcription (IVT) and purification by ultrafiltration/diafiltration (UFDF-1). The saRNA is then enzymatically capped and purified by chromatography and a final UFDF-2, followed by final filtration and dispense.

TABLE 28
RSV F 847A saRNA construct comprising in order 5′cap-5′UTR-
nsP1-nsP2-nsP3-nsP4-Subgenomic promoter-RSV [ORF]-3′UTR-
polyA tail (encoding RSV F protein having SEQ ID NO: 4)

| RNA | Start | End | |
|---|---|---|---|
| Construct | [SEQ ID: 15] | [SEQ ID: 15] | SEQ ID |
| 5′-CAP | 1 | 1 | — |
| 5′-UTR | 2 | 45 | 52 |
| NSP1 | 46 | 1650 | 55 |
| NSP2 | 1651 | 4032 | 56 |
| NSP3 | 4033 | 5703 | 57 |
| NSP4 | 5704 | 7527 | 58 |
| Subgenomic | 7528 | 7562 | 54 |
| promoter | | | |
| RSV [ORF] | 7563 | 9290 | 11 |
| 3′-UTR | 9291 | 9407 | 24 |
| Poly-A tail | 9408 | 9487 | 26 |

TABLE 29
RSV F 847B saRNA construct comprising in order 5′cap-5′UTR-
nsP1-nsP2-nsP3-nsP4-Subgenomic promoter-RSV [ORF]-3′UTR-
polyA tail (encoding RSV F protein having SEQ ID NO: 6)

| RNA | Start | End | |
|---|---|---|---|
| Construct | [SEQ ID: 16] | [SEQ ID: 16] | SEQ ID |
| 5′-CAP | 1 | 1 | — |
| 5′-UTR | 2 | 45 | 52 |
| NSP1 | 46 | 1650 | 55 |
| NSP2 | 1651 | 4032 | 56 |
| NSP3 | 4033 | 5703 | 57 |
| NSP4 | 5704 | 7527 | 58 |
| Subgenomic | 7528 | 7562 | 54 |
| promoter | | | |
| RSV [ORF] | 7563 | 9290 | 12 |
| 3′-UTR | 9291 | 9407 | 24 |
| Poly-A tail | 9408 | 9487 | 26 |

### Example 16. RSV Antigens

Sequences of the RSV antigens/polypeptides, RSV DNA and RSV RNA of the present disclosure are provided in Tables 30-32. The sequences may comprise any stop codon, including but not limited to the stop codons provided in the Tables.

TABLE 30
RSV Polypeptides

| | SEQ |
|---|---|
| ID | ID NO: |
| Full Length F0 of Native RSV A2 (GenBank GI: 138251; | 1 |
| Swiss Prot P03420) Amino acid | |
| Full Length F0 of Native RSV B (18537 strain; GenBank | 2 |
| GI: 138250; Swiss Prot P13843) Amino acid | |
| RSV A WT F Amino acid | 3 |
| RSV A 847 F Amino acid | 4 |
| RSV B WT F Amino acid | 5 |
| RSV B 847 F Amino acid | 6 |

TABLE 31
RSV DNA

| | SEQ |
|---|---|
| | ID |
| ID | NO: |
| RSV A WT F DNA TGA stop codon (Amino acid SEQ ID NO: 3) | 7 |
| RSV A 847 F DNA TGA stop codon (Amino acid SEQ ID NO: 4) | 8 |
| RSV B WT F DNA TGA stop codon (Amino acid SEQ ID NO: 5) | 9 |
| RSV B 847 F DNA TGA stop codon (Amino acid SEQ ID NO: 6) | 10 |

TABLE 32
RSV RNA

| | SEQ |
|---|---|
| | ID |
| ID | NO: |
| RSV 847A F RNA UGA stop codon (Amino acid SEQ ID NO: 4) | 11 |
| RSV 847B F RNA UGA stop codon (Amino acid SEQ ID NO: 6) | 12 |
| 847A_modRNA | 13 |
| 847B_modRNA | 14 |
| saRNA 847A | 15 |
| saRNA 847B | 16 |

### Example 17: Preclinical Trials of the Combination of the Quadrivalent Influenza Nucleoside-Modified RNA Encoding Four HA Proteins, Formulated in LNPs, and the RSV Bivalent Subunit Vaccine Candidate, which is Composed of the Prefusion F Proteins from RSV Subtypes A and B

A preclinical study was performed to test the immunogenicity of a combination vaccine candidate containing both influenza virus and respiratory syncytial virus (RSV) antigens in a mouse model. The influenza vaccine material used in this study was either a quadrivalent influenza nucleoside-modified RNA (modRNA) vaccine candidate encoding the four hemagglutinin (HA) proteins recommended for the 2022-23 northern hemisphere (NH) season, formulated in lipid nanoparticles (LNPs), or a licensed NH 2022-23 adjuvanted influenza vaccine comparator (Fluad). The RSV bivalent subunit vaccine candidate used in this study includes the prefusion F proteins from RSV subtypes A and B. Mice (10 animals/group) were immunized with two doses 4 weeks apart of either a standalone influenza or RSV vaccine material or a combination of the candidate respiratory vaccines (mixed prior to injection or separately administered in each leg) to assess potential interference in immunogenicity. 2.5 μg of the RSV vaccine preparation and 1.2 μg of the influenza modRNA vaccine preparation were used, which both equate to 1/50^{th} of the expected human doses for each vaccine candidate in the 60+ year old population. 2.4 μg of the Fluad control was used ( 1/25^{th} of the human dose). Neutralization titers against RSV subtypes A and B and against each of the four influenza strains were evaluated using sera collected at Day 21 post prime and at Day 42 (14 days post boost). The animal study groups are shown below:

TABLE 33

| Group | | | |
|---|---|---|---|
| # | Vaccine Preparation(s) | Dose level(s) | Dose Vol/Route |
| 1 | Saline | — | 50 μl/IM |
| 2 | Quadrivalent influenza modRNA | 1.2 μg | 50 μl/IM |
| 3 | Bivalent RSV subunit | 2.5 μg | 50 μl/IM |
| 4 | Bivalent RSV subunit and | 2.5 μg (RSV) + | 50 μl/IM |
| | Quadrivalent influenza modRNA | 1.2 μg (Flu) | |
| | (bedside mix) | | |
| 5 | Bivalent RSV subunit and | 2.5 μg (RSV) | 50 μl/IM RSV in leg #1 |
| | Quadrivalent influenza modRNA | 1.2 μg (Flu) | 50 μl/IM Flu in leg #2 |
| | (separately administered in each leg) | | |
| 6 | Bivalent RSV subunit and | 2.5 μg (RSV) | 50 μl/IM RSV in leg #1 |
| | Fluad (NH 22-23) | 2.4 μg (Fluad) | 50 μl/IM Fluad in leg #2 |
| | (separately administered in each leg) | | |
| 7 | Fluad (NH 22-23) | 2.4 μg | 20 μl/IM |

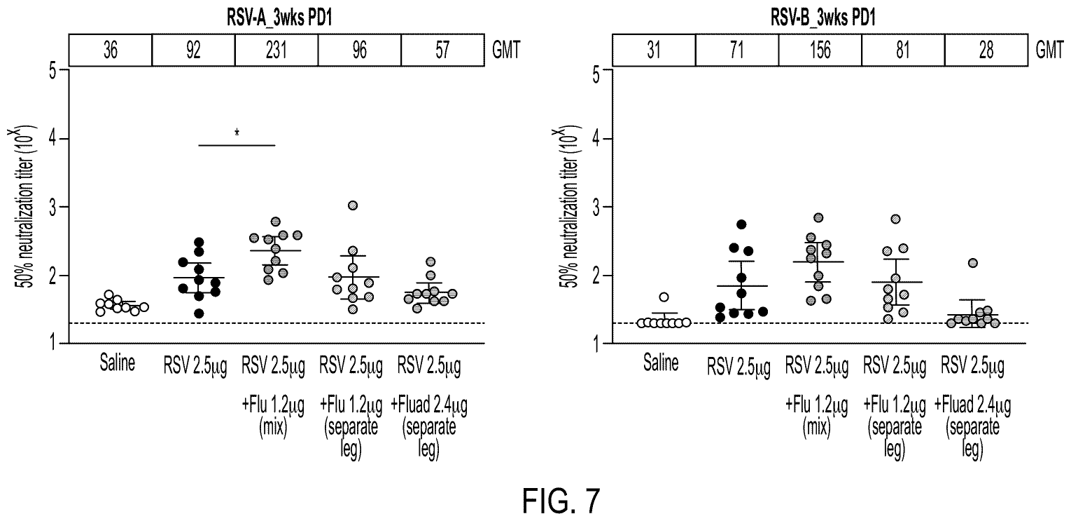

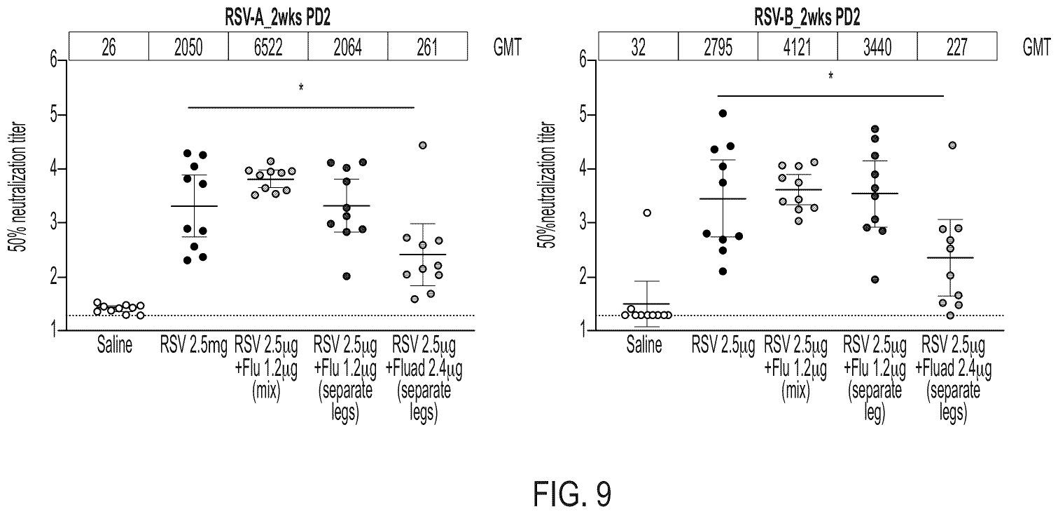

The influenza modRNA/RSV subunit combination vaccine candidate elicited similar or higher levels of virus neutralizing antibodies against RSV subtypes A and B compared to the RSV vaccine alone at 3 weeks post-dose 1 (FIG. 7) or at 2 weeks post-dose 2 (FIG. 9) in mice. When the RSV subunit vaccine was combined with a licensed adjuvanted influenza vaccine comparator, Fluad, lower levels of neutralizing antibodies to both RSV A and B subtypes were elicited compared to the RSV vaccine alone.

FIG. 7 depicts data wherein Balb/c mice were immunized IM with 2 doses 4 weeks apart with the following vaccine preparations: 2.5 μg bivalent RSV subunit vaccine; 2.5 μg RSV subunit vaccine in combination with 1 (0.2 μg quadrivalent modRNA flu vaccine that was either mixed prior to injection or given separately in each leg, or 2.5 μg RSV subunit vaccine in combination with 2.4 μg licensed adjuvanted quadrivalent influenza vaccine comparator, Fluad, given separately in each leg. Virus neutralization responses against RSV subtypes A and B were measured using sera collected on Day 21) at 3 weeks post-dose 1 (FIG. 7) or on Day 42 at 2 weeks post-dose 2 (FIG. 9).

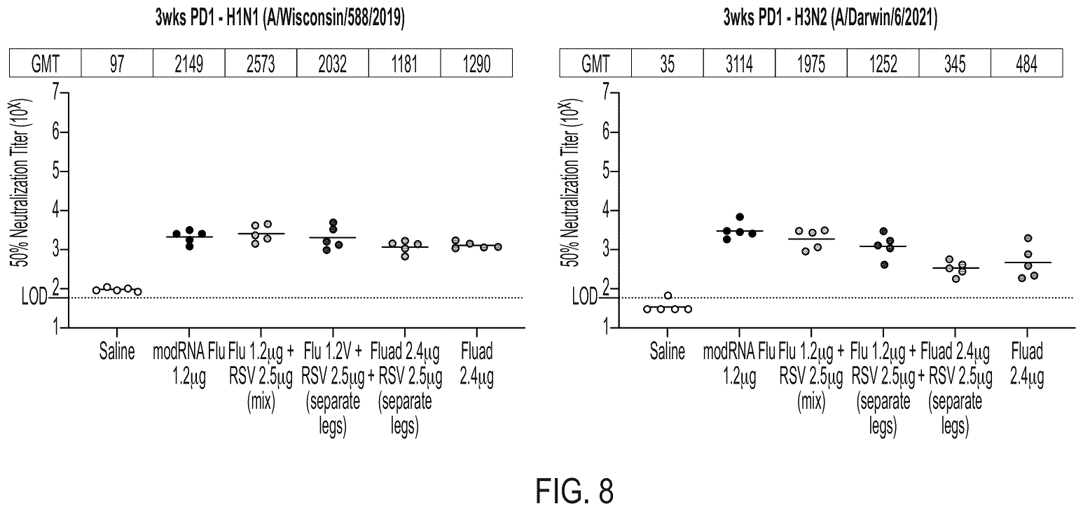

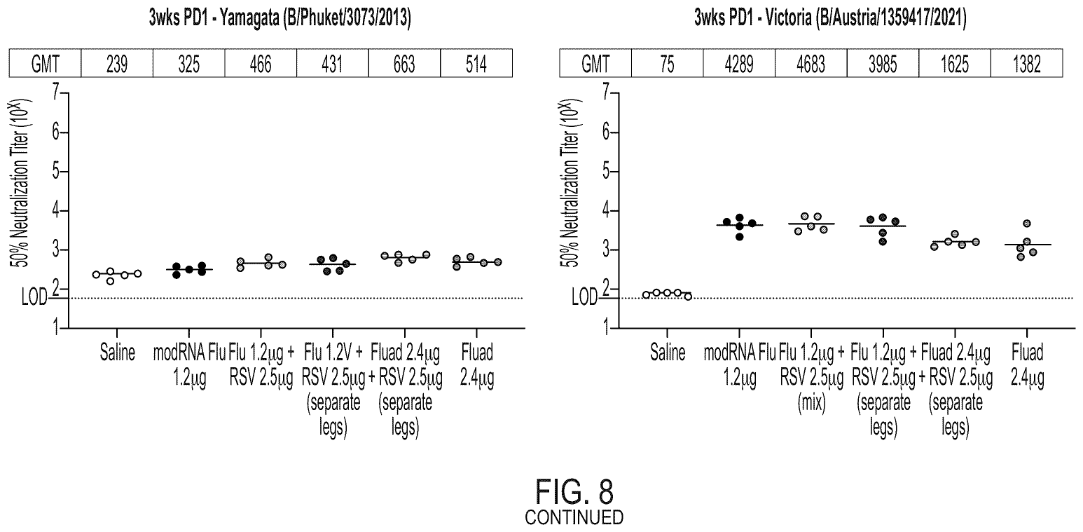

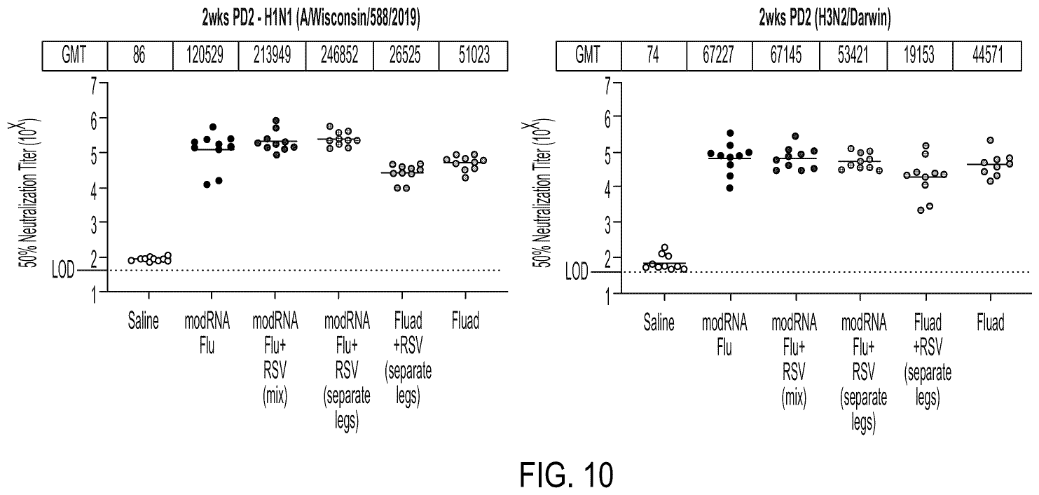

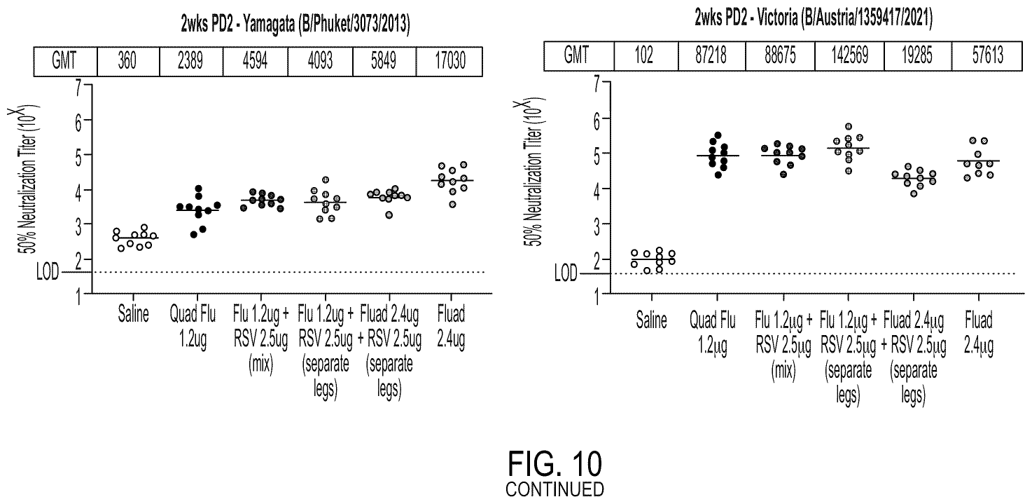

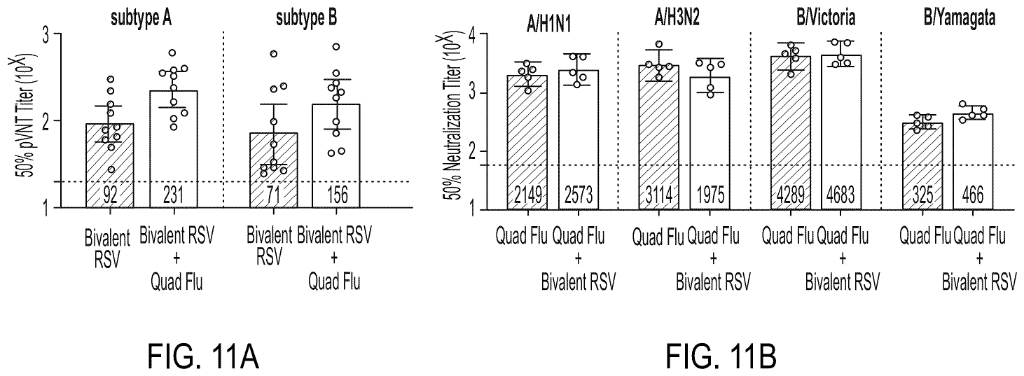

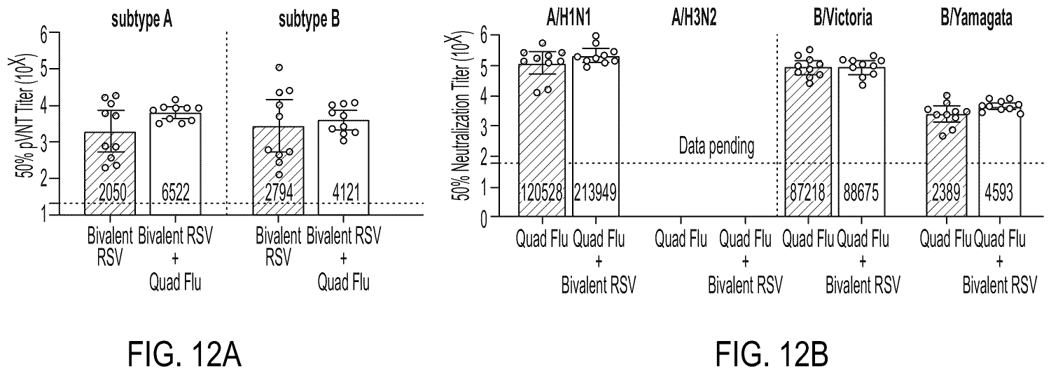

The influenza modRNA/RSV subunit combination vaccine candidate had comparable immunogenicity to the quadrivalent modRNA influenza vaccine alone at 3 weeks post-dose 1 (FIG. 8) and 2 weeks post-dose 2 (FIG. 10). Neutralizing titers to each of the influenza strains were comparable whether the influenza and RSV vaccines were co-administered in the same leg or injected into separate legs of the mice. Overall, these data indicated no interference in immunogenicity for influenza modRNA and RSV subunit combination vaccines in mice.

FIG. 8 depicts data wherein Balb/c mice were immunized IM with 2 doses 4 weeks apart with the following vaccine preparations: 1.2 μg quadrivalent modRNA flu vaccine, 1.2 μg quadrivalent modRNA flu vaccine in combination with 2.5 μg RSV subunit vaccine that was either mixed prior to injection or given separately in each leg, or 2.4 μg licensed adjuvanted quadrivalent influenza vaccine comparator, Fluad, given alone or in combination with 2.5 μg RSV subunit vaccine (co-administered separately in each leg). Virus neutralization responses against the four influenza vaccine strains were measured using sera collected on Day 21 (3 weeks post-dose 1).

FIG. 10 depicts data Balb/c mice were immunized IM with 2 doses 4 weeks apart with the following vaccine preparations: 1.2 μg quadrivalent modRNA flu vaccine, 1.2 μg quadrivalent modRNA flu vaccine in combination with 2.5 μg RSV subunit vaccine that was either mixed prior to injection or given separately in each leg, or 2.4 μg licensed adjuvanted quadrivalent influenza vaccine comparator, Fluad, given alone or in combination with 2.5 μg RSV subunit vaccine (co-administered separately in each leg). Virus neutralization responses against the four influenza vaccine strains were measured using sera collected on Day 42 (2 weeks post-dose 2).

### Example 18: a Study to Evaluate the Safety, Tolerability, and Immunogenicity of Respiratory Combination Vaccine Candidates in Adults 60 Years of Age and Older

Influenza, RSV, and SARS-CoV-2 viruses are cocirculating infections. An RSV vaccine, RSVpreF, was recently found to be efficacious against RSV-associated LRTI in older adults. Concurrent administration of vaccines against these respiratory viral infections potentially reduces the need for multiple healthcare or pharmacy visits and may improve vaccine uptake. A combination vaccine with RSVpreF and qIRV would potentially involve 1 injection at a healthcare provider visit. A study of combined vaccination may be used to investigate potential immunological interference in addition to safety and tolerability.

The present study will investigate the administration of combined RSVpreF with qIRV to assess the safety, tolerability, and potential for immunological interference. This evaluation will initially be conducted as a Phase 1b substudy, labeled as Substudy A, in healthy participants ≥60 years of age. The substudy is expected to provide useful data to inform future vaccine studies of RSVpreF in combination with mRNA-based vaccines, and further substudies may be added.

The “RSVpreF” composition includes 120 μg RSVpreF without any adjuvants, as described in Example 3. RSVpreF is supplied as a lyophilized white cake, packaged in a clear glass 2-mL vial with a rubber stopper, aluminum overseal, and flip-off cap. It is reconstituted with a PFS of sterile water diluent for injection.

The “qIRV” composition refers to 1 dose of qIRV encoding 2 influenza A strains and 2 influenza B strains, 60 μg (15 μg per strain), suitable for administration to participants 18 through 64 years of age and for participants ≥65 years of age, as described in Example 2. qIRV is supplied as a white to off-white suspension in a 2-mL type 1 clear glass vial, sealed with a 13-mm serum stopper and 13-mm aluminum flip-off seal. Fill volume is 0.68 mL.

This study is designed to evaluate the safety, tolerability, and immunogenicity of RSVpreF and a modified RNA vaccine (qIRV) against RSV and influenza when administered as a combined vaccine and when administered alone. This 3-visit study will initially be conducted as a Phase 1b, multicenter, parallel-group, randomized, placebo-controlled, observer-blinded substudy.

Healthy adults ≥60 years of age will be randomized 1:1 to either Group 1: combination (RSVpreF+qIRV) (Visit A101) followed by placebo (Visit A102) or Group 2: sequential administration of qIRV (Visit A101) followed by RSVpreF (Visit A102) administered 1 month apart. Randomization will be stratified by 3 age groups (60-64, 65-79, and >80 years) within each study site.

In participants in compliance with the key protocol criteria (evaluable participants), geometric mean ratio (GMR) of neutralizing titers (NTs) for each RSV subgroup (A or B) 1 month after vaccination with combination RSVpreF+qIRV will be compared to that with RSVpreF alone (sequential-administration group, Visit A103) to describe the immune responses (i.e., RSV A and RSV B NTs) elicited by combination RSVpreF+qIRV in comparison with those elicited by RSVpreF alone.

In participants in compliance with the key protocol criteria (evaluable participants), GMR of strain-specific HAI titers 1 month after vaccination with combination RSVpreF+qIRV will be compared to qIRV alone (sequential-administration group, Visit A102) to describe the immune responses (i.e., strain-specific HAI titers for the current seasonal strains (2×A, 2×B) recommended by WHO for recombinant or cell-based influenza vaccines) elicited by combination RSVpreF+qIRV in comparison with those elicited by qIRV alone. Further assessments may include determining: (a) GMTs of NTs for each RSV subgroup (A or B) at each applicable time point; (b) GMFRs of NTs for each RSV subgroup (A or B) from before vaccination to each applicable postvaccination time point; (c) GMTs of strain-specific HAI at each applicable time point; (d) GMFRs of strain-specific HAI from before vaccination to each applicable postvaccination time point; (e) The percentage of participants with strain-specific HAI titers ≥1:40 at each applicable time point; (f) The percentage of participants achieving strain-specific HAI seroconversion (Seroconversion is defined as an HAI titer <1:10 prior to vaccination and ≥1:40 at the time point of interest, or an HAI titer of ≥1:10 prior to vaccination with a 4-fold rise at the time point of interest) at each applicable postvaccination time point; (g) The percentage of participants with HAI composite response (composite response is defined as an HAI titer ≥1:40 for all 4 influenza strains included in the vaccine) at each applicable time point; and (h) The percentage of participants with HAI composite seroconversion (composite seroconversion is defined as seroconversion for all 4 influenza strains included in the vaccine) at each applicable time point.

For the purposes of this protocol, study intervention refers to the following products:
Combination RSVpreF+qIRV
RSVpreF+qIRV, which is a combination of the following vaccine candidates:
   120 μg RSVpreF containing 2 stabilized RSV prefusion F antigens, in equal amounts from virus subgroups A and B
   60 μg qIRV, ie, encoding HA of 4 strains as recommended for the influenza season
   (2 A strains and 2 B strains, at a dose of 15 μg per strain) RSVpreF
   120 μg RSVpreF containing 2 stabilized RSV prefusion F antigens, in equal amounts from virus subgroups A and B
qIRV
   60 μg qIRV, ie, encoding HA of 4 strains as recommended for the influenza season (2 A strains and 2 B strains, at a dose of 15 μg per strain)

The placebo is 0.9% normal saline for injection. All study interventions are to be administered as an IM injection into the deltoid muscle, preferably of the nondominant arm. Substudy A (SSA) investigated the safety, tolerability, and immunogenicity of RSVpreF and qIRV when combined (RSVpreF+qIRV), and when administered alone. Healthy adults ≥60 years of age received either the combination vaccine followed by a placebo injection 1 month later, or a sequential administration of qIRV vaccine followed by the RSVpreF vaccine 1 month later. The combination vaccine was a bedside mix of RSVpreF lyophilized cake reconstituted with water then mixed with qIRV, yielding a final injection volume of 1.0 mL. In the sequential administration group, the RSVpreF lyophilized cake was reconstituted with water for injection yielding a final injection volume of 0.5 mL.

Substudy B (SSB) investigated 2 formulations of RSVpreF+qIRV, differing in volume and osmolality, as the development of hypertonic formulations is sometimes required for injectable products to demonstrate efficacy, safety, and/or stability, but this may affect the tolerability. The first combination was the same formulation as in SSA. The second combination was a bedside mix of RSVpreF lyophilized cake directly reconstituted with qIRV to yield a final injection volume of 0.5 mL. The 0.5-mL direct reconstitution combination would result in a hyperosmotic (855 mOsm/kg) solution. These combinations were evaluated for safety, tolerability, and immunogenicity, with the goal of selecting a formulation for further study. To better assess the relationship among the effects of age/sex and the tolerability of the study intervention new formulation, the study population included older adults 50 years and above.

3.1. Overview of Study Design: Substudy A. Substudy A was a Phase 1b, single-site, parallel-group, randomized, placebo-controlled, observer-blinded substudy of RSVpreF and a nucleoside-modified RNA vaccine (qIRV) against RSV and influenza when administered as a combined vaccine (RSVpreF+qIRV) and when administered alone. Approximately 250 (n=125 per group) healthy adults ≥60 years of age were randomized 1:1 to either: ● Group 1: combination (RSVpreF+qIRV) (Visit A101) followed by placebo (Visit A102) or ● Group 2: sequential-administration of qIRV (Visit A101) followed by RSVpreF (Visit A102) administered 1 month apart. Randomization was stratified by 3 age groups (60-64, 65-79, and >80 years) within the study site. There were 3 scheduled study visits, each 1 month apart (FIG. 1). Immunogenicity was evaluated with blood samples collected prior to vaccination at Visit 1 (A101) and Visit 2 (A102), and at Visit 3 (A103). Blood samples for the combination RSVpreF+qIRV group were collected at 2-months for evaluation of both RSV and influenza. Local reactions and systemic events occurring within 7 days after each vaccination were recorded in an e-diary device or smartphone app. AEs and SAEs were collected from the signing of informed consent through study completion

TABLE 34
Substudy A design schema, C5401001

| Healthy | | Visit A102 | Visit A103 |
|---|---|---|---|
| adults ≥60 | | (month 1) | (Month 2) |
| years of age | Visit A101 | 28-35 days after | 28-25 days after |
| 125 per group, | (day 1) | Vaccination 1 is | Vaccination 2 |
| randomized 1:1 | Vaccination 1 | Vaccination 2 | Follow-up |
| Group 1: | Combination | Placebo | Blood draw |
| Combination | (RSVpreF + | | |
| RSVpreF + | qIRV) | | |
| qIRV/placebo | | | |
| Group 2: | qIRV | RSVpreF | Blood draw |
| qIRV/RSVpreF | | | |

Discussion of Study Design. Substudy A. Substudy A was designed to evaluate the safety, tolerability, and immunogenicity of RSVpreF and a modified RNA vaccine (qIRV) against RSV and influenza when administered as a combined vaccine and when administered alone. The dose and formulation of RSVpreF (120 μg without any adjuvants) selected reflects the approved dose. The dose of qIRV 60 μg, encoding 2 A strains and 2 B strains (15 μg per strain), was selected based on safety and immunogenicity data from Phase 1 and Phase 3 studies. Healthy adults ≥60 years of age were randomized 1:1 to either: ● Group 1: combination (RSVpreF+qIRV) 1.0-mL (Visit A101) followed by placebo (Visit A102) or ● Group 2: sequential administration of qIRV (Visit A101) followed by RSVpreF (Visit A102) administered 1 month apart. Randomization was stratified by 3 age groups (60-64, 65-79, and ≥80 years) within each study site. To ensure a broad representation of age groups (participants at least 65 years of age) for the current recommendation on seasonal influenza vaccination, no more than 40 participants 60 through 64 years of age will be vaccinated.

Enrolled in Substudy A were healthy participants ≥60 years of age with preexisting stable disease, defined as disease not requiring significant change in therapy or hospitalization for worsening disease during the 6 weeks before enrollment.

Study Interventions Administered. All study interventions for Substudy A and Substudy B, presented below, were administered as IM injection given in the deltoid muscle as a single injection. Substudy A Vaccine Components (Combination RSVpreF+qIRV and Placebo) RSVpreF+qIRV, is a combination of the following vaccine candidates: ● 120 μg RSVpreF containing 2 stabilized RSV prefusion F antigens, in equal amounts from virus subgroups A and B●60 μg qIRV, encoding HA of 4 strains as recommended for the influenza season (2 A strains and 2 B strains, at a dose of 15 μg per strain) The Placebo is 0.9% normal saline for injection.

Immunogenicity Analysis—Substudy A and Substudy B Substudy A ● Influenza strain-specific GMRs of HAI titers 1 month after vaccination (Visit A102 in the combination RSVpreF+qIRV group to Visit A103 in the sequential-administration group) and GMRs of RSV NTs 1 month after vaccination (Visit A102 in the combination RSVpreF+qIRV group to Visit A103 in the sequential-administration group) are provided along with the associated 2-sided 95% CIs.
Substudy A Primary Immunogenicity. In Substudy A, at 1 month after vaccination, a robust immune response to the target antigens (RSV subtypes and influenza strains) were shown in the combination RSVpreF+qIRV group compared to RSVpreF or qIRV alone in the sequential administration group (Table 35). At 1 month after RSVpreF administration, the ratios of RSV 50% neutralizing GMT were ≥0.79 and the lower bounds of the 2-sided 95% Cl were >0.5. At 1 month after qIRV, for the 4 influenza strains included in qIRV the ratios of HAI GMT were ≥0.74 and the lower bounds of the 2-sided 95% Cl were >0.5. Therefore, all 6 antigens (RSV and HAI) met a 2-fold margin based on (lower bound of the 2-sided descriptive 95% Cl for GMR >0.5) for combination RSVpreF+qIRV compared to RSVpreF or qIRV alone in the sequential administration group. GMTs and GMFRs were evaluated by age group for the evaluable RSV and qIRV immunogenicity populations. However, two age groups (60-64 and ≥80 years of age) included limited number of participants. Overall, there were no meaningful differences in the neutralizing titers between the age groups (Table 36). Percentages of participants reaching >60% “seroprotection” achieved for both A strains and B/Victoria (Table 35). Percentages of participants reaching >30% seroconversion achieved for A strains in both vaccine groups and for B strains in the sequential groups (Table 35).

### Substudy a Evaluation of Response to Study Intervention

In Substudy A, robust immune responses to the target antigens (RSV subtypes and influenza strains) were shown in the combination group and the sequential-administration group. These responses were comparable and characterized by:

### Immune Response to RSV

Both RSV A and RSV B antigens met the 2-fold noninferiority margin (lower bound 95% Cl was >0.5).
RSV GMTs showed a robust response in both the combination RSVpreF+qIRV and RSVpeF alone (sequential-administered) groups for RSV A and RSV B.
RSV A and RSV B NT GMFRs were >10 after RSVpreF+qIRV (combination) and after RSVpreF alone (sequential-administered) groups. GMFRs for the combined RSV A/B NTs at 1 month after vaccination were >11 for both vaccine groups.
   Immune Response to qIRV
GMFR for Strain-specific HAI titers showed a high response for both the combination and qIRV alone groups. Noninferiority was met based on the 2-fold noninferiority margin (LBCl >0.5).
HAI seroconversion rates at 1 month after vaccination were similar for the combination (range: 24% to 97%) and qIRV alone (range: 34% to 93%) groups. HAI Seroprotection was similar between combination (range: 17% to 116%) and qIRV alone (13% to 109%) groups.

TABLE 35
HAI and RSV Neutralizing Titer GMTs/GMRs at 1 Month After Vaccination -
Evaluable HAI Immunogenicity Population and Evaluable RSV Immunogenicity Population
Vaccine Group (as Randomized)

| Assay: | Combination | | | | | | | |
|---|---|---|---|---|---|---|---|---|
| Strain or | (RSVpreF + qIRV)/Placebo | | | qIRV or RSVpreF Alone | | | Comparison | |
| Subgroup | N^{a} | GMT^{b} | (95% CI^{b}) | N^{a} | GMT^{b} | (95% CI^{b}) | GMR^{c} | (95% CI^{c}) |
| HAI: H1N1 | 119 | 194 | (164.3, 227.9) | 112 | 238 | (196.5, 288.7) | 0.81 | (0.632, 1.044) |
| A/Wisconsin^{d} | | | | | | | | |
| HAI: H3N2 A/Darwin^{d} | 119 | 161 | (136.9, 189.9) | 112 | 219 | (178.1, 269.0) | 0.74 | (0.567, 0.957) |
| HAI: B/Austria^{d} | 119 | 53 | (44.6, 62.1) | 112 | 56 | (47.7, 66.3) | 0.94 | (0.741, 1.181) |
| HAI: B/Phuket^{d} | 119 | 22 | (18.5, 26.4) | 112 | 24 | (20.2, 29.1) | 0.91 | (0.707, 1.174) |
| NT: RSV A^{e} | 119 | 16553 | (14120.9, 19403.1) | 112 | 19740 | (16555.5, 23536.4) | 0.84 | (0.662, 1.062) |
| NT: RSV B^{e} | 119 | 11795 | (9961.1, 13966.7) | 112 | 14959 | (12408.8, 18032.4) | 0.79 | (0.614, 1.013) |

Abbreviations: GMR = geometric mean ratio; GMT = geometric mean titer; HAI = hemagglutination inhibition assay; LLOQ = lower limit of quantitation; NT = neutralizing titer.
Note:
The LLOQ values were 10 for HAI titer, 242 for RSV A NT, and 99 for RSV B NT. Assay results below the LLOQ were set to 0.5 × LLOQ.
^{a}N = number of participants with valid and determinate assay results for the specified assay in respective evaluable immunogenicity population.
^{b}GMTs and 2-sided CIs were calculated by exponentiating the mean logarithm of the titers and the corresponding CIs (based on the Student t distribution).
^{c}GMRs and 2-sided CIs were calculated by exponentiating the mean differences of the logarithms of the titers (combination minus sequential-administration) and the corresponding CIs (based on the Student t distribution).
^{d}Based on evaluable HAI immunogenicity population.
^{e}Based on evaluable RSV immunogenicity population.

TABLE 36
HAI and RSV Neutralizing Titer GMTs/GMRs at 1 Month After Vaccination, by Age Group -
Evaluable HAI Immunogenicity Population and Evaluable RSV Immunogenicity Population

| | | Vaccine Group (as Randomized) | | | | | | | |
|---|---|---|---|---|---|---|---|---|---|
| | Assay: | Combination | | | | | | | |
| Age | Strain or | (RSVpreF + qIRV)/Placebo | | | qIRV or RSVpreF Alone | | | Comparison | |
| Group | Subgroup | N^{a} | GMT^{b} | (95% CI^{b}) | N^{a} | GMT^{b} | (95% CI^{b}) | GMR^{c} | (95%CI^{c}) |
| 60-64 | HAI: H1N1 | 21 | 177 | (115.3, 270.7) | 20 | 237 | (161.3, 347.8) | 0.75 | (0.428, 1.300) |
| Years | A/Wisconsin^{d} | | | | | | | | |
| | HAI: H3N2 A/Darwin^{d} | 21 | 140 | (86.3, 227.7) | 20 | 184 | (116.7, 289.4) | 0.76 | (0.401, 1.451) |
| | HAI: B/Austria^{d} | 21 | 80 | (53.0, 120.7) | 20 | 55 | (35.6, 83.8) | 1.46 | (0.825, 2.600) |
| | HAI: B/Phuket^{d} | 21 | 24 | (14.6, 38.0) | 20 | 21 | (13.2, 32.6) | 1.14 | (0.603, 2.154) |
| | NT: RSV A^{e} | 21 | 17168 | (11500.3, 25629.5) | 22 | 23282 | (15431.2, 35127.2) | 0.74 | (0.423, 1.287) |
| | NT: RSV B^{e} | 21 | 11738 | (6944.1, 19842.0) | 22 | 17856 | (12671.5, 25161.9) | 0.66 | (0.357, 1.211) |
| 65-79 | HAI: H1N1 | 89 | 198 | (164.2, 239.9) | 86 | 241 | (191.7, 303.7) | 0.82 | (0.612, 1.106) |
| Years | A/Wisconsin^{d} | | | | | | | | |
| | HAI: H3N2 A/Darwin^{d} | 89 | 158 | (131.7, 189.4) | 86 | 227 | (178.2, 290.4) | 0.69 | (0.513, 0.939) |
| | HAI: B/Austria^{d} | 89 | 49 | (40.2, 58.7) | 86 | 57 | (47.3, 68.7) | 0.85 | (0.655, 1.109) |
| | HAI: B/Phuket^{d} | 89 | 22 | (18.3, 27.3) | 86 | 25 | (20.2, 30.6) | 0.90 | (0.673, 1.196) |
| | NT: RSV A^{e} | 89 | 16217 | (13442.1, 19563.6) | 84 | 18994 | (15458.4, .6) | 0.85 | (0.647, 1.126) |
| | NT: RSV B^{e} | 89 | 11872 | (9855.5, 14300.7) | 84 | 14028 | (11183.3, 17596.0) | 0.85 | (0.632, 1.132) |
| ≥80 Years | HAI: H1N1 | 9 | 187 | (93.3, 373.4) | 6 | 202 | (61.5, 661.2) | 0.93 | (0.267, 3.215) |
| | A/Wisconsin^{d} | | | | | | | | |
| | HAI: H3N2 A/Darwin^{d} | 9 | 274 | (153.2, 491.1) | 6 | 226 | (75.1, 681.9) | 1.21 | (0.388, 3.784) |
| | HAI: B/Austria^{d} | 9 | 43 | (22.0, 85.0) | 6 | 50 | (15.4, 165.3) | 0.86 | (0.248, 2.961) |
| | HAI: B/Phuket^{d} | 9 | 17 | (6.9, 42.8) | 6 | 28 | (9.4, 85.2) | 0.61 | (0.169, 2.170) |
| | NT: RSV A^{e} | 9 | 18619 | (10208.9, 33957.5) | 6 | 18482 | (6908.0, 49446.7) | 1.01 | (0.356, 2.851) |
| | NT: RSV B^{e} | 9 | 11187 | (5471.6, 22874.1) | 6 | 19211 | (6036.9, 61133.6) | 0.58 | (0.171, 1.985) |

### Substudy a Exploratory Immunogenicity-RSV: A and B Subgroup Neutralization

In the evaluable RSV immunogenicity population, RSV A and B 50% NT GMTs were substantially increased from before to 1 month after RSVpreF vaccination for combination RSVpreF+qIRV and RSVpreF alone (sequential administration group). For 50% NTs for RSV A and RSV B, GMFRs from before vaccination to 1 month after vaccination were similar for both groups (range: 10.9 to 15.9). At the 2 month time point (for the combination group (RSVpreF+qIRV) only), the immune response remained elevated. Results were similar in the mITT immunogenicity population.

HAI Titers for the 4 Influenza Strains Included in qIRV. For the evaluable qIRV immunogenicity population at 1 month after vaccination, HAI GMRs were lowest for H3N2 and H1N1 strains, though >0.73, with a LBCl >0.56. HAI titers showed a higher response for both the combination RSVpreF+qIRV and qIRV alone (sequential administration) groups for H1N1 and H3N2, with the lowest response observed for B/Phuket followed by B/Austria. HAI GMFR was higher in qIRV alone sequential group, though B/Austria and B/Phuket GMFR were similarly lowest.

Age Subgroup Analysis—RSV NTs and HAI Titers. Subgroup analysis for the evaluable RSV and HAI immunogenicity populations by age showed noninferiority of immune responses in the combination group across all 3 age groups (60-64, 65-79, and >80 years).

Differences in Seroprotection and Seroconversion. HAI titer seroprotection (defined as HAI titer ≥1:40) was similar for combination RSVpreF+qIRV and qIRV alone (sequential administration group). The proportion of participants achieving seroprotection for combination RSVpreF+qIRV was highest for H1N1 (97.5%), followed by H3N2 (96.6%), B/Austria (80.7%), and lowest for B/Phuket (44.5%).In the combination group, 244.5% of participants achieved titers associated with seroprotection at 1 month after vaccination across strains. In the sequential-administration group, 246.4% of participants achieved titers associated with seroprotection at 1 month after vaccination across strains. The difference between groups was ≤6.1% for all strains for participants who received the combination (RSVpreF+qIRV) and qIRV.

HAI titer seroconversion (defined as ≥4-fold rise from before vaccination if the HAI titer is ≥1:10 before vaccination, or HAI ≥1:40 if HAI is <1:10) at 1 month after vaccination was observed. In the combination RSVpreF+qIRV group, 220.2% of participants achieved titers associated with seroconversion at 1 month after vaccination across strains. In the qIRV alone (sequential-administration) group, 230.4% of participants achieved titers associated with seroconversion at 1 month after vaccination across strains with the greatest differences observed for for B/Austria (−12.0%), B/Phuket (−10.2%), and H1N1 (−9.7%).

In Substudy A, the combination of RSVpreF+qIRV was found to be safe and well-tolerated compared to the sequential administration of qIRV alone and RSVpreF alone. The combination vaccine elicited robust immune responses that were similar to those elicited by the individual vaccine component alone administered sequentially, as measured by RSV A and B NTs and strain-specific influenza HAI titers.

### Example 19: The Combination of modRNA Flu and RSV Subunit Immunogenic Compositions Support Stability for at Least 4 Hours at Up to 30° C. after the RSV Subunit Immunogenic Composition is Reconstituted

The combination of modRNA Flu and RSV subunit immunogenic compositions support stability for at least 4 hours at up to 30° C. after the RSV subunit immunogenic composition is reconstituted. In preferred embodiments, the percentage of high molecular weight species observed following reconstitution of the RSV subunit immunogenic composition is less than or equal to 25%, and more preferably less than 10%.

This study is designed to examine the post reconstitution behavior and in-use stability of RSV Vaccine reconstituted with qIRV mRNA directly. This combination would result in a hyperosmotic (+800 mOsm/Kg) solution and is being evaluated from a Safety and Tolerability perspective.

The quadrivalent influenza mRNA vaccine & RSV bivalent vaccine materials to be used in the reconstitution (co-formulation) study is listed in the Table below.

TABLE 37

| Use | Material/Component | Description |
|---|---|---|
| Active DP | PF-07871992 | 0.12 mg/ml Flu mRNA in 10 mM Tris |
| (Quadrivalent | | buffer, 300 mM sucrose, pH 7.4 |
| Influenza) | | |
| Active DP (RSV | PF-06928316 | 120 μg of RSV bivalent antigen, 1.15 mg Tris, |
| Bivalent) | | 11.3 mg sucrose, 22.5 mg mannitol, 1.1 mg NaCl, |
| | | 0.08 mg polysorbate 80 (PS80), pH 7.4 |
| Luer-lok Syringes | 1 mL polycarbonate | Disposable sterile polycarbonate; 1 mL |
| | syringe | |
| Stainless Steel | 21G × 1″ stainless | 21G 1″ Becton Dickenson (BD) |
| Needles (for sample | steel needle | PRECISIONGLIDE ® |
| drawing) | | |
| Stainless Steel | 25G × 1.5″ stainless | 25G 1.5″ MONOJECT ® |
| Needles | steel needle | |
| (for capping) | | |

The planned Phase I presentation for the PF-07941314 Influenza mRNA & RSV Combination Vaccine includes:
Quadrivalent Influenza mRNA Vaccine:
   Quadrivalent Influenza mRNA drug product, Suspension for Injection (60 mcg/vial)
RSV Bivalent Vaccine:
   RSV Bivalent drug product, Powder for Solution for Injection (120 mcg/vial)

This study evaluated the behavior and stability of prepared quadrivalent influenza and RSV bivalent combination vaccine when reconstituted and held in contact with vials. The evaluated concentration were based on a Phase 1 clinical doses of 180 mcg. Table 38 below provides information on the preparation configuration. To evaluate this preparation, the dosage mixing configuration for this study was designed to prepare 177.4 mcg doses as bulk preparations, as summarized in Table 39.

TABLE 38
Dose Calculations for Dosage Form Configurations of
PF-07941314 Combination Vaccine for IM Injection

| | | Final | | | Vials per |
|---|---|---|---|---|---|
| | Final Dose | concen- | | Volume (mL) | prepa- |
| Active DP | (mcg) | tration | | per prep | ration |
| Quadrivalent | 57.4^{a} | 114.7 | μg/mL | 0.65 | 2 |
| Influenza | | | | | |
| (0.12 mg/mL) | | | | | |
| RSV Bivalent | 120^{b} | 240 | μg/mL | — | 1 |
| (0.24 mg/mL) | | | | | |
| Total | 177.4 | 354.7 | μg/mL | 0.68 | 1 |

^{a}Based on assumption of 0.68 mL dose volume after reconstitution (0.68 mL when performed with WFI)
^{b}Accounts for reconstitution with 0.65 mL (similar to reconstituting with 0.65 mL (0.69 mL fill) of WFI as intended for RSV Vx alone
Note:
Extractable volume for Flu is 0.5 mL so need 2 vials per prep; using pooled DP needed 33 vials

TABLE 39
Bulk Prep Calculations Per Sample

| Active DP | Metrics | Bulk Prep 177.4 mcg dose | |
|---|---|---|---|
| Quadrivalent | Volume | 0.65 | mL |
| Influenza | Number of Vials | 33 vials (+1 for | |
| (0.12 mg/mL) | | ARD) = 34 vials | |
| RSV | Volume (mL) | 0.5 | mL |
| Bivalent | Number of Vials | 25 vials (+1 for | |
| (0.24 mg/mL) | | ARD) = 26 vials | |
| Combined Vaccine | Volume (mL) | 0.68 | mL |
| | | 24 | vials |

Quadrivalent influenza vials were transferred directly from −80° C. freezer and thawed at room temperature under ambient light. Upon complete thaw, the vial contents were mixed by inversion for 10 times. RSV bivalent vaccine was then reconstituted with the qIRV material. Observations were made and recorded upon reconstitution. The prepared solutions were held at 30° C. for up to 4 hours, which supports the commercial RSV vaccine temperature hold guidance. After the incubation, the preparations were observed for particulates and quality attributes were analyzed. The aliquots were submitted in real-time for immediate analysis (except for IVE and ELISA which were flash-frozen and stored at −80° C. until testing) by various assays.

TABLE 40
Test Results for PF-07941314 Influenza mod
mRNA & RSV Combination Vaccine (180 μg dose)

| | | | 30° C. | | | |
|---|---|---|---|---|---|---|
| Test | Study Criteria | T0 | T2H Syringe | T4H Syringe | T2H Vial | T4H Vial |
| HMMS (SEC) | ≤25% | 2.4 | 6.1 | 5.9 | 4.0 | 5.7 |

Abbreviations:
HMMS = high molecular weight species;
SEC = size exclusion chromatograph

TABLE 41
Test Results for PF-07941314 Influenza mod mRNA & RSV Combination Vaccine (180 μg dose)

| | | | 30° C. | | | |
|---|---|---|---|---|---|---|
| | Study | | T2H | T4H | | |
| Test | Criteria | T0 | Syringe | Syringe | T2H Vial | T4H Vial |
| Appearance | White to off- | WOWS | WOWS | WOWS | WOWS | WOWS |
| | white | | | | | |
| | suspension | | | | | |
| Appearance | Essentially | EFVP | EFVP | EFVP | EFVP | EFVP |
| (Visible | Free from | | | | | |
| Particles) | Visible | | | | | |
| | Particles | | | | | |
| pH | 7.4 ± 0.5 | 7.3 | 7.2 | 7.3 | 7.3 | 7.3 |
| RNA Content | T0 ± 20% | 62 | 59 | 54 | 58 | 53 |
| (RiboGreen) | | | | | | |
| RNA Integrity | ≥50% Intact | 77 | 78 | 77 | 79 | 78 |
| (CGE by | RNA | | | | | |
| fragment | | | | | | |
| analyzer) | | | | | | |
| RNA | ≥80% | 93 | 88 | 83 | 86 | 83 |
| Encapsulation | | | | | | |
| (RiboGreen) | | | | | | |
| LNP Size (DLS) | 40-120 nm | 68 | 68 | 69 | 68 | 70 |
| LNP PDI (DLS) | ≤0.3 | 0.15 | 0.15 | 0.16 | 0.15 | 0.18 |
| In vitro | Report | A/Wisc: 93% | A/Wisc: 91% | A/Wisc: 91% | A/Wisc: 92% | A/Wisc: 98% |
| Expression | Result | [22 ng/well], | [22 ng/well], | [22 ng/well], | [22 ng/well], | [22 ng/well], |
| | | A/Darwin: 95% | A/Darwin: 94% | A/Darwin: 94% | A/Darwin: 96% | A/Darwin: 97% |
| | | [7 ng/well], | [7 ng/well], | [7 ng/well], | [7 ng/well], | [7 ng/well], |
| | | B/Austria: 91% | B/Austria: 89% | B/Austria: 85% | B/Austria: 85% | B/Austria: 86% |
| | | [22 ng/well], | [22 ng/well], | [22 ng/well], | [22 ng/well], | [22 ng/well], |
| | | B/Phuket: 85% | B/Phuket: 85% | B/Phuket: 83% | B/Phuket: 87% | B/Phuket: 95% |
| | | [67 ng/well] | [67 ng/well] | [67 ng/well] | [67 ng/well] | [67 ng/well] |
| Protein | T0 ± 20% | 115 | 103 | 107 | 102 | 104 |
| Concentration | (mcg/mL) | | | | | |
| HMMS (SEC) | ≤25% | 2.4 | 6.1 | 5.9 | 4.0 | 5.7 |
| LMMS (SEC) | ≤5% | 1.6 | 1.7 | 2.0 | 1.9 | 1.9 |
| Trimer (SEC) | ≥70% | 96.0 | 92.2 | 92.1 | 94.1 | 92.4 |
| Relative | 50-150% | 81 | 88 | 89 | 83 | 105 |
| Prefusion | | | | | | |
| Content | | | | | | |
| (Prefusion F | | | | | | |
| ELISA)^{a} | | | | | | |

Abbreviations:
WOWS = White to off-white suspension;
EFVP = Essentially Free of Visible Particles;
LNP = Lipid Nano Particle;
CGE = capillary gel electrophoresis;
DLS = Dynamic Light Scattering;
HMMS = high molecular weight species;
LMMS = low molecular weight species;
SEC = size exclusion chromatograph

### Example 20: a Study to Determine the Feasibility of Co-Formulating and Freeze-Drying A Subunit Drug Product and an Lnp Encapsulating mRNA, in A Tris-Sucrose Buffer

One of the goals of this study was to determine the feasibility of co-formulating and freeze-drying RSV subunit (described in Example 3) and modFlu (Phuket strain; formulation is described exemplarily in Example 1) in Tris-sucrose buffer. The objectives included evaluating the impact of salt (NaCl) in the prelyo matrix on RSV and mRNA-LNP stability; Post freeze-drying; Post-reconstitution (in sWFI, 0.45% NaCl and 0.9% NaCl); and Stability (2-8° C., 25° C. and 35° C.).

TABLE 42

| | | | | | | | |
|---|---|---|---|---|---|---|---|
| | NaCl | modRNA | Sucrose | RSV | PS80 | Tris | Osmo |
| Formulation | (mM) | (mg/mL) | (%) | (mg/mL) | (%) | (mM) | (mOsm/kg) |

Pre-lyo formulation

| | | | | | | | |
|---|---|---|---|---|---|---|---|
| 1 | 0 | 0.260 | 10.3 | 0.52 | 0.02 | 21.67 | 323 |
| 2 | 50.00 | 0.260 | 10.3 | 0.52 | 0.02 | 21.67 | 416 |

Post-recon formulation

| | | | | | | | | Recon | Recon |
|---|---|---|---|---|---|---|---|---|---|
| | | | | | | | Recon | with | with |
| | | | | | | | with | 0.45% | 0.9% |
| | | | | | | | WFI | NaCl | NaCl |
| 1 | 0.00 | 0.12 | 5 | 0.24 | 0.01 | 10 | 149 | 292 | 435 |
| 2 | 23.08 | 0.12 | 5 | 0.24 | 0.01 | 10 | 192 | 335 | 478 |

TABLE 43

| | |
|---|---|
| Fill Vol (mL) | 0.3 |
| Recon Vol (mL) (total vol) | 0.63 (0.65) |
| Inj Vol (mL) | 0.5 |

The exemplary freeze-drying cycle parameters tested on the composition comprising a combination of modRNA flu and RSV subunit compositions are shown below:

TABLE 44

| | Target Cycle | Time, | Cumulative |
|---|---|---|---|
| Process Step | Parameters | hours | time, hours |
| Freezing including | Ramp to −45° C. in 130 | 2.17 | 2.17 |
| annealing at −10° C. | min | | |
| for 270 mins | Hold at −45° C. for 60 | 1.00 | 3.17 |
| | min | | |
| | Ramp to −10° C. in 70 | 1.17 | 4.33 |
| | min | | |
| | Hold at −10° C. for 180 | 3.00 | 7.33 |
| | min | | |
| | Ramp to −45° C. in 70 | 1.17 | 8.50 |
| | min | | |
| | Hold at −45° C. for 60 | 1.00 | 9.50 |
| | min | | |
| Chill condenser and | Tcond <−50° C.; | 0.00 | 9.50 |
| pull vacuum | Pch = 40 mT | | |
| Primary Drying | Ramp to −36° C. in 10 | 0.17 | 9.67 |
| | min | | |
| | Hold at −36° C. for 2700 | 45.00 | 54.67 |
| | min | | |
| Secondary Drying | Ramp to 35° C. in 355 | 5.92 | 60.58 |
| | min | | |
| | Hold at 35° C. for 360 | 6.00 | 66.58 |
| | min | | |

Formulations prepared and tested included:
a) Formulation 1: Prelyo: 0.26 mg/mL mRNA/0.52 mg/mL RSV F Protein Vx in 21 mM Tris, 10.3% sucrose, 0.02% Polysorbate 80, pH 7.4
b) Formulation 1 Postlyo: 0.12 mg/mL mRNA/0.24 mg/mL RSV F Protein Vx in 10 mM Tris, 5% sucrose, 0.01% Polysorbate 80, pH 7.4
c) Formulation 2 Prelyo: 0.26 mg/mL mRNA/0.52 mg/mL RSV F Protein Vx in 21 mM Tris, 10.3% sucrose, 50 mM NaCl, 0.02% Polysorbate 80, pH 7.4
d) Formulation 2 Postlyo: 0.12 mg/mL mRNA/0.24 mg/mL RSV F Protein Vx in 10 mM Tris, 5% sucrose, 23 mM NaCl, 0.01% Polysorbate 80, pH 7.4

Data relating to the four formulations listed above, a)-d), and that of additional formulations that were prepared and tested are shown below:

TABLE 45
Pre-Lyo

| | NaCl | modRNA | Sucrose | RSV | PS 80 | Tris | Osmo |
|---|---|---|---|---|---|---|---|
| Formulation | (mM) | (mg/mL) | (%) | (mg/mL) | (%) | (mM) | (mOsm/kg) |
| 1 | 0 | 0.022 | 10.3 | 0.52 | 0.02 | 21.67 | 322 |
| 2 | 0 | 0.022 | 20.5 | 0.52 | 0.02 | 21.67 | 621 |
| 3 | 50.00 | 0.022 | 20.5 | 0.52 | 0.02 | 21.67 | 714 |
| 4 | 0 | 0.260 | 10.3 | 0.52 | 0.02 | 21.67 | 621 |
| 5 | 50.00 | 0.260 | 20.5 | 0.52 | 0.02 | 21.67 | 415 |
| 6 | 0 | 0.607 | 10.3 | 0.52 | 0.02 | 21.67 | 322 |
| 7 | 0 | 0.607 | 20.5 | 0.52 | 0.02 | 21.67 | 621 |
| 8 | 50.00 | 0.607 | 20.5 | 0.52 | 0.02 | 21.67 | 714 |

TABLE 46
Post-Recon

| | NaCl | modRNA | Sucrose | RSV | PS 80 | Tris | Osmo |
|---|---|---|---|---|---|---|---|
| Formulation | (mM) | (mg/mL) | (%) | (mg/mL) | (%) | (mM) | (mOsm/kg) |
| 1 | 0.00 | 0.01 | 5 | 0.24 | 0.01 | 10 | 150 |
| 2 | 0.00 | 0.01 | 9 | 0.24 | 0.01 | 10 | 288 |
| 3 | 23.08 | 0.01 | 9 | 0.24 | 0.01 | 10 | 331 |
| 4 | 0.00 | 0.12 | 5 | 0.24 | 0.01 | 10 | 193 |
| 5 | 23.08 | 0.12 | 9 | 0.24 | 0.01 | 10 | 193 |
| 6 | 0.00 | 0.28 | 5 | 0.24 | 0.01 | 10 | 150 |
| 7 | 0.00 | 0.28 | 9 | 0.24 | 0.01 | 10 | 288 |
| 8 | 23.08 | 0.28 | 9 | 0.24 | 0.01 | 10 | 331 |

The following is a stability data summary for a composition comprising a combination of modRNA flu and RSV subunit immunogenic compositions:
A) Results from formulations tested without NaCl, wherein 0.9% Saline was used as the solution for reconstitution

TABLE 47

| | | | | | | T1M | | |
|---|---|---|---|---|---|---|---|---|
| Quality Attribute (mRNA-LNP) | | BDP | T0 | T1W/35° C. | T2W/35° C. | 5° C. | 25° C. | 35° C. |
| Appearance | | White to off-white suspension, EFVP | | | | | | |
| pH | | 7.4 | 7.3 | 7.3 | 7.3 | | | |
| DLS | Size | 63 | 75 | 75 | 75 | | | |
| | PDI | 0.09 | 0.13 | 0.11 | 0.16 | | | |
| Ribogreen | Concentration (mg/mL) | 0.26 | 0.12 | 0.13 | 0.13 | | | |
| | Encapsulation (%) | 97 | 77 | 77 | 78 | | | |

| | | | | | | | |
|---|---|---|---|---|---|---|---|
| FA | 83 | 62 | 74 | 68 | | | |
| IVE (%) | 98 | 95 | 97 | 97 | | | |
| Quality Attribute (RSV) | | | | | | | |
| Concentration | 540.4 | 247 | 230.3 | 227.7 | | | |
| Relative prefusion content | 111 | 89 | 90 | 81 | 88 | 85 | 80 |

The following is a stability data summary for a composition comprising a combination of modRNA flu and RSV subunit immunogenic compositions:
B) Results from formulations tested with NaCl, wherein 0.9% Saline was used as the solution for reconstitution

TABLE 48

| | | | | | | T1M | | |
|---|---|---|---|---|---|---|---|---|
| Quality Attribute (mRNA-LNP) | | BDP | T0 | T1W/35° C. | T2W/35° C. | 5° C. | 25° C. | 35° C. |
| Appearance | | White to off-white suspension, EFVP | | | | | | |
| pH | | 7.3 | 7.3 | 7.3 | 7.2 | | | |
| DLS | Size | 63 | 74 | 78 | 77 | | | |
| | PDI | 0.12 | 0.11 | 0.12 | 0.11 | | | |
| Ribogreen | Concentration (mg/mL) | 0.26 | 0.12 | 0.13 | 0.13 | | | |
| | Encapsulation (%) | 97 | 72 | 70 | 71 | | | |

| | | | | | | | |
|---|---|---|---|---|---|---|---|
| FA | 84 | 57 | 74 | 72 | | | |
| IVE (%) | 97 | 97 | 97 | 98 | | | |
| Quality Attribute (RSV) | | | | | | | |
| Concentration | 550.8 | 248.7 | 244.3 | 229.5 | | | |
| Relative prefusion content | 118 | 93 | 92 | 87 | 91 | 91 | 86 |

### Example 21: Description and Composition of an RSV Subunit and Influenza mRNA Combination Drug Product

The RSVpreF subunit and Influenza mRNA Combination Vaccine (PF-07941314) for intramuscular injection was prepared by mixing the bivalent RSVpreF (847A and 847B) (PF-06928316) and the modRNA Quadrivalent Influenza vaccine (Wisconsin, Phuket, Austria and Darwin) (PF-07871992) at the time of administration, for co-administration. The present Example describes a co-administration, which differs from Example 20, for example, which describes a co-formulated composition.

The RSVpreF & Influenza mRNA Combination composition was prepared by mixing two drug products, the RSVpreF and Influenza modRNA Quadrivalent compositions (Wisconsin, Phuket, Austria, and Darwin). The RSVpreF composition is a preservative-free sterile lyophilized powder that comprises equal amounts of two stabilized drug substance protein antigens, 847A and 847B. Prior to use for intramuscular injection, the lyophilized drug product was reconstituted with a prefilled syringe containing sterile water diluent. The Influenza modRNA Quadrivalent composition is a preservative-free, sterile dispersion of lipid nanoparticles (LNPs) in aqueous cryoprotectant buffer for intramuscular injection. The sterile water reconstituted RSVpreF vaccine formulation is 0.24 mg/ml drug product in 15 mM Tris buffer, 37.5 mM NaCl, 2.25% sucrose, 4.5% mannitol, and 0.015% PS80 at pH 7.4. The Influenza modRNA Quadrivalent vaccine is formulated at 0.12 mg/ml RNA in 10 mM Tris buffer, 300 mM sucrose, pH 7.4.

The target compositions of the respective vaccines, RSVpreF and Influenza mRNA are provided in Table 49 and Table 50, respectively. The target composition of the RSV diluent is provided in Table 49.

TABLE 49

| | | Amount/dose |
|---|---|---|
| Ingredient | | (mg) |
| 847A DS | Active Ingredient | 0.06 |
| 847B DS | Active Ingredient | 0.06 |
| Tromethamine base | Buffer | 0.11 |
| Tris-Hydrochloride | Buffer | 1.04 |
| Sucrose | Cryoprotectant | 11.25 |
| Mannitol | Bulking agent | 22.50 |
| Polysorbate 80 | Surfactant | 0.08 |
| Sodium Chloride | Tonicifier | 1.1 |
| Water for Injection | Solvent | q.s to 0.5 mL |

TABLE 50
0.12 mg/mL Quadrivalent Drug Product (PF-07871992) Composition

| | | | Unit | Nominal Amount |
|---|---|---|---|---|
| | Grade/Quality | | Formula | or Net Quantity |
| Name of Ingredient | Standard | Function | (mg/mL) | (Net mg/vial) |
| PF-07829855 Drug | In-house | Active | 0.03 | 0.015 |
| Substance (Wisconsin) | specification | ingredient | | |
| PF-07871853 Drug | In-house | Active | 0.03 | 0.015 |
| Substance (Darwin) | specification | ingredient | | |
| PF-07872963 Drug | In-house | Active | 0.03 | 0.015 |
| Substance (Austria) | specification | ingredient | | |
| PF-07836259 Drug | In-house | Active | 0.03 | 0.015 |
| Substance (Phuket) | specification | ingredient | | |
| ALC-0315^{a} | In-house | Functional lipid | 1.72 | 0.86 |
| | specification | | | |
| ALC-0159^{b} | In-house | Functional lipid | 0.21 | 0.11 |
| | specification | | | |
| DSPC^{c} | In-house | Structural lipid | 0.37 | 0.19 |
| | specification | | | |
| Cholesterol | Ph. Eur., NF | Structural lipid | 0.74 | 0.37 |
| Sucrose | Ph. Eur., NF | Cryoprotectant | 102.69 | 51.35 |
| Tromethamine (Tris | Ph. Eur., USP | Buffer | 0.20 | 0.10 |
| base) | | component | | |
| Tris (hydroxymethyl) | In-house | Buffer | 1.32 | 0.66 |
| aminomethane | specification | component | | |
| hydrochloride (Tris HCl) | | | | |
| Water for Injection | Ph. Eur., USP, | Solvent | q.s.^{d} | q.s.^{d} |
| | JP | | to 1.00 mL | to 0.50 mL |

^{a}ALC-0315 = ((4-hydroxybutyl)azanediyl)bis(hexane-6,1-diyl)bis(2-hexyldecanoate)
^{b}ALC-0159 = 2-[(polyethylene glycol)-2000]-N,N-ditetradecylacetamide
^{c}DSPC = 1,2-Distearoyl-sn-glycero-3-phosphocholine
^{d}q.s. is an abbreviation for quantum satis meaning as much as is sufficient.

Dosage verification studies were performed to demonstrate that RSVpreF and Influenza modRNA Quadrivalent compositions were compatible when mixed for administration, and that all drug product and dosing solutions were compatible with the administration components for a period of time adequate to perform the dose preparation and administration operations. The dosage verification study design as detailed in Table 52 was based on a bracketing approach designed to support lowest and highest final clinical doses of 180 μg (1:2 ratio of Influenza mRNA:RSVpreF) and 210 μg (1:1.33 of Influenza mRNA:RSVpreF). The stability of the combined compositions in polycarbonate (PC) syringes, polypropylene (PP) syringes, and glass vials held at 30° C. up to 4 hours was assessed. Doses were prepared by mixing the appropriate volumes of the individual vaccines and administering as a single injection of the combined vaccines.

TABLE 51
Dose Calculations for Dosage Form Configurations of
PF-07941314 Combination Vaccine for IM Injection

| | 180 μg Dose (Group 1) | | 210 μg Dose (Group 2) | |
|---|---|---|---|---|
| | (1:2 | | (1:1.33 | |
| | Influenza:RSV) | | Influenza:RSV) | |
| | Final | Dose | Final | Dose |
| | Dose | Volume | Dose | Volume |
| | (μg) | (mL) | (μg) | (mL) |
| Influenza modRNA | 60 | 0.5 | 90 | 0.75 |
| vaccine (0.12 mg/mL) | | | | |
| RSVpreF (0.24 mg/mL) | 120 | 0.5 | 120 | 0.5 |
| Total | 180 | 1 | 210 | 1.25 |

Samples were tested according to a pre-determined experimental plan and analyzed by assays. Dosing solution samples were compared with the study criteria and their respective T0 samples.

TABLE 52
Dosing Verification Results for PF-07941314 Influenza mod mRNA & RSV Combination
Vaccine Group 1 [180 μg dose (1:2) Preparation]

| | Study | | 30° C. | | |
|---|---|---|---|---|---|
| Test | Criteria | T0 | T2H PP | T4H PP | T2H PC |
| Appearance | White to off- | WOWS | WOWS | WOWS | WOWS |
| | white | | | | |
| | suspension | | | | |
| Appearance | Essentially | EFVP | EFVP | EFVP | EFVP |
| (Visible | Free from | | | | |
| Particles) | Visible | | | | |
| | Particles | | | | |
| pH | 7.4 ± 0.5 | 7.3 | 7.3 | 7.2 | 7.2 |
| RNA Content | T0 ± 20% | 62 | 59 | 61 | 59 |
| (Fluorescence) | | | | | |
| RNA Integrity | ≥50% Intact | 77 | 79 | 77 | 78 |
| (CGE by | RNA | | | | |
| fragment | | | | | |
| analyzer) | | | | | |
| RNA | ≥80% | 93 | 90 | 86 | 88 |
| Encapsulation | | | | | |
| (Fluorescence) | | | | | |
| LNP Size | 40-120 | 68 | 69 | 69 | 68 |
| (DLS) | nm | | | | |
| LNP PDI | ≤0.3 | 0.2 | 0.2 | 0.2 | 0.2 |
| (DLS) | | | | | |
| In vitro | Report | A/Wisc: 93%, | A/Wisc: 92%, | A/Wisc: 92%, | A/Wisc: 91%, |
| Expression | Result | A/Darwin: 95%, | A/Darwin: 95%, | A/Darwin: 95%, | A/Darwin: 94%, |
| | | B/Austria: 91%, | B/Austria: 90%, | B/Austria: 89%, | B/Austria: 89%, |
| | | B/Phuket: 85% | B/Phuket: 86% | B/Phuket: 86% | B/Phuket: 85% |
| RSV Protein | T0 ± 20% | 115 | aa | 113 | 103 |
| Concentration | (mcg/mL) | | | | |
| RSV HMMS | ≤25% | 2.4 | 5.3 | 4.5 | 6.1 |
| (SEC) | | | | | |
| RSV LMMS | ≤5% | 1.6 | 2.1 | 1.9 | 1.7 |
| (SEC) | | | | | |
| RSV Trimer | ≥70% | 96.0 | 92.6 | 93.5 | 92.2 |
| (SEC) | | | | | |
| RSV Relative | 50-150% | 81 | 94 | 80 | 88 |
| Prefusion | | | | | |
| Content | | | | | |
| (Prefusion F | | | | | |
| ELISA) | | | | | |

| | 30° C. | | |
|---|---|---|---|
| Test | T4H PC | T2H Vial | T4H Vial |
| Appearance | WOWS | WOWS | WOWS |
| Appearance | EFVP | EFVP | EFVP |
| (Visible | | | |
| Particles) | | | |
| pH | 7.3 | 7.3 | 7.3 |
| RNA Content | 54 | 58 | 53 |
| (Fluorescence) | | | |
| RNA Integrity | 77 | 79 | 78 |
| (CGE by | | | |
| fragment | | | |
| analyzer) | | | |
| RNA | 83 | 86 | 83 |
| Encapsulation | | | |
| (Fluorescence) | | | |
| LNP Size | 69 | 68 | 70 |
| (DLS) | | | |
| LNP PDI | 0.2 | 0.2 | 0.2 |
| (DLS) | | | |
| In vitro | A/Wisc: 91%, | A/Wisc: 92%, | A/Wisc: 98%, |
| Expression | A/Darwin: 94%, | A/Darwin: 96%, | A/Darwin: 97%, |
| | B/Austria: 85%, | B/Austria: 85%, | B/Austria: 86%, |
| | B/Phuket: 83% | B/Phuket: 87% | B/Phuket: 95% |
| RSV Protein | 107 | 102 | 104 |
| Concentration | | | |
| RSV HMMS | 5.9 | 4.0 | 5.7 |
| (SEC) | | | |
| RSV LMMS | 2.0 | 1.9 | 1.9 |
| (SEC) | | | |
| RSV Trimer | 92.1 | 94.1 | 92.4 |
| (SEC) | | | |
| RSV Relative | 89 | 83 | 105 |
| Prefusion | | | |
| Content | | | |
| (Prefusion F | | | |
| ELISA) | | | |

Abbreviations:
PP = polypropylene;
PC = polycarbonate;
WOWS = White to off-white suspension;
EFVP = Essentially Free of Visible Particles;
LNP = Lipid Nano Particle;
CGE = capillary gel electrophoresis;
DLS = Dynamic Light Scattering;
HMMS = high molecular weight species;
LMMS = low molecular weight species;
SEC = size exclusion chromatograph

TABLE 53
T Dosing Verification Results for PF-07941314 Influenza mod mRNA & RSV Combination
Vaccine Group 2 [210 μg dose (1:1.33) Preparation]

| | Expected | | 30° C. | | |
|---|---|---|---|---|---|
| Test | Results | T0 | T2H PP | T4H PP | T2H PC |
| Appearance | White to | WOWS | WOWS | WOWS | WOWS |
| | off-white | | | | |
| | suspension | | | | |
| Appearance | Essentially | EFVP | EFVP | EFVP | EFVP |
| (Visible | Free | | | | |
| Particles) | from | | | | |
| | Visible | | | | |
| | Particles | | | | |
| | (EFVP) | | | | |
| pH | 7.4 ± 0.5 | 7.2 | 7.2 | 7.3 | 7.2 |
| RNA Content | T0 ± 20% | 75 | 71 | 70 | 81 |
| (Fluorescence) | | | | | |
| RNA Integrity | ≥50% | 81 | 83 | 80 | 81 |
| (CGE by | Intact | | | | |
| fragment | RNA | | | | |
| analyzer) | | | | | |
| RNA | ≥80% | 90 | 89 | 89 | 85 |
| Encapsulation | | | | | |
| (Fluorescence) | | | | | |
| LNP Size | 40-120 | 67 | 68 | 67 | 68 |
| (DLS) | nm | | | | |
| LNP PDI | ≤0.3 | 0.16 | 0.16 | 0.14 | 0.15 |
| (DLS) | | | | | |
| In vitro | Report | A/Wisc: 89%, | A/Wisc: 87%, | A/Wisc: 87%, | A/Wisc: 90%, |
| Expression | Result | A/Darwin: 86%, | A/Darwin: 86%, | A/Darwin: 83%, | A/Darwin: 87%, |
| | | B/Austria: 88%, | B/Austria: 87%, | B/Austria: 88%, | B/Austria: 87%, |
| | | B/Phuket: 80% | B/Phuket: 79% | B/Phuket: 77% | B/Phuket: 74% |
| Protein | T0 ± 20% | 85 | 85 | 84 | 78 |
| Concentration | (mcg/mL) | | | | |
| HMMS (SEC) | ≤25% | 1.7 | 4.6 | 6.1 | 11.7 |
| LMMS (SEC) | ≤5% | 1.9 | 2.1 | 1.7 | 1.8 |
| Trimer (SEC) | ≥70% | 96.4 | 93.3 | 92.2 | 86.5 |
| Relative | 50-150% | 90 | 96 | 95 | 90 |
| Prefusion | | | | | |
| Content | | | | | |
| (Prefusion F | | | | | |
| ELISA) | | | | | |

| | 30° C. | | |
|---|---|---|---|
| Test | T4H PC | T2H Vial | T4H Vial |
| Appearance | WOWS | WOWS | WOWS |
| Appearance | EFVP | EFVP | EFVP |
| (Visible | | | |
| Particles) | | | |
| pH | 7.2 | 7.2 | 7.2 |
| RNA Content | 71 | 73 | 76 |
| (Fluorescence) | | | |
| RNA Integrity | 81 | 81 | 81 |
| (CGE by | | | |
| fragment | | | |
| analyzer) | | | |
| RNA | 87 | 89 | 88 |
| Encapsulation | | | |
| (Fluorescence) | | | |
| LNP Size | 68 | 69 | 69 |
| (DLS) | | | |
| LNP PDI | 0.16 | 0.17 | 0.18 |
| (DLS) | | | |
| In vitro | A/Wisc: 87%, | A/Wisc: 88%, | A/Wisc: 90%, |
| Expression | A/Darwin: 83%, | A/Darwin: 85%, | A/Darwin: 85%, |
| | B/Austria: 83%, | B/Austria: 82%, | B/Austria: 80%, |
| | B/Phuket: 71% | B/Phuket: 74% | B/Phuket: 71% |
| Protein | 81 | 85 | 84 |
| Concentration | | | |
| HMMS (SEC) | 7.7 | 4.2 | 3.1 |
| LMMS (SEC) | 1.7 | 1.6 | 1.9 |
| Trimer (SEC) | 90.6 | 94.2 | 95.0 |
| Relative | 100 | 107 | 96 |
| Prefusion | | | |
| Content | | | |
| (Prefusion F | | | |
| ELISA) | | | |

Abbreviations:
PP = polypropylene;
PC = polycarbonate;
WOWS = White to off-white suspension;
EFVP = Essentially Free of Visible Particles;
LNP = Lipid Nano Particle;
CGE = capillary gel electrophoresis;
DLS = Dynamic Light Scattering;
HMMS = high molecular weight species;
LMMS = low molecular weight species;
SEC = size exclusion chromatograph

### Example 22: a Study to Evaluate the Safety, Tolerability, and Immunogenicity of Respiratory Combination Vaccine Candidates in Older Adults (C5401001)

Substudy B will investigate 2 formulations of RSVpreF+qIRV of differing volumes and osmolarities. These formulations will be evaluated for safety, tolerability, and immunogenicity.

The formulation development of combination vaccines includes consideration of both volume administered and tonicity to determine whether the ultimate product achieves the desired effects in a safe and tolerable manner. In some cases, consideration of exceeding the theoretical threshold of 600 mOsm/kg is necessary to ensure that adequate antigen and excipients are contained within the combination formulation.

This is a Phase 1b study in healthy participants ≥50 years of age. Substudy B will investigate 2 formulations of RSVpreF+qIRV of differing volumes and osmolarities. The first combination is a bedside mix of RSVpreF lyophilized cake reconstituted with WFI and mixed with qIRV, yielding a final injection volume of 1.0 mL (theoretical osmolarity of 406 mOsm/kg).

The second combination is a bedside mix of RSVpreF lyophilized cake directly reconstituted with qIRV to yield a final injection volume of 0.5 mL. The 0.5-mL direct reconstitution combination would result in a hyperosmotic (855 mOsm/kg) solution with the potential to influence reactogenicity at the injection site.

These combinations will be evaluated for safety, tolerability, and immunogenicity, with the goal of selecting a formulation for further study.

Substudy B will be conducted as a Phase 1b, parallel-group, randomized, observer-blinded substudy of RSVpreF and a nucleoside-modified RNA vaccine (qIRV) against RSV and influenza administered as a combined vaccine in a 1.0-mL formulation and when administered in a 0.5-mL formulation.

Healthy adults ≥50 years of age will be randomized 1:1 to either Group 1: combination (RSVpreF+qIRV) 1.0-mL formulation; or Group 2: combination (RSVpreF+qIRV) 0.5 mL formulation. Randomization will be stratified by 6 age/sex groups (female 50-64, female 65-79, and female ≥80 years; male 50-64, male 65-79, and male ≥80 years) within the study site. Approximately 100 participants will be randomized within the 50- through 64-year-old group, and 100 participants ≥65 years of age will be randomized.

### Study Interventions for Substudy B

TABLE 54

| | Vaccine Details | | |
|---|---|---|---|
| GROUP 1 | | | |
| COMBINATION | Vaccine | RSVpreF | qIRV (modRNA) |
| 1.0 mL | Component | | |
| (RSVpreF + | | 2 Stabilized RSV prefusion | Encoding HA of 4 strains as |
| qIRV) | | F antigens, in equal amounts | recommended for the influenza |
| | | from virus subgroups A and B | season (2 A strains and 2 B |
| | | | strains) |
| | Dose | RSV vaccine 120 μg/vial | Quadrivalent influenza |
| | Formulation | powder for solution for injection | suspension for injection, |
| | | | 0.12 mg/mL (qIRV) |
| | Product for | Sterile water as diluent for | N/A |
| | Reconstitution | injection (0.65 mL/syringe) | |
| | Unit Dose | 120 μg | 60 μg |
| | Strength(s) | | |
| | Route of | After combining both vaccines, a single 1-mL IM injection is | |
| | Administration | given into the deltoid muscle of the nondominant arm | |
| | | (preferred). | |
| | Packaging and | RSVpreF is supplied as a lyophilized white cake, packaged in a | |
| | Labeling | clear glass 2-mL vial with a rubber stopper, aluminum overseal, | |
| | | and flip-off cap. For the 1.0-mL formulation, the white cake is | |
| | | reconstituted with a PFS of sterile water diluent for injection | |
| | | (supplied) and then combined with the qIRV suspension. | |
| | | qIRV is supplied as a white to off-white suspension in a 2-mL | |
| | | type 1 clear glass vial, sealed with a 13-mm serum stopper and | |
| | | 13-mm aluminum flip-off seal. Fill volume is 0.68 mL. | |
| GROUP 2 | | | |
| COMBINATION | Vaccine | RSVpreF | qIRV |
| 0.5 mL | Component | 2 Stabilized RSV prefusion | Encoding HA of 4 strains as |
| (RSVpreF + | | F antigens, in equal amounts | recommended for the |
| qIRV) | | from virus subgroups A and B | influenza season (2 A strains |
| | | | and 2 B strains) |
| | Dose | RSV vaccine 120 μg/vial powder | Quadrivalent influenza |
| | Formulation | for solution for injection | suspension for injection, |
| | | | 0.12 mg/mL (qIRV) |
| | Product for | qIRV suspension | N/A |
| | Reconstitution | | |
| | Unit Dose | 120 μg | 60 μg |
| | Strength(s) | | |

| | |
|---|---|
| Route of | After combining qIRV suspension with the RSVpreF cake, a |
| Administration | single |
| | 0.5-mL IM injection is given into the deltoid muscle of the |
| | nondominant arm (preferred). |
| Packaging and | RSVpreF is supplied as a lyophilized white cake, packaged in a |
| Labeling | clear glass 2-mL vial with a rubber stopper, aluminum overseal, |
| | and flip-off cap. For the 0.5-mL formulation, the white cake is to |
| | be directly reconstituted with the qIRV suspension. |
| | qIRV is supplied as a white to off-white suspension in a 2-mL |
| | type 1 clear glass vial, sealed with a 13-mm serum stopper and |
| | 13-mm aluminum flip-off seal. Fill volume is 0.68 mL. |

### Clauses

Several exemplary embodiments of the invention are described by the following enumerated clauses:
1. A composition comprising: (i) a first ribonucleic acid (RNA) polynucleotide comprising an open reading frame encoding a first antigen, said antigen comprising at least one influenza virus antigenic polypeptide or an immunogenic fragment thereof, wherein the first RNA polynucleotide is formulated in a lipid nanoparticle (LNP); and (ii) a first RSV F protein trimer in the prefusion conformation.
2. The composition according to Clause 1, further comprising (iii) a second RNA polynucleotide comprising an open reading frame encoding a second antigen, said second antigen comprising at least one influenza virus antigenic polypeptide or an immunogenic fragment thereof.
3. The composition according to any one of Clauses 1-2, wherein the antigens comprise hemagglutinin (HA), or an immunogenic fragment or variant thereof.
4. The composition according to any one of Clauses 1-3, wherein the antigens each comprise an HA, or an immunogenic fragment thereof, that are from different subtypes of influenza virus.
5. The composition of any one of Clauses 1-4, further comprising a third RNA polynucleotide comprising an open reading frame encoding an antigen comprising at least one influenza virus antigenic polypeptide or an immunogenic fragment thereof.
6. The composition according to Clause 5, wherein the third antigen is from an influenza virus different from the strain of influenza virus of both the first and second antigens.
7. The composition of Clause 6, wherein the first, second and third RNA polynucleotides are formulated in a lipid nanoparticle.
8. The composition of Clause 7, further comprising a fourth RNA polynucleotide comprising an open reading frame encoding a fourth antigen, said antigen comprising at least one influenza virus antigenic polypeptide or an immunogenic fragment thereof.
9. The composition according to Clause 8, wherein the fourth antigen is from influenza virus but is from a different strain of influenza virus to the first, second and third antigens.
10. The composition of Clause 8, wherein the first, second, third, and fourth RNA polynucleotides are formulated in a lipid nanoparticle.
11. The composition of any one of Clauses 1-10, wherein the RNA polynucleotides are present in about equal ratios.
12. The composition of any one of Clauses 1-11, wherein any one of the RNA polynucleotides comprises a modified nucleotide.
13. The composition of Clause 12, wherein the modified nucleotide is selected from the group consisting of pseudouridine, 1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.
14. The composition of any one of Clauses 1-13, wherein each RNA polynucleotide comprises a 5′ terminal cap, a 5′ UTR, a 3′UTR, and a 3′ polyadenylation tail.
15. The composition of Clause 14, wherein the 5′ terminal cap comprises:

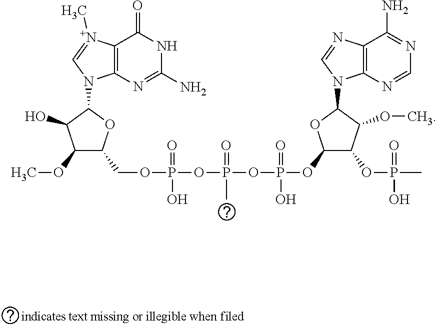

16. The composition of Clause 14, wherein the 5′ UTR comprises SEQ ID NO: 1.
17. The composition of Clause 14, wherein the 3′ UTR comprises SEQ ID NO: 2.
18. The composition of Clause 14, wherein the 3′ polyadenylation tail comprises SEQ ID NO: 3.
19. The composition of any one of Clauses 1-18, wherein the RNA polynucleotide has an integrity greater than 85%.
20. The composition of any one of Clauses 1-19, wherein the RNA polynucleotide has a purity of greater than 85%.
21. The composition of any one of Clauses 1-20, wherein the lipid nanoparticle comprises 20-60 mol % ionizable cationic lipid, 5-25 mol % neutral lipid, 25-55 mol % cholesterol, and 0.5-5 mol % polymer-modified lipid.
22. The composition of any one of Clauses 1-21, wherein the cationic lipid comprises:

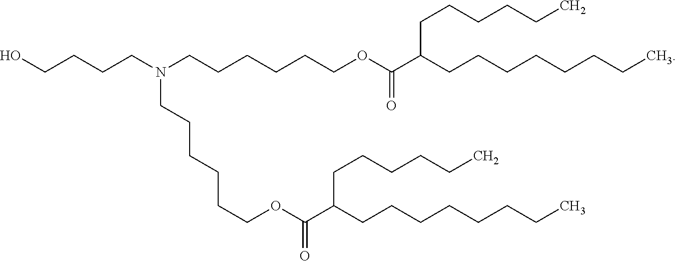 [SMILES: CCCCCCCCC(CCCCCC)C(=O)OCCCCCCN(CCCCO)CCCCCCOC(=O)C(CCCCCC)CCCCCCCC]

23. The composition of any one of Clauses 1-22, wherein the PEG-modified lipid comprises:

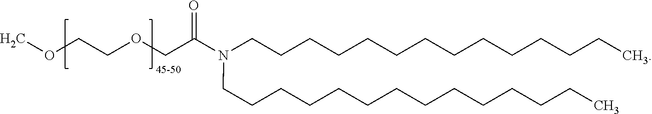

24. The composition of any one of Clauses 1-23, wherein the first antigen is HA from influenza A subtype H1 or an immunogenic fragment or variant thereof and the second antigen is HA from a different H1 strain to the first antigen or an immunogenic fragment or variant thereof.
25. The composition of any one of Clauses 1-24, wherein the first and second antigens are HA from influenza A subtype H3 or an immunogenic fragment or variant thereof and wherein both antigens are derived from different strains of H3 influenza virus.
26. The composition of any one of Clauses 1-25, wherein the first and second antigens are HA from influenza A subtype H1 or an immunogenic fragment or variant thereof and the third and fourth antigens are from influenza A subtype H3 or an immunogenic fragment or variant thereof and wherein the first and second antigens are derived from different strains of H1 virus and the third and fourth antigens are from different strains of H3 influenza virus.
27. The composition of any one of Clauses 1-26, wherein at least the first and second RNA polynucleotides are formulated in a single lipid nanoparticle.
28. The composition of any one of Clauses 1-27, wherein the first, second, and third RNA polynucleotides are formulated in a single lipid nanoparticle.
29. The composition of any one of Clauses 1-28, wherein the first, second, third, and fourth RNA polynucleotides are formulated in a single LNP.
30. The composition of any one of Clauses 1-26, wherein each of the RNA polynucleotides is formulated in a single LNP, wherein each single LNP encapsulates the RNA polynucleotide encoding one antigen.
31. The composition of Clause 30, wherein the first RNA polynucleotide is formulated in a first LNP; and the second RNA polynucleotide is formulated in a second LNP.
32. The composition of Clause 30, wherein the first RNA polynucleotide is formulated in a first LNP; the second RNA polynucleotide is formulated in a second LNP; and the third RNA polynucleotide is formulated in a third LNP.
33. The composition of Clause 30, wherein the first RNA polynucleotide is formulated in a first LNP; the second RNA polynucleotide is formulated in a second LNP; the third RNA polynucleotide is formulated in a third LNP; and the fourth RNA polynucleotide is formulated in a fourth LNP.
34. The composition of any one of Clauses 1-30, for use in the eliciting an immune response against influenza in a subject.

The composition of according any one of Clause 1-33, wherein the first RSV F protein is an F protein of subtype A or B.
35. The composition of according to any of Clauses 1-34, wherein the first RSV F protein comprises a combination of mutations relative to the corresponding wild-type RSV F protein, wherein the combination of mutations is selected from the group consisting of: (1) combination of 103C, 148C, 1901, and 486S, preferably A103C, 1148C, S1901, and D486S; (2) combination of 54H, 55C, 188C, and 486S, preferably T54H, S55C, L188C, and D486S; (3) combination of 54H, 103C, 148C, 1901, 2961, and 486S, preferably T54H, A103C, 1148C, S1901, V2961, and D486S; (4) combination of 54H, 55C, 142C, 188C, 2961, and 371C, preferably T54H, S55C, L142C, L188C, V2961, and N371C; (5) combination of 55C, 188C, and 486S, preferably S55C, L188C, and D486S; (6) combination of 54H, 55C, 188C, and 1901, preferably T54H, S55C, L188C, and S1901; (7) combination of 55C, 188C, 1901, and 486S, preferably S55C, L188C, S1901, and D486S; (8) combination of 54H, 55C, 188C, 1901, and 486S, preferably T54H, S55C, L188C, S1901, and D486S; (9) combination of 155C, 1901, 290C, and 486S, preferably S155C, S1901, S290C, and D486S; (10) combination of 54H, 55C, 142C, 188C, 2961, 371C, 486S, 487Q, and 489S, preferably T54H, S55C, L142C, L188C, V2961, N371C, D486S, E487Q, and D489S; (11) combination of 54H, 155C, 1901, 290C, and 2961, preferably T54H, S155C, S1901, S290C, and V2961, and (12) combination of 155C, 190F, 290C, and 207L, preferably S155C, S190F, S290C, and V207L.
36. The composition according to Clause 35, wherein the first RSV F protein comprises a combination of mutations relative to the corresponding wild-type RSV F protein, wherein the combination of mutations is selected from the group consisting of: (1) combination of 215P and 486N, preferably S215P and D486N, (2) combination of 66E, 215P, and 486N, preferably K66E, S215P, and D486N, (3) combination of 66E, 76V, 215P, and 486N, preferably K66E, 176V, S215P, and D486N, and, (4) combination of 66E, 671, 76V, 215P, and 486N, preferably K66E, N671, 176V, S215P, and D486N.
37. The composition according to any one of Clauses 1-36, wherein the first RSV F protein comprises a trimerization domain.
38. The composition according to any one of Clauses 1-37, wherein the composition further comprises a second RSV F protein trimer in the prefusion conformation.
39. The composition according to Clause 37, wherein the second RSV F protein is an F protein of subtype A or B.
40. The composition according to Clause 39 or 40, wherein the second RSV F protein comprises a combination of mutations relative to the corresponding wild-type RSV F protein, wherein the combination of mutations is selected from the group consisting of: (1) combination of 103C, 148C, 1901, and 486S, preferably A103C, 1148C, S1901, and D486S; (2) combination of 54H, 55C, 188C, 486S, preferably T54H, S55C, L188C, and D486S; (3) combination of 54H, 103C, 148C, 1901, 2961, and 486S, preferably T54H, A103C, 1148C, S1901, V2961, and D486S; (4) combination of 54H, 55C, 142C, 188C, 2961, and 371C, preferably T54H, S55C, L142C, L188C, V2961, and N371C; (5) combination of 55C, 188C, and 486S, preferably S55C, L188C, and D486S; (6) combination of 54H, 55C, 188C, and 1901, preferably T54H, S55C, L188C, and S1901; (7) combination of 55C, 188C, 1901, and 486S, preferably S55C, L188C, S1901, and D486S; (8) combination of 54H, 55C, 188C, 1901, and 486S, preferably T54H, S55C, L188C, S1901, and D486S; (9) combination of 155C, 1901, 290C, and 486S, preferably S155C, S1901, S290C, and D486S; (10) combination of 54H, 55C, 142C, 188C, 2961, 371C, 486S, 487Q, and 489S, preferably T54H, S55C, L142C, L188C, V2961, N371C, D486S, E487Q, and D489S; (11) combination of 54H, 155C, 1901, 290C, and 2961, preferably T54H, S155C, S1901, S290C, and V2961, and (12) combination of 155C, 190F, 290C, and 207L, preferably S155C, S190F, S290C, and V207L.
41. The composition according to any one of Clauses 39-41, wherein the second RSV F protein comprises a combination of mutations relative to the corresponding wild-type RSV F protein, wherein the combination of mutations is selected from the group consisting of: (1) combination of 215P and 486N, preferably S215P and D486N, (2) combination of 66E, 215P, and 486N, preferably K66E, S215P, and D486N, (3) combination of 66E, 76V, 215P, and 486N, preferably K66E, 176V, S215P, and D486N, and, (4) combination of 66E, 671, 76V, 215P, and 486N, preferably K66E, N671, 176V, S215P, and D486N.
42. The composition according to any one of Clauses 39-42, wherein the second RSV F protein comprises a trimerization domain.
43. The composition according to any one of Clauses 1-43, wherein the first RSV F protein trimer is subtype A; wherein the composition further comprises a second RSV F protein trimer in the prefusion conformation, said second RSV F protein trimer is subtype B.
44. The composition according to any one of Clauses 1-44, wherein the composition further comprises sodium chloride at a concentration of between about 20 mM and about 250 mM; (iii) at least one of sucrose, mannitol and glycine at a concentration of between about 5 mg/mL and about 100 mg/mL; and (iv) a buffer; wherein the pH of said composition is between about 7 and about 8.
45. The composition according to any one of Clauses 1-45, wherein the LNP is in a liquid state and the first RSV F protein trimer is lyophilized.
46. The composition according to Clause 46, wherein the osmolality of the composition is at most 500 mOsm/kg.
47. The composition according to any one of Clauses 1-47, wherein each RNA has at least 50% integrity, as measured by fragment analyzer.
48. The composition according to any one of Clauses 1-48, wherein each LNP has at least 80% encapsulation efficiency for at least 4 hours.
49. The composition according to any one of Clauses 1-49 further comprising sodium chloride at a concentration of between about 20 mM and about 250 mM; (iii) at least one of sucrose, mannitol and glycine at a concentration of between about 5 mg/mL and about 100 mg/mL; and (iv) a buffer; wherein the pH of said composition is between about 7 and about 8.
50. The composition according to any one of Clauses 1-50, wherein the RNA polynucleotide is purified and is substantially free of contaminants comprising short abortive RNA species, long abortive RNA species, double-stranded RNA (dsRNA), residual plasmid DNA, residual in vitro transcription enzymes, residual solvent and/or residual salt.
51. The composition according to any one of Clauses 1-51, wherein the RSV polypeptide is purified and is substantially free of contaminants.
52. The composition according to any one of Clauses 1-52, wherein the composition further comprises an adjuvant.
53. The composition according to Clause 53, wherein the adjuvant is in association with the first RSV protein trimer and/or the second RSV protein trimer.
54. The composition according to any one of Clauses 1-52, wherein the composition does not comprise an adjuvant.
55. The composition according to any one of Clauses 1-55, wherein the adjuvant comprises aluminum.
56. A composition comprising: (i) a first ribonucleic acid (RNA) polynucleotide comprising an open reading frame encoding a first antigen, said antigen comprising at least one influenza virus antigenic polypeptide or an immunogenic fragment thereof, wherein the first RNA polynucleotide is formulated in a lipid nanoparticle (LNP); (ii) a second RNA polynucleotide comprising an open reading frame encoding a second antigen, said second antigen comprising at least one influenza virus antigenic polypeptide or an immunogenic fragment thereof; (iii) a third RNA polynucleotide comprising an open reading frame encoding an antigen comprising at least one influenza virus antigenic polypeptide or an immunogenic fragment thereof; (iv) a fourth RNA polynucleotide comprising an open reading frame encoding a fourth antigen, said antigen comprising at least one influenza virus antigenic polypeptide or an immunogenic fragment thereof; and (v) an RNA polynucleotide comprising at least one open reading frame encoding at least one respiratory syncytial virus (RSV) antigenic polypeptide or an immunogenic fragment thereof.
57. The composition according to Clause 57, wherein the RSV antigenic polypeptide has at least 90%, 95%, 96%, 97%, 98% or 99% identity to the amino acid sequence selected from SEQ ID NO: 1 to 6.
58. The composition according to any one of Clauses 57-58, wherein the RNA polynucleotide comprising at least one open reading frame encoding at least one respiratory syncytial virus (RSV) antigenic polypeptide or an immunogenic fragment thereof comprises the sequence set forth in any one of SEQ ID NO: 13, SEQ ID NO: 14, SEQ ID NO: 15, and SEQ ID NO: 16.
59. The composition according to any one of Clauses 57-59, wherein the RSV antigenic polypeptide is derived from RSV subtype A and/or RSV subtype B.
60. The composition according to any one of Clauses 57-60, wherein each of said RNA polynucleotide comprises a 5′ cap, 5′ UTR, 3′ UTR, and poly-A tail.
61. The composition according to any one of Clauses 57-61, wherein any one of said RNA polynucleotide comprises at least one modified nucleotide selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine OR 2′-O-methyl uridine.
62. The composition according to any one of Clauses 57-62, wherein each of said RNA polynucleotide is encapsulated in a lipid nanoparticle (LNP).
63. The composition according to any one of Clauses 57-63, wherein the LNP comprises a cationic lipid, a polymer-lipid, a neutral lipid, and a steroid or steroid analog.
64. A method of eliciting an immune response against influenza in a subject, comprising administering an effective amount of a composition according to any one of Clauses 1-64.
65. A method of eliciting an immune response against influenza and RSV in a subject, comprising administering an effective amount of a composition according to any one of Clauses 1-64.
66. A method of preventing, treating or ameliorating an infection, disease or condition associated with influenza and/or RSV in a subject, comprising administering to a subject an effective amount of a composition according to any one of Clauses 1-64.
67. The method according to any one of Clauses 65-67, wherein the subject is less than about 1 year of age, about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older.
68. The method according to any one of Clauses 65-67, wherein the composition according to any one of Clauses 1-64 is administered by intradermal or intramuscular injection.
69. An article of manufacture comprising a vial having (a) a first chamber that comprises a first composition comprising an RNA polynucleotide; (b) a second chamber that comprises a lyophilized composition comprising a polypeptide; and (c) a septum separating the first and second chambers and impermeable to the gaseous medium; and (d) actuating means effective to bring the first composition and the second composition into contact by breach of the septum.
70. A method of eliciting an immune response against a disease associated with influenza in a subject, comprising administering to the subject an effective amount of the composition according to any one of Clauses 1-64, wherein the subject is selected from a human population of at least 18 years of age, wherein the vaccine is provided in a dose volume of between about 0.05 and about 0.2 ml and wherein the vaccine induces a protective immune response in the human population of at least 18 years of age.
71. The method according to Clause 71, wherein the composition is administered intradermally to the subject.
72. The method according to any one of Clauses 71-72, wherein said protective immune response meets at least one criterion chosen from the group of: (1) a protection rate of >70% in adults of 18-60 years of age or of >60% in elderly people of above 60 years of age; (2) a seroconversion rate of >40% in adults of 18-60 years of age or of >30% in elderly people of above 60 years of age; or (3) a conversion factor of >2.5 HI titres in adults of 18-60 years of age or of >2.0 HI titres in elderly people of above 60 years of age, for each influenza strain. The method according to any one of Clauses 71-72, wherein said protective immune response meets at least one criterion chosen from the group of: (1) a protection rate of >70% in adults of 18-60 years of age or of >60% in elderly people of above 60 years of age; (2) a seroconversion rate of >40% in adults of 18-60 years of age or of >30% in elderly people of above 60 years of age; or (3) a conversion factor of >2.5 HI titres in adults of 18-60 years of age or of >2.0 HI titres in elderly people of above 60 years of age, for each influenza strain.
73. The method according to any one of Clauses 71-73, wherein said protective immune response meets at least two criteria chosen from the group of: (1) a protection rate of >70% in adults of 18-60 years of age or of >60% in elderly people of above 60 years of age; (2) a seroconversion rate of >40% in adults of 18-60 years of age or of >30% in elderly people of above 60 years of age; or (3) a conversion factor of >2.5 HI titres in adults of 18-60 years of age or of >2.0 HI titres in elderly people of above 60 years of age, for each influenza strain.
74. The method according to any one of Clauses 71-74, wherein said protective immune response comprises the geometric mean titer ratio is ≥0.74, as measured by a hemagglutination inhibition assay (HAI).
75. The method according to any one of Clauses 71-75, wherein the method further comprises eliciting an immune response against RSV.
76. The method according to Clause 76, wherein the immune response against RSV comprises eliciting 50% neutralizing geometric mean titer ratios ≥0.79.
77. A method of eliciting an immune response against a disease associated with RSV in a subject, comprising administering to the subject an effective amount of the composition according to any one of Clauses 1-64, wherein the subject is selected from a human population of at least 18 years of age, wherein the vaccine is provided in a dose volume of between about 0.05 and about 0.2 ml and wherein the vaccine induces a protective immune response in the human population of at least 18 years of age.
78. The method according to Clause 78, wherein the composition is administered intradermally to the subject.
79. The method according to any one of Clauses 78-79, wherein the immune response comprises the induction of antibodies specific for the RSV F protein in the pre-fusion conformation, and wherein the geometric mean titer (GMT) increase of antibodies specific for RSV F protein in the pre-fusion conformation is higher than the geometric mean titer (GMT) increase of antibodies specific for RSV F protein in the post-fusion conformation, in enzyme linked immunosorbent assays (ELISAs).
80. The method according to any one of Clauses 78-80, wherein the immune response comprises the induction of antibodies specific for the RSV F protein in the pre-fusion conformation, and wherein the geometric mean titer (GMT) increase of antibodies specific for RSV F protein in the pre-fusion conformation is higher than the geometric mean titer (GMT) increase of antibodies specific for RSV F protein prior to the administration.

All of the methods disclosed and claimed herein can be made and executed without undue experimentation in light of the present disclosure. While the compositions and methods of this disclosure have been described in terms of certain aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

The contents of all cited references (including literature references, issued patents, published patent applications, and GENBANK® Accession numbers as cited throughout this application) recited in the application, to the extent that they provide exemplary procedural or other details supplementary to those set forth herein, are hereby specifically and expressly incorporated by reference. When definitions of terms in documents that are incorporated by reference herein conflict with those used herein, the definitions used herein govern.

1. A method of eliciting an immune response against a disease associated with influenza in humans, comprising administering to the subject an effective amount of the composition, wherein the composition comprises (i) a first ribonucleic acid (RNA) polynucleotide comprising an open reading frame encoding at least one influenza virus hemagglutinin (HA) polypeptide or an immunogenic fragment thereof, wherein the first RNA polynucleotide is formulated in a lipid nanoparticle (LNP), wherein the LNP comprises an ionizable cationic lipid, a polymer-conjugated lipid, a neutral lipid, and a steroid; and (ii) a first RSV F protein trimer in the prefusion conformation.

2. (canceled)

3. The method according to claim 1, wherein the human is 18 years of age or older.

4. The method according to claim 1, wherein the RNA polynucleotide comprises at least one modified nucleotide selected from the group consisting of pseudouridine, N1-methylpseudouridine, N1-ethylpseudouridine, 2-thiouridine, thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine OR 2′-O-methyl uridine.

5. The method according to claim 1, wherein the LNP is in a liquid state and the first RSV F protein trimer is lyophilized.

6. The method according to claim 1, wherein each RNA has at least 50% integrity, as measured by fragment analyzer.

7. The method according to claim 1, wherein each LNP has at least 80% encapsulation efficiency for at least 4 hours.

8. The method according to claim 1, wherein said immune response meets at least a criterion of (1) a protection rate of >70% in adults of 18-60 years of age or of >60% in elderly people of above 60 years of age;

9. The method according to claim 1, wherein said immune response meets at least a criterion of a seroconversion rate of >40% in adults of 18-60 years of age or of >30% in elderly people of above 60 years of age;

10. The method according to claim 1, wherein said immune response meets at least a criterion of a conversion factor of >2.5 HI titers in adults of 18-60 years of age or of >2.0 HI titers in elderly people of above 60 years of age, for each influenza strain.

11. The method according to claim 1, wherein said immune response meets at least one criterion chosen from the group of: (1) a protection rate of >70% in adults of 18-60 years of age or of >60% in elderly people of above 60 years of age; (2) a seroconversion rate of >40% in adults of 18-60 years of age or of >30% in elderly people of above 60 years of age; or (3) a conversion factor of >2.5 HI titers in adults of 18-60 years of age or of >2.0 HI titers in elderly people of above 60 years of age, for each influenza strain.

12. The method according to claim 1, wherein said immune response meets at least two criteria chosen from the group of: (1) a protection rate of >70% in adults of 18-60 years of age or of >60% in elderly people of above 60 years of age; (2) a seroconversion rate of >40% in adults of 18-60 years of age or of >30% in elderly people of above 60 years of age; or (3) a conversion factor of >2.5 HI titers in adults of 18-60 years of age or of >2.0 HI titers in elderly people of above 60 years of age, for each influenza strain.

13. The method according to claim 1, wherein said immune response comprises the geometric mean titer ratio is ≥0.74, as measured by a hemagglutination inhibition assay (HAI).

14. The method according to claim 1, wherein the method further comprises eliciting an immune response against RSV.

15. The method according to claim 14, wherein the immune response against RSV comprises eliciting 50% neutralizing geometric mean titer ratios ≥0.79.

16. The method according to claim 1, wherein the vaccine is provided in a dose volume of between about 0.05 and about 0.2 ml.

17. The method according to claim 1, wherein the composition is administered intramuscularly to the subject.

18. The method according to claim 14, wherein the immune response comprises the induction of antibodies specific for the RSV F protein in the pre-fusion conformation, and wherein the geometric mean titer (GMT) increase of antibodies specific for RSV F protein in the pre-fusion conformation is higher than the geometric mean titer (GMT) increase of antibodies specific for RSV F protein in the post-fusion conformation, in enzyme linked immunosorbent assays (ELISAs).

19. The method according to claim 14, wherein the immune response comprises the induction of antibodies specific for the RSV F protein in the pre-fusion conformation, and wherein the geometric mean titer (GMT) increase of antibodies specific for RSV F protein in the pre-fusion conformation is higher than the geometric mean titer (GMT) increase of antibodies specific for RSV F protein prior to the administration.

20. The method according claim 14, wherein the immune response against RSV comprises eliciting 50% neutralizing geometric mean titer ratios ≥0.79.

21. A composition comprising (i) a first ribonucleic acid (RNA) polynucleotide comprising an open reading frame encoding at least one influenza virus hemagglutinin (HA) polypeptide or an immunogenic fragment thereof, wherein the first RNA polynucleotide is formulated in a lipid nanoparticle (LNP) comprising an ionizable cationic lipid, a polymer-conjugated lipid, a neutral lipid, and a steroid; and (ii) a first RSV F protein trimer in the prefusion conformation.

22. The composition according claim 21, wherein the RNA polynucleotide comprises at least one modified nucleotide selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine OR 2′-O-methyl uridine.

23. The composition according to claim 21, wherein the LNP comprises 20-60% ionizable cationic lipid, 0.5-15% polymer-conjugated lipid, 5-15% neutral lipid, and 25-55% steroid.

24. The composition according to claim 21, wherein the LNP has a mean diameter of 50-200 nm.

25. The composition according to claim 21, wherein the LNP is in a liquid state and the first RSV F protein trimer is lyophilized.

26. The composition according to claim 21, wherein each RNA has at least 50% integrity.

27. The composition according to claim 21, further comprising a sugar selected from the group consisting of lactose, sucrose, mannitol, and sorbitol.


SEQUENCE LISTING (56 sequences)

SEQ ID NO: 1 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - protein, 574 aa, synthetic construct
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 3 - protein, 574 aa, synthetic construct
MELPILKTNAITTILAAVTLCFASSQNITEEFYQSTCSAVSKGYLSALRTGWYTSVITIELSNIKENKCNGTDAKVKLIKQELDKYKNAVTELQLLMQSTPAANSRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLALIAVGLLLYCKARSTPVTLSKDQLSGINNIAFSN

SEQ ID NO: 4 - protein, 574 aa, synthetic construct
MELPILKTNAITTILAAVTLCFASSQNITEEFYQSTCSAVSKGYLSALRTGWYTSVITIELSNIKENKCNGTDAKVKLIKQELDKYKNAVTELQLLMQSTPACNSRARRELPRFMNYTLNNTKNTNVTLSKKRKRRFLGFLLGVGSACASGIAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELLHNVNAGKSTTNIMITTIIIVIIVILLALIAVGLLLYCKARSTPVTLSKDQLSGINNIAFSN

SEQ ID NO: 5 - protein, 574 aa, synthetic construct
MELLIHRSSAIFLTLAINALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 6 - protein, 574 aa, synthetic construct
MELLIHRSSAIFLTLAINALYLTSSQNITEEFYQSTCSAVSRGYFSALRTGWYTSVITIELSNIKETKCNGTDTKVKLIKQELDKYKNAVTELQLLMQNTPACNNRARREAPQYMNYTINTTKNLNVSISKKRKRRFLGFLLGVGSACASGIAVSKVLHLEGEVNKIKNALLSTNKAVVSLSNGVSVLTIKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSSEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 7 - DNA, 1728 bp, synthetic construct
  source 1..1728: mol_type = other DNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgccgccaatagcagagccagacgggaactgcctagattcatgaactacaccctgaacaacaccaagaacaccaacgtgaccctgagcaagaagcggaagcggagattcctgggctttctgctcggagtgggaagcgccattgcctctggaatcgccgtgtctaaagtgctgcacctggaaggcgaagtgaacaagatcaagagcgccctgctgagcaccaacaaggccgtggtgtctctgagcaatggcgtgtccgtgctgaccagcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcgacgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatga

SEQ ID NO: 8 - DNA, 1728 bp, synthetic construct
  source 1..1728: mol_type = other DNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatga

SEQ ID NO: 9 - DNA, 1728 bp, synthetic construct
  source 1..1728: mol_type = other DNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgccgccaacaaccgggccagaagagaagcccctcagtacatgaactacaccatcaacaccaccaagaacctgaacgtgtccatcagcaagaagcggaagcggcggttcctgggctttctgcttggagtgggaagcgccattgccagcggaatcgccgtgtctaaagtgctgcacctggaaggcgaagtgaacaagatcaagaatgccctgctgagcaccaacaaggccgtggtgtctctgagcaatggcgtgtccgtgctgacctccaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcgacgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatga

SEQ ID NO: 10 - DNA, 1728 bp, synthetic construct
  source 1..1728: mol_type = other DNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatga

SEQ ID NO: 11 - RNA, 1728 nt, synthetic construct
  source 1..1728: mol_type = other RNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatga

SEQ ID NO: 12 - RNA, 1728 nt, synthetic construct
  source 1..1728: mol_type = other RNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatga

SEQ ID NO: 13 - RNA, 2156 nt, synthetic construct
  source 1..2156: mol_type = other RNA
agaataaactagtattcttctggtccccacagactcagagagaacccgccaccatggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - RNA, 2156 nt, synthetic construct
  source 1..2156: mol_type = other RNA
agaataaactagtattcttctggtccccacagactcagagagaacccgccaccatggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - RNA, 9487 nt, synthetic construct
  source 1..9487: mol_type = other RNA
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - RNA, 9487 nt, synthetic construct
  source 1..9487: mol_type = other RNA
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - DNA, 47 bp, synthetic construct
  source 1..47: mol_type = other DNA
agaataaactagtattcttctggtccccacagactcagagagaaccc

SEQ ID NO: 18 - RNA, 47 nt, synthetic construct
  source 1..47: mol_type = other RNA
agaataaactagtattcttctggtccccacagactcagagagaaccc

SEQ ID NO: 19 - RNA, 37 nt, synthetic construct
  source 1..37: mol_type = other RNA
agaaaaacagaccggccccacagaccagagagaaccc

SEQ ID NO: 20 - DNA, 295 bp, synthetic construct
  source 1..295: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 21 - RNA, 295 nt, synthetic construct
  source 1..295: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 22 - RNA, 294 nt, synthetic construct
  source 1..294: mol_type = other RNA
ccgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 23 - DNA, 117 bp, synthetic construct
  source 1..117: mol_type = other DNA
atacagcagcaattggcaagctgcttacatagaactcgcggcgattggcatgccgccttaaaatttttattttatttttcttttcttttccgaatcggattttgtttttaatatttc

SEQ ID NO: 24 - RNA, 117 nt, synthetic construct
  source 1..117: mol_type = other RNA
atacagcagcaattggcaagctgcttacatagaactcgcggcgattggcatgccgccttaaaatttttattttatttttcttttcttttccgaatcggattttgtttttaatatttc

SEQ ID NO: 25 - RNA, 117 nt, synthetic construct; identical to SEQ ID NO: 24
  source 1..117: mol_type = other RNA

SEQ ID NO: 26 - RNA, 80 nt, synthetic construct
  source 1..80: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27-44 - skipped (18 entries)

SEQ ID NO: 45 - protein, 27 aa, synthetic construct
GYIPEAPRDGQAYVRKDGEWVLLSTFL

SEQ ID NO: 46 - DNA, 45 bp, synthetic construct
  source 1..45: mol_type = other DNA
gataggcggcgcatgagagaagcccagaccaattacctacccaaa

SEQ ID NO: 47 - RNA, 45 nt, synthetic construct
  source 1..45: mol_type = other RNA
gataggcggcgcatgagagaagcccagaccaattacctacccaaa

SEQ ID NO: 48 - RNA, 45 nt, synthetic construct; identical to SEQ ID NO: 47
  source 1..45: mol_type = other RNA

SEQ ID NO: 49 - RNA, 35 nt, synthetic construct
  source 1..35: mol_type = other RNA
cctgaatggactacgacatagtctagtccgccaag

SEQ ID NO: 50 - RNA, 1605 nt, synthetic construct
  source 1..1605: mol_type = other RNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggcc

SEQ ID NO: 51 - RNA, 2382 nt, synthetic construct
  source 1..2382: mol_type = other RNA
ggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgt

SEQ ID NO: 52 - RNA, 1671 nt, synthetic construct
  source 1..1671: mol_type = other RNA
gcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgca

SEQ ID NO: 53 - RNA, 1824 nt, synthetic construct
  source 1..1824: mol_type = other RNA
tacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaa

SEQ ID NO: 54 - RNA, 52 nt, synthetic construct
  source 1..52: mol_type = other RNA
gaataaactagtattcttctggtccccacagactcagagagaacccgccacc

SEQ ID NO: 55 - RNA, 295 nt, synthetic construct; identical to SEQ ID NO: 21
  source 1..295: mol_type = other RNA

SEQ ID NO: 56 - RNA, 107 nt, synthetic construct
  source 1..107: mol_type = other RNA
aaaaaaaaaaaaaaaaaaaaaaaaaaaaaagcaagacaaaaaaaaaaaaaaaaaaaaaaaaaaaaaaaaaaaaaaaaaaaaaaaaaaaaaaaaaaaaaaaaaaaaaa